SEQUENCE LISTING (96 sequences)

SEQ ID NO: 1 - protein, 93 aa, Homo sapiens
  misc_feature: Incyte ID No 1629602CD1
MVQAGPSSCSISGDPGLPRRWRPAQVVRPGRLRIRGWSRRIPKAEVGSPGDSQLLSLWRRGPVTEAPFSNPGAAFSPYRKSDGLMTSWLAAER

SEQ ID NO: 2 - protein, 281 aa, Homo sapiens
  misc_feature: Incyte ID No 2100360CD1
MILTKAQYDEIAQCLVSVPPTRQSLRKLKQRFPSQSQATLLSIFSQEYQKHIKRTHAKHHTSEAIESYYQRYLNGVVKNGAAPVLLDLANEVDYAPSLMARLILERFLQEHEETPPSKSIINSMLRDPSQIPDGVLANQVYQCIVNDCCYGPLVDCIKHAIGHEHEVLLRDLLLEKNLSFLDEDQLRAKGYDKTPDFILQVPVAVEGHIIHWIESKASFGDECSHHAYLHDQFWSYWNRFGPGLVIYWYGFIQELDCNRERGILLKACFPTNIVTLCHSIA

SEQ ID NO: 3 - protein, 292 aa, Homo sapiens
  misc_feature: Incyte ID No 5166833CD1
MSISLSSLILLPIWINMAQIQQGGPDEKEKTTALKDLLSRIDLDELMKKDEPPLDFPDTLEGFEYAFNEKGQLRHIKTGEPFVFNYREDLHRWNQKRYEALGEIITKYVYELLEKDCNLKKVSIPVDATESEPKSFIFMSEDALTNPQKLMVLIHGSGVVRAGQWARRLIINEDLDSGTQIPFIKRAVAEGYGVIVLNPNENYIEVEKPKIHVQSSSDSSDEPAEKRERKDKVSKETKKRRDFYEKYRNPQKKKEMMQLYIRVSEITTFLYYFLYLVYILLYVDCFVFLQEY

SEQ ID NO: 4 - protein, 270 aa, Homo sapiens
  misc_feature: Incyte ID No 7494963CD1
MEAEELEQEREQLRLQLWEAYCQVRYLCSHLRGNDSADSAVSTDSSMDESSETSSAKDVPAGSLRTALNELKRLIQSIVDGMEPTGSRRLDDDSLEEQIRQTSEDSRALRELMEGERGKLRQSLEELQRLHSQVTLLSVEMTALKEERDRLRVTSEDKEPKEQLQKAIRDRDEAIAKKNAVELELAKCRMDMMSLNSQLLDAIQQKLNLSQQLEAWQDDMHRVIDRQLMDTHLKERSQPAAALCRGHSAGRGDEPSIAEGKRLFSFFRKI

SEQ ID NO: 5 - protein, 447 aa, Homo sapiens
  misc_feature: Incyte ID No 7644881CD1
MQESQETHISNHLDEVVAAVSITHRKKFQNKLLQTALFQPPREKLHLCEEKAKSYSNSHEYKQAVHELVRCVALTRICYGDSHWKLAEAHVNLAQGYLQLKGLSLQAKQHAEKARQILANSIVPPYSENTDVFKFSIELFHTMGRALLSLQKFKEAAENLTKAERLSKELLQCGRIIKEEWIEIEARIRLSFAQVYQGQKKSKEALSHYQAALEYVEISKGETSRECVPILRELAGVEQALGLHDVSINHFLQAHLIILSRSPSQVEAADSAHIVAHAAVASGRHEHHDVAEQYFQESMAHLKDSEGMGRTKFLSIQDEFCHFLQMTGQKERATSILRESLEAKVEAFGDFSPEVAETYRLLGGADLAQGNHSGARKKLKKCLQIQTLLYGPQDKRTLATQQAMGMLSTAPKVASKPRQASKAKVAFCTSIPQDTLLGKARPGTTAD

SEQ ID NO: 6 - protein, 757 aa, Homo sapiens [sequence omitted]

SEQ ID NO: 7 - protein, 1014 aa, Homo sapiens
  misc_feature: Incyte ID No 3846110CD1
MIMQEILTDDIPWKGLYGSVVKKAVVSGNYLEADVRLPKPYYDIVKSGIHVKHKDRTMNLQDIRYILKNDLKDFTGAQRTQPTESPRVQRYGLHPDVNVYLGLTSEHPRETPDMEIIELKEMGSQPHSPRVHSLFTEGTLDPQAPDPCLMARETQNQDAPCPAPFMAEEASSPSTGQPSLCSFEINEIYSGCLILEDDIEEPPGAASSLEADGPNQVDELKSMEEELDKMEREACCFGSEDESSSKAETEYSFDDWDWQNGSLSSLSLPESTREAKSNLNNMSTTEEYLISKCVLDLKIMQTIMHENDDRLRNIEQILDEVEMKQKEQEERMSLWATSREFTNAYKLPLAVGPPSLNYIPPVLQLSGGQKPDTSGNYPTLPRFPRMLPTLCDPGKQNTDEQFQCTQGAKDSLETSRIQNTSSQGRPRESTAQAKATQFNSALFTLSSHRQGPSASPSCHWDSTRMSVEPVSSEIYNAESRNKDDGKVHLKWKMEVKEMAKKAATGQLTVPPWHPQSSLTLESEAENEPDALLQPPIRSPENTDWQRVIEYHRENDEPRGNGKFDKTGNNDCDSDQHGRQPRLGSFTSIRHPSPRQKEQPEHSEAFQASSDTLVAVEKSYSTSSPIEEDFEGIQGAFAQPQVSGEEKFQMRKILGKNAEILPRSQFQPVRSTEDEQEETSKESPKELKEKDISLTDIQDLSSISYEPDSSFKEASCKTPKINHAPTSVSTPLSPGSVSSAASQYKDCLESITFQVKTEFASCWNSQEFIQTLSDDFISVRERAKELDSLLTSSETPPSRLTGLKRLSSFIGAGSPSLVKACDSSPPHATQRRSLPKVEAFSQHHIDELPPPSQELLDDIELLKQQQGSSTVLHENTASDGGGTANDQRHLEEQETDSKKEDSSMLLSKETEDLGEDTERAHSTLDEDLERWLQPPEESVELQDLPKGSERETNIKDQKVGEEKRKREDSITPERRKSEGVLGTSEEDELKSCFWKRLGWSESSRIIVLDQSDLSD

SEQ ID NO: 8 - protein, 342 aa, Homo sapiens
  misc_feature: Incyte ID No 1878279CD1
MMCSRVPSEQSSGTSLLPKDGAPFSWDSLDEDGLDDSLLELSEGEEDDGDVNYTEEEIDALLKEDDPSYEQSSGEDDGGHVEKGERGSQILLDTPREKNSSYSLGPVAETPDLFKLPQLSTSSGHGPAHTKPLNRRSVLEKNLIKVTVAPFNPTVCDALLDKDETDSSKDTEKLSSLGEEMREDGLSPNESKLCTESEGISPNNSAWNGPQLSSSNNNFQQTVSDKNMPDSENPTSVFSRISDHSETPNMELSCRNGGSHKSSCEMRSLVVSTSSNKQDVLNKDSGKMKGHERRLGKVIPVLQTKTRTNVPTFSQSNLEQQKQLYLRSVIAHIEDPEDTNQG

SEQ ID NO: 9 - protein, 415 aa, Homo sapiens
  misc_feature: Incyte ID No 1848891CD1
MRAADSGSWERVRQLAAQGEPAPSCGAGAGPARPPGPAACEQCVDAAGPGDRPRAGVPRVRADGDCSQPVLLREEVSRLQEEVHLLRQMKEMLAKDLEESQGGKSSEVLSATELRVQLAQKEQELARAKEALQAMKADRKRLKGEKTDLVSQMQQLYATLESREEQLRDFIRNYEQHRKESEDAVKALAKEKDLLEREKWELRRQAKEATDHATALRSQLDLKDNRMKELEAELAMAKQSLATLTKDVPKRHSLAMPGETVLNGNQEWVVQADLPLTAAIRQSQQTLYHSHPPHPADRQAVRVSPCHSRQPSVISDASAAEGDRSSTPSDINSPRHRTHSLCNGDSPGPVQKNLHNPIVQSLEDLEDQKRKKKKEKMGFGSISRVFARGKQRKSLDPGLFDGTAPDYYIEEDADW

SEQ ID NO: 10 - protein, 665 aa, Homo sapiens
  misc_feature: Incyte ID No 2500251CD1
MAGLSGAQIPDGEFTALVYRLIRDARYAEAVQLLGRELQRSPRSRAGLSLLGYCYYRLQEFALAAECYEQLGQLHPELEQYRLYQAQALYKACLYPEATRVAFLLLDNPAYHSRVLRLQAAIKYSEGDLPGSRSLVEQLLSGEGGEESGGDNETDGQVNLGCLLYKEGQYEAACSKFSATLQASGYQPDLSYNLALAYYSSRQYASALKHIAEIIERGIRQHPELGVGMTTEGFDVRSVGNTLVLHQTALVEAFNLKAAIEYQLRNYEVAQETLTDMPPRAEEELDPVTLHNQALMNMDARPTEGFEKLQFLLQQNPFPPETFGNLLLLYCKYEYFDLAADVLAENAHLTYKFLTPYLYDFLDALITCQTAPEEAFIKLDGLAGMLTEQLRRLTKQVQEARHNRDDEAIKKAVNEYDETMEKYIPVLMAQAKIYWNLENYPMVEKIFRKSVEFCNDHDVWKLNVAHVLFMQENKYKEAIGFYEPIVKKHYDNILNVSAIVLANLCVSYIMTSQNEEAEELMRKIEKEEEQLSYDDPNRKMYHLCIVNLVIGTLYCAKGNYEFGISRVIKSLEPYNKKLGTDTWYYAKRCFLSLLENMSKHMIVIHDSVIQECVQFLGHCELYGTNIPAVIEQPLEEERMHVGKNTVTDESRQLKALIYEIIGWNK

SEQ ID NO: 11 - protein, 622 aa, Homo sapiens
  misc_feature: Incyte ID No 55026561CD1
MNHFRKMEVINLTTLPMIPVDEHLAVSLVARNTMVKTVRKELENNPPSCLIGSMHQVNQKIADINLRTEPSANSLAIERFELEKKALREKTRSSPEDKVKRQRKSQYSCKGSELRHARSSVIKRKTADKNLLAELYQYSNFNSSKPNKLPNGVDFCDMVGNVVRAERDCLSGKHFCSGRELEKFLSSSSPRAIWLDSFWWIFHERYQPNKELQNNLFDRIAQHYALLLFRVPKSHSEEALLKRLPSLLSKAVYTSFCCCFPQSWFDTHEFKSDICNTMSLWISGTYPSPQSYDSWDYSELDPERFRREELMLYRRRLTKGREFSLFAGKRAFSQKPAQSRKFYHPQSSSANSPSEKTSSAKQNSEKSLRMQNTAKEHHCQTLVLKKPTQEVKRISEARECENMFPKKSCAACKSPELTSNLFNIYGKSPLIVYFLQNYASLQQHGKNVLIVRREKTTSTPDCTPTYTDVISETLCSMKKRKDNLNQLYQHHWTEWNYFDKHLKELQDNFSREMKNIGPKAADTKKANHMFIPPSAVNEESPDKKTKGSLQREIEFKGCSNKNHGKGRSVNMRGKEEREREEKQKLNISFHSLPSPEELHNLEPGSAYRIRDISATRWSGTRK

SEQ ID NO: 12 - protein, 242 aa, Homo sapiens
  misc_feature: Incyte ID No 7502593CD1
MDDDDAKLKAEIEAELDKLSISSLEKEDIESDAKSETQSDDSDTDSVELPESVLHCINIIKNRSKAVEELILQDLEDTDILSCSYGAVSNNHMHLRTGLSTEYEESSEQLIKILSEIEKEEFMRSKTDCATPDFVPEPSPHDLPMDEHVLPDDADINFGYCEVEEKCRQSFEAWQEKQKELEDKEKQTLKAQRDREEKQFQEKKKKRHCWMKQFKVEKKKLENIQKVFCFCFSCIFKISSYL

SEQ ID NO: 13 - protein, 408 aa, Homo sapiens
  misc_feature: Incyte ID No 7503957CD1
MMCSRVPSEQSSGTSLLPKDGAPFSWDSLDEDGLDDSLLELSEGEEDDGDVNYTEEEIDALLKEDDPSYEQSSGEDDGGHVEKGERGSQILLDTPREKNSSYSLGPVAETPDLFKLPQLSTSSGHGPAHTKPLNRRSVLEKNLIKVTVAPFNPTVCDALLDKDETDSSKDTEKLSSLGEEMREDGLSPNESKLCTESEGISPNNSAWNGPQLSSSNNNFQQTVSDKNMPDSENPTSVFSRISDHSETPNMELSCRNGGSHKSSCEMRSLVVSTSSNKQDVLNKDSGKMKGHERRLGKVIPVLQTKTRTNVPTFSQSNLEQQKQLYLRSVIAHIEDPEDTNQGISGELCALMDQVHHMQHSKWQHPSDLTTRNYARRQKHLQRYSLTQWVDRNMRSHHRFQRLPDFSYS

SEQ ID NO: 14 - protein, 820 aa, Homo sapiens
  misc_feature: Incyte ID No 7504415CD1
MTDTRRRVKVYTLNEDRQWDDRGTGHVSSGYVERLKGMSLLVRAESDGSLLLESKINPNTAYQKQQDTLIVWSEAENYDLALSFQEKAGCDEIWEKICQVQGKDPSVDITQDLVDESEEERFDDMSSPGLELPSCELSRLEEIAELVASSLPSPLRREKLALALENEGYIKKLLELFHVCEDLENIEGLHHLYEIIKGIFLLNRTALFEVMFSEECIMDVIGCLEYDPALSQPRKHREFLTKTAKFKEVIPISDPELKQKIHQTYRVQYIQDMVLPTPSVFEENMLSTLHSFIFFNKVEIVGMLQEDEKFLTDLFAQLTDEATDEEKRQELVNFLKEFCAFSQTLQPQNRDAFFKTLSNMGILPALEVILGMDDTQVRSAATDIFSYLVEYNPSMVREFVMQEAQQNDDDILLINLIIEHMICDTDPELGGAVQLMGLLRTLVDPENMLATANKTEKTEFLGFFYKHCMHVLTAPLLANTTEDKPSKDDFQTAQLLALVLELLTFCVEHHTYHIKNYIINKDILRRVLVLMASKHAFLALCALRFKRKIIGLKDEFYNRYIMKSFLFEPVVKAFLNNGSRYNLMNSAIIEMFEFIRVEDIKSLTAHVIENYWKALEDVDYVQTFKGLKLRFEQQRERQDNPKLDSMRSILRNHRYRRDARTLEDEEEMWFNTDEDDMEDGEAVVSPSDKTKNDDDIMDPISKFMERKKLKESEEKEVLLKTNLSGRQSPSFKLSLSSGTKTNLTSQSSTTNLPGSPGSPGSPGSPGSPGSVPKNTSQTAAITTKGGLVGLVDYPDDDEDDDEDEDKEDTLPLSKKAKFDS

SEQ ID NO: 15 - protein, 34 aa, Homo sapiens
  misc_feature: Incyte ID No 7504074CD1
MSVPGPSSPDGALTRPPYCLEAGEPTPDRTKLYC

SEQ ID NO: 16 - protein, 938 aa, Homo sapiens
  misc_feature: Incyte ID No 7502257CD1
MAEKRPLRTLGPVMYGKLPRLETDSGLEHSLPHSVGNQDPCTYKGSYFSCPMAGTPKAESEQLASWTPYPPLYSTGMAGPPLQADNLLTNCLFYRSPAEGPEKMQDSSPVELLPFSPQAHSYPGPPLAAPKPVYRNPLCYGLSTCLGEGAVKRPLDVDWTLATGPLLPSADPPCSLAPAPSKGQTLDGTFLRGVPAEGSSKDSSGSFSPCQPFLEKYQTIHSTGFLASRYTGPYPRNSKQAMSEGPSSPWTQLAQPLGPPCQDTGPTHYPPPHHPPPHPPQALPCPPACRHPEKQGSYSPALPLQPLGGHKGTGYQAGGLGSPYLRQQAAQAPYIPPLGLDAYPYPSAPLPAPSPGLKLEPPLTPRCPLDFAPQTLSFPYARDDLSLYGASPGLGGTPPSQNNVRAVPQPGAFQRACQPLPASQPCSEPVRPAQEAEEKTWLPSCRKEKLQPRLSEHSGPPIVIRDSPVPCTPPALPPCARECQSLPQKEDARPPSSPPMPVIDNVFSLAPYRDYLDVPAPEATTEPDSATAEPDSAPATSEGQDKGCRGTLPAQEGPSGSKPLRGSLKEEVALDLSVRKPTAEASPVKASRSVEHAKPTAAMDVPDVGNMVSDLPGLKKIDTEAPGLPGVPVTTDAMPRTNFHSSVAFMFRKFKILRPAPLPAAVVPSTPTSAPAPTQPAPTPTSGPIGLRILAQQPLSVTCFSLALPSPPAVAVASPAPAPAPSPAPARAQAPASARDPAPAPAPVAGPAPASTSAPGDSLEQHFTGLHASLCDAISGSVAHSPPEKLREWLETAGPWGQAAWQDCQGVQGLLAKLLSQLQRFDRTHRCPFPHVVRAGAIFVPIHLVKERLFPRLPPASVDHVLQEHRVELRPTTLSEERALRELALPGCTSRMLKLLALRQLPDIYPDLLGLQWRDCVRRQLGEHGAAPVATGAV

SEQ ID NO: 17 - protein, 253 aa, Homo sapiens
  misc_feature: Incyte ID No 1315136CD1
MMMGCGESELKSADGEEAAAVPGPPPEPQVPQLRAPVPEPGLDLSLSPRPDSPQPRHGSPGRRKGRAERRGAARQRRQVRFRLTPPSPVRSEPQPAVPQELEMPVLKSSLALGLELRAAAGSHFDAAKAVEEQLRKSFQIRCGLEESVSEGLNVPRSKRLFRDLVSLQVPEEQVLNAALREKLALLPPQARAPHPKEPPGPGPDMTILCDPETLFYESPHLTLDGLPPLRLQLRPRPSEDTFLMHRTLRRWEA

SEQ ID NO: 18 - protein, 723 aa, Homo sapiens
  misc_feature: Incyte ID No 1379785CD1
MAEEEETAALTEKVIRTQRVFINLLDSYSSGNIGKFLSNCVVGASLEEITEEEEEEDENKSAMLEASSTKAKEGTFQIVGTLSKPDSPRPDFAVETYSAISREDLLMRLLECDVIIYNITESSQQMEEAIWAVSALSEEVSHFEKRKLFILLSTVMTWARSKALDPEDSEVPFTEEDYRRRKSHPNFLDHINAEKMVLKFGKKARKFAAYVVAAGLQYGAEGGMLHTFFKMAWLGEIPALPVFGDGTNVIPTIHVLDLAGVIQNVIDHVPKPHYLVAVDESVHTLEDIVKCISKNTGPGKIQKIPRENAYLTKDLTQDCLDHLLVNLRMEALFVKENFNIRWAAQTGFVENINTILKEYKQSRGLMPIKICILGPPAVGKSSIAKELANYYKLHHIQLKDVISEAIAKLEAIVAPNDVGEGEEEVEEEEEEENVEDAQELLDGIKESMEQNAGQLDDQYIIRFMKEKLKSMPCRNQGYILDGFPKTYDQAKDLFNQEDEEEEDDVRGRMFPFDKLIIPEFVCALDASDEFLKERVINLPESIVAGTHYSQDRFLRALSNYRDINIDDETVFNYFDELEIHPIHIDVGKLEDAQNRLAIKQLIKEIGEPRNYGLTDEEKAEEERKAAEERLAREAAEEAEREHQEAVEMAEKIARWEEWNKRLEEVKREERELLEAQSIPLRNYLMTYVMPTLIQGLNECCNVRPEDPVDFLAEYLFKNNPEAQ

SEQ ID NO: 19 - protein, 253 aa, Homo sapiens
  misc_feature: Incyte ID No 2011166CD1
MSFSVHNQKGSKRPLPLEPLLFLQVPRSNYLHFQEEKQRLHLKKFLLDRMFLVAKIQANVERKDVADYYEQMFQSVLKHHLGEAVTGLLLIYPTSILHILESSSDTLYKVLLDYIGHVKDETVFFIQQMKIIVISHNIPMRLFMQWHVSVIKVPVMYLDDVTQSQSLKEVITDFLTQTHKLSLYLCQTMKVGTKGPGDNLHQVAPDLLLPEQIIKYLCKSEEFMDPATFINMYNRPIHITLDSEVVWPAPSRF

SEQ ID NO: 20 - protein, 154 aa, Homo sapiens
  misc_feature: Incyte ID No 3434684CD1
MSVGNSVNNSPAAPQSDFQLLPAQGSSLTNFFPDVGFDQQSMRPGPAFPQQVPLVQQGSRELQDSFHLRPSPYSNCGSLPNTILPEDSSTSLFKDLNSALAGLPEVSLNVDTPFPLEEELQIEPLSLDGLNMLSDSSMGLLDPSVEETFRADRL

SEQ ID NO: 21 - protein, 566 aa, Homo sapiens
  misc_feature: Incyte ID No 5134056CD1
MRRQWGSAMRAAEQAGCMVSASRAGQPEAGPWSCSGVILSRSPGLVLCHGGIFVPFLRAGSEVLTAAGAVFLPGDSCRDDLRLHVQWAPTAAGPGGGAERGRPGLCTPQCASLEPGPPAPSRGRPLQPRLPAELLLLLSCPAFWAHFARLFGDEAAEQWRFSSAARDDEVSEDGEADQLRALGWFALLGVRLGQEEVEEERGPAMAVSPLGAVPKGAPLLVCGSPFGAFCPDIFLNTLSCGVLSNVAGPLLLTDARCLPGTEGGGVFTARPAGALVALVVAPLCWKAGEWVGFTLLCAAAPLFRAARDALHRLPHSTAALAALLPPEVGVPWGLPLRDSGPLWAAAAVLVECGTVWGSGVAVAPRLVVTCRHVSPREAARVLVRSTTPKSVAIWGRVVFATQETCPYDIAVVSLEEDLDDVPIPVPAEHFHEGEAVSVVGFGVFGQSCGPSVTSGILSAVVQVNGTPVMLQTTCAVHSGSSGGPLFSNHSGNLLGIITSNTRDNNTGATYPHLNFSIPITVLQPALQQYSQTQDLGGLRELDRAAEPVRVVWRLQRPLAEAPRSKL

SEQ ID NO: 22 - protein, 234 aa, Homo sapiens
  misc_feature: Incyte ID No 5281724CD1
MEPSKTFMRNLPITPGYSGFVPFLSCQGMSKEDDMNHCVKTFQEKTQRYKEQLRELCCAVATAPKLKPVNSEETVLQALHQYNLQYHPLILECKYVKKPLQEPPIPGWAGYLPRAKVTEFGCGTRYTVMAKNCYKDFLEITERAKKAHLKPYEEIYGVSSTKTSAPSPKVLQHEELLPKYPDFSIPDGSCPALGRPLREDPKTPLTCGCAQRPSIPCSGKMYLEPLSSAKYAEG

SEQ ID NO: 23 - protein, 268 aa, Homo sapiens
  misc_feature: Incyte ID No 7502391CD1
MFVELNNLLNTTPDRAEQGKLTLLCDAKTDGSFLVHHFLSFYLKANCKVCFVALIQSFSHYSIVGQKLGVSLTMARERGQLVFLEGLKSAVDVVFQAQKEPHPLQFLREANAGNLKPLFEFVREALKPVDSGEARWTYPVLLVDDLSVLLSLGMGAVAVLDFIHYCRATVCWELKGNMVVLVHDSGDAEDEENDILLNGLSHQSHLILRAEGLATGFCRDVHGQLRILWRRPSQPAVHRDQSFTYQYKIQDKRRVLFCQRNVSCCSVT

SEQ ID NO: 24 - protein, 694 aa, Homo sapiens
  misc_feature: Incyte ID No 7502544CD1
MATKTAGVGRWEVVKKGRRPGVGAGAGGRGGGRNRRALGEANGVWKYDLTPAIQTTSTLYERGFENIMKRQNKEQVPPPAVEPKKPGNKKQPKKVATPPNQNQKQGRFRSLEEALKALDVADLQKELDKSQSVFSGNPSIWLKDLASYLNYKLQAPLSEPTLSQHTHGLWALDYPYSLVSRELRGIIRGLLAKAAGSLELFFDHCLFTMLQELDKTPGESLHGYRICIQAILQDKPKIATANLGKFLELLRSHQSRPAKCLTIMWALGQAGFANLTEGLKVWLGIMLPVLGIKSLSPFAITYLDRLLLMHPNLTKGFGMIGPKDFFPLLDFAYMPNNSLTPSLQEQLCQLYPRLKMLAFGAKPDSTLHTYFPSFLSRATPSCPPEMKKELLSSLTECLTVDPLSASVWRQLYPKHLSQSSLLLEHLLSSWEQIPKKVQKSLQETIQSLKLTNQELLRKGSSNNQDVVTCDMACKGLLQQVQGPRLPWTRLLLLLLVFAVGFLCHDLRSHSSFQASLTGRLLRSSGFLPASQQACAKLYSYSLQGYSWLGETLPLWGSHLLTVVRPSLQLAWAHTNATVSFLSAHCASHLAWFGDSLTSLSQRLQIQLPDSVNQLLRYLRELPLLFHQNVLLPLWHLLLEALAWAQEHCHEACRGEVTWDCMKTQLSEAVHWTWLCLQDITVAFLDWALALISQQ

SEQ ID NO: 25 - protein, 519 aa, Homo sapiens
  misc_feature: Incyte ID No 2858465CD1
MAPIPKTVGRIKLDCSLRPSCPLEVAAAPKLCKEFGPEDYGEEDIVDFLRRLVESDPQGLHRIHVDGSSGRLQLWHHDYLLGHLDDEGKSTGQSDRGKGAEGLGTYCGLRKSFLYPPQESEPCPQSPSASATFPSVSDSLLQVAMPQKLLVTEEEANRLAEELVAEEERMKQKAEKKRLKKKRQKERKRQERLEQYCGEPKASTTSDGDESPPSSPGNPVQGQCGEEEDSLDLSSTFVSLALRKVGDWPLSARREKGLNQEPQGRGLALQKMGQEEESPPREERPQQSPKVQASPGLLAAALQQSQELAKLGTSFAQNGFYHEAVVLFTQALKLNPQDHRLFGNRSFCHERLGQPAWALADAQVALTLRPGWPRGLFRLGKALMGLQRFREAAAVFQETLRGGSQPDAARELRSCLLHLTLQGQRGGICAPPLSPGALQPLPHAELAPSGLPSLRCPRSTALRSPGLSPLLHYPSCHRSHPNQPLSRTQSRRPHPLKPQDPSKGWDILGLGLQHLSQAR

SEQ ID NO: 26 - protein, 216 aa, Homo sapiens
  misc_feature: Incyte ID No 7503455CD1
MALNKNHSEGGGVIVNNTESILMSYDHVELTFNDMKNVPEAFKGTKKGTVYLTPYRVIFLSKGKDAMQSFMMPFYLMKDCEIKQPVFGANYIKGTVKAEAGGGWEGSASYKLTFTAGGAIEFGQRMLQVASQEFYPGPPMMDGAMGYVQPPPPPYPGPMEPPVSGPDVPSTPAAEAKAAEAAASAYYNPGNPHNVYMPTSQPPPPPYYPPEDKKTQ

SEQ ID NO: 27 - protein, 110 aa, Homo sapiens
  misc_feature: Incyte ID No 7503479CD1
MAVCIAVIAKENYPLYIRSTPTENELKFHYMVHTSLDVVDEKISAMGKALVDQRELYLGLLYPTEDYKMFRKLHNSYTDVMCNPFYNPGDRIQSSRAFDNMVTSMMIQVC

SEQ ID NO: 28 - protein, 642 aa, Homo sapiens
  misc_feature: Incyte ID No 7218127CD1
MGVDSRTSCSPQKAQEANKARPSAWEPAAGNSPARASVPAAPNPAATSATSVHVRSPARPSESRLAPTPTEGKVRPRVTNSSPMGWSSAAPCTAAAASHPAVPPSAPDPRPATPQGGGAPRVAAPQTTLSSSSTSAATVDPPAWTPSASRTQQARNKFFQTSAVPPGTSLSGRGPTPSLVLSKDSSKEQARNFLKQALSALEEAGAPAPGRPSPATAAVPSSQPKTEAPQASPLAKPLQSSSPRVLGLPSRMEPPAPLSTSSTSQASALPPAGRRNLAESSGVGRVGAGSRPKPEAPMAKGKSTTLTQDMSTSLQEGQEDGPAGWRANLKPVDRRSPAERTLKPKEPRALAEPRAGEAPRKVSGSFAGSVHITLTPVRPDRTPRPASPGPSLPARSPSPPRRRRLAVPASLDVCDNWLRPEPPGQEARVQSWKEEEKKPHLQGKPGRPLSPANVPALPGETVTSPVRLHPDYLSPEEIQRQLQDIERRLDALELRGVELEKRLRAAEGDDAEDSLMVDWFWLIHEKQLLLRQESELMYKSKAQRLEEQQLDIEGELRRLMAKPEALKSLQERRREQELLEQYVSTVNDRSDIVDSLDEDRLREQEEDQMLRDMIEKLGLQRKKSKFRLSKIWSPKSKSSPSQ

SEQ ID NO: 29 - protein, 489 aa, Homo sapiens
  misc_feature: Incyte ID No 1688943CD1
MNKLSPVLLFLNQQNYQIDKDVEDKRQKAIEEFFTKDVIVPSPWTDHEGKQLSQCHSSKCTNINSDSPVGKKLTIHSEKSDAACQTLLSLPVDFNLENILGDYFRADEFADQSPGNLSSSSLRRKLFLDGNGSISDSLPSASPGSPHSGVQTSLEMFYSIDLSPVKCRSPLQTPSSGQFSSSPIQASAKKYSLGSITSPSPISSPTFSPIEFQIGETPLSEQRKFTVHSPDASSGTNSNGITNPCIRSPYIDGCSPIKNWSPMRLQMYSGGTQYRTSVIQIPFTLETQGEDEEDKENIPSTDVSSPAMDAAGIHLRQFSNEASTHGTHLVVTAMSVTQNQSSASEKELALLQDVEREKDNNTVDMVDPIEIADETTWIKEPVDNGSLPMTDFVSGIAFSIENSHMCMSPLAESSVIPCESSNIQMDSGYNTQNCGSNIMDTVGAESYCKESDAQTCEVESKSQAFNMKQDHTTQRCWMKTASPFQCSSP

SEQ ID NO: 30 - protein, 184 aa, Homo sapiens
  misc_feature: Incyte ID No 2369350CD1
MSNERGFENVELGVIGKKKKVPRRVIHFVSGETMEEYSTDEDEVDGLEKKDVLPTVDPTKLTWGPYLWFYMLRAATSTLSVYDFLGEKIASVLGISTPKYQYAIDEYYRMKKEEEEEEEENRMSEEAEKQYQQNKLQTDSIVQTDQPETVISSSFVNVNFEMEGDSEVIMESKQIQSLSHHKMK

SEQ ID NO: 31 - protein, 520 aa, Homo sapiens
  misc_feature: Incyte ID No 2722979CD1
MLQITEWRFLARDEGESAVAEDPTWGEDEEPSACTTDSWAQGSVPVLHASTSEGLENFQGEVHSSGASPDSSAIAPALPFPTSHCPSAFPQDPGGVDRIPLGRSWMGRGSQEQMESWEPSPQLRVTSAPPPTSELFQEAGPGGPVEEADGQSRGLSSAGSLSASFQLSVEEAPADDADPSLDPYLVASPQASTGRGHPLGFHLSLEDLYCCMPQLDAAGDRLELRSEGVPCIASGVLVSYPSVGGATRPSASCQQQRAGHSDVRLSAHHHRMRRKAAVKRLDPARLPCHWVRPLAEVLVPDSQTRPLEAYRGRQRGEKTKARAEPQALGPGTRVSPAAFFPLRPGIPFRDLDSGPALLFPTLNLGLSSPSLESKLPLPNSRIRFLTTHPVLPDVARSRSPKLWPSVRWPSGWEGKAELLGELWAGRTRVPPQGLELADREGQDPGRWPRTTPPVLEATSQVMWKPVLLPEALKLAPGVSMWNRSTQVLLSSGVPEQEDKEGSTFPPVEQHPIQTGAPKPR

SEQ ID NO: 32 - protein, 255 aa, Homo sapiens
  misc_feature: Incyte ID No 60140470CD1
MASSDLEQLCSHVNEKIGNIKKTLSLRNCGQEPTLKTVLNKIGDEIIVINELLNKLELEIQYQEQTNNSLKELCESLEEDYKDIEHLKENVPSHLPQVTVTQSCVKGSDLDPEEPIKVEEPEPVKKPPKEQRSIKEMPFITCDEFNGVPSYMKSRLTYNQINDVIKEINKAVISKYKILHQPKKSMNSVTRNLYHRFIDEETKDTKGRYFIVEADIKEFTTLKADKKFHVLLNILRHCRRLSEVRGGGLTRYVIT

SEQ ID NO: 33 - protein, 231 aa, Homo sapiens [sequence omitted]

SEQ ID NO: 34 - protein, 492 aa, Homo sapiens
  misc_feature: Incyte ID No 7161479CD1
MLPARWFCYYNSHPKYRRCSVPEEQELTDEDLCLSKAKKQEQTVEEKKKMPMENENHQVFSNPPKILTVQEMAGLNNKTIGYEGIHSPSVLPSGGEESRSPSLQLKPLDSSVLQFSSKYKWILGEEPVEKRRRLQNEMTTPSLDYSMPAPYRRVEAPVAYPEGENSHDKSSSERSTPPYLFPEYPEASKNTGQNREVSILYPGAKDQRQGSLLPEELEDQMPRLVAEESNRGSTTINKEEVNKGPFVAVVGVAKGVRDSGAPIQLIPFNREELAERRKAVESWNPVPYSVASAAIPAAAIGEKARGYEESEGHNTPKLKNQRELEELKRTTEKLERVLAERNLFQQKVEELEQERNHWQSEFKKVQHELVIYSTQEAEGLYWSKKHMGYRQAEFQILKAELERTKEEKQELKEKLKETETHLEMLQKAQVSYRTPEGDDLERALAKLTRLRIHVSYLLTSVLPHLELREIGYDSEQVDGILYTVLEANHILD

SEQ ID NO: 35 - protein, 85 aa, Homo sapiens [sequence omitted]

SEQ ID NO: 36 - protein, 178 aa, Homo sapiens
  misc_feature: Incyte ID No 7502390CD1
MGETWKNICSTVRHGWWLRDHRMAGLPIPPEIVKEAEVPQAALGVPAQGTGDNGHTPVEEEVGGIPVPAPGLLQVTERRQPLSSVSSLEVHFDLLDLTELTDMSDQELAEVFADSDDENLNTESPAGLHPLPRAGYLRSPSWTRTRAEQSHEKQPLGDPERQATVLDTFLTVERPQED

SEQ ID NO: 37 - protein, 665 aa, Homo sapiens
  misc_feature: Incyte ID No 7502872CD1
MGDILAHESELLGLVKEYLDFAEFEDTLKTFSKECKIKGKPLCKTVGGSFRDSKSLTIQKDLVAAFDNGDQKVFFDLWEEHISSSIRDGDSFAQKLEFYLHIHFAIYLLKYSVGRPDKEELDEKISYFKTYLETKGAALSQTTEFLPFYALPFVPNPMVHPSFKELFQDSWTPELKLKLEKFLALISKASNTPKLLTIYKENGQSNKEILQQLHQQLVEAERRSVTYLKRYNKIQADYHNLIGVTAELVDSLEATVSGKMITPEYLQSVCVRLFSNQMRQSLAHSVDFTRPGTASTMLRASLAPVKLKDVPLLPSLDYEKLKKDLILGSDRLKAFLLQALRWRLTTSHPGEQRETVLQAYISNDLLDCYSHNQRSVLQLLHSTSDVVRQYMARLINAFASLAEGRLYLAQNTKVLQMLEGRLKEEDKDIITRENVLGALQKFSLRRPLQTAMIQDGLIFWLVDVLKDPDCLSDYTLEYSVALLMNLCLRSTGKNMCAKVAGLVLKVLSDLLGHENHEIQPYVNGALYSILSVPSIREEARAMGMEDILRCFIKEGNAEMIRQIEFIIKQLNSEELPDGVLESDDDEDEDDEEDHDIMEADLDKDELIQPQLGELSGEKLLTTEYLGIMTNTGKTRRKGLANVQWSGDEPLQRPVTPGGHRNGYPV

SEQ ID NO: 38 - protein, 551 aa, Homo sapiens
  misc_feature: Incyte ID No 7505443CD1
MARAGPRLVLSEEAVRAKSGLGPHRDLAELQSLSIPGTYQEKITHLGHSLMSLTGLKSLDLSRNSLVSLEGIQYLTALESLNLYYNCISSLAEVFRLHALTELVDVDFRLNPVVKVEPDYRLFVVHLLPKLQQLESRHLLSPQLVQYQCGDSGKQGRETRRSSCRGCCLEKMPWSQLCGELPPLYGAEPEASRAPRPHTYFTPHPDSMDTEDSASSQKLDLSGEMVPGPLPAPGKCRKRRMPVGRFQTFSDQEGLGCPERTHGSSVPKESLSRQDSSESRNGRTLSQPEASETEEQRSRGVTDTREPSPGSHSALPGKKTALQAALLETLLDLVDRSWGGCRSLHSNEAFLAQARHILSSVEEFTAAQDSSAMVGEDVGSLALESKSLQSRLAEQQQQHAREMSEVTAELHHAHKELDDLRQHLDKSLEENSRLKSLWLSMKKEVKSADTAATLNLQIAGLQTSVKRLCGEIVELKQHLEHYDKIQELTQMLQESHSSLVSTNEHLLQELSQVRAQHRAEVEQMHWSYQELKKTMALFPHSSASHGGCQAC

SEQ ID NO: 39 - protein, 148 aa, Homo sapiens
  misc_feature: Incyte ID No 8032443CD1
MGPEEKTIMTERSAAVFIQAWWRGMLVRRTLLHAALRAWIIQCWWRQVLEKLLAKRRRMVLEFYVQQEWAAVRLQSWVRMWCVRQRYCRLLNAVRIIQVYWRWHSCHSRVFIEGHYELKENQLNIQLEISLGLQACKVQQCIPLPLKE

SEQ ID NO: 40 - protein, 342 aa, Homo sapiens
  misc_feature: Incyte ID No 7704916CD1
MKTVKEKKECQRLRKSAKTRRVTQRKPSSGPVCWLCLREPGDPEKLGEFLQKDNISVHYFCLILSSKLPQRGQSNRGFHGFLPEDIKKEAARASRKICFVCKKKGAAINCQKDQCLRNFHLPCGQERGCLSQFFGEYKSFCDKHRPTQNIQHGHVGEESCILCCEDLSQQSVENIQSPCCSQAIYHRKCIQKYAHTSAKHFFKCPQCNNRKEFPQEMLRMGIHIPDRRWCLILCATCGSHGTHRDCSSLRSNSKKWECEECSPAAATDYIPENSGDIPCCSSTFHPEEHFCRDNTLEENPGLSWTDWPEPSLLEKPESSRGRRSYSWRSKGVRITNSCKKSK

SEQ ID NO: 41 - protein, 194 aa, Homo sapiens
  misc_feature: Incyte ID No 2013440CD1
MFLTAVNPQPLSTPSWQIETKYSTKVLTGNWMEERRKFTRDTDKTPQSIYRKEYIPFPDHRPDQISRWYGKRKVEGLPYKHLITHHQEPPHRYLISTYDDHYNRHGYNPGLPPLRTWNGQKLLWLPEKSDFPLLAPPTNYGLYEQLKQRQLTPKAGLKQSTYTSSYPRPPLCAMSWREHAVPVPPHRLHPLPHF

SEQ ID NO: 42 - protein, 126 aa, Homo sapiens
  misc_feature: Incyte ID No 2503512CD1
MGGQAGLRTGRTKRGEERPHTCSDIKSILLHRYFRCQGLQAGSPQLLPGTSPTGDCRALGWVTPPAPRKTSSLATPRPLSSKQSARSSSGSPRNRAPCRTSTADRPRLADLPSIRFLWKQDQKEIN

SEQ ID NO: 43 - protein, 474 aa, Homo sapiens
  misc_feature: Incyte ID No 277396CD1
MKTKCICELCSCGRHHCPHLPTRIYDETEKPCLLSEYTENYPFYHSYLPRESFKPRREYQKGSIPMEGLTTSRRDFGPHKVAPVKVHQYDQFVPSEENMDLLTTYKKDYNPYPVCRVDPIKPRDSKYPCSDKMECLPTYKADYLPWNQPRREPLRLEHKYQPASVRFDNRTTHQDDYPIKGLVKTISCKPLAMPKLCNIPLEDVTNYKMSYVAHPVEKRFVHEAEKFRPCEIPFESLTTQKQSYRGLMGEPAKSLKPLARPPGLDMPFCNTTEFRDKYQAWPMPRMFSKAPITYVPPEDRMDLLTTVQAHYTCPKGAPAQSCRPALQIKKCGRFEGSSTTKDDYKQWSSMRTEPVKPVPQLDLPTEPLDCLTTTRAHYVPHLPINTKSCKPHWSGPRGNVPVESQTTYTISFTPKEMGRCLASYPEPPGYTFEEVDALGHRIYKPVSQAGSQQSSHLSVDDSENPNQRELEVLA

SEQ ID NO: 44 - protein, 341 aa, Homo sapiens
  misc_feature: Incyte ID No 3044046CD1
MSVLDALWEDRDVRFDLSAQQMKTRPGEVLIDCLDSIEDTKGNNGDRGRLLVTNLRILWHSLALSRVNVSVGYNCILNITTRTANSKLRGQTEALYILTKCNSTRFEFIFTNLVPGSPRLFTSVMAVHRAYETSKMYRDFKLRSALIQNKQLRLLPQEHVYDKINGVWNLSSDQGNLGTFFITNVRIVWHANMNDSFNVSIPYLQIRSIKIRDSKFGLALVIESSQQSGGYVLGFKIDPVEKLQESVKEINSLHKVYSASPIFGVDYEMEEKPQPLEALTVEQIQDDVEIDSDGHTDAFVAYFADGNKQQDREPVFSEELGLAIEKLKDGFTLQGLWEVMS

SEQ ID NO: 45 - protein, 287 aa, Homo sapiens
  misc_feature: Incyte ID No 3808420CD1
MDMHSARLDSFLSQLRWELLCGRDTGSPSMPGPLQPTSQTGPDVQPSHQLRASGALEEDSVCCVEEEEEEEEEAVVTEDRDAALGGPREHALDWDSGFSEVSGSTWREEELPVSQRPAPSAQPLRRQCLSVSGLPMPSRAPVASVPPVHHPRPKSTPDACLEHWQGLEAEDWTAALLNRGRSRQPLVLGDNCFADLVHNWMELPETGSEGGDGGGHRARARPPQFLLGLSEQLRRRLARARRTAMAGKRLSCPPRPEPELPADVSRFAALMSCRSRQPIICNDVSYL

SEQ ID NO: 46 - protein, 644 aa, Homo sapiens
  misc_feature: Incyte ID No 7504028CD1
MNLLPKSSREFGSVDYWEKFFQQRGKKAFEWYGTYLELCGVLHKYIKPREKVLVIGCGNSELSEQLYDVGYRDIVNIDISEVVIKQMKECNATRRPQMSFLKMDVTQMEFPDASFQVVLDKGTLDAVLTDEEEKTLQQVDRMLAEVGRVLQVGGRYLCISLAQAHILKKAVGHFSREGWMVRVHQVANSQDQVLEAEPQFSLPVFAFIMTKFRPVPGSALQIFELCAQEQRKPVRLESAERLAEAVQERQQYAWLCSQLRRKARLGSVSLDLCDGDTGEPRYTLHVVDSPTVKPSRDNHFAIFIIPQGRETEWLFGMDEGRKQLAASAGFRRLITVALHRGQQYESMDHIQAELSARVMELAPAGMPTQQQVPFLSVGGDIGVRTVQHQDCSPLSGDYVIEDVQGDDKRYFRRLIFLSNRNVVQSEARLLKDVSHKEIPLALLVVGLGGGSLPLFVHDHFPKSCIDAVEIDPSMLEVATQWFGFSQSDRMKVHIADGLDYIASLAGGGEARPCYDVIMFDVDSKDPTLGMSCPPPAFVEQSFLQKVKSILTPEGVFILNLVCRDLGLKDSVLAGLKAVFPLLYVRRIEGEVNEILFCQLHPEQKLATPELLETAQALERTLRKPGRGWDDTYVLSDMLKTVKIV

SEQ ID NO: 47 - protein, 914 aa, Homo sapiens
  misc_feature: Incyte ID No 7766880CD1
MIQRISCFSWIDVFPRGRLLSDERNILSNVDDILAATAAACGVTPTDFSKSTSNETMQAVEDGDSKSHFQQSLDVRHVTSDFNSMTATVGKPQNINDTSLNGNQVTVNLSPVPALQSKMTLDQQHIETPGQNIPTKVTSAVVGPSHEVQEQSSGPFKKQSATNLESEEDSEAPVDSTLNNNRNQEFVSSSRSISGESATSESEFTLGGDDSGVSMNPARSALALLAMAQSGDAVSVKIEEENQDLMHFNLQKKRAKGKGQVKEEDNSNQKQLKRPAQGKRQNPRGTDIYLPYTPPSSESCHDGYQHQEKMRQKIKEVEEKQPEVKTGFIASFLDFLKSGPKQQFSTLAVRMPNRTRRPGTQMVRTFCPPPLPKPSSTTPTPLVSETGGNSPSDKVDNELKNLEHLSSFSSDEDDPGYSQDAYKSVSTPLTTLDATSDKKKKTEALQVATTSPTANTTGTATTSSTTVGAVKQEPLHSTSYAVNILENISSSESSKPIELDGLPSDQFAKGQDTVAIEGFTDEEDTESGGEGQYRERDEFVVKIEDIETFKEALKTGKEPPAIWKVQKALLQKFVPEIRDGQREFAATNSYLGYFGDAKSKYKRIYVKFIENANKKEYVRVCSKKPRNKPSQTIRTVQAKPSSSSKTSDPLASKTTTTKAPSVKPKVKQPKVKAEPPPKKRKKWKEEFSSSQSDSSPEIHTSSSDDEEFEPPAPFVTRFLNTRAMKETFKSYMELLVSIALDPDTMQALEKSNDELLLPHMKKIDGMLNDNRKRLLLNLHLDQSFKNALESFPELTIITRDSKAKSGGTAISKIKMNGKAYNKKTLRTSKTTTKSAQEFAVDPEKIQLYSLYHSLHHYKYHVYLICKDEISSVQKKNEDLGQEEIVQLCMKNVKWVEDLFEKFGELLNHVQQKCS

SEQ ID NO: 48 - protein, 148 aa, Homo sapiens
  misc_feature: Incyte ID No 90089609CD1
MHGARLSDKLWRVMELWLGRAGRLGRKGLRGRRAGRAGHTGLHGGAAHLRHPGVPAAVAFLHLPQDISVQQTLDLVPVLEGLAVSTHPALQLRRLACGGWQTRPQAAAPPLWDFSQGRRADGVSVTASYRALGGGPCSRSRPLPRPHT

SEQ ID NO: 49 - DNA, 882 bp, Homo sapiens
  misc_feature: Incyte ID No 1629602CB1
cagcggccggccgcggggcctttttgtcctgagggccagagaaagggagaagggggtggggggacagccacgtggccgcaggaggatttacaacattttctttcgccatcgatgttatcgcaaaatgtgtgagagaagcggctgcgcagcccggacgggagcgtgagggtgcgggccaggtaagcagccccggcggtttcgccgcatacgggactgcggggcgaccgcgggcaccagccacgcgcagcggctccgcggggtctcggccgggtccgcgctctgaaggatctcgagagccatggatggtgcaggcgggaccctcgagctgcagcatctccggtgacccggggttgccgaggaggtggagaccagcacaggtggtccggcccgggcgcctccgaatccgggggtggtcaagacggatccccaaggctgaggtcggcagtcccggggactcgcagctgttgagcctgtggagacgcggccccgtgaccgaggcacccttcagcaacccgggggcagcgttttccccctaccggaaatctgatgggcttatgacatcatggctggctgctgagcgatgaagtggatgccacaaagaaatccgacatatcagatagattctgaaatcggtttccctccagctgtagtaacaggcgtgaagtcaggagaatttgagctttgtttaaaaaataaataaataaataaataaaccataacaaagtcttgccctgtattaaatgcaattttcttaaaaacaagcaaaccttttggacatcattttattttaatagaaatgctgagttttatgaaactaaagtggctaataaatcagacctgaagctttgtgtgagtgttccaaaaaaaaaaaaaaaaaaaatg

SEQ ID NO: 50 - DNA, 2489 bp, Homo sapiens
  misc_feature: Incyte ID No 2100360CB1
gggcactctggtgtacagccagtccccgccgcggaggtgccggtggagcctgggaccgggcgagtctccgccccgcttttgcagctaggggtgtgtttcaggggggattggggcaagccaagcaggcgaggacctgggcctgtgccgctttgcctacccctcatccctcggcaccaaggctacttgagccccagggtgttttttccttgttcccgccacctcctggtccctggcccaacatgatactgaccaaagctcagtacgacgagatagcccagtgcctagtgtctgtgccgcctaccaggcagagcctgaggaagctgaagcagaggtttcccagtcaatcgcaggccactctgctgagcatcttctcccaggagtaccagaaacacattaaaagaacacatgccaaacatcatacttcggaagcaattgaaagttattaccagaggtacctgaatggagtggtgaaaaatggagctgccccagtgctcctggacctggccaatgaggtggactatgcgccctcattaatggctcggcttatactggagaggtttctacaggaacacgaggaaactccaccctccaagtctattataaatagtatgctacgggacccttctcagattccagatggagttctagcaaatcaggtctatcagtgcattgtgaacgactgctgttacggaccactagtggactgcatcaagcatgccattggtcatgagcatgaggtcctgctgagagacttgcttctagagaaaaacctgtccttcctagatgaagatcagcttcgtgcaaagggttatgacaaaacaccagacttcattttacaagtaccagttgctgtagaagggcacataattcactggattgaaagcaaagcctcatttggtgatgaatgtagccaccacgcctacctgcatgaccagttctggagctactggaatagatttgggccaggcttagtcatctattggtatggatttatccaggagctggactgcaaccgggaaaggggcatcctgctcaaagcctgtttccccacgaacattgtcaccttatgccacagcatagcttgaccctgaagatcctggaagagaagctgggaggaaaagagacccagcattgcattaccatcgtggaataatctagcgcaaacctaggaaagctgaagccacaaagtccaaagccacctttgtactcacctgcagagctccagaagaccttgatggcagcctgcctatgctgtgtgtttgctatattcaatctttacggcttcctgacttctgtgacagtaagccaagtgcaaaaatacacttgatgagaatttcctcttttaataatgttatttgaacaccacatattttagatttatcttatttgaaagtattagttccattgtgcctggaaaccacactcctttagattgggggccgagaggcgacaacccaacattgaggagagtttatttttaaacatggctagttgtcagtatgtacgtgagctagtatttttatgagtcgagttttttaaaggcacattctgtatactgcttagtatatgcattttataccatgtaattataaaacactcgagtaagttcagcattagaaatgtttagctttgtatgaactgagtgtgccagaaataaacctggagcaatttttaaataagcaaaataaaggagatttttctatttgttcactttaatttattcacttttgtgtacttttatgtactgcaaatcagatttcagtctaaagcgaaacatcagtaagttaataataaacctatctttcgggaagttgaatattaatctgtacccaaaacgtatttagtaaaatatttgcccccgccaccctgccatgctgacataacaacttttataatgttgaatagatgatatgggaaatactaataacaacaatgtaatttttgcagacagctttaacttatatacattgcttgatttttttcaaaagactaaatatgtcatttatactttgtttattttctaccaaagaaggtttgtaaaaatatgcctgctgcttttccttttgaaggacacaaacctggtcccaacatgtgtggattttaactctgagtggggtgcattaaatcaaaagagagaggcagaagatgaaatgctaaagaagggtcaggcaaacttctgtttcagtataaaattcatcatgcaggcttctgagtgaaatagaatgatttgaaaccactactgtattgcctggatacacacacacacacacacacactttatacaaaaatgttaaaagcaggtttcctggcatgttctaaactgttttttctttaggaataaattacatttatctgcacagatgttgaaaatcctgttaaacccttgtcaaggatttgtttattttacattaaacaaatttattatgatgaacgtgaacaaataaattaaaaaataaaaaaggtaaaaaaaaaaaaaaaaaaaaattcgg

SEQ ID NO: 51 - DNA, 1115 bp, Homo sapiens
  misc_feature: Incyte ID No 5166833CB1
ctcgaacttggtcggggcgcggatcccgagagggaaagtcataacaaccgcacgagggagttcgactggcgaactggaaggccacgcctcctcccgcctgccccctcagccctgtggctgggggcagagctcagactgtcttctgaagattgatgtctatttccttgagctctttaattttgttgccaatttggataaacatggcacaaatccagcagggaggtccagatgaaaaagaaaagactaccgcactgaaagatttattatctaggatagatttggatgaactaatgaaaaaagatgaaccgcctcttgattttcctgataccctggaaggatttgaatatgcttttaatgaaaagggacagttaagacacataaaaactggggaaccatttgtttttaactaccgggaagatttacacagatggaaccagaaaagatacgaggctctaggagagatcatcacgaagtatgtatatgagctcctggaaaaggattgtaatttgaaaaaagtatctattccagtagatgccactgagagtgaaccaaagagttttatctttatgagtgaggatgctttgacaaatccacagaaactgatggttttaattcatggtagtggtgttgtcagggcagggcagtgggctagaagacttattataaatgaagatctggacagtggcacacagataccgtttattaaaagagctgtggctgaaggatatggagtaatagtactaaatcccaatgaaaactatattgaagtagaaaagccgaagatacacgtacagtcatcatctgatagttcagatgaaccagcagaaaaacgggaaagaaaagataaagtttctaaagaaacaaagaagcgacgtgatttctatgagaagtatcgtaacccccaaaaaaaaaaagaaatgatgcaattgtatatcagagtgagtgagatcactactttcctttactattttctttaccttgtatatattttattatatgtagattgttttgtttttcttcaagaatattaatttctttatttgtcatcatttatttcccatggtcgtctacttggattaaatgggtttttaaattcaaaaaaaaaaaaaaaaaaa

SEQ ID NO: 52 - DNA, 2434 bp, Homo sapiens
  misc_feature: Incyte ID No 7494963CB1
cttgcaatgttttgttaaggaaatggagttgtttgtcctatagaattttcttttttctttttttctttttgaggtggcgtctcgcactgtcacccaggttggagtgcaatggcgtgatcttagctcattgcaacgtccgcctcctgggttcaagcgattctcctgcctcattccccaagtagctgggatttcaggtacccgccaccacgcccagctaatttttgtatttttagtagaggcagggtttcatcatgttggccaggctggtctcatactcctgaccttaggtgatctgcccgcctcggcctcccaaagtgctgggattacaggggtgagccaccgcgcccagtcagaattttctagatctggattttgctgagtgcatctcctgatgtagtttaacatgttcttctctcctatttatttttctataaatggtggttggatccagaagcatgatcaggttgagggtcagttgtcttggttttgtgtttggtaaacctcaaccatttgctctttctttgaaagctgcaccaaagccagctggagcttcaggaggtgcgtctctcctgccgacagctgcaggtgaaggtggaagaactcactgaggagaggagtctgcagagctctgccgccaccagcacatccctcctgtcagagatcgagcagagcatggaggctgaggagctggagcaggagcgagagcagctgagactgcagctctgggaagcctactgccaggttcgctatctgtgctcacaccttcgaggcaatgacagtgctgactcagccgtctccacggactcctccatggacgagtcttcagaaacctcgtccgccaaggatgtgccagccggcagcttgcgcactgccctcaatgagctcaagagactgatacagagcattgtggatggcatggagcccacgggctcccggagacttgatgatgactccttagaagaacagataaggcagaccagtgaggactcgagagccctaagggagctcatggagggagagaggggtaaactgaggcaaagcctagaagagctgcagcgactccacagtcaggtgacactgctgagtgtggagatgactgccctaaaagaggagagagaccgactcagagtcacttctgaggacaaggagccaaaggagcagcttcagaaggccatcagggaccgcgacgaggccattgcaaagaagaatgctgtggagctggaacttgccaagtgcagaatggatatgatgtctctgaacagccagttgctggatgccattcagcagaaactgaacctctcgcagcagctggaagcttggcaggatgacatgcacagggtcattgaccggcagctgatggacacgcacctgaaagaacggagccagccggctgctgccctctgcaggggccacagcgctgggcggggggatgagcccagcatcgctgaaggcaaacgactcttctcattcttcaggaaaatttaagttgggaggagtcaggccaccaaagatgggtggactggaggcagctggaaaggcggtgcaggcaaggcctcccctgcagcttgcacctcagcagctgccctgcccctcatgctagggccccatgggtccgggagggcctgctccctttcgtcggtggggatggagacctagaggtgggggcctgccttggccactgaaggcttcccttggcccaccgcctggccaagcccacgcctgggcttctccaggaccacgtgcttgagcagggttaggccacctcccagaggggccccttggtgttgggctttgcagctcacacccaacagatcgcagcccacccccaggcactgctgcctccttgattttagcaaatggggaacagaaggaatggaggcccttctctgcatgcctcaggaggcctgagccccaggggcctagacctgtgggggcagcgggccaggcctgagcctccattccttccccagcccctggcccagggtcaaaggagagatggcagcccctcccccgcatgcatgcacctcagctggcaggaggccaagcctctggccgcagggtctaagagccggggcttacccaagctcagctgaggccacccgagccccagggaggaagaaggccctgtccccctgtcgccactgctctccctcccagccttcagtctctgccccttagcagggcctggccaggcagagtgttatcaccagtcatctgcaggctttagccatccagccctttcccctgctcagggctggggttggacggggtctcctcctcccacagctccctcctccacccctcacatacatacataatttcttggcctagccaaacaagtccaggccactgaatggcaccagaggagtctgtggtcagccaccccaccttgagggcagcacaggcaccacggggtcgaggggagggggaggct

SEQ ID NO: 53 - DNA, 3492 bp, Homo sapiens
  misc_feature: Incyte ID No 7644881CB1
gctactttagactttttcatggttatcaatctgtacaaagaatcaccaaactgataaagcaggaaccagagggcaaatcacgctgccaagacaactgtgtaattcgctcgaaaaagaaacgatggagtctcgctctgtcacccaggctggagtatagtggtgtgatcttggctcactgcaacctctggcctcccaggttcaagcgattctcctgcctcagcctctcgagtagctggaattacaggtctggcagaaggaacagtatcaactgactgagtaggtctcattggcagttgtgattcagagacctagaaagctgaacccacggctggcaagaagaggatggtttgtgggacctgggctgatgtctgatgaaattttaagccccagctatagctactacaaagaaaagtggctgatgataagcatgtaactcaaaaagacaatgtatataaaaatatgcaagaatcacaggaaacccacatatccaaccacctagatgaagttgttgctgctgttagcatcactcatagaaagaagttccaaaacaagctgcttcagacagcactattccagcctcctcgagagaaactccacctctgtgaagagaaagcaaagtcctattccaacagtcatgagtacaaacaggccgtccatgagcttgtgcgttgcgtagcactgacaagaatttgctatggagactcacattggaaactagcagaggcacatgttaatctggctcaaggctacctccagctgaaaggactgtcactgcaagcaaaacaacatgcagaaaaagccagacaaatcctcgccaactccattgtgcctccctatagtgagaatacagatgttttcaagttttccattgagcttttccataccatgggcagagctttactctcccttcaaaaatttaaggaagctgcagagaatttgacaaaagcagagagactttcaaaggagctgctacaatgtggaagaattataaaggaagaatggatagaaattgaagcacggatcagattatcatttgcacaggtgtatcaaggtcagaagaagtcaaaagaagctttgtcccactatcaagcagctttggaatatgttgagatcagtaaaggtgaaacaagtcgtgagtgtgtacccatattgagagaattagcaggtgtagagcaagccctgggactccacgatgtatccatcaaccacttcctccaggcacatctcatcatcctgagtagaagcccctctcaagtggaggcagcagactcggcacacatcgtcgcccatgctgctgtcgcttcagggagacacgagcaccatgatgtagctgagcagtattttcaagagagcatggctcatcttaaggattctgaagggatgggaagaaccaaatttctttcaattcaagatgaattttgccattttctacaaatgactggacaaaaagagagagcaacctcgatcctgagagagtccctggaagccaaagtggaagcatttggcgatttcagtcccgaggtggcagagacataccggctcctgggaggagcagacctggcgcaggggaaccacagtggggcccgcaagaaactgaagaagtgtctccagatccagaccctcttatatggaccgcaggacaaaaggactctggccacccagcaggccatgggcatgctgtccacggcccccaaggttgcttcgaagccaaggcaggcatcaaaagccaaagtggccttctgcaccagcatccctcaggacaccctgctggggaaggcccggcccggcacaacagcagactgaggcccccaccctgaaaaagcctaggacattcctgggcactgtcatttagggtgctgtacaaatcacctccgcctagaaaatggaattcaacagtcaggatacagatttccaaggccaactgttggccccaacatgcaacagtgagaccataagcctcccgtgggccacattttgacagtggatgcccttcagggtgatatatgctataaagcagttttctatcacctaagtggtttttcttgccaacaagaatttttacccatcagcactactgtggctgaaaaacttctcttcaacagttcagtgcgccctgtgcaggagtcagcccggcatctgcttgtacacacagctccttgcataggtgtggagttagatctggacagtgaacttcaggaagtcctttcttataggaggctaatagggattgagaataacatgagaagaaaacgctaataaagggaaacctgaacacgctgctgtcagcatgtgttttcaaagtgcagcctgcctcagagttcttcggagcctgaaaaggggtttgagaaagagcccagtaggaggggcaggaggccgacacacctgacttggcctggggcccaggaggcaggtgtaagggagtgaaaagaaaggctagccggaggctgcggggggaagaccgcagactccctgctgcttcgcatccctcctgtggcctccactgcaggcaggacaaacctggatgccacctggagctgcttcctgagttggcacactatcgtgtacacagcagtcttcagccccctggaaggaggccatagtcgtgtgaggatggcaaagtcgaacaggaagctttgagtgccttcctccacgatgtcaacgaggagatccagtgccagatcgaggtggatggaacacccaggggtaggggtgcaggtgtgggcagtgatgtcccttcccctccctcccctggtcccacagactgtggccatgaggctgcaggctggtgctatgacagcagattgcagcacagggccctcccctccagcccccagtgggacatcaaaaccaccctggggccatttgtgcagggcaccacctccagtattgatggggaaaataaactcagtagagccacgacagggtggagagaagcagggaccattgtcttcctcaggagcgtgacagctgaccccacagaccatgcttgctggtacacactggtcccagacccaggcctgtcggacatcagcagtgtgctaaaaacgtgtaagatgtggtcactactcaccgtgtgtcctatctagttgacatgggtggagtcagctaaggggtgaatgttcatatgctcccaattcacgttgaagccctaatccccaaagggatggtattgggggtggggtcttggagaggtgattaggttatgagggtggagccctgatgaatgggattagtgctttataaggagagacaccagagagatgatctctctctccaccatgtgaggacacagtgagaagacagccgtctgcaggcccggaagagagccctcaccaggaaatgaaactgttggcaccttgagacttcccagcttccagaactgtaagaaataaatgtttgttgtttaagcctttcaggctacggctttctgttacagcagcctgaactgagagtccatgccgagtttttgaaataaatgtgaattctgatgtt

SEQ ID NO: 54 - DNA, 3141 bp, Homo sapiens
  misc_feature: Incyte ID No 3790383CB1
tccacacccgctggctggctgatgtttgattctgtaactataccacgcccagaattctctcaaaagggaataaaacacaggtcaaattcctcacccacacactccacagttcaacccctgccagggaaaccaaaagcaggaaaggatctccagcggcgccattctcatttccggtcccagcaccccgcctccatgacgtcaacgcgccgcgccaccgggctgcgtcatctcggcgcgccgctgccagggctgtacacctgctggctgccatggctgaggtgggccgtaccgggatcagctacccaggcgcgcttctcccacagggcttctgggctgcggtcgaagtgtggctggagaggccgcaggtggcaaacaaacggctttgcggcgcccgcctggaggcccgctggagcgccgccctgccctgcgcggaggcccgcggccccgggactagcgcaggctcggagcagaaggagcggggtccgggacccggccagggttcccccggagggggcccgggtcccaggtcgctatcaggacccgagcagggcacggcatgttgcgaacttgaggaggcccagggccagtgccagcaagaggaggcacagagggaagccgcctcagtgcccctgagggactccgggcaccccggccatgctgaaggaagggagggcgacttccccgccgcagatctggattcgctttgggaggatttctcccaaagtctcgcccgtggcaattcggagttgctggccttcctcaccagctccggggcgggatcgcagccagaggcgcagcgtgagctcgacgtggttctcagaaccgtcatcccgaaaactagcccacattgcccccttacaactcccaggagggaaatagtcgtgcaagatgtcctcaatggaaccataacgtttttgcctttggaagaagatgatgaggggaacctaaaggttaagatgagcaatgtgtatcaaattcagctcagtcatagcaaagaagaatggttcatatctgttttaattttctgtccagaaagatggcattcagatggaatcgtgtatcccaaacccacgtggcttggagaagagttgctggccaagttggccaagtggtctgtagagaacaagaagagtgactttaaaagcaccctttccctcatctccattatgaagtatagcaaggcttaccaggaacttaaagagaagtataaggaaatggttaaggtgtggcctgaagtcactgatcctgagaagttcgtgtatgaagatgtggctatcgcagcatacctgctgattctatgggaagaagaaagggctgagaggggactaactgccaggcagtcctttgtggacctgggatgtggaaatggcctcctggtccacatcctgagcagtgaggggcatccaggcagagggattgatgtccgaagaagaaaaatctgggacatgtatggaccacaaactcagttagaggaagatgcaatcacacccaatgataagacccttttccctgatgttgattggttaatcggtaaccattctgatgaactcacaccatggatacctgtcattgcagccaggtcttcctacaattgccgcttctttgtcctcccctgctgcttctttgacttcattggaagatactcccggaggcagagtaagaagactcagtaccgggaataccttgacttcattaaagaagtgggcttcacctgtgggtttcacgtggacgaagactgcctcaggattccttcaaccaaaagagtctgtctcgttggaaaatccagaacatacccttcctccagagaagcttccgtggatgaaaagaggactcagtacattaagagcaggcggggctgccctgtaagcccacctggctgggagctttccccttctccacgctgggttgctgctggcagtgctggtcactgtgacggtcagcaagctctggacgccagggtcgggtgtgtaaccagggcctgggccgctgagcatggagcagggccccaggctgaaggaccctggctacctggatttcatcccagagaaaaggctgagcgtgtgaggaactgtgccgccctgccacgagattttattgaccaagtggttttgcaagtagcgaatttactgttaggtggaaagcaattaaacacaagaagttctcgaaatgggagtttgaagacctggaatgggggagagagcctatctctggcagaagtagccaacgagctggacacggagaccctgcggaggctgaagcgggagtgtgggggcctgcagacgctgctccggaacagccaccaggtgttccaagttgtgaatgggagagttcacatccgcgactggcgagaggagacactgtggaagacaaagcaaccggaagcgaaacagagactgctctctgaagcctgcaaaacccgcctctgctggttcttcatgcatcaccctgatggctgcgctctgtccacggactgctgcccgtttgcccatgggcctgcggagctgcggccaccccggaccaccccgaggaagaagatttcatgagctgcatccttgccagccgaggcctggttggggaggccaaaccaaggagagcttccccagcagtcgtcagtgctgtggtctctgctctggctgtgtttcagcccacctcctcccagctttctccacatcctcacagtgatgaaccgtatttcataaacatcacacgccagagaagccacagttactcggaagcccccagctgactgcctggcttgtttcagatgcagccgcttgaaacgtgcgcagcatcttcatatcataaagattgtgcacggatccttacaatgtctcctgggggagagcggctgaggctgccttgcacaggcccttcccagggcgctgtccgacgcctgccccaccatgtccacatctgtgaagaggatggggctcctcgagaagtaagaccgtatctgccagcgtttctcaccacactggagagcagctgctctggagcagggatccaccagattggtatttttaaaaaaggtgtcaggcttgctatgttgaggttgtttttagagttacagagaataaaaacactcataatttcctgaaaaaaaaaaaaaaaaaaaaaaaaaaaaaaaaaaaaaaaaaaagg

SEQ ID NO: 55 - DNA, 3491 bp, Homo sapiens
  misc_feature: Incyte ID No 3846110CB1
cacgaggcgcagctatacaactgggccgcaccagaagtgatcttacagaaggcagccacagtgaaatcagacatctacagcttttctatgatcatgcaggagattttaacagatgacataccctggaagggcttatatggctcagttgttaaaaaagccgtagtctcggggaattatttagaagctgatgtcaggcttccgaaaccttactatgatattgttaagtcaggcatccacgtcaagcataaagaccgaactatgaaccttcaagatatccggtatattctgaagaatgacttaaaggattttactggagcccagagaactcaaccaaccgagagccccagagtgcagagatacggactccatcccgatgtcaatgtctatctaggactgacttcagaacaccccagagagacacctgacatggaaatcatagaactaaaggaaatgggcagtcaacctcattcaccaagggttcactctttattcactgaggggacactagatcctcaggccccagatccatgtctgatggccagggagactcagaatcaagatgctccttgccctgctccatttatggcagaagaggccagcagccccagcacaggtcagccaagcctctgcagtttcgaaatcaacgagatctactcaggctgcttgattttggaagatgacatagaagagcctccaggagctgcttcatctttggaggcagacggacctaaccaggtagatgaactgaaatccatggaagaagagctggataagatggagagagaggcgtgttgttttggcagtgaggatgagagctcttcaaaagctgagacagagtactcttttgatgactgggactggcaaaacggttcactcagttcactcagccttcctgagtcaaccagagaagccaagagcaatttgaacaacatgtccacgactgaggagtatctcatcagtaagtgtgtgctggatctaaagattatgcagacaataatgcacgagaatgatgataggctgaggaatatcgagcagatattagatgaagtcgagatgaaacagaaggaacaggaagagcgcatgtctttatgggccacttcaagagagtttacaaatgcctacaagttacctctggccgtgggccctccatctttaaactatattcctcctgtcctacagctttcagggggtcagaagccagacaccagtggcaactacccaaccctaccaagatttccaagaatgctgccgactctttgtgaccctggaaaacagaacacagatgaacaatttcagtgcactcaaggagccaaggacagtttggaaacaagcaggatccaaaataccagtagccagggaagacctagagagtccactgcccaagccaaagccacacagtttaatagtgcactcttcactctgtcaagccaccggcagggaccttctgcatcacccagctgtcactgggactctaccaggatgagtgtggaacctgtttcttctgaaatctataatgcagagtccagaaataaagatgatggaaaggtacacttaaaatggaaaatggaggtgaaagaaatggcaaagaaagcagctactggacagctcacagtacctccttggcatcctcagagtagtctgactttagagagcgaggctgaaaatgagcccgacgccctgctgcagccccccattaggagcccagaaaacacggattggcagcgagttattgagtatcatagggaaaatgatgagcccagaggaaatggcaagtttgacaagacgggcaacaatgactgtgacagtgaccagcatggcagacagcccaggcttggaagcttcaccagtatcaggcacccatctcccagacaaaaggagcaaccagagcatagtgaagccttccaagcaagttctgacacattggtggctgtagagaaatcttacagtacctcgagtcccatagaagaggactttgaaggaatacaaggtgcatttgcccaacctcaagtctctggtgaggaaaagttccaaatgagaaaaattcttggaaagaatgctgagattttgcccaggtctcaatttcaacctgtacgaagtactgaagatgaacaagaagagacatcaaaggagtcaccaaaggaactgaaagagaaagacatatcattgacggatattcaagacctgtctagtatctcctatgaaccagacagctcttttaaggaagcttcatgcaaaacacccaaaataaaccatgcacctaccagtgtcagcactccactcagcccagggtccgtttcttcagctgccagtcagtataaagactgccttgaaagtatcacatttcaggttaagacagagtttgcctcttgctggaacagtcaagaatttattcaaactttgtctgatgactttataagtgtccgagagagagcaaaggaactggattctctccttacttcctctgaaactcccccttcaagactgactggtcttaaaagattgtcttcatttattggggctggatcccccagccttgttaaggcatgtgactcatcaccaccccatgccacccagagaaggagcctgcctaaagtagaagccttctcacagcatcacattgatgagctgccaccaccatctcaggagctacttgatgacattgagctcttgaaacagcagcagggctcatccacggtgttgcatgagaacacagcaagtgatggaggaggcactgcaaacgatcaaaggcacttagaagaacaagaaactgacagtaaaaaagaagatagtagtatgcttttgtccaaagaaactgaagatcttggagaggacacagagagagctcactctactctggatgaggacctggaaagatggctgcagccacctgaggagagcgtggagctacaagaccttcccaagggctctgaaagggagacaaatatcaaagatcaaaaagttggtgaagagaaaagaaaaagggaagatagcattacaccagagagaaggaaatcagagggtgttctagggacttctgaagaagatgaactaaaatcctgtttttggaagcgactaggttggtccgaatcatccaggataatcgtgctggatcagagtgacttgtcagactgattggaattggatcatagacggactcctggcctgagtttgagtgtcctggttgtaagctcctttcttctctttctgcttcagttgctgtcagggcagcagttccagttctgtaagtctcactttgttcagctgccacaatagacatcatcgtttggccctctctgttagcagcacattcaaccatttgttttcagtcagatttctgaaaagtgagaggtagttttgatagtaaaaatttttggttgtgcctagaatggctttggttttgttgatgttaattttcaaaaactttaactcttgttatataataaaatgtttaattttaataacagaaaaaagggggtccactagtttagaga

SEQ ID NO: 56 - DNA, 4312 bp, Homo sapiens
  misc_feature: Incyte ID No 1878279CB1
tcttatgccatgcctctagtgtgccacatttatgttcacgatctcatttaattcttgccacaaccctatcaggaaggtgggagtcaatcattttgacaagtctcctgaaaggaacagctagcaggaactgaaacctttttccatttggtctcgtggcaaaggcagagattgctccagcagctccacacaaaatgatgtgctcacgggtgccctctgaacagtcttctggtacctctctcttgcctaaagacggtgccccattttcttgggattccttggatgaggatggattggatgactccttgctggagctgtcagagggagaagaagatgatggtgatgtaaattacacagaggaagagattgatgcactgttgaaggaagatgacccatcatatgagcagtcttctggggaagatgatggtgggcatgttgagaagggagaaagagggagtcaaattctacttgatactccccgagagaaaaattcatcgtacagcctgggaccagtagctgagactcctgacctcttcaaactacctcagctaagtacatcaagtggtcatggaccagctcatactaaaccattaaacagacgctctgtactagaaaagaatcttataaaagtaactgttgcaccatttaatccaacagtttgtgatgctctgcttgataaggacgagactgattcgtccaaagatactgaaaaactctcttcccttggagaagagatgagagaagatggtcttagcccaaatgaaagcaaactttgtactgaatctgaagggatcagccccaataactctgcctggaatgggccccagctctcttcttcaaacaataactttcaacagactgtctctgataaaaatatgcctgacagtgagaaccctacgtctgtattctctcggatctcagaccattcagagactcctaatatggagttatcctgcagaaatggtggttcacacaagtcaagttgtgaaatgagatctctggttgtttccacctcatcaaacaaacaggatgttcttaacaaggattctgggaagatgaaaggccatgagagaagactaggcaaagtcattcctgttctacaaactaagaccaggactaatgttccgacgttttcacagtcaaatctagaacagcagaagcagctttatctcaggagtgtcattgctcatatagaagacccagaggacactaaccaaggatgaatccctagaagtggaattgctgggtatctcgggggagctttgtgccttgatggatcaagttcatcatatgcagcactcaaaatggcagcatccttcggacctcaccacgcgaaactacgcccgccgacagaaacatctgcaaagatacagtctgactcagtgggttgacaggaacatgcgaagccaccatcggttccagcgtctcccagacttctcgtacagttaatttgtgtcatcccatcagcaatgaaggtccctatccagggtcctgcttggagcagcatttcatgttcttttgctgttttgtgctttgccgattttggattttatttttcacaaaatttttatttaaaaaactcgtcaccttttggaaatgcccattgccgacttgaatttttttgtatgaagtccctcctgattttgtgtgtgtgtgtctgtgtttaagcaagcgttcggttggtatagtttttttttgtttttttaatttaaattgaaggtagctgcctcctgaaagccagcattaagccagaacacccaggttcaagcaaaagacccacctctctgcagaggcaaagtctactttctggtacctcaaagaaatcattgttcaatcttccataaggaagagattctttaccaggctgtgagccagtgttagataacttgtgaatggatataagttacttttaacaacccctcttacttttttatttgaatcctctgaatacctgtcagtattttaaagttggcaatccaggacattataagtaggatggagcaggagaagaatcctattgaaaggacaaattaaaatagtaaatcctctcctctcccttctgtaggtactagctgccttgatttttttttgtgggtgggggggattttttcattccttattgaacttattttgcacaatagtgtttacaaatcttatacatcttactttgacagaagacttataataccttgtggtcttgagactggttgtctcctttgcctttcttcaaggattttcctttttctttttaccccccagtgaccactctgctctttaggattctagtggcagtattcttgtaacctgttagacgtggatatacctcttcaggatggcctccttgctgtctttattgaagagctgcccacccagagtcttgactctagggcaggctagtgctattgtggtgtcagttgatatttagtttcatttgctgaaccccaaatttgagtgttagttagggagcctccattctgtgggagagtgtggaaaggcactcttttttctccatattagttaacaagaggtgtctttaggccctggatgttaccatctcatttggtcaagcccctgatacctagtttcacatggatgctaacgtggatgtaatatagttgtgacagcctatgcacaaaagcattacctgatcttaaggtttggtattctaaccaaagttgacagactgtgcgtctgtcttaatggatgtcttaggagttggttattctctctttttttttgagacggagtttcgctgttgttgcccaggctggagtgcaatggcgtatctccactcaccacaacctccgcctcctgggttcaagggattctcctgtcttagcctcctgagtagctgtgattacaggcatgcgccaccatgcctggctaatttcatattttttagtagagacaggatttctccatgttggtcaggctggtcttgaactcctgacctcaggtgatctgcccacctcggcctcctaaagtgctgggattacaggcatgagccactgcacctggccggttattctctctttacagatagctatagacatcattttaggaagtgttgcagtctggcatttgtgctattgttcattctctgtgaaggctgttcatagttgctatagcctgtgtttagttttgtgatttcatcaatcccatctttctgtgtgagtaatgcattctaaacatcctaccccactttagaaacggacgtggggaacgcttggtcatttaagccaacaataaatttaggtgaatgtccctaagtgtttactgtttttatccagtcaaggatttgcttttccttgaacatttgttttaaattctggggccaaaatgcaaaggagaagttctattcaaaggcagtagttgaaatctattattttagttagcctacttggcatttactacatcggtcacttctccaggctgccctaaattaggttgatggagtgagacatgccaaacatccacctttgggaccatagcatagttaaaattaaatgtagttggaatagctagcattgcagctacagtagggaactgtagtctagttccctacagaaaacccaaggagtgaagggacaggattttgcctaggcaaaaatctaagactcgtgccctcctggtacatggggttttaagactgaatgtgtaataggagcactgcctttgccaaatcaaatgagtgacaggttaactagaaaatgtgacaatcacatttcctcttagctcaaataattctgtttttccaaagctttagcagcttaattaaatctgttggactgggggaggagagagctgttctctagtggttaacatggtattctttaagaagaaaaaacaaagccaaagaaaactcattatctggcatgttcgccttaaagatggtactgggtagaatctggagttttcatctcttttcaaagctgcatatctctcatatttggtattggcctctaagtctaatattgcagttggaattcttgctgtattattttttaagcaagtgttaggtgcatttaactgctttcttcatccatgacgacattcccaccatgggggtcttgacaaagcagagtaaaaatatgctgtttacattgtttacttacaagtaaggagcctgaaataacctgtagtttcgaatgcaggccctgatttactggcgttgtcagtttcaattatgaaactgaagtttggtgcctcctctttatcatgttttttcccttgtagcagttgtgtttaatgtcattaaaaagaaataaaagttctttgtcagtgacaaaaaaaaaaaaaaaaaaaaaaaaaattg

SEQ ID NO: 57 - DNA, 3860 bp, Homo sapiens
  misc_feature: Incyte ID No 1848891CB1
gggactgagagccgttcccacgtgagaggctccgcggccgaattcctcgcgtgcagcaggcgcggaccgcccggcgtccggccggactgagagccctggtccggcgcgcgccgccggccgggcgggaggcgggggcgggctcggctgcgccccgatgcggcggcgacctccgggtctgtgagcccggcgcgcgccgtcggagcccctcgcgcagccgctggtagcgtccccccgggcacccggcatgcgggcggccgactcgggctcgtgggagcgcgtccgccagctcgcggcgcagggcgagccggcgccttcctgcggggcgggggccgggcccgcgcggcccccgggacccgcagcctgcgagcagtgcgtggacgcggcggggcccggcgatcggccccgcgccggggttccccgggtccgagcggatggcgactgcagccagcccgtgctcctgcgggaagaagtgtcgcggctccaggaggaagttcaccttctccggcagatgaaggagatgttggcgaaggacctggaggagtcgcagggcggcaagtcctctgaggtcctctcggccaccgagctcagggtccagctggcccagaaggagcaggagctagccagagccaaagaagccttgcaggccatgaaagctgatcggaagcgcttaaagggcgagaagacagacctggtgagccagatgcagcagctgtatgccacactggagagccgcgaggagcagctccgagacttcatccgcaactatgagcagcaccgcaaggagagcgaggatgcggtcaaagcgctggccaaggagaaggacctgctggagcgtgagaagtgggagctgcggcgccaagccaaggaggccacagaccacgccacggcactgcgctcccagctggacctcaaggacaaccggatgaaggagctggaggccgagctggccatggccaaacagtccttagctacgctgaccaaggacgtccccaagcggcattccctcgccatgccgggcgagacggtgctcaatggcaaccaggagtgggtggtgcaggcggacctcccgctgaccgcagccatccggcagagtcaacagactctctaccactcacacccccctcaccctgcggaccggcaagcggtcagggtgagcccctgccactcccggcagccctctgtcatctccgacgcatctgccgccgaaggcgaccggtcgtccacaccgagcgacatcaactcccctcgacaccggacacactccctctgcaacggcgacagtcccggcccagttcagaagaacctgcacaaccctattgtacagtcactagaggatcttgaagaccaaaaacggaaaaagaagaaagagaagatgggattcggctccatctcccgcgtcttcgccagagggaagcagcggaagtccctcgaccccggcctctttgatggtaccgcccctgattattacatagaggaggacgcggactggtgatacgcgctcccctgcgcctgctgcccgcaggcgtgtctgtgcgtgtgggcgtgtgtgcaagcgagcgtgggtgcgcgtgtggccgtgcgtggggtgcgtgtgcacgtgtgcgctggcacacatgggtgctgggtgtggccgagcgcctctaacaagtgaaaacacgagtgtgaacctctctcccctgcgtcgccacctctgtaattgatgtacataccgcaaaccgtgtgtgaacctgtcaactctctgtcgtctttggagcgatacagttgtgttgttaatctggtttattatttttcttcagtgtttggtttttctttttctttttgtttggttcgtcggtttgtttttgtttttgtttttttccccctttctcctcccctcctcctttttatgaaacttgaaaacttgaaggactgctgtgtatttgtaaataacaaaactattgtgcactctgtgcttgtaaatgtccctcgtccaaaccgctactcctggagcccgtctggcagaggatgtggtctgtttttgatgtcccccctcccgcccccctggtgtgaacgtgtgggaccagaccctgtcctgggggtgcgcccaagtcactttaaccacaaaacgccatcgtcgtcagggtaagctctgctctctacaaagactcgcgagccgggccaaggggccttgtcttggctgggtttgtcagaggtcaaaccggctcttttaaacggcctaccagtttttaaattgcattgccgtttctttctttatgaaaaaaaagaaaaaaagaaaattgtttcatttaatttatttgcacaaatgctgaaaacttattctatctaaattattacataaatattggaatgtctatttttccatggggtgggcgggaggtgggtgtctctgttgacttgtctgttctgttaccatgctgctacccaactgtgcaaagtagtttagggtggccagaacccagggaccattggatttcaaagcttgctttttctgttggttcttctctctccttctctctctgtctctcccattctcctgcccatgcatgaaaggatcctccaccttcttcccacccagagctccctccaggcctttctctatatatttatttatctgacatacagaacacgactttagtgagcagagtgctgacagtcatggtccccttctttgggtctgtcttttgagagaggttgagttcagggcaagacagcctgccacacatccaagggtacgaccagtgggaccctggcctgagtctgttctccgaggggcctccagcagcttctgttcctccctgcagctgtgtctttcttgtcctgggtttaggatgcaggtgggccaggcaggtgttgttaggggaggcacccacttcaaaaggagggccacagtggggacacagagtccagcacctgagccctccaccctccccattctggttggttccatcagccacacttagaatctcccaggatcccttggactccgtcccccaacttgtcacagcagcctgagcctacccaccccaggagacaagagctggcaggaacatctgcctatatggggggtggcgttggccccaaagaggcttcaccagcaaaggaactgtgtgtattttaatgccaggggacggaggatgtgtgagctgttcagcaaggtctgcccaaggcctaaaactcaatttccttattcttttgcttctgctcgtccttaacaactaacagctcaacccacacctctagacaacagtagtcgtgctttctgctaacggtgacattttcagctcttaaaaagaagcaaggagattttcaaatgctagagtatctctatcagaaggcataggacattgtgtccaaagtctcaagaacaaacaacactttccttctgacctggtccaaaagtcagcaaacagcaagcaggcagaggccctcataaggacttttctgtcctcctttgaccaaactgtttaaccgagcctaggggtgacggggagcgacccaagctggcatctttctctacggagacagattttagaaaatacttttcttgcccatgaatttcttttctggttgatttttatcattttccctttacttacaagaaaataagattgcaaccactcctgctaatgatttagtagttccttttcatttcagtttttgtaaattaggagataattctaagagttactaaaggatgatttatttaagagaactacgtcaaatagcgaatgagttatgggtaacattagacgaaaataacctttcccgtgggaaaggtttctcgaaggcatggatgcaaatataaaatattaaaaaaaatctaaataaagcttattttaaaatatgaaaaaaaaaaaaaa

SEQ ID NO: 58 - DNA, 3742 bp, Homo sapiens
  misc_feature: Incyte ID No 2500251CB1
cgcacgcataacagccgtggtggttatggctggtctgagcggcgcgcagatccccgacggggagtttaccgcgctagtgtaccggctcatccgcgatgcccgctacgccgaggcggtgcagctgctgggccgagaactgcagcggagccccaggagccgtgccggcctgtcgctgctaggctactgctactaccgcctgcaggagttcgcgctggcggccgagtgctatgagcagctgggccagctgcacccggaactggagcagtaccgcctgtaccaggcccaggccctgtacaaggcctgcctttatccggaggccactcgggtcgccttccttctcctggataaccccgcctaccacagccgggtcctccgcctgcaagctgccatcaagtatagcgagggcgatctgccagggtccaggagcctggtggagcagctgctgagtggggaagggggagaagaaagtggaggcgacaatgagaccgatggccaggtcaacctgggttgtttgctctacaaggagggacagtatgaagctgcatgctccaagttttctgccacactgcaggcctcgggctaccagcctgacctttcctacaacctggctttggcctattacagcagccgacagtatgcctcagcactgaagcatatcgctgagattattgagcgtggcatccgccagcatcctgagctaggtgtgggcatgaccaccgagggctttgatgttcgcagtgttggcaacaccttagttctccatcagactgctctggtggaagccttcaaccttaaggcagccatagaataccaactgagaaactatgaggtagctcaagaaaccctcaccgacatgccacccagggcagaggaagagttggaccctgtgaccctgcacaaccaggcactaatgaacatggatgccaggcctacagaagggtttgaaaagctacagtttttgctccaacagaatccctttcctccagagacttttggcaacctgttgctgctctactgtaaatatgagtattttgacctggcagcagatgtcctggcagaaaatgcccatttgacgtataagttcctcacaccctatctctatgacttcttagatgccctgatcacttgccagacagctcctgaagaggctttcattaagcttgatgggctagcagggatgctgactgagcagcttcggagactcaccaagcaagtacaggaagcaagacacaacagagatgatgaagctatcaaaaaggcagtgaatgaatatgatgaaaccatggagaaatacattcctgtgttgatggctcaggcaaaaatctactggaatcttgaaaattatccaatggtggaaaagatcttccgcaaatctgtggaattctgtaacgaccatgatgtgtggaagttgaatgtggctcatgttctgttcatgcaggaaaacaaatacaaagaagccattggtttctatgaacccatagtcaagaagcattatgataacatcctgaatgtcagtgctattgtactggctaatctctgtgtttcctatattatgacaagtcaaaatgaagaagcagaggagttgatgaggaagattgaaaaggaggaagagcagctctcttatgatgacccaaataggaaaatgtaccatctctgcattgtgaatttggtgataggaactctttattgtgccaaaggaaactatgagtttggtatttctcgagttatcaaaagcttggagccttataataaaaagctgggaacagatacctggtattatgccaaaagatgcttcctgtccttgttagaaaacatgtcaaaacacatgatagtcattcatgacagtgttattcaagaatgtgtccagtttttaggacactgtgaactttatggcacaaacatacctgctgttattgaacaacccctcgaagaagaaagaatgcatgttgggaagaatacagtcacagatgagtccagacaattgaaagctttgatttatgagattataggatggaataagtagttatgactgatagtggcttttttcaaaatggctttcttacgtaccacacttttttttatctgtatttagccttggcatctttatatttgtcttattttgaatcttatccactttgtaagaacaagtttatgtttgagcaactttttcatttaatccagaagggtagggactatgcagtgtaagctgcatcacttctgctttcttcctactagtgacaatcatctggtcttgccctcaagcaacaattgctagagtaacatctttgtataagcaagtaaccccagatagagttgacgtttcagctttgggctgtcaaaagggtatgtcatggaccaaagcactgttagtacgggtatgtttgcatttggtcactgatatgtaaatgactgctagcccacggctggaccacttctcaatcagcaaataaagccatgtctattttgctatctcagcatagactatgctgtctgataaatctaattcttaactctatttctccagttttttagtcctttaactttctggattgcaacgaagtctagtttagacctctaagcccttttagaagtacaagtataatgggaatttcttttcttggttcttttcaggttatgaggtttggtcagtgacaaaattttttttcataatttggttgattggttgcttcttaagttttataataaacgtttttcttcatgttctatttttgattttacataaatgattttgcctccttgtggatactgacatatattaagtgtggaagcttattaatatttttggttttttaaaaactgaaatttttaatttttactttttaattttttaggaaaaaataagcactgaactgagaatgagaagaataaaagtatgagttccataccttctaattttaggctgtcagaaattcctttattctttgggatttcacaatcatttgaactatcagaagcctttacaattacttttagctgtaacatccgattctgtataagccacatagaaaaaagttgcctttctttttttatgacctggatatataagcaaatcagctaggaaatatataattgtattttatattaatgttttctaggattttggcttacagtaaatgttagcccctatggtaagtgattgttattgttggatgttatactgattattaataagaaatttggatttttgcctttttacctggaatttttgcttacagccgtagctatgaatatatatagggtggtccccagtctctgttatggttgcgcataaattaataattttataagtatttagaaatggtataattctcttaacttcctctttcagtttttgtactaatgtttcgtttttgttcgggaagaggagattcgcttttaagtgcttccaaaaaatgatgaaccaccgttccattcagaaaaaaaggaacagcacaacgcaacacgcaaaggagacaactatgcggagcaacttcttgcaaacaacaatggatgaggggacgcggggccaattaaaacacacaacaacatatcagcagagtataagacaaaaaaaaaaggagggcgggaaagataaacaaaaaacacgatatatggaccacggcaaacacaaggaggggccgaggcacacaaatcagcaaaataggtagttatcgagcgccacagagggcagtaaaaacgaggggggagaccggcggtcactttcntgaccctcctccacaga

SEQ ID NO: 59 - DNA, 2160 bp, Homo sapiens
  misc_feature: Incyte ID No 55026561CB1
gcaaaatgggcctctccccaactcccctcctcatcccccagcacaaggcaaagggcaccccttgctgtgagtttgggggcaccggctgtcgggggccgacccagaactttgcggggtcccctgagggacccgtggcgggaccggccccgggttcgtcaaattccgccgatgctttcaggaggctgattaaacaaacgacaagattggagtgggaaatgagaggctcttaaaagtggaagctttttgtttgtccatttacaagaaattggacaatgaatcacttcaggaagatggaggtcatcaacctcaccaccctacctatgataccagtggatgagcacctggctgtctcgcttgtcgcacggaatacaatggtgaagactgtgaggaaggagttagagaacaatccaccctcatgccttattggctccatgcaccaggtgaaccaaaagattgctgacataaatctgcgtaccgagccgtcggccaacagcctggcaattgagagatttgagttggagaagaaggctttaagagagaaaactcgcagcagtccagaagacaaagtcaagagacaaaggaaatctcagtattcctgcaaaggctccgaactcagacatgccagatcttctgttataaaaaggaaaacagcagataaaaatctgctggcagagctgtaccagtattccaacttcaacagctccaagccaaacaagcttccgaatggcgtggacttctgtgacatggtgggcaacgtggtccgggctgagagagactgccttagtggcaagcatttctgttcaggtagagaattagagaagtttctctcttcttcttctccaagagccatctggctggatagcttttggtggatatttcatgagaggtaccagccaaacaaggagctccagaataatctgtttgaccggatagcccagcactatgccttacttttgtttcgtgtacccaagtcccactctgaagaggcgctcttaaaaaggctgccatcacttctcagcaaagccgtgtacaccagcttctgttgctgctttccacagtcctggttcgacacgcacgaattcaagtctgacatctgtaacacaatgagcctgtggatttcaggcacctatcctagcccacagagctatgacagctgggactactcggaactagacccagagcgattccgcagagaagaattaatgttgtacagaagaagactgacaaaggggagagagttttctttgtttgctggtaagagagccttctcccagaagccagcccagagcaggaaattctaccaccctcagtcttctagtgcaaattcacccagtgaaaaaacctcttcggccaagcagaactcagaaaaaagcttacgaatgcagaatactgcaaaagagcatcattgtcagaccctggtcttgaagaaacctacgcaagaagtcaagaggatatcagaagcaagagaatgtgagaatatgtttcctaaaaagtcgtgtgctgcctgcaaaagccctgagctgacttcaaacctcttcaacatttatgggaagagccctctgattgtgtactttctccagaactatgccagtctgcagcagcacggcaagaatgtgttgatagtcagaagggaaaagaccacgagcacccctgactgcaccccaacgtatactgatgtcatcagcgagaccctgtgcagcatgaagaagcggaaagacaacctcaatcagttgtaccagcatcattggactgaatggaattattttgacaagcatctaaaggagctgcaagacaacttctccagggaaatgaagaatattggtccaaaagcagcagatacaaaaaaggcaaaccacatgttcatcccaccttcagccgtcaatgaggaatcacctgacaagaaaactaagggaagtctccaaagagaaattgagtttaagggttgttcgaacaaaaaccatgggaaaggtagaagtgtgaacatgaggggaaaggaggagagggaaagagaagagaaacagaagttgaacatttctttccactcacttccaagccctgaggagctccacaacctagaaccaggaagcgcctacagaatccgtgatatttctgctacgaggtggagtggaacaagaaaataagaaaattgtggccaaagac

SEQ ID NO: 60 - DNA, 1840 bp, Homo sapiens
  misc_feature: Incyte ID No 7502593CB1
gtcttacaacaaagccaaggaatctcgctgctgagggcagttctgtgctttattatgaagaataatggacgatgatgatgcaaagctcaaagcagaaatagaagctgaattggataaactcagcatttcctccttggaaaaagaagacattgagagtgatgcaaaatcagaaacccagagtgatgatagtgatacagattcagttgaattaccagaatcagttcttcactgtattaacatcataaagaacaggagtaaagctgttgaagagctcattcttcaggacctggaagatactgatattttaagctgtagttatggagcagtttctaataatcatatgcatttaagaacaggactatcaactgaatatgaagaaagttcagagcaattaattaagatattatctgaaatagaaaaagaagaatttatgagaagtaaaaccgattgtgccactcctgattttgttcctgagcctagtcctcatgacttgcctatggatgaacatgttttaccagatgatgctgatataaattttggatactgtgaagtggaagaaaaatgtagacagtcttttgaggcttggcaagagaaacagaaggaattagaagataaagagaaacaaactctcaaagctcagagggatagagaagaaaaacaatttcaagaaaaaaaaaaaaagcgacattgctggatgaaacaatttaaagttgaaaagaagaaattagagaacattcagaaggtattttgcttttgtttttcatgtatttttaaaatcagtagctacctctgaagaatattgatatttggattgtgttgcagattgagcttaatagtatgacttttaattactctctgatagaggaagaaattgactgtgaattaacagtatttaatatgaggtccttgattagtttttattgaaattcattggaaggtcagacacacttctgatcttgggggccagtggagtctatgaatttcaagggtcatcatggatagcctgaaatttacaatataaattgttgtttttctttatatgcttctaaacattggaaaaagcaatgaaaaattcttgagtatgcattaaccttgctctgtggtctggcatatgacatgtaacacatattggaaaagtagttcaaccatttagagtcagtctgcgccccaccttcatgaattaagctacttcctttgaactggcaactttttggtgtgaagttttgtactgttttcatttctgtcgtatgtgttgttggtgttgttttccccctaggagatccacacaatcagatcatagtggtggtatatcggtattgagaaactggtggacttttaggacaccacttgcaggtcatgagaaccttagaatatttggataacaggcatgactgacttctctatctttcatgattaaagttgatttatacattatccagaacttgctgggtttgagtgttgcatataaagaacattagcttgttcaacattttttaatcctccatgttgaggacatttcatttcttgactgtgtagaaaataaacacattaaaggctgggcgcggtggctcacgcctgtaatcccagcaccttgggaggccgagacgggtgtataacgaggtcaggagatcgagatcatcctggctaacacggtgaaaccccgtctctactaaaaatagagaaaattagccaggcatagtggcgggcgcctctagtcccagctacccgggaggctgaggcaggagaatggcgtgaacccgggaggcagaccttgcagtgagccaagatcgtgccactgcactccagcctgggcgacagagcaagactccgtctcaaaaaa

SEQ ID NO: 61 - DNA, 1808 bp, Homo sapiens
  misc_feature: Incyte ID No 7503957CB1
tcttatgccatgcctctagtgtgccacatttatgttcacgatctcatttaattcttgccacaaccctatcaggaaggtgggagtcaatcattttgacaagtctcctgaaaggaacagctagcaggaactgaaacctttttccatttggtctcgtggcaaaggcagagattgctccagcagctccacacaaaatgatgtgctcacgggtgccctctgaacagtcttctggtacctctctcttgcctaaagacggtgccccattttcttgggattccttggatgaggatggattggatgactccttgctggagctgtcagagggagaagaagatgatggtgatgtaaattacacagaggaagagattgatgcactgttgaaggaagatgacccatcatatgagcagtcttctggggaagatgatggtgggcatgttgagaagggagaaagagggagtcaaattctacttgatactccccgagagaaaaattcatcgtacagcctgggaccagtagctgagactcctgacctcttcaaactacctcagctaagtacatcaagtggtcatggaccagctcatactaaaccattaaacagacgctctgtactagaaaagaatcttataaaagtaactgttgcaccatttaatccaacagtttgtgatgctctgcttgataaggacgagactgattcgtccaaagatactgaaaaactctcttcccttggagaagagatgagagaagatggtcttagcccaaatgaaagcaaactttgtactgaatctgaagggatcagccccaataactctgcctggaatgggccccagctctcttcttcaaacaataactttcaacagactgtctctgataaaaatatgcctgacagtgagaaccctacgtctgtattctctcggatctcagaccattcagagactcctaatatggagttatcctgcagaaatggtggttcacacaagtcaagttgtgaaatgagatctctggttgtttccacctcatcaaacaaacaggatgttcttaacaaggattctgggaagatgaaaggccatgagagaagactaggcaaagtcattcctgttctacaaactaagaccaggactaatgttccgacgttttcacagtcaaatctagaacagcagaagcagctttatctcaggagtgtcattgctcatatagaagacccagaggacactaaccaaggtatctcgggggagctttgtgccttgatggatcaagttcatcatatgcagcactcaaaatggcagcatccttcggacctcaccacgcgaaactacgcccgccgacagaaacatctgcaaagatacagtctgactcagtgggttgacaggaacatgcgaagccaccatcggttccagcgtctcccagacttctcgtacagttaatttgtgtcatcccatcagcaatgaaggtccctatccagggtcctgcttggagcagcatttcatgttcttttgctgttttgtgctttgccgattttggattttatttttcacaaaatttttatttaaaaaactcgtcaccttttggaaatgcccattgccgacttgaattttttggtatgaagtccctcctgattttgtgtgtgtgtgtctgtgtttaagcaagcgttcggttggtatagttacggtttttaatttaaattgaaggtagctgctcctgaaagccagcattaaggccagaacacccaggttcaagcaaaagacccacctctctgtcagaggcaaagtctaatttctggtactcaaagaaattcattgtccatcttccataag

SEQ ID NO: 62 - DNA, 3941 bp, Homo sapiens
  misc_feature: Incyte ID No 7504415CB1
ggcggcggttttggctgtgtgaggaagacggaagagacggcggcggagggaaaccgacttccactagtccgggtcgcttgggcggccgggggccctcagagtctcccgggcagtggtagcagttgcagcaggatcaggcgcctgtcggcttctgacgtttaaaacagggggagcggaagggagccactggccgcggtggcagggccaggtataaggaaggaaaatatggcggcggcggcggcggcctgaggaggcggcggcggcgcgggaagctgcttcgcggagatcatggcggaggcgggagcagggcagtgacgggagccccgagttcctagcgctgcggggcgggaggctacgaagcgctgcgcggccccctcggggctgccgggcgccgggctcgccaggcctggacaatagcgccggggagccggaggcgaggaaaggcggcggcccagagctcggtccctggagcgggccatgcaggcggcggcgcggccccgcggcgcccagcggcggcagtgaggccggggagccctccgctcgcgggcgccctcacgcctcgcccctcgcctctccagggcccctttcctgggcgtctactggcggggcccccgccccggttcccgggcggcacgatgaccgacacccggcggcgggtgaaggtgtacacgctcaacgaggaccggcagtgggacgaccggggcaccgggcatgtgtcgtctggctacgtggagcggctgaagggcatgtccctgcttgtcagggctgagagcgacggttctctacttttagagtcgaaaataaatcctaacactgcataccagaaacaacaggacactctgattgtgtggtctgaagcagaaaattatgacttggcccttagctttcaagaaaaagctggatgtgatgaaatttgggagaaaatatgtcaggttcaaggaaaggacccttccgtggacatcactcaggaccttgtggatgaatctgaagaggagcgttttgatgatatgtcatcgccaggcttagaattgccatcttgtgaattaagtcgccttgaagaaattgcagaacttgtggcatcatctttaccttcacctcttcgtcgtgaaaaacttgcactggcactagaaaatgagggttatattaaaaagctcctggagctttttcatgtgtgtgaagatttggaaaatattgaaggactgcaccacttgtatgaaattatcaaaggcatctttctcttgaatcgaactgctctttttgaagttatgttctctgaagaatgtataatggacgtcattggatgtttagaatatgatcctgctttatcacaaccacgaaaacacagggaatttctaacaaaaacagccaagtttaaagaagtgattcccatatcagatcctgagctgaaacaaaaaattcatcagacatacagagttcagtatatacaagatatggttctaccaactccttcggtctttgaagaaaacatgttatcaacacttcactcttttatctttttcaataaggtagagattgttggcatgttgcaggaagatgaaaaatttctgacagatttgtttgcacaactaacagatgaagcaacagatgaggaaaaaagacaggaattggttaactttttaaaagaattttgtgcgttttcccaaacgctacagcctcaaaacagagatgcttttttcaagactttgtcaaacatgggcatattaccagctttagaagtcatccttggcatggatgatacacaggtgcgaagtgctgctactgatatattctcatacttggttgaatataatccatccatggtacgagagtttgtcatgcaggaggcacaacagaatgatgatgatattttgctcatcaacctcattatagaacatatgatttgtgatacagatcctgaacttggaggagcagtccagcttatgggcctgcttcgaactttagttgacccagagaacatgctagccactgccaataaaacagaaaagactgaatttctgggtttcttctacaagcactgtatgcatgttctcactgctcctttactagcaaatacaacagaagacaaacctagtaaagatgattttcagactgcccaactattggcacttgtattggaattgttaacattttgtgtggagcaccatacctaccacataaagaactacattattaataaggatatcctccggagagtgctagttcttatggcctcgaagcatgctttcttggcattatgtgcccttcgttttaaaagaaagattattggattaaaagatgagttttacaaccgctacataatgaaaagttttttgtttgaaccagtagtgaaagcatttctcaacaatggatcccgctacaatctgatgaactctgccataatagagatgtttgaatttattagagtggaagatataaaatcattaactgctcatgtaattgaaaattactggaaagcactggaagatgtagattatgtacagacatttaaaggattaaaactgagatttgaacaacaaagagaaaggcaagataatcccaaacttgacagtatgcgttccattttgaggaatcacagatatcgaagagatgccagaacactagaagatgaagaagagatgtggtttaacacagatgaagatgacatggaagatggagaagctgtagtgtctccatctgacaaaactaaaaatgatgatgatattatggatccaataagtaaattcatggaaaggaagaaattaaaagaaagtgaggaaaaggaagtgcttctgaaaacaaacctttctggacggcagagcccaagtttcaagctttccctgtccagtggaacgaagactaacctcaccagccagtcatctacaacaaatctgcctggttctccgggatcacctggatccccaggatctccaggctctcctggatccgtacctaaaaatacatctcagacggcagctattactacaaagggaggcctcgtgggtctggtagattatcctgatgatgatgaagatgatgatgaggatgaagataaggaagatacgttaccattgtcaaagaaagcaaaatttgattcataataatggcaacggcctaggatcagtacctgttgaaaaaaactggttctccacccctcccccatacaaaatccacaaaaaagcgcagtggtctcttgtgaatgactgacacagatcagcctcttacacttgacttctgctcatcaagtgccaattcaatggagcaggaggaggggatatcatatatttaggggaaagacttaagcctttgagctctccagcttggaccacacattgcccttttctcagggaaggaaatggaaacaaaaagccaacagggcaggggttttgtaagtggaactctggattgactggtcagttgctacaatcagaatatgctttcttggaccatgtttgagactcagaagaatgggcctttctgccataattcttcactagtcaagaatgccagcagtttctttgtataaagagacctgcctttaaaatcatacattctgaacattttagtcaagctacaacaggtttggaaaacctctgtgggggaggggcgagtataaagttttcctcttttttaactgttccctttgcccttcaaactgcagatatttttttttttaagtggggacttctccctacttgattaaagattgagtggaattctagatgtggtcatttgtgtcataattttttggttttatttggttttggatttttttttccctcccctgagttgtttgcttagttgtggcagttcttcttttttggcggacctttaaaacttttttggatgtatttataccctaacgttgtgctggtccctgttttcccatgtgtattttggtctacatccagttcttattggttcagagtttctgacgctgctca

SEQ ID NO: 63 - DNA, 1933 bp, Homo sapiens
  misc_feature: Incyte ID No 7504074CB1
cctaaggtagcgacttgctttctgacgagccacacgtttgcttcttccctgtgttcccagctggagggacatgagtgtccctgggccgtcgtctccggacggggccctgacacggccaccctactgcctggaggccggggagccgacgcctgacagaaccaagttgtattgttagacacactggaacaagagatttcaaaatttaaagaatgtcattctatgttggatattaatgctttgtttgctgaggctaaacactatcatgccaagttggtgaatataagaaaagagatgctgatgcttcatgaaaaaacatcaaagttaaaaaaaagagcacttaaactgcagcagaagaggcaaaaagaagagttggaaagggagcagcaacgagagaaggagtttgaaagagaaaagcagttaactgccagaccagccaaaaggatgtgaaaagttgtgtttgtgtgttttcttctcctgtcccatatttgggttatgatgactcaagtgtagactgaagttgaggtagtgccttatgccattatgtcatatgttgaaatccttattccggtattactgtgtctccatgccttttttccaagtagcagacgtcatgttgcatggtttttgatatttatatgtaagtttttcaaattttgcttaattttaaaatttattattttgatcttgaattatttataaactggaaagtggtttgattattgtgagtcaaaactctaagtggttaaaaattagtatgaattttttagcttcttaatgaatatggatttaaaactctccagttcttattttatgaaatgacttgcctttctggtaatacaatgctgattttttagtaattgccttttcattactttgttaagaagaaatgccagctgtttaatcacacctacccctggaaaagaggtaaaccttttgaacagttgaatttcatcagaagctctatagctttttggtgagaggaagtgatactctttattacaagaaacaaggaattaacaaaaataaggaacttgttgctacctttcttttctgttgaacattaaaaataatttggtttgtggactgggcatggtggcttatggaaaagaggtgagcctttgtgaagaacataatggaaaagtgcatgtacgggaaataacagcactttaggtgtagttcaggtgagcaagatgccagatagtttctactaaccatatactctttctgcttcttagcgtcgttgtcactgcttctgtggcttttgggttcaaagtagattatatatattcctaaagcttggtggaggtagtggagctatcacacactcaaatgagcttgtatatttataatacaatatagtattatagtaacagttatcatacactgagatatagtttctgtgcttgtaaaaaattcttgaagtgaggccgggtgcggtggctcatgcctgtaatcccaacgctttgggacgccgaggcgggtggatcatgaggtcaaaagattgagaccatcctggccaacatggtgaaaccccgtctctactagaaatacaaaaattagctgggtgtgggggcgtgcgcctgtagtcccagctacttgggaggctgaggcaggagaattgcttgaacccaggaggcagaggttgcagtaagccaagatcgtgccactgcactccagccttggtgacagagcaagactccgtctccaaaaaaaaaaaaaaaaaatttctctgaagtggaactgatgtacaccaaatacccactttcatagcataattacagggatggtgaccaattcagtacaatagcttcaccaaaagaggcagcgtggtaccttttaggtgtttctagaatctcttctggatattctatgtaagaaattcatcgttcgagggaactacgagggtttcattgtctaaaacatcacgcaagcg

SEQ ID NO: 64 - DNA, 4460 bp, Homo sapiens
  misc_feature: Incyte ID No 7502257CB1
gcggcgcgaacggcagctaggagggttgctccgggcttggtgctcactgcgacttcccgcgcagggcccggtcggactaggacccgcggcctgagagacgctggaggatgcggacgcggaggccgcctggggtagcggcggcgggagtcctggcgctctgcaggtcagaagttgagtagcaggggcctaggagggctcgaagccttcacagcgatggcagagaagcgacccctgagaaccctggggcctgtgatgtatggcaagctgccccgcttagagacagactccgggctcgagcacagcctgccccactctgttggtaaccaggatccctgcacctacaaggggtcctacttctcctgccccatggcgggtactcctaaggccgagtctgagcagttggcgtcctggaccccatacccacccttgtactctaccggtatggcaggacccccacttcaggcagacaacctgctgaccaactgcctgttctaccgctcgccagcagaaggccctgagaagatgcaggactccagccctgttgagctcctgcccttcagtccccaggctcactcctacccaggcccaccactggcagcacccaaacctgtctaccgcaaccctctgtgctatgggctctcaacttgtctgggggaaggagcagtgaagaggccactggatgttgactggactctggcgactgggcccctgttgccctcagctgacccaccctgctctctggccccagctcctagcaagggccagactctggatggcaccttcttgcggggggtgccagctgaggggtccagtaaagactcctcagggagcttctccccatgccagcccttcctggagaaatatcagaccatccacagcacgggcttcctggcctccaggtacacaggtccttaccctaggaactccaagcaagcaatgtctgaggggccctcaagtccttggacccagctggcccagcccctggggccaccctgtcaggacaccgggcccacccactacccaccaccccaccacccaccaccccaccctccacaggccctgccttgccctccagcctgtcgccacccagagaagcagggcagctacagcccagcactcccactgcagcctctggggggccacaaggggaccgggtaccaggctggtgggctgggcagcccctacctgaggcagcaggcagcccaggcaccttacattcccccactggggctggacgcttacccctacccctctgcccctctcccagcaccctctccaggcctcaagctggagccgcctctcactccacggtgcccattggactttgccccccagacactgagttttccttatgcccgggatgacctctctctctatggagcatcccctgggcttggagggacaccaccttcccagaacaatgtgagggctgtgccacagcccggtgccttccagagggcatgccagcctttgccagcgagccagccctgctcagagcctgtgaggcctgcacaggaagccgaagagaagacctggctgcccagctgcaggaaagagaagctccagccccggctcagtgagcactctgggccgcccatcgtcatccgagacagtccagttccctgtacccccccagcactgcccccctgtgcccgggagtgccagtctcttccacagaaggaggacgcaaggccacccagctctccaccaatgcctgtcattgacaatgtcttcagcctggccccctaccgtgactatctggatgtgccggcacccgaggccacaactgagcctgactctgccacagctgagcctgactcagccccagccaccagtgaaggtcaggacaaaggctgcagggggaccctgcctgcccaggagggcccctcagggagtaaacccctaaggggctcacttaaggaggaggtagccctggatttgagtgtgaggaagcccacagcagaggcctcccctgtcaaggcttcccgttctgtggagcatgccaagcctactgcagccatggatgtgccagatgtgggcaacatggtgtcagatctgccaggcctgaaaaagatagacacagaagcaccaggcttgcctggggtgccagtgaccacagatgccatgccaaggaccaacttccacagctctgtggccttcatgttccgaaagttcaagatcctccgtccggcacctttgcctgcagccgtggtcccgtccacgcccacctcagctcctgctcccacacagcctgcacccacccccacatctgggcccattggactgcggattctcgctcaacagcccttgtctgtgacctgcttcagcctggcactgcccagccctccagccgtagctgtggcctcccctgcccctgctccagctccatcccctgctccggctcgagctcaggctccagcttcagcccgggatccagctccagctccagctccagttgcaggccctgctccagcatctacttcagccccaggggactccctggagcagcattttacaggactacatgcgtccctgtgtgatgctatttctggctccgtcgcccactctcctccagagaagcttcgcgagtggctagagacggctgggccctggggccaggctgcgtggcaggactgccagggtgtgcaggggctgctggccaagctgctgtctcagctgcagcgcttcgatcgcacccaccggtgccccttcccccatgtggtgcgagctggcgccatcttcgtgcccattcacctggtgaaggagcggctcttccctcggctgccacccgcttctgtggaccatgtgctgcaggagcatcgtgtggagctgcggcccaccacgctgtcggaggagcgggcactgcgggagctcgccctgccaggctgcacctcacgcatgctgaagttactggcgctgcgccagctgccggacatttaccccgaccttctcggcctgcagtggcgcgactgtgtacgccgccagctgggtgagcatggggcagccccagtggccaccggagctgtgtgagcaagtgacaggtgactttgacactgaggctggagctgtgtcctcctcagagcccactgtggccagagatgagccagagagcctagccctggctcagaagtcaccggcccccaaggtcaggaagccaggcaggaagccaccaacccctggcccggagaaagcagaggcagctgctggggaagagtcctgtggtgcctcccctacccctgctaccagtgccagcccacctggccccacactgaaggcccgcttccgcagtctgctggagaccgcctggctcaatggcctggctctgcccacctggggccacaagtcctaaagaccagaccagccctcaccctgcccacagctgctggacagccagagccatcacctgtagcactggttgccagtgctgtgtgtatagcagtcactctccacccttcccttctgcctgcccagctgccccggggccacgagtggatgctggggctgtggctgctcccctggaggggttccatctctgaccctgtggcccattcagggtgggctgaagagcccctgagcttttaacgtgagggtctttattggataggactactccctatttcttgcctagagaacacacatgggctttggagcccgacagacctgggcttgaatcccggctcgtgttcttgctgcaggacctgggcaagaaacttcacctctgctgagccctcattccccatgtgtaaaatgggacaacgcaacctacctcacagggttgttgtggggatgctgcctgatacataccctgtcaccatttggtctctgcttcctctctgggacagggcctagaattggaggcagagaaccttcctatagaaagtcttcgtgtgtcctaggacttggctatcgtagagtggtaccttaggcagtggatgtgactcacactttcaggagtcaccccccagcatttggggttgggttggccctactccagcctggagctccctgagggagcctgcactccctgctcccaatccccgctactggtgcagggatgcagcctggagctggcgtccttgttctgggcctgctgctgccgccaccccaggaggccccaggcctgtcctgaattgacatcagtgcttccctgaactgcctcccccacccctggcattatcccaggaaacttatgttttctagaagctaagcagctgctgggactcagggactggtgcaggtaggctgagtggcagctcagtcctagaaggtctctgaagatctggactgaggaccctgctactccccaagccagagcccatcagccaggcctgctgtgagccacctgcctgtggagtgctgagctcaaccaaaggctggcaagctctgggcctcatttaagggattctgatgagccgatgggccctggaggcagcccattaaagcatctggctcgtttctgg

SEQ ID NO: 65 - DNA, 1214 bp, Homo sapiens
  misc_feature: Incyte ID No 1315136CB1
gaggaggagtggccctgatgaggacccgtagggcttcaaccgacccccagctcaaaaacacttcggcttgggggcggtgccaaggctgtgagtgcttccaacacttcggcttgggggcggtgccaaggctgtgagtgcggaagcctgccggaaacctggggacggagcgagggaaatgacgcctgggagcgacaacagggtgggtggggctgctgactccgcccccgaaggaaagggttaacggtttccggtagcggcgtctaggaggggcgggggaaaggaggcggcagccaggctgtgtcccctgaccgttggagcgtctgcgacccccgcatccccgcaccctcaaggcacctccaaagatgatgatgggttgtggggagtcagagctgaagtcggcggacggggaagaagccgcggcggtcccggggccacccccggagccccaagtcccgcaactccgagccccagtgcccgagcccggcctggacttgagcctgagcccgcggcccgacagccctcagccgcggcacggcagccccgggcggcggaaggggcgggcggagcggcggggcgcggctcggcagcggcggcaggtccgcttccgcctgacgccgccctccccggtgcggtccgagccgcagcctgcggtgccgcaggagctggagatgcccgtgctgaagagcagcctggccttgggcctggagctgcgggccgcagccgggagccactttgatgctgcgaaggccgtggaggaacagctgagaaagtcgttccagatccgctgcggcctggaggagagcgtgtccgaggggctgaacgtgccgcgctccaagcggctcttccgggacctggtgagcctgcaggtgccggaggaacaggttctgaatgccgcgctcagggagaaattggctctcctgccgccacaggctcgagccccgcacccaaaggagccacctgggcctgggccagacatgaccatcttgtgtgacccagaaacgctattttatgaatctccacacctgaccctggacggtctgccccctctccgacttcaactccggccccgcccttcagaggacaccttcctcatgcaccggacactgaggcgatgggaagcgtagaccccaaagatccctggagggctagttcgtatttttgtgttaaactatttgttagaataaagtaattttgctaataaaaaaaaaaaaaaa

SEQ ID NO: 66 - DNA, 2843 bp, Homo sapiens
  misc_feature: Incyte ID No 1379785CB1
agcgaccggcacagcctgccccgtcccgcctggcacgccctgattagcgccgggcacgacacgcaccgccccgcccggcactccctgcttcgcccacgacacgcccctccccgcccggcaaactgcccggagccaaggccccgcccccagagagaacctgccccgcctccctggcccgggccccgcctccttggcaagcgagtggcgctttcaccttagcaaccagcgcggctcccaccatggctgaagaagaggaaactgctgctctcacggagaaggttatccggacccagagggtgtttataaacctgttggattcctacagcagcggaaacatcgggaagtttctatctaactgtgtagttggggcttcgcttgaagaaattacagaggaagaggaagaggaagatgaaaataagtcagctatgctggaagcttcctcaaccaaagcgaaggaaggcacattccagattgtgggcacgctgtccaagcctgacagcccgcggcctgactttgcggtggagacgtactctgccatctctcgagaagaccttctcatgcgcctgctggagtgtgatgttattatttataacatcactgagagctcacagcaaatggaggaagccatctgggcagtctctgcactcagtgaagaagtcagccactttgaaaagcgaaagctatttattttactgtcgacggtgatgacttgggcgcgctccaaagccctggaccccgaggattctgaggttccattcactgaagaagattatcgaagaagaaagtctcatcctaattttctggaccacataaatgctgaaaaaatggttctcaaatttggaaaaaaggccagaaaatttgcagcatacgtagttgctgctggactccagtatggagcggaaggaggcatgttacacacattttttaagatggcttggttgggcgagattcctgcattaccagtttttggcgatggaacaaatgtaattccaacaatccatgttcttgatctagcaggagtgatacaaaacgtcatagatcacgtgccaaagcctcactacctggttgctgtggatgagtctgttcataccctggaagacatagtcaagtgtatcagtaaaaatactggccctgggaaaatccagaaaatacccagagaaaatgcatacctaaccaaggacttaacgcaagattgtcttgaccatttactggtcaacttaagaatggaagcgctctttgtgaaggagaattttaatattcgatgggctgcccaaacaggatttgtggaaaatatcaacactatcctcaaggagtacaagcaaagcagaggattgatgccaatcaagatctgcattcttggtccccctgctgtgggaaaatccagtattgctaaagaattggccaactactacaaactgcatcacatccaactgaaggatgtcatttctgaagccatagcaaaactggaggcgattgttgcccctaacgatgtaggggaaggagaagaagaagtcgaagaggaagaggaggaggagaatgtggaagatgcacaggagctcctagatggcatcaaggagagcatggagcagaatgcaggtcaactagacgatcaatatataattagatttatgaaagaaaagctaaaatcaatgccttgcaggaatcaaggttatattttggatggattcccaaagacctatgatcaagcaaaagacctgttcaatcaggaagatgaggaggaggaagatgatgtcagaggcagaatgtttccctttgataaattaattatacctgaattcgtttgtgcactggatgcttcggatgagtttctgaaggagcgtgtgataaaccttcctgagagcatcgtggcggggacccactacagccaagaccgattcctccgggctctgagcaactaccgggacatcaatatcgacgatgagactgtcttcaactattttgatgaacttgaaattcacccgatacatattgatgtaggaaaacttgaagatgctcagaatagacttgctatcaaacagctcatcaaagagattggggagcctcgaaattatggtttaacagacgaagaaaaggcagaagaggagcggaaggctgcggaggagcggctggccagggaggctgctgaggaagcagaacgcgagcaccaggaggccgtggagatggcagagaagatagctcgctgggaggagtggaataaacgactggaggaagtgaaaagagaagaaagagaattactggaggctcagtcaattcccctgagaaactatttaatgacctatgtgatgccaactcttattcagggcctgaatgaatgttgcaacgtccgacccgaagaccctgttgattttctggcagaatatctcttcaagaacaatcctgaagcacagtgaaacttgaaagatctggtattatctacctttacagaaccacagatcacttattatactttgaaaaattgctttgaaaaatgcttttccagttcttagaaaattctttttttgtagacaaatattctataaactagaatctctattaaaagctatatgacatgactatgtcattaaaactatatttgaaatgtaaattgataaagacatttgtgcatagctcatgagacaaatactgatttaatattttattctttagtcagatctaaatataccgcttctgtacactaatgtttataggtatttatagcatgaagaaaatcagactatatattgtagactatgtattattctaacatgtaggctaatttacatgacttgttatcgccccaataacaatgttattagaaatggaaataaattgaagtgatttat

SEQ ID NO: 67 - DNA, 1021 bp, Homo sapiens
  misc_feature: Incyte ID No 2011166CB1
agcggagntgggagcctgatggaggacaagtagggcctcgaggacaggtgcgtgacagaagcacaggaaaaaaaagaaaaatgaagaaataaaaacacgagttcatcagtaaagaggtaccctggcagcataaatattatgataagctaaaagctggaatcatctggaaaaataaataagactcctcatgtccttttcggtccataaccagaagggcagcaaaaggcctttgccactggaacctcttctttttctccaagtcccacgtagcaattacctgcactttcaagaagagaaacaacgactacacctaaagaaattccttcttgataggatgtttctagtggccaagatacaagcaaatgtagaaagaaaagatgttgctgactactatgaacaaatgtttcagtcagttttgaaacatcacctaggagaagcagtgacaggattgctgctcatctatcccacttccattctgcatatcctcgagtcctccagcgacactctctacaaagttcttttagattatattggccatgtcaaagatgaaacagtattttttattcaacaaatgaaaattatagtcatttctcataacattccaatgaggctttttatgcaatggcatgtttcagtgataaaagttccagttatgtatctcgacgatgtgacacagtcacagtccctaaaggaggtcatcacagattttctcacacaaactcataaactgtcactctacctttgccagactatgaaagtaggcactaaaggaccaggcgataacttacaccaagttgcacctgacctactcctcccagaacaaatcataaagtacttgtgcaaatccgaagaattcatggacccggcaacatttataaacatgtataatagacccatacacatcactctggattctgaggtggtatggcctgctccttcacgtttctaggattgagagggataatgtgcccatgtctcttaaggagtttgtgctacttaaataaaaaaaacatttttaaag

SEQ ID NO: 68 - DNA, 4074 bp, Homo sapiens
  misc_feature: Incyte ID No 3434684CB1
ggcggccgagcgaaatataactcatatatggcgaacttgtgcttctagatcatgcgtcgagcggcggacgccagttgtgcgtgcaaacgggatagacgggtgggccgaggtacaggccccacggccgccgtctcccgcttctgcccgcgcagagtccgcgccatggccgcctcgccgggctcgggcagcgccaacccgcggaagttcagtgagaagatcgcgctgcacacgcagagacaggccgaggagacgcgggccttcgagcagctcatgaccgacctcaccctgtcgcgggttcaatttcagaagcttcagcaactgcgccttacacagtaccatggaggatccttaccaaatgtgagccagctgcggagcaatgcgtcagagtttcagccgtcatttcaccaagctgataatgtcggggaacccgccatcacgggctggtggagaggccatccaggaaccgttccaccccctccaccgaaggtctggggacaagccaggtcgacaatttgatggtagtgcttttggagcccaattattcctcacagcctctggatgagagttggccaaggcagcagcctccttggaaagacgaaaagcatcctgggttcaggctgacatctgcacttaacaggaccaattctgattctgctcttcacacgagtgctctgagtaccaagccccaggacccctatggaggagggggccagtcggcctggcctgccccatacatgggtttctgtgatggtgagaataatggacatgggaagcatctttccactggcccattgacaagaagagaatctgttaaatgttccgaagccactgccaaaacaactgtgggagaccacggagattcagtcctgtcaggacgccctcgatcctgtgatgttggaggtggcaatgcttttccacataatggtcaaaaacctaggcctctcacccttcttggggaccttgaacacttggagggtcattgccagatctaaccaaaccccactactcgacacccctgccagcctccctggacaccaccgaccaccactttggcagtatgagtgtggggaatagtgtgaacaacagcccagcagccccacagtcagactttcagcttctcccggcccagggctcatctttgaccaacttcttcccagatgtgggttttgaccagcagtccatgaggccaggccctgcctttcctcaacaggtgcctctggtgcaacaaggttcccgagaactgcaggactcttttcatttgagaccaagcccgtattccaactgcgggagtctcccgaacaccatcctgccagaagactccagcaccagcctgttcaaagacctcaacagtgcgctggcaggcctgcctgaggtcagcctgaacgtggacactccatttccactggaagaggagctgcagattgaacccctgagcctggacggactcaacatgttaagtgactccagcatgggcctgctggacccctctgttgaagagacgtttcgagctgacagactgtgaacagaaggcagtggaacagaagaatgtttttctgcaacagccaaaatagaatggaatagaatgaagccagctgataccacgggctttcgttatcttgacatagaaggaagcaatgccacggctccagggtttcagatgagatcccatctcagacactgtggcttcctccagatcacacagctttgtactgcctctcccgcctgtggccaaagtcgtgttgcagcaggcaggctgcttggagcttcccatgaactggaaagctcacctccactgcatctttttactggccatccagtcagccgatgtgtaagagtaggaaatactgtgtcactggaggcctccgtagcattgtgtagtgtgctcagaaccactgatctccgtccgcaccgaaggcgggcccggagtgggaggctcggcctggggcggcggcaccggagagggcacctcgatgcctgctctgacctgacccagagggcgaggccctccagcgggggacattcccaggctgagtggaccccacggctctctcccacgcctggattacgacatgaagtttttaccacaagcccgagggcaggcttgagttaggcagactgaaggctaattttcattttctcccagctggtttctgctgcttcagaaaagtacaccttttccttatggaccagaggaagaggaagaccattttatcagtcactgaaaagagtcccctggcactgatgagcctgaagagaactgtgctcctcctcgggggctggggaaggaagacagggactgcaggtgtctcatactcagtggcctccagacaaactccagacaagcacagacctccccactaagagcagccagagggagctggtgaggccctaaccccacccaccgagcaactgagcttccccatccctccccagagctgtgtctctgtgggctgggagtctaatgtcacccccctagaccgtaagctccttgagggcagggacagtgccttatcattgttgaacccgtagcacctaacacgtgcgtggcagcacagtcgtgtgctggagtgagtgctgcagaaccgtgcgtgcagcgcatgatgaatgagtgcgtccgccatgccgtaaggcaggctcacctgtagctatccccttccctgccagatcttctcagagctttagcttttctagcactcgtgcctatggtgaagcatgcactttaatatgcttttaacactaggtgaccaaatcacagtgaagccgggcactgcattctccttgggctgtgctccacgcgggtgggtgggagctgtcttctgagtacatccggaagggctgagcaggtgagtgccctgagcatcctggctgggccccacccaggaagatcttccttctcagatccacgtttggctctaaattgcttcaagtagagattcattctttgaggttgaaaaaatagtttatagtaaaacgaaggcctaattcatggaagcatcattagtcatcaataccttctcataaaatagatgggccagatttccaccaccgcctgcctcctctaacttgggtgatgccagtaggtttgaagggggcagagcactgcagggggaggggggggtctaggctgtgagaggacagggtggagaggaggagaaccctgaaggagagacggaagatgccaggacctttgcttggacagccattgcccttccaggagaccctggagtgtactgagggttgctggactgttccacccagaggagcaaggctgtacaatgagggtctgaatctggcacacctgtcccttatgtaaaaggagtcgtggtcacaagaccctgggctggttagcctctcccgacctcattccatttctatcttctgaccttgcctctcatctttaaaataaccctcatggggtgcccctccaccttcctctggaatccaagtattcctgtttcacatttgccccaaatctttgctgtggaattgggaaatcaaaccagagtcctcctcgcctgatttccagctcaggaagggcctgctggcctgccctgttcccagttacactttcagatccctttgtgctcaagatctcagagggggtggctttttgttaaagagccttcagtcgcaatgctacccagcaccccatgtgccaaaagaaaccagctcctgtgtcaaagggcttccaaacctgatctcactctcaacaggcgatggtgctgatgtttcaagaattgtgtttttataaaacagaggtctcagcatagtcactcttcacatagtgccttacccatgggtatcgtcatatccgggtcccagttcagtttgtctgcacagcagccaccctgcctggcaacagagaccccaagactacacagtgaaccctactgccccaaaggcgttctccaggtgacttgtgaaaacagacctccggggaagtgatttatgggggtgtacactgggggcatatggtttagcactgaattcaatttgtcttaggtctatgagtgagtcgatcttttcttgtgaaggtttgggctcgttacaaccactctgcctagaggtgtgggg

SEQ ID NO: 69 - DNA, 2551 bp, Homo sapiens
  misc_feature: Incyte ID No 5134056CB1
gcgctaggcagcttcagccggaccgggtaggggtcctcgctcgctagcttgctgtttctcggagaagctcccgagtgtccggcctagaggccatgagaaggcagtgggggtctgccatgagggcggccgagcaggcgggctgcatggtgagcgcctcccgggccggacagcccgaggcgggcccgtggagctgcagcggggtaatcctgagccgtagcccgggcctggtgctttgccacgggggcatcttcgtccccttcctgcgagctggcagcgaagtcctgaccgcggccggcgccgtcttcctgcctggcgacagttgcagggacgacctgcgcctgcacgtgcagtgggccccaacggccgcgggtcccgggggcggcgcggagcggggccgcccagggctgtgcacgccccagtgcgcgagcctcgagcccggcccacctgccccgtcccgcgggcgtcccctgcagccccggcttcctgctgagctgctgctgctgctgagctgcccggccttctgggcccacttcgcgcgcctcttcggggacgaggcagcggaacagtggcgcttctcgagcgcggcgcgggatgacgaagtgtcggaggacggggaggcggatcaactgagagcgctgggctggtttgcgctgctgggcgtgcggctaggccaggaggaggtggaggaggagcgcgggccagccatggcggtgtcgcctctcggggccgtgcccaagggtgcgccattgctggtctgcggctcccctttcggcgccttctgccccgacatctttctcaacacgctgagctgcggggtgctcagcaacgtggccggcccactgctgcttaccgacgcacgctgcctgcccggcaccgagggcggcggcgtgttcaccgcgcggcccgcgggggcgctggtggcgctggtggtggcgccgctctgttggaaggccggcgaatgggtgggcttcacgctgctctgcgccgccgccccccttttccgcgccgcccgcgacgcgcttcaccgcctgccgcacagcaccgctgccctggccgcccttctgccgccagaggtgggcgtcccgtggggtctgcccctccgagactccgggcccctgtgggcagccgcggcagtgttggtggagtgcggcaccgtatggggctccggagtggctgtggcaccccgccttgtagtgacctgtcggcacgtgtcccctcgggaagcagccagggtcctggtgcgctccaccacccccaagagtgtggccatctggggccgtgtggtatttgccactcaggagacatgtccctatgacatagcagtggtgagcctggaggaggacctggatgatgtccccatccctgtgcccgctgagcacttccatgaaggcgaggctgtgagtgtggtgggctttggcgtctttggccagtcttgcgggccctcggtgacctcaggcatcctttcggctgtggtgcaggtgaatggcacgcccgtaatgctgcagaccacgtgtgctgtgcacagcggctccagtgggggacccctcttctccaaccactcaggaaacctccttggcataatcaccagcaacacccgggacaataatacgggggccacctacccccacctgaacttcagcattcccatcacggtgctccagccggccctgcagcagtacagccagacccaagacctaggtggcctccgtgagctggaccgcgctgctgagccagtcagggtggtgtggcggttgcagcggcccctggcagaggccccgcggagcaagctctgaggctgtgttaccacctttggaaagaagagtgacctttttctgctgtaggaagtgatgttgaggtgacggtggcctcaggattcagggcccagcccctgcaggggcccaggctgcctctcatctccacccactgactgcagactgggctttgggctctggggcaaacttctcttcagccccatggatccttaacctggcagcccgttttggggtgctttcttgagcccccagttctctgtcccctagcactagactcagctgtattgtttttccttctggggagcccactccaactgcacagaagttctgggcctgacaggtagattccagctggaaggcaggcccgtgcctggttttgcgtctgttcccctgagggccatcgtcatcctggagcttcaatggggccttggctcctgtctgcctctcagtcagagtcagggctgacaaaggactcagcttccttagcatctcagcagaaaccttgctctgaagaccagagacagaagggacagaaacaggagtgcctcctgctgtgccaggcccatgggcagtgcaggcagatccctgaaggtcagcactcctgggtcttcatatgccaacaggggcgctcttgacactgtgccttcattttccagcccacagcctgggtctcagggatcttgaggggtagaacatgtctggttggggcttgggaataaacatgatctattgaaaaacctctgtcaaaaaaaacaaaaaaaaacaacaaaacagaaaat

SEQ ID NO: 70 - DNA, 1115 bp, Homo sapiens
  misc_feature: Incyte ID No 5281724CB1
ggcgccgcagtgtctacacccgcaaaaacgcccgctttgacttagagccctccgcatatccttcccctggctggggcagccaaggcgcggaggacagctccgaggccagatgtttactgcgcagatgcccgactttacaccggggtccgggctgtgccgggcgctgggggaagcgcccaccttccagagagcgaaatcatggagccttccaagaccttcatgagaaacctgccaatcacaccaggctatagcggctttgtgccattcctcagctgccaaggaatgtccaaggaggatgacatgaaccactgtgtgaaaaccttccaggagaaaacacagcgctataaagaacagctgcgggaattgtgctgcgcagtggccactgccccgaaactgaaacctgtcaactccgaggagacggtcctgcaggccctgcaccagtacaatctgcagtaccaccccctgatcctggaatgcaaatatgtaaagaaacctctccaggagcccccgatccctggctgggcaggctacctgccgagagccaaggtcactgaatttggctgtggcacgagatacactgtcatggccaaaaactgctacaaggacttcctggagatcacggagagggccaagaaggcacatctgaaaccatatgaagaaatatatggagttagctccacaaaaacttctgctccgtctccaaaagttttgcagcatgaagagctgctgccaaaatatcccgatttttctattccagatggaagctgccctgcccttggaaggcccctgagagaggaccccaaaactccgctgacatgtggctgtgctcagaggccaagtataccatgcagtgggaagatgtatctagagccactgtcctccgcaaagtatgcagaaggctagaagcgcagagtctcccaaggaggtgaactttaagtggggcttccaaaacctgccattctcatgttggaatcacgcccagtgagcaataaagaaatttagtaacaagaattttttatctgccgcctgcatcctgagtggttcccggttgcatgtcattaatgataaaggccgttctttgtcatgtcggaataaagagggtgcttctccgcaaa

SEQ ID NO: 71 - DNA, 1334 bp, Homo sapiens
  misc_feature: Incyte ID No 7502391CB1
cgcgagccgggctgtcgggtgtgttttgctctccatcctccgtcgtctctgcagcactccgggttctcctccagagcgctagtcccaggagctcggaatgttcgtggaacttaataacctgcttaacaccacccccgacagggcggagcaggggaaactgactctactctgtgatgccaagacagatgggagtttccttgtacaccactttctctccttctatctcaaagctaattgtaaagtctgctttgtggcactcatccagtccttcagccactacagtatcgtgggacagaagctgggtgtcagcctgaccatggcgcgggagcgtgggcagcttgtgttccttgagggactcaagtctgcagtggacgtcgtcttccaggctcaaaaggagccacaccccctgcagtttctcagggaggctaatgctgggaacttgaaaccattgtttgagtttgtacgggaggccctgaagccagtagacagtggagaggctcggtggacgtacccggtgctgttggtggacgacctcagtgtgctcctgagcctgggcatgggggcggtggctgtgctagacttcattcactactgcagagccaccgtgtgctgggaactaaagggaaacatggtggtccttgtgcacgacagtggagatgcggaggatgaggagaatgacatcctgctgaatggcctcagtcatcagagccatctgatactgcgggctgagggcctggccactggcttctgcagggatgtgcacgggcagctgaggatcctgtggaggagaccatcgcagcccgcagtccaccgggatcagagcttcacttaccagtataagatacaggacaaaaggcgtgtccttttttgccaaaggaatgtctcctgctgttctgtgacctgatttcggagcagctgaagctacataggactgtttttggacgtggaagatagagcaacatagcaagaatgggtctttctcctctgtagtaatatttcaggctggaccggcgactccactgtgaccagagggttgagtgctgcagtgatggcatgccttggctgcctgggccctgttcagaaaacacaagggaccacaatcctgcctttgctgagagagaggctggatgctagacccaagtgaaaggggtcctttggagcctttgtttaaatatgccttagccccagctgcccatttttggttgacaagcctttcagagccagagtgggtatagatgtgccagccaggagatggcaccggatggcaggtgtgcaaggtgacaactaggataatcatggctggaataaagtaagtttccacactggaaaaaaaa

SEQ ID NO: 72 - DNA, 2387 bp, Homo sapiens
  misc_feature: Incyte ID No 7502544CB1
cgctcgcgccggaccggaaagccggggaagtggccgaggagggagggctgcgagccatggcgaccaagacggcgggcgtggggcggtgggaggtagtgaagaagggtcggcggcctggggtcggcgccggcgccggcggccgaggaggcggcaggaaccgcagggcgctcggggaagcaaacggagtgtggaaatacgacctgacccctgcaatccagaccacaagcaccctttatgagcggggctttgagaatatcatgaagcggcagaataaggagcaggtcccaccccctgctgtggaacctaagaaaccagggaacaagaagcagccaaagaaggtggcaactcctcccaaccaaaaccagaagcagggccgcttccgcagcctggaggaagcactgaaagctctggatgtggcagacctgcagaaggaactggacaagagccagagtgtgttctctggaaacccatccatatggttgaaggacctggccagctatctcaactacaagctacaagctcctctaagtgaacccacgctgagccagcatactcatggcctatgggccttagattatccctacagcctggtgagccgggagctacgtgggatcatccgagggctgctggcgaaggcagcagggtctctggagctcttttttgaccactgtctgttcaccatgttgcaagagctggataagacaccaggggagtcactacatggttaccgcatctgtatccaggccatcctgcaagacaagcccaagattgccacggcaaacctaggcaagttcctggaactgctgaggtcccaccagagccgaccagcaaagtgtctcaccatcatgtgggccctgggtcaagcaggttttgccaacctcaccgagggactgaaagtgtggctggggatcatgctgcctgtgctgggcatcaagtctctgtctccctttgccatcacatacctggatcggctgctcctgatgcatcccaaccttaccaagggcttcggcatgattggccccaaggacttcttcccacttctggactttgcctatatgccgaacaactccctgacacccagcctgcaggagcagctgtgtcagctctacccccgactgaaaatgctggcatttggagcaaagccggattccaccctgcatacctacttcccttctttcctgtccagagccacccctagctgtccccctgagatgaagaaagagctcctgagcagcctgactgagtgcctgacggtggaccccctcagtgccagcgtctggaggcagctgtaccctaagcacctgtcacagtccagccttctgctggagcacttgctcagctcctgggagcagattcccaagaaggtacagaagtctttgcaagaaaccattcagtccctcaagcttaccaaccaggagctgctgaggaagggtagcagtaacaaccaggatgtcgtcacctgtgacatggcctgcaagggcctgttgcagcaggttcagggtcctcggctgccctggacgcggctcctcctgttgctgctggtcttcgctgtaggcttcctgtgccatgacctccggtcacacagctccttccaggcctcccttactggccggttgcttcgatcatctggcttcttacctgctagccaacaagcgtgtgccaagctctactcctacagtctgcaaggctacagctggctgggggagacactgccgctctggggctcccacctgctcaccgtggtgcggcccagcttgcagctggcctgggctcacaccaatgccacagtcagcttcctttctgcccactgtgcctctcaccttgcgtggtttggtgacagtctcaccagtctctctcagaggctacagatccagctccccgattccgtgaatcagctactccgctatctgagagagctgcccctgcttttccaccagaatgtgctgctgccactgtggcacctcttgcttgaggccctggcctgggcccaggagcactgccatgaggcatgcagaggtgaggtgacctgggactgcatgaagacacagctcagtgaggctgtccactggacctggctttgcctacaggacattacagtggctttcttggactgggcacttgccctgatatcccagcagtaggccctgccttcctggccactgatttctgcatgggtagaccatccaagactgcagcgggtagaaggtggcagttcttcatgggagtctttttaacttggtgcctgagttctctcctaggcaagtggccagttgcctccgcctcagttcttccatctttggtggggacagggcccagcagcatctcagcctcctacccacaattccactgaacacttttctggccctactgcacatggcccccagcct

SEQ ID NO: 73 - DNA, 2919 bp, Homo sapiens
  misc_feature: Incyte ID No 2858465CB1
gtagatgcgatggcgccgattccaaagactgtggggcggatcaagctagactgctctctacggcccagctgcccactggaggtcgctgctgcacccaaactttgcaaggaattcggtccagaggattacggcgaagaggacatagtggattttcttcgacggcttgtggagagtgatccccagggcctgcaccggatccatgtggatgggagcagcgggcggctgcagctgtggcaccatgattacctcctgggccacttggatgatgaagggaaatcaactggacagagtgacaggggcaagggggctgagggactgggcacctactgtggtctccgcaagtccttcctgtatcctccccaagagtctgagccctgccctcaaagcccctctgcctctgccaccttccccagtgtctcagacagcctgcttcaggtggccatgccccagaagctcctggtgacagaagaggaagccaatcgcctggctgaggagctggtggctgaggaggagcgcatgaaacagaaagcagagaaaaagcgactcaagaagaagcgtcaaaaggaacggaagcgacaggagcgtttggagcagtactgtggggagcccaaggccagcactacctcagatggagatgagagccccccatccagccctggaaacccagttcagggacagtgtggtgaagaagaggactcactggatctatctagcacttttgtgtctctggctttgcgcaaggttggggattggcccctcagtgcccgcagagagaagggactgaaccaggagccccaaggcaggggtctggccctccagaagatgggtcaagaggaagagagccctccaagagaggagaggccccagcagagtccaaaggtacaggcatctccgggactgctggcagctgccttacaacagagccaggaactggcaaagttgggtaccagctttgctcaaaatggtttctaccatgaggccgtggtcctcttcacccaggccttgaagctcaacccccaggaccaccggttatttggaaatcgttccttctgccatgagcggttgggtcagccagcgtgggccctggctgatgcccaggtggcccttaccctacggcctggctggccccggggcctcttccgcctgggcaaggccttgatgggactacagcgcttcagagaggcagctgctgtgtttcaggaaactctgagaggtgggtcccagcctgacgcagcccgagagctccgctcttgccttctccacctcacactgcagggtcagcgaggaggaatctgtgcaccacctctgtcacctggggccctccagccacttccccatgctgagctggcaccctcaggcctaccttccctcaggtgccctcgaagcactgctttgaggtcccctggcctgtctccactcttgcattatccttcatgtcaccgaagccaccccaaccagcccctctcccggactcagagtagaaggccccatcctctcaagccccaggacccttcaaagggctgggacatcctgggacttgggctccagcatctgtctcaggccagatgagggggcaccggtccctcatagggcagggccatgtatatatcccttggtgggggacatagtgtggtgacagttcactgcatattttgagaccttattctctagatccatagttaatgatgccctggcagtcattcctcttgccatggggaagcttctgatgagagaaaggagccccacatccactgaaacatcctttggttctcaagcttcttctggaggcagtaaggaaaaataaaacccaccaaggctcaagaagggaactatagaaaagttcaggtttttaggctatagcagagacagtgagaaagcatctgggcctttctcttcctcttggtccaggggacctcattcaccaactagagcttggtgtacaggaacggggtcacagtgctgagggggcttgagtcccacctttcagcttgatggatgctcacctcttctcagccccagctcgtgccctgtttttctagccatagcccccagattactcacagctcctcatgccatttcctgtccagattgctatgtatgactctgacctctcttgtccagtggtctggtgctcacctcctctcactgctagaatattcaccaagggtttgcatttgggaagtcccttaccagctcctgcttagagctggtagggccatacatgtccacactcccaactggtggctctcccgctgaatggggcctcagcaggtgcccaagctgctacaaccttggccactctgtttctccaccccagcactgggcatggtaattagcctttccccatgttaatttattcagttttttcaagggtcaactgaattccccacttcctgggtaagaagcatgatctccttttaatttcacgtctaagatcctggcagcttcccctagctggttcctctgtagtcctgctgggactgtcagctcatttaaatgtgggtctgcagaaggctttaggtctcccccaacccccttacctttcacagaggaacctttcatcaggataaatgattattgctgccctgtgggtcttgctcaatactgttcatacctggagagagaaggtattgaaacatctcctttatgtgtgactttcccaaatttttaaaaattgtttatggtttaggccccttaaatactgtgtagcaggatgaagtctaccattaccagctgggtcaccttggatgggtctgtcaacatctaagcctcagttccctcacctgtaaaaatgagggtagtccctacctcataagggatattgtgaggatggaaagcgaaagtgtgagaaaatacctcccaagtgcctggtacatagtgggtgctaaataaaccactttttgtctgcaaaaaaaaaaa

SEQ ID NO: 74 - DNA, 1414 bp, Homo sapiens
  misc_feature: Incyte ID No 7503455CB1
ggcgaagaggggcgcaagctcattgcgttttgagtctcgggacccctgttggagagactatggcgctcaacaagaatcactcggagggcggcggagtgatcgtcaataacaccgagagcatcctaatgtcctatgatcacgtggaactcacattcaatgacatgaagaacgtgccagaagccttcaaagggaccaagaaaggcactgtctaccttaccccttaccgggtcatctttctgtccaagggcaaggatgccatgcagtccttcatgatgccattttatctcatgaaagactgtgagatcaagcagcccgtatttggtgcaaactacatcaagggaacagtgaaggcggaagcgggaggtggctgggaaggctctgcttcctacaagttgactttcacggcagggggcgccattgagttcggacagcggatgctccaggtggcatctcaagagttctatccaggaccccccatgatggacggggccatgggatacgtgcagcccccaccaccgccctaccctgggcccatggaacctccggtcagcggccccgatgtcccctccactcctgcagccgaagccaaggccgcagaagcagccgccagcgcctattacaacccaggcaatcctcacaacgtctacatgcccacgagccagccgccgccacctccctactacccaccggaagataagaagacccagtaggccctcctgcctccctgcctcccaccctcatctctctaccctacccctcccatcggggctgtgctggggcttggggaggggagggggcgccttgttctccctccaggtctgatcataaacaattaccaggaactagcattgtgggacattagggcccccggcctcgggagaggtgccgcccagcttcccatgccagcccggagcccacagtgctgcccagcgtacctccctcaccgtctggggctcttctgggagcacggagcatcccctgttcctgtttcactctcagcttctcccctcgaagggactctctggccacctcctccaccgcagtccagctccctcagtctggcacccactgctacactcagcctcatgagccacttcagaccagccaggtgtcttcccgggccctgccagaccctgctcacattccctctgctggtctgtgctggtctcagaaggccaccgcgcccgcattccactcagccagggtccagctgcagcccccgccacccttccttcccttccctgtcctgggtcatgttgttgccaccctgtgtgacttttgaagctgtaaaatgagcttccagggcttggggtggcgtcggggcagggccgccgaggctggggaggaagcccttctgccttttgctggtgtttctggaatttgctttccctcacctctcacttccttctagaag

SEQ ID NO: 75 - DNA, 672 bp, Homo sapiens
  misc_feature: Incyte ID No 7503479CB1
ggcgggtcacgtgacgcggtgcctggcgccgagcctcccaagatggcggtgtgcatcgcggtgattgccaaggagaattaccccctctacattcgcagcacccctacggagaacgagctgaagttccactacatggtgcacacatctctggacgtggtggatgagaagatctccgcaatggggaaggccctggtcgaccagagggagctgtacctgggcctgctctaccccacggaggactacaagatgttccggaagctacacaactcctacacagacgtgatgtgcaaccccttctacaacccgggggaccgcatccagtccagcagggcctttgataacatggtgacgtcgatgatgatacaggtgtgctgagtgagctgtgctgccagccatcgcagaggagcccgcgcacgactgtggtggggccgtcggtctgttctggttgcctcttcctgaatgggacgcctggggctttcagggcaggcagctgtgcatgttctctcaactaaaggtcttgtgagaggagatttggctttttccttccgtgtcagccaaggacttaattaagaagaattcaactaaggacttttctggggttgtgggcagaggttgggatcagatggcgcacgtagcctgtctcagttgcccaaaggggcagagcagggtgca

SEQ ID NO: 76 - DNA, 3056 bp, Homo sapiens
  misc_feature: Incyte ID No 7218127CB1
ccggtcgggggcggggccgggctcggcttctcttagggacccggcactcgtcgccctcagtccggctcagctggggctgggcgccgtgggtctggcggttccgtagcggtcccagcgtctgtcccgccggccgggcggtcgcggcacagccgcgggaaggtgtcggagggcggttccgccgcgcggcgggcgcgccgcccacatggcggccatcagggcgctgcaacagtggtgccggcagcagtgcgagggctaccgcgacgtgaatatctgcaacatgaccacgtcgttccgcgacggcctggctttctgcgccatcctgcaccgccaccggcccgacctcataaacttcagtgctctcaagaaggaaaatatttatgaaaacaataaactggccttccgcgtggccgaggagcacttgggcatcccagccttgctggatgccgaggacatggtggccttgaaggtgcctgaccggctgagcatcttgacctacgtgtcccagtattacaactacttccacggccgctcccccattgggggcatggcaggcgtgaagagggcctcggaggactctgaggaggagccgtcagggaagaaggctccagtccaggcggccaagctgccctcgcccgccccagcccggaagcctccactatctccagcccagacaaaccctgtggtccagaggaggaatgagggtgcagggggcccgccccccaagactgaccaggcattggcgggcagcttggtcagcagcacctgcgggtctgcggcaagcacgtgcacctggtacagcggcacctggccgacgggaggctttaccaccggagctgcttcaggtgtaagcagtgctcctgcacgctgcactcgggggcctacaaggccacaggagagccgggcaccttcgtctgcaccagccacctccccgcagccgcctctgcaagccccaagttgacgggtctggtcccccgacagccaggggccatgggtgtggattccaggacctcctgttccccacagaaggcccaggaggcaaacaaggccagaccgtcggcctgggagcctgctgcgggcaactcgcctgccagggcttccgttccagctgcacccaaccctgcagccaccagcgccacgtccgtccacgtgaggagcccagccaggccctctgagagccgcctggcccccactcccacggaggggaaagtccgccctcgtgtgaccaatagctccccgatgggctggtcgtcagctgccccgtgcacagcagcggctgcctcccatcccgccgtgcccccgagtgccccagaccctcgcccggccacaccccagggcgggggagccccccgagtggcagctcctcaaaccacactcagttcaagctccacatctgcagccacggtggaccccccagcctggaccccgtccgcctccaggacccagcaggcccggaataagtttttccaaacatcagcagtgccccccggcaccagcctttctggcagaggtcccaccccgtcacttgttctatccaaggacagcagcaaggagcaggcgcggaacttcctcaagcaggccctctcagcgctggaagaggctggcgctccggcgcctggcaggccctccccagccactgccgctgttcccagttctcagcccaaaactgaagcaccacaagcaagtcccttagccaagccgttacagtcctcgtctccccgggtgcttggcctgccttcgaggatggaaccgccagccccgctgagcacgagcagtacctctcaggcatccgcgttgcccccggcaggcaggaggaacttggctgaatcctcaggggtcggcagggtgggtgctggctccaggccgaagccagaggccccgatggcaaagggtaaaagcaccaccttaacgcaggacatgagcaccagcctccaggaaggccaggaggacgggccggcaggatggagagcgaatctgaagcccgtggacaggagaagcccagctgagaggactctgaagcccaaggaaccacgggccctggcagagccgagggcgggggaggcccccaggaaggtctcaggcagctttgctgggagtgtccacatcaccctgacccccgtgaggcctgacaggaccccacgcccagccagcccaggacccagcctcccagccaggtccccctccccaccccgccgcaggagactggccgtccctgccagcctcgacgtttgtgacaactggcttcggccggagccccctggccaggaagcccgagtgcagagctggaaggaggaggagaagaaacctcaccttcagggcaaaccagggagacccttgtccccggccaatgtccctgctctgcctggcgagacggtgacctccccagtcaggctgcaccccgactacctctccccggaggagatacagaggcagctgcaggacatcgagaggcggctggacgccctggagctccgcggcgtggagctggagaagcgactgcgggcggccgagggagatgacgctgaggatagcctcatggtggactggttctggctcattcacgagaagcagcttctgctgagacaggagtcagagctgatgtacaagtccaaggcccagcgtctggaggagcagcagctggacatcgagggcgagctgcgccggctcatggccaagcccgaggctctgaagtcactgcaggagcggcggcgggagcaggagctgctggagcagtacgtgagcaccgtgaacgaccgcagtgacatcgtggactcgctggacgaggaccggctccgggaacaagaggaggatcagatgctgcgggacatgattgagaagctgggcctccagaggaagaagtccaagttccgcttgtccaagatctggtcaccaaaaagcaaaagcagcccctcccagtagtagccagtagggccgtgggctcggcccggacctggcatccggacttggactccgggtcaagcccatggccccgagtccacgtccgactgccaggtccggagcgcgacgcgtggaaagccgaattccagcacactcgggcc

SEQ ID NO: 77 - DNA, 1891 bp, Homo sapiens
  misc_feature: Incyte ID No 1688943CB1
gtggaagctggcctggcccccggagctccctggagtcggtactgggggcttcgttttgtacgcaccgttttctctctgtgctatgggagatgtcaaggaatcaaagatgcaaataacaccagaaactccaggaaggatccctgttttaaatccttttgaaagtcctagtgattattctaatctccatgaacaaactctcgccagtccttctgtttttaaatcaacaaaattaccaaatagataaagatgtggaagacaaaagacaaaaagccattgaagagtttttcactaaagatgtcatcgtaccctctccttggactgatcatgaagggaaacagctttcacaatgtcattccagtaaatgcactaacataaatagtgactctccagttggaaaaaagctgaccattcattctgagaaaagcgatgctgcttgtcagacattgctgtctcttcctgtggattttaatttagaaaatatattaggtgactattttagagctgatgaatttgcagatcaatctcctggaaacctcagttcttcatccctcagaagaaagctgtttttagatgggaacggaagcatctccgactccttaccttcggcttctcccggaagtcctcacagtggtgttcaaacatcactagagatgttttattcaatagatttgtctcctgtaaagtgtaggagccccttgcagacaccaagttcggggcagttttcttctagccctattcaggctagtgcaaaaaaatacagcttgggaagcataactagtccttcgcctatttcttcacccactttctcaccaattgaatttcagataggagagactccactctcagaacaaaggaagtttactgttcattctcctgatgcttcatctggaacaaattctaatgggataactaatccgtgtatcagaagtccttatatagatggctgctcgccaattaaaaattggtctcctatgagacttcagatgtatagtggtggtactcagtatcggacctcagtgattcagataccttttactcttgagactcaaggtgaagatgaggaagataaagagaatattccttccacagatgtctcatcacccgccatggatgctgctggaatacacctacggcagtttagtaatgaggcttctacccatggtacacatttggttgtgactgccatgtctgttacacaaaatcagtccagtgcttctgagaaagaattagcactgttgcaggatgttgaaagggagaaagacaataacactgtggatatggttgatcctatagagatagcagatgagaccacttggattaaggagccggttgataatggcagtttacccatgactgattttgtaagtggcattgccttcagtattgaaaactctcatatgtgcatgtcacctcttgctgaaagcagtgtcattccttgtgaaagcagtaacattcagatggatagtggctataatacgcagaattgtggaagcaatattatggatacagttggggcagaaagttactgcaaagaaagtgatgcacaaacatgtgaagttgagagtaaatctcaagcatttaatatgaagcaagaccacacaacacagaggtgttggatgaaaacagcaagcccttttcaatgcagcagtccatagaatacctctgtcagaatcaaagactaagcttaagagttcctcgcatatatcgttgtgcacaggatcaacatgatggtgactgggaaaaaattacttcaagtaacatgcttagctttccctccttaatgtgaaaaatcaagggcttactgacataggaacaacagaaatgctcctggaacttcaagttgctgaattataagtttattttttatcaataaatatttttatacttaaaa

SEQ ID NO: 78 - DNA, 2221 bp, Homo sapiens
  misc_feature: Incyte ID No 2369350CB1
ggtggggccgccgaggtgtacgtctgtggagccgagccgcctccttgccttctccgcgggccgctcttttagcctgcgcggccgtcctccgagcaggccgccccagctttggagttcctcctcactgcacccctgtttctgctcactgagaccagcttgtgggaaggagcgccccccgcctcactcaccaaccccttggctggcagcacagcagactctggggcggcctcgggttcctggggtgatagtccagcttctggtgtaccagaggaagaagaaaggcgtgtcccccaaaggctgtccttgcagtagctggaagccagatgagtaacgaaagaggctttgaaaatgtagaactgggagtcataggaaaaaagaagaaagtcccaaggagagtcatccactttgttagtggtgaaacaatggaagaatatagcacagatgaagacgaagttgatggcctggagaagaaagatgttttgcctactgttgatccgacaaaacttacctggggtccctacttatggttttacatgcttcgggctgctacatcaactctctcagtgtatgacttccttggagagaagattgcatctgttttgggtatcagcaccccaaagtaccaatatgccattgatgaatattatcggatgaagaaggaggaagaagaagaagaagaagaaaacaggatgtctgaagaagcagaaaaacaatatcaacagaataaattgcagactgattccattgttcagacagatcaaccagagacagtgatatccagctcatttgtgaatgtcaattttgaaatggagggagacagtgaagtaattatggaaagcaagcaaatccagtctctgtcccaccataaaatgaaatgactatcaagcttccaactcttaagttttttttttttaatacaaaactttcacattctttattcagtgggacttaatacaattatttatattttaaattattaaagtatctggaaagggaaaatgttttcttcatttttaggatctatctagcaaagccagatctgaaattcagatatttgtactgtttttactgtgtatagaaattagtgctttggttttaaaatgatcttttaaaaaagttaaggacatcctagagccttaatagttaagaagagttaaattatcaagcctatttgtgcatttgctttttttgaaaaaggtaagttgctgattaagtctaattggaattgataattccatagtcttagattaaaatgaggatattttctcctagattttctcatgttatgccatgcatttatatatctaaccattaatttcacactaaggatgcttcaccatataataaaaggagcaagatggaagcactttgaattttctttcattgagaataactgttttatgtaagaatctgtatttataacaccagatattaagataggcttccattttttaatgcaagccacttacttaatcttgtattctttttcaggactcaaataactagctttgaacataatattaaaacactacttatagaatagatttattaatgttaatacctagtgaatatccatgtggcatcctggttatgttatcggttcagcgttaatcctatagaaaagtggtttggaggggattgggggatagtgggacaggtatagatttaatccatcaggagcaattagatattgtataaggtgcaatgatagcctaatgaaattacccgtcattcatcatttagaagtagcaacagtgaagactggacagtttacttgaatctggttggccactcctctacctacttggttatttgtaaaccttacaaatgtatatattgtgaagctaattttgaaaatattcctaaatatggccaggtacggtgctcacacctgtaatcccaaagtgctgggattacaggcatgagccaccgcacctggccttccctgcttcctctctagaatccaattagggatgtttgttactactcatattgattaaaacagttaacaaacttttttctttttaaaatgtgagatcagtgaactctggttttaagataatctgaaacaaggtccttgggagtaattaaaattggtcacattctgtaaagcacattctgtttaggaatcaacttatctcaaattgtaactcggggcctaactatatgagatggctgaaaaaataccacatcgtctgttttcactaggtgatgccaaaat

SEQ ID NO: 79 - DNA, 2039 bp, Homo sapiens
  misc_feature: Incyte ID No 2722979CB1
tgacccggtccaaggcggaaaaagtgcggccgcccactgtgccagtgccgcaggtggatattgtgcctgggcggctcagtgaggccgagtggatggcgcttacagccctcgaggagggcgaggacgtcgtaggggacatcttggccgacttgctggctcgagtcatggactctgctttcaaagtctacctgactcagcaggtgggccgggatccgggtccttcagactcgtctccctctcccgcccctccctgccgacctgagatcctctctcgcctccgcagtgcattccattcaccatcagccaggcccgggaggccatgctgcagatcaccgagtggcgcttcctggcccgggacgagggagaatctgcagtagctgaggaccccacatggggtgaggacgaggagccttcggcatgcacgacggactcctgggctcagggttcagtgcccgtgctgcacgcgtccacctcggagggcctggagaacttccaaggcgaagtacactcctcaggagcctctccggactcctctgccattgctcctgctctcccctttccgacatctcactgcccgagtgcatttccccaggaccctgggggcgtggaccggatccctttaggaaggtcgtggatgggtcgaggctcccaggagcagatggaatcttgggagccttctccgcagctgagagtcacgtcggcccctcctcccacatcagagctgtttcaggaggcagggcccggaggtcctgtagaggaagcggacggccagtctagaggcctctcctcggccgggtccttgagcgcgagcttccaactgtcggtggaggaggcgcctgccgacgatgccgacccttctctggatccgtacctggtagccagcccccaggcctcaactgggaggggacaccccctcggcttccatttgtcgttggaagacctctactgttgcatgcctcaactggacgcggctggggatcggctggaactcaggtcagagggggtgccctgcatcgcctcgggcgtgttggtgtcctacccctctgtgggcggcgccacccgcccctccgcgtcctgccagcagcagcgggccgggcactcggatgtgcggctgagcgcccaccaccacaggatgcgccgcaaggcggccgtgaaacgcctggaccctgcgaggctcccgtgccactgggtgcgccctctggctgaggtcctggtcccagactctcaaacacgccccttggaagcctaccgcggacgccagcggggcgagaagaccaaggcccgggccgaaccccaagccctcggccccggcacccgtgtctccccggcagcgttcttccctctccggccaggcattcctttccgtgacttggactcgggccccgcactcctgttccccactttaaatttaggcctatcgtcgccatccctcgagtcaaagctgccactcccaaactccaggatccgcttcctcaccacacacccggtgctccctgatgtggcccgcagccgcagccccaagctgtggcccagtgtcaggtggcccagcggttgggaggggaaggccgagctgctgggcgagctgtgggctggccggacccgcgtgcctccacagggtctggagctggcagacagggagggccaggatcctggcagatggcctcgaaccacacccccggtccttgaagccacttcccaggtgatgtggaagcccgtgttgctgccagaagccctgaagctggcccctggtgtgagcatgtggaaccggagcacccaggtgttgctcagctctggtgtgcctgaacaagaggacaaagaaggtagcacctttcctcccgttgagcaacatcccatccagacaggtgccccaaagcccaggtgaccgtagcacagctaatgaagaactcagcccccaaagtgtggtcacgctcctctaagcctgctgcctccctctgatccctgagcctctggcagtagtaactggtccctccctctgctagccagaaataaacacctgagttgccttaggaactaaaaaa

SEQ ID NO: 80 - DNA, 1254 bp, Homo sapiens
  misc_feature: Incyte ID No 60140470CB1
agcggctgagcctgcgcttcggaagcatggatgtgcgcctgcgctgcgctagggcgcggcgggcggtttgaattttgcttacagagtcccgtctcaccatcctgggcttccaacggagactgcggtatccgcggctggagacccagcggcgagtagccttttgctcccggacggacttgagaggcttaaaggatggcctcgtcagatctggaacaattatgctctcatgttaatgaaaagattggcaatattaagaaaaccttatcattaagaaactgtggccaggaacctaccttgaaaactgtattaaataaaataggagatgagatcattgtaataaatgaacttctaaataaattggaattggaaattcagtatcaagaacaaaccaacaattcactcaaggaactctgtgaatctcttgaagaagattacaaagacatagaacatcttaaagaaaacgttccttcccatttgcctcaagtaacagtaacccagagctgtgttaagggatcagatcttgatcctgaagaaccaatcaaagttgaagaacctgaacccgtaaagaagcctcccaaagagcaaagaagtattaaggaaatgccatttataacttgtgatgagttcaatggtgttccttcgtacatgaaatcccgcttaacctataatcaaattaatgatgttattaaagaaatcaacaaggcagtaattagtaaatataaaatcctacatcagccaaaaaagtctatgaattctgtgaccagaaatctctatcacagatttattgatgaagaaacgaaggataccaaaggtcgttattttatagtggaagctgacataaaggagttcacaactttgaaagctgacaagaagtttcatgtgttactgaatattttacgacactgccggaggctatcagaggtccgagggggaggacttactcgttatgttataacctgagtcccttgtgaacttttgaacataccaacagggtatagagtatagaggctatttctataattttcttatatataatttttttaacttttaatcttttttgtttcctttttttttttttttgagacaggatcttgctttgtcacccaggggcttgctttgtcacgcaggctagagtgcagtggcgcaaacatggctcactgcagcctcaacctaccaggctcaagtgatcctcccacctcagccccctgaatggctgggactacaagcgtgcgccaccatgcctggctaatttta

SEQ ID NO: 81 - DNA, 1879 bp, Homo sapiens
  misc_feature: Incyte ID No 70623603CB1
tccacctgccagcggagtttaaagtttccgaggctcagaggaacacaatgacttggatcaaacagcctaaatgggaagaaggacatttttgctgcatcaaggaagccgttaaactcctgctaagctaactagctcttttttatgggtccatgcacacgaccgaactcctctttcactgaccagagattatttctgacaacccaggatatcccgaaagcttggaggcatatggctggaaaatgaaacgacccaggacatcgtttctggctgcatcattattttgtgtcgcgtagtaccagatgggcagtcagtgagcggcgcagggatgtgaacggacggttttataatgtgaaaattttcccttggtaaagctaaaacagatttaatttccctctcttttctttcactacttccccctctttattccccctctgtctgcaatatcagtgaactcaactttgcagtgaggtggccaaaaagagagagaatgaggagatcttgatcatcttagtgtcggaggagtcgcagcggactgggaactgcagctgcgaccccccgcgtcctgtgcggatttcagggctgataccgcataggcggttatggaaaggacggtacaccggagcggcggaggatagagaccctggcccccggagaggtctgctgatttcgcagcagccttcgaagccgtggctgcctttcatctgctgcgttttattactattatcgccgttccggaaaagtcatggaagacagcccgctgccagacctcagagacatcgagctgaagctggggcgcaaagtacccgagagtctagtgcgctctctccgtggggaggagccggttcccagggaaagggacagggacccctgcggggggagcggtggtggtggcggcggcggcggcggcggcggcggctgcagtagcggcagcagctactgcagcttccctccctccttgtcgtcctcctcttcgtcctccccaacctctggctccccacgaggtagccactctagcgccctggagaggctagaaaccaagcttcacctcctcaggcaagagatggttaacctcagagccacagacgtcaggctcatgcgccagttgcttgtaatcaatgagagcatcgagtccatcaagtggatgatcgaagaaaaagccaccattaccagcagaggcagcagcctcagtggcagcctgtgcagtttgttggagagtcagagcacctccttacgtggcagctacaacagcctacacgatggcagtgatgggctggatggcatttccgtgggaagttatctggacacgttggcggatgatgtcccaggccatcagaccccttcagacttggaccaattcagtgacagctccctcatagaggactcacaggcactacacaagcgtcctaaattggattctgaatactactgctttggctagtgacagttttttgcatgggactggtgtgcaatgaacttgtatttatccttcttctccgctgctatatttttggtgtgatttttattttaataagatgacctttttaaaagaagctgattttgaaactgcttaatggtattgctgttgctcctaatacttctcatctgagctgatttatttttctctgttacatctctattttttatttattacaatgattttctcccttcttttacagtagcacaaacaaagtagggggaaaagaataagcaataattatgtttttgcttttgttttcagagcaatgggtcagggattacaagaaaaactttgctaaattttacaataaaccaaagtctgataacagttaatgttgctgcttgcgtccttaaatgacttaaggtttcatcttccagaagatattgagatatattactgttt

SEQ ID NO: 82 - DNA, 2767 bp, Homo sapiens
  misc_feature: Incyte ID No 7161479CB1
cccataatatcatatgctcaatctattctgcatttggcctccgtcatagtcttaatgtcaagtacccaaacactccaggattcaacaagccctgaaaacttaatagaactctacttttttagcacttgtgaccatttcaccttttcaaccaaagtgactgttcagtgttacaactttgtcattttgacatgtagcttgcaaaggcaatggattcccatcttggtttccttaaagtgtagatgttcaaaaatatgccagatactctaaattattggtgtctgttaaatgggaacttctcaaagatcaaagaattgctcaacaagtgctaaactttgacatacttcttgcattttctttcaggctaacaataaatgcccttgcccagaagctcaatgcgtactggaaggaaaaaacatctcaagataattttgagacctcaactgtagccaggccaataccgaaggttcctgaccagacatgggttcagtgtgatgagtgtcttaaatggagaaagcttcctgggaagattgatccatccatgttacctgcaagatggttttgttattataattcccatccaaagtacaggagatgctctgttccagaggaacaagaactcactgatgaagacctgtgcttgagcaaagctaagaaacaagaacaaactgttgaggagaagaagaagatgcctatggaaaatgagaaccaccaggtattcagtaatccaccaaagatccttactgttcaagaaatggctggattgaataacaagacaattggatatgagggaattcatagccctagtgtgcttccttctggtggagaagaaagcagatcaccatctcttcaacttaagcctctggattccagtgttttacagttttccagtaagtacaaatggatcctaggtgaagaaccggtggagaaacgaagaaggctccagaatgagatgacaacaccttctctagattattccatgcctgctccttacaggagggtagaagcacctgttgcctacccagaaggggagaacagccatgataaatcgagttctgagagaagtacaccaccataccttttcccagaatacccagaagcaagcaagaatacaggtcagaatagggaggtttcaattctgtatccaggggccaaagaccaacgccaggggtccctgcttcctgaagaattagaagatcagatgccaagattggtggcagaagaatctaacagaggtagcacaaccataaacaaagaagaagtcaacaagggaccttttgtagctgttgtgggtgttgccaaaggtgttagagattcaggagctcccattcagctgatcccttttaacagagaggagcttgctgagagacgaaaagcagttgaatcctggaacccagtgccttattctgtggcctctgctgcaatccctgctgcagccattggggagaaagcaagaggctatgaggagagcgaaggtcataatacaccaaagttgaagaaccagagagagctggaagaattgaagagaaccacagaaaaattggaacgtgttttggctgaaaggaatttgttccagcaaaaggtggaggagctggaacaggagaggaatcactggcagtctgaattcaagaaagtccaacatgaattggtgatctacagtacccaggaggcggaaggcttgtactggagcaagaaacacatgggttatcgccaagctgaattccagattctgaaagctgagctggaaagaaccaaagaggaaaagcaagagttaaaagagaaactgaaggaaacagagacacacctggaaatgctgcagaaggctcaggtctcctaccggaccccagagggagatgacctagaaagggctttggcaaagcttacgcggctacgtatccacgtcagctatctccttacttctgtcctccctcacttggagcttcgtgagatcgggtatgactcagaacaagtggatgggatcctgtacacggtgctggaggcaaatcacatactggattgagcaccagactgtatacccttctcttctcttatcttctgtctgttctcttttctctccctccctcacgtctctctctctctctctctctctctctctctctctctctcaccctcacctttatgccttatatagagaatctctgtgtaaatcctggctcataatcagtctcctttttatcagttttggtgtggagaaagaggccagtttaaataggctttcaagagtctagggtcagaaaagcaatagtcactaagctaggtgacctgaaagctttaattttcatgacctggatatgtggtctattgtatatctttttctgaaatggttgtattcatttaggttagatcaatcagcagatattgggtccggtataccaggtattattttggggtaagctaacaagtacaactcatgtttgcagccttcgaagatgtaacaatctcgttggaaacataagacatacatcacattatacacaaaagtgtatgatatgtacaactgagtggtacagatatgacatggaagatctgggggaggaagttcaaggaaggcaccacaccagaaatgggacctaaattaaggcttaaagaatgagcatggccacactgtcagtgattgttctttaggtggtaggactatggttgatatttttcttcttcctatcttcctgcctttttcagattttcaatattaaacttgttattcttacattggaaaaataatggttttaaccgaaaaaaaaaaaagggg

SEQ ID NO: 83 - DNA, 2364 bp, Homo sapiens
  misc_feature: Incyte ID No 7502313CB1
tcaccacctgccataccccttaaataagcccctcaccttgctgcctcaggaccttcaagattccatctgtgggctggccggcaagatggcaccagtggggacccacaccctggctgggcagaggtgctgctagcaacctctcttcctctataagaggaaatggaaaatgcagggtgtggaattgccctttggggtccttccttaattgaaggccaccttctcacaggtttcattctgcagggatttactggaatctattggtgctgctgcatgagtctgctgacaacctgactgcacaaggactgggtagcagactcctcagagtcctcttgacacaaatgtcagatttgtgtcactcttctgccttcgtgaaaagccaatagcactctcagatatcaggggattttagttccaagcagggaccctggtttccatactgccctcagctggagtttggatccaaaggctctggctaagtcattatgtcactttttcacaggaatgtaaatttgactgtcacctctgaatttgttcagtgtcccaccatggtctatgagaagtacactggaagcgtggggggaacacatgacatgatttgtgaatatcatcatctttgccagacaagtctccaggggatccctgtttcccaactgaaaggtgtgaacggacacacacacagcctggatgacgccttggctgttctaaggggctgtaaggtgggctctgggccttccagctaggctctcaagcacagcagaagcctcactgggctgctatgtctctgtatttgtggcttgtgtggtagcctcagaagcagagctgtttggcagactggctggagaaattccctctaggagacttgcctgtgctgtgcttccaggtcacagagccccccggaaactcacaggggccctcttcccagaaaagaatctattctatcacttcagaatcaggacactcaagctctggcagaggaaggccaagttactttcatggtcttaccctctgcttttcccctttttgcaaaaaaccactggccaaatccgaaccattgcccttgtttcccccacgttctctctcagatctttgtctcgaagggaaaacatagtggatgaaaaggtgtggcaggctttggcaccttgttaaaatttctagtcatctgtggatgttaccttgcttgtccacagcagccagtcaccctggccagtcccacttcctggataattctctaccctcaccccacagagccatctctctccagaccaaaagctggaaggagagttgctttgagagcttgtttttacaactgcatgtttattatgatactttctctccaaaggaaacttttaaatcaatgggaacaattagcaacagaaagagcacagtccctgcttttgactgggttcctattttaagcacaaatgagagctctggagccagaatgccagggttctaacttcagcattcacttactagctgtatgatcttggccaagtcacttcacctccctgagccccaattcccaagtttgtgaaatggcaacaatacctatgtgtcactggattattggttaaaacagaatgagattccttgtgtgaaaatagctattatacctgacacactcatcgtatgggctctgcaaagggatattccccaacctgtccttcccgacaggaagcatagggcactgcagatggggaagcatgtcaccttggcagtgactcggtggcttcccaagcaggagtgtcaggggaaccatgagagagagtctaggagccaaacacatcaccaccctgagcagatacaggagtggggagggggctgtaactcagtgagtggcttccaggggccccaggccctgctggatgtgggccaagccctacagcttccctaggcagtaagtaaaaacattctcctagcattaaaatggtttccataactacttttgtcctggcttcttaatactgggtacctggcatgcagccaagaactctgcttttccgtggtgcttatgtattaagtagattagctggggagggatattccttgtttaatggcagatccaggacactccggaagctctgcccaccaacttcaccttaccaggcgagagtagcactgcttggaaggctgctcctgccttttaaagcctgtctacgtattagctcctccaccaaggaaaagaatttgctgttagatggctagggcaggacacggacagtcatcaggggatctatgtttggcttatggcaagtggcttcactcccacggctcaggtgccattaggggatattgagcccgcttactaaccccactgaccacctctgcatcatttggaaaatggagagtctccctgccattctc

SEQ ID NO: 84 - DNA, 2597 bp, Homo sapiens
  misc_feature: Incyte ID No 7502390CB1
acgctcacaccttgtaatcctagcactttgggaggctgaggcgggcggatcacctgaggtcaggagttcagcctggccaacatggtgaaaccccgtctctactaaaaatacaaaaattagccgggcgtgctggtgcatgcctgtaatcccatctactcagggggatgaggcaggagaatcgctcaaacccaaggggcagaggttgcagtgagccgagattgcaccactgcactccagcctgggtgacagagcgagactccatctaaaaaaaataaataaacatgggagaaacctggaagaatatttgctcaactgtgaggcatggttggtggctaagggatcacagaatggcaggcctgcctattcctcctgagatcgttaaggaggctgaggtgccgcaggctgcgctgggcgtcccagcccaggggacaggggacaatggccacacgcctgtggaggaggaggtcgggggcatcccagtaccagcaccggggctcctgcaggtcacggagaggaggcagcctctgagcagcgtctcctctctggaggtccacttcgacctcctggacctcactgagctcaccgacatgtcggaccaggagctggccgaggtctttgctgactcggacgacgagaacctcaacaccgagtccccagcaggtctgcacccgctgccccgggccggctacctgcgctccccttcctggacgaggacaagggctgagcagagccacgagaagcagcccctaggcgaccccgagcggcaggccacagtcctggacacgtttctcactgtggagaggccccaggaggactagaccatctccacctgccccagctcctgcagggatggggtccgaacacgatggcagatctgggcagtgctgacccagcagacacacttcacccgcccacgaggctccagccgtcacctcctgacacacaccctgggggcagctctctgccagccccgagaccggccttgtctgctgggcacgggtcttcgcctcacttggagaccagccggctttcctgggggacacacggggcccccggatgcctctgggagccccagcacaagcacagcccagtggccttacgtccagctcgttcctgggccccgagtcaggaagacagcgtcacggagtcactgccaggaacgtgctgaggaatggagtggcccacggcggccttggggtgaaggggacccaggcctgtgacagccactccaggaactcctgggggtgctccaacctccgcgttttcctgtgctgccaagctcagaagccagaggcgggtttggtagtggctaatgggacaatgtgctgtccagcaaagcacacatggagaagcggccccaaaattcccaccttgatttccatcctgccccttcttctactccacggaggcgctgtctcactagggtcccctccccaaggctcagctctaagacctgcacctgcttctcttggcccctgcgtgacagacaagtccattccctccttagctcagaacaccaaatatcaccagactgcctaagagacttgatgacacctcccggaatgctctcggggtggggttcacctctccttgtcctgcacccactgctaggccacattctcgtttctgctcacatcccattgcccggctacaaggcctgcccacggcccttaaacttgctgggcaggtttggagcccatgggaccccgtgggtctctgtccaggagcagcagaggaggctgacaggccctgctccctctgctctgggggtgtctgggagccccagctcacaccctcccaatgcttatatgctgaagctcacagaatgggcttcttgcctgacagcaagtcaaagaatgagtttaatatcaaagtgtaagcttactttccatccccaagccagcctgccccctgccccatttcccatgagcacacttctggggaaggaaaacaggctcctggccttcactctcagcagagctttggagatgccccaggcatgccctgagctccttctgtgtacctgctcccacttctgagccacccgctgcccctccgcactgctggcaaacccagttcctgcctcagccaggtctccttccctggtttccagtcacacagagcccagcagctttctctttcagtcccataagggcagccttgtgtccctggccacacttccacccgccagggtcttcctccccatctttccatccttcctgctgagcttccacagagctcgtttgcaaacagggggattaaagcatcactgcgcattcgaaggcctggccacagtctcttcctttccatagcagagtgaacgaggtgcctgctgaggggttgtgagctgagctgcctgggctccgtgcccggaccattctccagctgcagcagcctgagggctctgctgtgctcacttgggtcacatgtgggggatgagtgacctggatttccagtcccaaagtcacacacagaaggaatcattaaagaagtgaaagtcccgccccacgtgcacgtaaaggtagcccaggctgggggagggagtcacagaagtcccaccccacgtgcatgtaaaggtagcccaggctgggggaggg

SEQ ID NO: 85 - DNA, 2229 bp, Homo sapiens
  misc_feature: Incyte ID No 7502872CB1
ggccgggctgggatagcgcgagtgtccgcggccgagcagcagagatttttgctgtgagaattaattaccagtaacagttcaatatgggggacattctggctcatgaatctgaattacttggactagtgaaagagtatttagattttgctgaatttgaagacaccttgaaaacattttcaaaagaatgcaaaataaaaggaaaaccattgtgtaaaacagtaggcggatctttcagagactccaaatcattgacaattcagaaggatcttgtcgctgcatttgacaacggagaccagaaggtgttcttcgatctgtgggaggagcacatttcaagttccatccgagatggggactcctttgcccagaagctggaattctatctccacatccattttgccatctatcttttgaagtactctgtggggagaccggacaaagaggagctggatgaaaagatttcctacttcaaaacctacctggagaccaaaggggcagccttgagccagaccacagagtttcttcctttctatgcccttccttttgttcccaaccctatggtgcacccctcatttaaagaactcttccaggattcctggactccagagttaaagttgaagttggaaaagtttctagctttaatatctaaagccagcaacacgccaaagcttttaacaatatataaggagaatggacaaagtaacaaagaaatcttgcagcagctccaccagcagctggttgaagctgaacgtaggtcagtgacatacctcaaacggtacaataagatccaggccgactaccacaatctcattggagtcacagcagagctggtggattctctagaggccacagtcagcggcaagatgatcacccctgagtacctccagagcgtctgtgtccgcctgttcagtaaccagatgcggcagagcctggcgcatagtgtggacttcacgaggcctgggacggcatccaccatgttacgagcctccttggcacccgtgaaattgaaggatgtcccattactgccctccttggattatgagaaactgaagaaggatttgattttggggagtgaccgcttgaaagccttcttgttgcaggctctgcgctggcgcttgaccacatcccatcctggagagcagagggagaccgttctgcaagcctacatcagcaatgacctcttggactgttatagccacaaccagaggagtgtgcttcagttgctgcactccacgagcgacgtggtgcggcagtacatggccaggctcatcaatgcttttgcgtcactggcagaaggtcgcctctaccttgcccagaacacaaaggtgctgcagatgctggagggaaggctgaaggaggaggacaaggatatcatcaccagggagaatgttcttggggccctgcagaagttcagtctcaggcgcccgctgcagacagcgatgattcaagacggcctcatcttctggctggttgatgttctgaaggaccctgactgcctgtctgactacacgctggagtactcggtggctttgctcatgaacctctgcctccgcagcacagggaagaacatgtgtgccaaggtggcaggcctcgtgctcaaagtcctttcggatcttcttggccatgaaaaccatgagatacagccgtatgtgaatggagctctgtacagcatcctttctgttccatccattcgtgaggaagcaagagcaatgggaatggaagacatcctacgctgcttcatcaaagaaggcaatgctgaaatgatccgccagatagaattcatcatcaagcagctaaattccgaagagctaccagatggtgttcttgaatctgatgatgatgaagatgaagatgatgaagaggaccatgacatcatggaagccgatctggacaaagacgaactgatccagccccagctcggagaactctcaggagagaagcttctgaccacggagtacctggggatcatgaccaacacggggaagacaaggcggaaggggctggctaatgtgcagtggagcggggatgagcccctgcaaaggcccgtcacccccggcggccacagaaacgggtacccagtgtaagtcagggctaaaggaagcgggaattgactttcttaagctttgttttgattacagtgtaagatgtatgtatttttaaaattcaaaataaagcattcattttgaaacaaaaaacaaaaaaaaacacacacaacaccacaacaacacaaaa

SEQ ID NO: 86 - DNA, 2504 bp, Homo sapiens
  misc_feature: Incyte ID No 7505443CB1
gtttgaaacatggcgcgggctggccctcggctggtgctgagcgaggaggcggttcgggcgaagagcggcttagggcctcaccgcgacctggctgagcttcagtcattgtctattcctggaacttaccaagagaagatcacccacctgggacattctctgatgagtttaacaggtctgaaatctttggatctctcgcgcaactccttggttagtctggagggcattcagtacctgactgcattggagagtctcaatctctactacaactgcatctcctcgttggcagaagtgtttcggctccacgccttaaccgagctcgtggatgtggacttccggctgaaccccgtggtgaaggttgagcctgactaccgcctttttgttgtgcacctgctccccaagctccagcagctggaatccagacatctgttgagcccgcagttggtacagtaccagtgtggggactctgggaagcagggccgtgagacgaggaggagcagctgcagagggtgctgtctggagaagatgccttggagccagctctgtggagagcttccgccactgtacggagcggagccagaggcctcccgtgcccccaggccacacacgtacttcaccccacacccagactccatggataccgaggactcggcctcttctcagaagttggatttgtcaggagaaatggtgcctggtcccctgccagcccccggaaagtgcaggaagcgaagaatgcctgttggaagattccagacgttttcggaccaggagggtttgggctgcccggagagaactcatgggtcctccgtgcccaaggagagcctgagcagacaggacagctcagaaagcaggaacgggaggaccttgtctcagcctgaggcctcggagactgaggagcagaggtctcggggtgtgaccgacaccagagagccgtctcccgggtcacactcggctctacccgggaagaagacggccctgcaggcggcgctcctggagacgctcttggacctggtggacaggagctggggcggctgcaggtccctgcacagcaacgaggcattccttgctcaggcaagacacatcttgtcatctgttgaagaattcacagcagctcaggacagctctgcgatggtgggtgaagatgtcggctccctggctctggagagtaagtccctgcaaagccgccttgctgagcagcagcagcagcacgcccgggagatgagcgaggtgacggcggagctgcaccacgcacacaaggagctggatgatttgagacaacatttagataaatctttggaagagaacagtaggttaaaatcgctttggttgagtatgaaaaaggaagtgaagagtgcagacactgcagccacgttaaatttgcagatcgctggacttcaaacaagtgtgaagaggctgtgtggcgagattgtggaactgaagcagcacctggagcactacgacaagatccaggagctcacgcagatgctgcaggagagccacagctccctggtcagcaccaatgaacacctgctgcaggagctgagccaggtgcgggcgcagcacagagccgaggtggagcagatgcactggagctaccaggagctcaagaagaccatggccctgtttccacacagcagcgccagccatggaggctgccaggcctgctgactcctgccgagaagctgggccaccccttaagcttcctggtaaagttacattgtctgcacctttgtacttctttactgagtgtactggctggcaagagttctctcttctgttggtaattatttaggatttttggaatgtattcaggacctgtagcttggttttctaaagcacctcgtaaaatgatatgattactccaagcccctctgcatgttttcagacagaacacattgacatattttgagacaaactgactattaatcttgtatccagtatcctgagatgaagtaaatgcagtgttctactgcctgatgtgaaagagagctatgtatgataattaaagaaaataattttctgtgtaacaagcaatctttatttaataaacaaataacattgttctgaaaagttaactttttcagtggctgtatacaaattataactgagtttgtcattgagttttttatagaacaagctgtgcaccatgataggtgagtgggaataattaagttcaaagacttaacacaagtgacacttatagatgtgggggaaaaaaccttaaaaatattgtcttatgatattaacatatcacagcggaaacatctacccaagcctggctcgctctcttgtttgcagtgtagttctgaaaattatgtcataataggacacaaaactagatttttagtttggaaacttctatatcttatgtctatacttgctgtgattggtaagacaaggtcattttttaaatgacgtacaactaatttgggaagaatgagcaggcctaagaagaaaatgctctgtgctagctctgtgtgtgtgtgtgcccttgctgtgtgtgtgcc

SEQ ID NO: 87 - DNA, 701 bp, Homo sapiens
  misc_feature: Incyte ID No 8032443CB1
ggggaccatctagcccctaaccatgggcccagaagagaagaccatcatgacagaaaggtctgcagctgttttcatccaggcctggtggcggggcatgctggtgcgacgcacactgctgcatgcagccctcagggcttggatcattcagtgctggtggaggcaggtgctggagaagctgctggcaaagaggcggaggatggtgttggagttctatgtgcagcaggaatgggcagcagtcaggctgcagtcctgggtccgcatgtggtgtgtccgccagcgttactgtcgtttgctcaacgctgtccgcatcatccaggtctattggcgctggcacagctgccattcccgtgtctttattgagggccactatgaactcaaagaaaaccaacttaatattcaacttgaaatctctttgggcttacaggcttgtaaggtgcaacaatgcataccccttccattaaaagaatgaccaggtctgctaaaaaaaaaaaaaataacagcagccatcagaagacagcagcaaaactaagcaaaaacaggggggggcgggcttacttagagagctccaggtagttgttaagcatttgtggagacatcgccctctattaggtggcccaaaaaatagacatgaggggggtgttttcgcgccgtgcggtggaaccccggtgtccccttttccgcgtggctccctcctgcgacaa

SEQ ID NO: 88 - DNA, 1569 bp, Homo sapiens
  misc_feature: Incyte ID No 7704916CB1
ttaacacatgtcaagtacctagaacagtcccttttattccatcacatgggttgcgaggtgaagtgcaccattgccttgaccctggggaacttgataaccaaatacagaattaaattgcttaccaaaccccatttatatttatagctggaagagcctgtattgtcctcacaatagtatagaagaattcaagagaggagagagagacagcaccgaatgaagactgtaaaagaaaagaaggaatgccagagattgagaaaatctgccaagactaggagggtaacccagaggaaaccgtcttcagggcctgtttgctggctatgccttcgagaacctggggatcccgaaaaattaggggaatttcttcagaaagacaatatcagcgtgcattatttctgtcttatcttatctagtaagctgcctcagaggggccagtccaacagaggtttccatggatttctgcctgaagacatcaaaaaggaggcagcccgggcttctaggaagatctgctttgtgtgcaagaaaaagggagctgctatcaactgccagaaggatcagtgcctcagaaacttccatctgccttgtggccaagaaaggggttgcctttcacaattttttggagagtacaaatcattttgtgacaaacatcgcccaacacagaacatccaacatgggcatgtgggggaggaaagctgcatcttatgttgtgaagacttatcccaacagagtgttgagaacatccagagcccgtgttgtagtcaagccatctaccaccgcaagtgcatacagaaatatgcccacacatcagcaaagcatttcttcaaatgtccacagtgtaacaatcgaaaagagtttcctcaagaaatgctgagaatgggaattcatattccagacaggaggtggtgcctcattctgtgtgctacatgcggatcccacggaacccacagggactgctcctctcttagatctaacagtaagaaatgggagtgtgaggagtgttcacctgctgcagccacagactacatacctgaaaactcaggggacatcccttgctgcagcagcaccttccaccctgaggaacatttctgcagagacaacaccttggaagagaatccgggcctttcttggactgattggccagaaccttccttattagaaaagccagagtcctctcgtggcaggaggagctactcctggaggtccaagggtgtcagaatcactaacagctgcaaaaaatccaagtaacaccttctgagtagctgctgtcccacacaatagggtatgaagctgcgctcctccatcgggtttggggagggagcactctgggactgtgagacaaggaagcagggccagcagtgagactatgagccaagcaaagagaagtctcagtggagcatgaggagggagcagtccagatgccaacaaggaaatgcgtttatggctacaagagtgcctctgctttctcctcctctcctcccaccaaggattcttccaccttaatcttgttttcatatgcctcttcttacttcacccatgtttgttgttatgcaaataaaggttttctctccaaaa

SEQ ID NO: 89 - DNA, 1052 bp, Homo sapiens
  misc_feature: Incyte ID No 2013440CB1
ggtagagaatccaagatagatcaactctccctaaaggctgacagtgaactcttggggccgttttattctctgaggttagcaaggagtcatctactagccattcaggaggccagctgggaagacaaaataggcaccccaaactcagcaacttcataacaccttcctctccccgcctgaagccttaaactgcatcaagtcaaagaaacctggggcaaatccttaacatgtttttgactgcagtaaatccacagccactctctactccgagctggcagattgagaccaagtattcaacgaaagtgctcactggaaattggatggaagagaggagaaagttcaccagagacactgacaagacaccccaatccatttacagaaaagaatacatccccttcccagaccacagaccagaccagatctccaggtggtatgggaagaggaaagttgaggggctaccttacaaacacctgatcacccaccaccaggagcccccacatcgctacctgatcagcacctatgacgaccattacaaccggcatggttacaacccggggctgcctccactccgcacttggaatggacagaagttgctgtggctgccagagaagtctgactttccccttcttgctccccctacaaactatggactctatgagcagctcaagcagagacagctcacacccaaggctggcctgaagcagagcacttatacttcatcctaccccagaccaccgttgtgcgctatgtcctggagggagcatgcggtcccggtccctccccatcgcctgcatcctctcccacacttctgagagctgccaccccaggagcagctcagatagaatcagctggagaccacagcatcactggacttgccagacaacaagtggcgcagataaactcagagtacgagatctggcccgtcaaaggtgctctcagaatcatcatctgcatttggcggtacctgtcccccctcaaaacccacaggttcctttcttttccatccaacaattaaagatctttgacactaaaaaaaaaaaaaaaaaaaaaaattg

SEQ ID NO: 90 - DNA, 1325 bp, Homo sapiens
  misc_feature: Incyte ID No 2503512CB1
ccctgaccctcagcagccacaggcctccgccgaagccccatttgctgccagagggatctactcggaggagatgccgtcggtggcccggcctcggcctgtcgggggtaccacaggctcccagatccagcacctgacacaggtggggattgccagcagaattggagctcagccagtggaaatcccgccaagcagaggcagccagtatggggggccaggctggccttcgtacggggaggacgaagcggggcgaagagaggccacacacatgctcggacatcaagagtattcttcttcaccgctatttcaggtgccaaggacttcaggcagggagccctcagctccttccgggaacctcccccaccggggactgcagggccctgggctgggttaccccaccagctccacggaagacctccagcctggccactcctcggcctctctcatcaaagcaatccgcgaggagctcctccggctctcccagaaacagagcaccgtgcagaacttccacagctgatcggcctcgcctcgcagatttgccaagtatccgcttcctgtggaagcaagaccaaaaggaaatcaactgagtgggtgtttggaagaggaaggagcaactctcgggcagcctgcccaagggagggagcaagttgcaatttagaagatgccatacgtcgtgtgacagctcatgagcctttcactgggctggcaattgtctgaacacttgggttcagttgaaatatatgtattttggccaaaagccagcagcacttcacaaaaacaaaacacaaacctaagctaacaaaatgactgcattcgtctcttttttaaaggtagagattaaactgtatagacagcatagggatgaaaggaaccaagcgtttctgtgggattgagactggtacgtgtacgatgaacctgctgctttgttttctgagaagaggtttgaagacattttattaacagcttaatttttctcttttactccataggaacttattttaatagtaacattaacaacaagaatactaagactgtttgggaattttaaaaagctactagtgagaaaccaaatgataggttgtagagcctgatgactccaaacaaagccatcacccgcattcttcctccttcttctggtgctacagctccaagggcccttcaccttcatgtctgaaatggaactttggctttttcagtggaagaatatgttgaaggtttcattttgttctagaaaaaaaaaatccctcccaaagtggggcaaaaagctttatatttatttgattatccaaaatacagatcaaagtttagatctacaaaaaaaaaaaaaa

SEQ ID NO: 91 - DNA, 2110 bp, Homo sapiens
  misc_feature: Incyte ID No 277396CB1
caagaattcggcaccggggactgcggaggccggggcggtggcgtgcagggtgaggtctcccgggctggcgcgcgtgcaagccccctgctgctacctcgcgctaggccagctccaggagggcaacagacgacaggacatttcaaaagggccacttaaattttccttcgagtaaacagaacacctgccaagaccctcgagcctgggcgtgccccaagacaggtggttggggtctaggagtcggctgcagctggcgatgctctgaggggacgtcagggcctcaggatcgcccctatgaagacgaagtgcatctgtgaactgtgctcctgcgggcggcatcactgtccacatctccctaccaggatttatgatgaaacagagaaaccatgtcttctctccgaatataccgagaactaccctttctatcactcctacctgcccagagagtccttcaagccaaggcgggagtaccagaaagggtctataccaatggaaggcctgactacatcaaggagagattttgggcctcacaaagtggcaccagtgaaggtccaccagtatgaccagttcgtcccgagtgaagagaatatggatttgctcacgacgtataagaaagattacaatccctaccctgtctgtcgagtggaccccatcaaacctcgggacagtaaatatccatgtagcgacaagatggagtgtttgcctacttataaagctgattatttgccttggaaccaaccaaggcgagagccacttcgtctggaacacaaataccagccggcatcagtcaggtttgataacagaaccacacaccaggacgattaccccataaaaggccttgtgaagaccataagctgtaaacctctggccatgccaaagctctgtaacatccccttggaggatgtgactaactacaagatgagctatgtggcccaccccgtggagaagcgctttgtgcatgaagcagagaagttcaggccctgtgaaatcccctttgaaagccttaccactcaaaaacaatcctaccggggcctgatgggggagcctgccaagagcttgaaacctctagccaggcctcctgggctagacatgcctttctgtaacaccactgagtttcgagataagtaccaagcttggccaatgccccggatgttctccaaagctcccatcacctacgtccctcccgaagacaggatggatcttctgacaacagtgcaggcccattacacatgccctaagggtgccccagctcagtcctgccgacctgcacttcagattaagaagtgcggtcgctttgaaggctcttccaccaccaaggatgactacaagcagtggtccagcatgcgcacagagccagtcaagcccgttccccagctggacttgcccaccgagcccctggactgcctgaccaccactcgggcccactatgtgccccacctgcctatcaataccaaaagctgtaagcctcattggtctggccctcgaggaaatgtccctgtggaaagccagaccacctacaccatcagctttactcccaaggaaatgggcaggtgcctagcttcatatcctgagcctcctggctacacctttgaggaagtggatgctttgggtcacaggatatacaaaccagtttcccaggcaggctctcagcagagcagccatctttctgtagatgattcagaaaaccccaaccagagggagttggaagtgttagcctgattttgaaaaatagtaatttagaaattacacagtacttttaaaagcagacaactgagaattatttgttggacaaaaaagaattccctaaaatgacaaaaaacaaaaaacaaaaaaccttcaccacttccagagcacttgaataaaatgagaatcacttgactcagggaaaatgacatttaatcaccggctaattaactccccttaccttctcctttactgcatccccaccccgtttcaggtcctttactttgtgcttatggaatcaaagttggtctgtgaggttttctctgatccagatgtattttattaatttaatcattgtatgaactgacataatgattaacccttgccaagtatgaagcgccaaagaataaattttattttggggggaatg

SEQ ID NO: 92 - DNA, 1927 bp, Homo sapiens
  misc_feature: Incyte ID No 3044046CB1
cggagccagagagacgcagctaggcctccacggctgtggagagatcctgccacgggccttgttcaccatgtcggtgctggatgcgctttgggaggatcgggatgtccgtttcgacctgtccgcgcagcaaatgaaaacaagacctggagaagtccttattgattgtttagattccattgaggacaccaaaggaaataatggagatagaggtagactcttggtaacaaatttaagaattctctggcactctttggcattatcaagagtcaatgtttctgtcggttacaattgcatattgaatattacaacaaggactgctaactctaaattacgaggccaaactgaagctctctatatactaacaaaatgtaacagtactcgttttgaatttatatttacaaatttggttcctggaagccctagactttttacttctgtgatggcagtacacagagcttatgaaacttctaaaatgtatcgtgattttaaattaagaagtgcactaattcagaacaagcaactaagactgttgccacaagaacatgtatatgataaaataaatggagtttggaatttatccagtgatcagggcaatttaggaaccttttttattaccaatgtgagaattgtgtggcatgcaaatatgaatgatagttttaatgtcagtataccatatctgcaaattcgttcaataaagattagagattcaaaatttggtttagctcttgtcatagaaagctctcagcagagtggtggatatgttcttggctttaaaatagatcctgtggaaaaactacaagaatcagttaaggaaatcaattcacttcacaaagtctattctgccagtcccatatttggagttgattatgagatggaagaaaagccccagccgctcgaagctctgacagtcgaacaaattcaagatgatgtagaaatagactctgatggtcacacggatgcttttgtggcttattttgctgatggcaataagcaacaagatcgtgaacctgtattttcagaagaactggggcttgcaatagagaaattgaaggatggattcaccctacagggactttgggaagtaatgagttgattgaccttgagttgagatggatttctattaaagatatctctagtttaaagatactagtcacctgccataagtcatggaatagtttttatatttacagcttttatatttaaaacttgtaagagtttttttaatgattgaggaaaaagtcatttagaaaacttcagttttcggccagcgcgtcgagggaggggccagcgacacatggcctagtaaccgtccggccgcggcgctggcttaagccatggctgagggtagctggattcctcaggcccgggcgctcctacagcagtgcctgcacgcccggctgcaaattcgcccagccgatggggacgtcgcggcccagtgggtggaggtccaaagaggactggtgatctacgtgtgctttttcaagggagctgataaagaacttcttcccaaaatggccgaagctggactgtactgctgccatctctggctcactgcaacctccctgcctgattctcctgcctcagcctgccgagtgcctgcgattacaggcgcgcgccgccacacctgactggttttcgtatttttttggtggagacggggtttcgctgtgttggccgggctggtctccagctcctaaccgcgagtgatccgccagcctcggcctcccgaggtgccgggattgcagacggagtctggttcacttagtgctcaatggtgcccaggctggagtgcagtggcgtgatctccgctcgctacaacctccacctcccagctgcctgccttggcctcccgaagtgccaagagtgcagcctctgcccggccgccaccccgtctgggaagtgaggagcgtctctgcctggctgccca

SEQ ID NO: 93 - DNA, 1051 bp, Homo sapiens
  misc_feature: Incyte ID No 3808420CB1
ggagtcgcgcaggcaggcggaggctgagggcgccgctggccggccctcccagccctctccgcgcggctccgccggggttccaagaggagctagtaggttcggtgggggccctggcatggacatgcacagcgctcggcttgacagcttccttagccagctccgctgggaactgttgtgtggtcgggacacaggctcaccctcaatgcctggtcccctgcagccaacctcccaaactggcccagatgtgcagcccagccaccagcttagggcctcgggtgccttggaagaggactcagtctgctgtgtggaggaggaggaagaggaggaggaggaagcagtggtgacagaagacagggatgcagccttgggaggccccagggagcatgccctggactgggactctggcttctcggaggtgtcaggcagcacatggcgagaggaagaactgcctgtatcccagcgcccagcaccctcagcacagccccttcgtaggcagtgcctctcagtcagtggcctccccatgcccagcagggcccctgtagccagtgtaccacctgtccaccatccacggcccaagtccaccccagacgcctgcctggagcactggcagggactggaagcagaggactggacagcagccctactgaacaggggtcgcagtcgccagcccctggtactaggggacaattgctttgctgacttggtgcacaactggatggagctgcctgagacagggagtgaagggggtgacggaggtgggcaccgtgcccgtgctcggccccctcagttcctgcttggcctctctgagcagcttcggcgccggctggccagggctcggcggacagctatggcaggaaagcggctgtcatgcccacctcgcccagaacctgaactgcctgcggatgtctcacgctttgcagctctcatgagctgtcgtagccgccagcccatcatctgcaatgatgtcagctacctctgaccctgccctccagcctgggacaataaaagccttttttctagacaaaaaaaaaaaaaaaaaaaaaaagggcgt

SEQ ID NO: 94 - DNA, 2328 bp, Homo sapiens
  misc_feature: Incyte ID No 7504028CB1
ggctcggaaatctagttcgggaaaagtgtgaggggctgttcacgtggggaaggaacagcaggcgcggaggagggggcaagcgtgtgtgagattcagtggtccatgcgtgcgtttgtcgtgtaagggtcattcctggggtttggagtgggggaacaaatcaatgtggctgtttttccgtggaaagaattcccactgcagtgtcccggagcctgcgtgtggtgggcaagctcctcagatggtatctcacagggaataggggagtcttgaaaacgcagcttcggcagtaggaacatgaacctcttacctaaaagttccagggagtttggctccgttgactattgggagaagttcttccagcagcgaggaaagaaagctttcgagtggtatggaacctacctggaactgtgcggggtgctacacaaatatatcaagcccagggaaaaggtgctggtgattgggtgtggcaactcagaactgagtgagcaactgtatgatgtgggctatcgggatatagtgaacatcgacatcagtgaggttgtcatcaagcaaatgaaggaatgtaatgccacccgacggccccagatgagcttcttgaagatggacgtgacgcagatggagtttcctgatgcctcgttccaggtggtgttggacaagggcaccctggatgctgtcctgacagatgaggaagagaagaccttacaacaggtggacaggatgctggctgaggttggccgtgtcctgcaggtgggcggtcgctatctctgcatctccctggctcaggctcacatcctgaagaaagcagtgggccacttctcccgggaggggtggatggtgagggtgcaccaagtggccaacagccaggaccaggtgttggaagcagagcctcagttctccttgcctgtctttgccttcatcatgaccaagttcaggccagtccctggctctgcccttcagatctttgagctgtgtgctcaggagcagcgcaagcctgtgcggctggagagtgccgagcggctggccgaggcggtgcaggagcgacagcagtatgcctggctgtgcagccagctgcgccgcaaggccaggctggggagtgtgtctctggacttgtgcgatggggacacgggggagccacgctacaccctccacgtggtggacagccccactgtgaaaccatcgcgggacaatcattttgcgattttcatcatccctcagggccgggagaccgagtggctctttggcatggatgagggccggaaacagctggcggccagtgctggcttcaggaggttgattacagtggcccttcaccgaggtcagcagtatgaaagcatggaccacatccaagctgagctgtcggctagagtcatggagctggccccagctgggatgcccacccagcagcaggtcccctttctgtctgtgggtggggacattggggtccggaccgttcagcaccaagactgcagccccttgagcggtgactatgtcattgaggatgtgcaaggggatgacaagcgatacttccgtcgactgatcttcctcagcaacaggaatgtggtgcagtccgaagccaggttgctgaaggatgtgtctcacaaagagatcccactggcattgttggtggtaggcctgggcgggggcagcctccccctctttgtccacgatcattttccaaagtcctgcattgatgctgtggagatcgatccctccatgttggaagtggccacccagtggtttggcttctcccagagtgaccgaatgaaggtccacattgcagatggcctggactatatcgccagcttggcaggaggaggagaagcacggccttgctacgatgtcataatgtttgatgttgacagtaaggacccaacactgggaatgagttgtccgcccccagcatttgtggagcaatcttttctacagaaggttaaaagcatcttgactcctgaaggtgtttttattctcaaccttgtgtgccgagacttggggctaaaagactcagtgctggctgggctcaaggcagtgttccccctcctatatgtccggcgaattgagggtgaagtgaatgagatcctgttctgtcagctgcaccctgagcaaaaacttgccacaccagagctcctagaaacagcccaggctttggagcggaccctgaggaagcctgggaggggttgggatgacacgtatgtcttgtcagatatgctcaagacggtgaaaattgtgtgactgcttaggccaagcagccctcctgcctagactgaccttggactcccagcctgccagagaatgaagaaatacaacgcacagtaaaaaaaaaaaaaaaggggg

SEQ ID NO: 95 - DNA, 4782 bp, Homo sapiens
  misc_feature: Incyte ID No 7766880CB1
caagaatcaacttataaggtgtcaaaggcagatgacagatattctcagagtgtaatcagaagtaattcccgtcttgaagatcaagttattggggttgctctgcaagcatcaaaaaaagaagaaagtgttgttggttcagtgacacaacttaaccaacaaattggccaagtcaataatgcagctacccttgatcttaagaactcaactaatttaatacagactccacaaataaggttgaatactaaagacttaaagcagcaacatcctctcatacttaaggtgcatgagtccaaggtccaggaacagcacgatcaaataattaatgcttcatctcagattcaaattccaaatcatgctttagggcatggccatcaggcatctcttcctaatacacaggtccttttagattctgcctgtgatttacaaattcttcagcagtcaatactgcaggcaggtttaggtcaagtaaaggcatctttacaagcacagcgtgttcaaagccctcaacaaatagtacatcccttccttcagatggaaggtcatgttattcaaagcaatggtgatcattctcagcagcaactccatcctcaaaattctgaagttatgaaaatggacctctctgagtcttcaaaaccattacaacaacatctaacaacaaagggccattttagtgaaacaaatcaacatgattcaaagaatcagttgtttctcttggatcgatgtgtttcccagaggcaggcttcttagtgatgaaagaaatattttatcaaatgtagatgatatcttagcagctacagcagcagcttgtggagttacacctactgatttttccaagtcaacttcaaatgaaaccatgcaggctgttgaagatggtgattctaaatctcattttcagcagtcattagatgtcaggcatgtgacttcagattttaactctatgacagctacagtaggaaagccacagaatataaatgatacttccttaaatggaaatcaggttactgtgaacctttcaccagtacctgcccttcagtcaaaaatgactcttgatcaacagcacattgaaacacctggtcaaaatataccaactaaagtaacttcagcagtggttggaccaagtcatgaagtccaggagcaaagttctggcccattcaagaaacagtctgctaccaatcttgaatctgaagaagacagtgaagctcctgttgatagtacattaaataataacagaaaccaagagtttgtttctagtagtagaagtataagtggagagagtgctacatcagagagtgaatttaccttagggggtgacgacagtggtgtgtcaatgaacccagctaggagtgcacttgcactgttggccatggcccaatctggggatgcagtcagtgtcaagattgaagaagaaaaccaagatttaatgcattttaaccttcaaaagaaaagagctaaaggaaaagggcaagttaaagaggaagacaacagtaatcagaaacagctgaaaagacctgcccaaggcaaacgccagaatccaaggggaacagatatttacttaccgtatactcctccttcctcagaaagctgccatgatggttatcagcatcaagaaaaaatgagacagaagatcaaagaggtggaggaaaaacaaccggaagtcaaaacaggatttattgcttctttcttagattttctgaaatccgggcccaagcagcagttttccactcttgctgtacgaatgcctaacaggactagacggccagggacccagatggttcgtacattttgtcccccaccacttcccaagccttcatctacaacacccacacctttagtgtctgaaactggcggtaacagtccatcagataaagttgataatgaacttaaaaacttggaacatttatcttcattttcttctgatgaagatgatcctggatatagtcaagatgcttataaaagcgtctctactcccttaactactttggatgctacttctgataaaaagaagaaaacagaagccctacaggtggcaactactagcccaactgccaatactactggtactgctactacttcctcaaccactgtgggtgcagttaagcaagaacctctccactctacttcatatgcagtaaatattctggaaaatataagctcttcagaatcctcaaagcccattgaacttgatggtcttccttcagaccagtttgcaaaaggacaggacactgttgccatagaaggttttacagatgaggaggacacagaaagcggaggagaaggccaatacagagagcgtgatgaatttgtggtaaagatagaagacatagagacttttaaggaggctttaaaaacaggaaaagaacctccagctatttggaaagtacaaaaagctttattacagaaatttgttcctgaaattcgagatggtcaaagagaatttgctgctacaaatagttatcttggatattttggagatgcaaagagtaaatacaaaagaatatatgtgaagttcattgaaaatgcaaacaagaaggaatatgtcagagtgtgttctaaaaagccaagaaataaaccttcacaaactatcagaactgttcaagctaagccaagtagtagcagtaaaacttctgatcctctagcatcaaaaactacaactacaaaagccccttccgtgaaacccaaagttaaacagccaaaagtaaaggctgagccaccaccaaagaaacggaaaaaatggaaagaagaattttcatcatcccaatctgactcatctcctgagatccatactagtagtagtgacgatgaggaatttgaacctcccgctccctttgtcactcgctttttgaacacaagagcaatgaaggaaacctttaagagctacatggaattgcttgttagcattgccttggaccctgacacaatgcaagccttagagaagagcaatgatgagctacttttacctcatatgaaaaaaatagatggcatgctaaatgataaccgaaagagacttcttttgaatcttcatttggatcaatcattcaagaatgctttggaaagttttcctgaactaacaataattactcgagattctaaagcaaagagtggaggaactgctatttctaaaatcaaaatgaatggcaaagcctataataagaaaactctaaggacttctaaaacaaccaccaaatctgcacaagagtttgctgtcgatccagagaaaatacagttgtattctttgtatcattcactccatcattataagtaccatgtttatctgatatgtaaggatgagatttcttcggtgcagaaaaaaaatgaagatttaggacaggaggaaattgttcaactttgtatgaaaaatgtaaaatgggtggaggacctctttgaaaaatttggagaacttctaaatcatgtacagcagaaatgttcctgacttttccacaaaaatcccatctttttatagcactaatgaaatggcagatatggggtggtcaaagataatcagatgtcaagtagtggccttctgcaggccggccgcttccatcatggaactgtcattaccacctctgctgaaggacagtggtgcggcctttaggaacgaagttagtcctctggaaatggacctaaatcccaccacatttttaccctaatgaatgatttttctattttgtaaaccattgggtaacttgagtcatattttcagaaacattttttgacaaatgatgaagcatgcactaagtataatttttttttattgctagagaagtaacacttaaagtaacgattttttttttctgactccggctaaacaccagaatgacagagaagtggcagaaaccatatgtttgtactcacatctggccacaaaaccagaaatactgtacattatgtaaagaggtctggtgtggtgtgacatcctgtataagaatatcatcaatttaaaatataaaatttggaaactattctgctttacagactccttttactcttaacatgttcaggaaactggatgtggaattggtgcaattctctgactgctttttgtgtcaaattatattgtgataaaaaaacaatgacatactattttccctatcgcaaagaaaagtattttcgtttatactgttttttcctttggaaaattttcaattgtacattttatttcactgatagttgtatttttcacaaggaaaatgttgtggttataattacgtttgatatatctctacaacaccttttgttattttcagtaaatcttagttatatgttgaatttctaatgtgaattctatcttgaggtaaccatttttttcatacagatttgcttcagtgttatccagaatatgcattcagtactagaattagtttagctttataaatagggctgtgttagacactgcagtaattttctaattcataaaataaacttcttactaaactagcacttgattaacttgttgaggtaaaaatctaactacatttacattttgaagaataaaactgataattagactatttgcagtgttaaacacagcttccttaactcttagaactggaagttgtagagctctccttttggtgcctttccagcctttatacacactattgtagctttcttaggtttgataggtagcgtttcaagtagtttagctgagacagtgaatgtattaggttcaacatgaccttgtgttttatttgtgtttgccaacaggatgccttatttgtttgagaaaaagatgtactagtgtcattctaaactatctccttttttaggattctaaagaagtta

SEQ ID NO: 96 - DNA, 1410 bp, Homo sapiens
  misc_feature: Incyte ID No 90089609CB1
aagaagtctgagcctgccttagctgccccccaaggccatccaagctgtcaccgccaccacctccagcccccctccctgccatcctgcttcccaggcagccctcactaccatccggcgaccactctaagtccaagcatccagcctccctgtcctaaccagccacccccatcggcccccatacagcactaaccaggagcatcttagcccagctgcctccctcccgcccacgaagctgtcctgccttcctggtgtcctggaacagagggaggccctaacctgcccattccagctcccagagacataccctcctctgggttccttcacacccaaccccctcttctccttgaccccacccatctccacccaccagtccctccccttccttcagggctggggactcgtgggtgcagctggcaccctagtcctgcctgaccccccaaggccaagggggtacgtggaaggggttacagaagcaagcagatgaggcgcagcccagcccccccggcaccttggtgagcgccccggggtggaaggtccagcgcgtctcgtggttgaactgcacgcgcacgtccccgcggtccgtgatacgatgcacggtgcccgtctgtccgataaactgtggcgggtcatggagctgtggctgggtcgggctggacgcttggggaggaaggggctgagggggcggagggctgggagggcagggcacacagggctccacggcggggcggctcacctccgccatcctggggttccagccgccgtggccttcctgcatctcccgcaggacatcagtgtccagcagacacttgaccttgtccccgtgctggaagggctggctgtcagcactcaccctgcgctgcagctccgccggcttgcctgcggagggtggcagaccaggcctcaggcagcggcccctcccctctgggacttcagccaaggcaggagggctgatggagtcagtgtgacagcctcataccgagccttgggaggtggtccttgtagtagaagccgcccgctgcctcgcccacacacttgaggtccaccttgcccttgtggcccacacggtacacattggtggtaccatcagcccacgtcacgctggccacactccggcctgtctccacatcccagccacggatgtccaccacacggcccggtttcccttcccctcctggggaaagaggcccccccagcaggctaaggctgagccatgagccagccacagcccagccctgaccccgcccctctgcctccactcaccatcctgtgagccccactcccagtcggggcctcgcaccaccttcgctccctggaagatgccccttagtgggatcctcgggaggccctggcggggactcagtgtgacactgcaagagggttcacaaataaagctctcagaagaaaaaaaaa

(19) United States
(12) Patent Application Publication
(10) Pub. No.: US 2006/0051836 A1
(43) Pub. Date: Mar. 9, 2006

(54) Molecules for disease detection and treatment

(76) Inventors: Y Tom Tang, San Jose, CA (US); Ian J Forsythe, Redwood City, CA (US); Brooke M Emerling, Palo Alto, CA (US); April J A Hafalia, Santa Clara, CA (US); Henry Yue, Sunnyvale, CA (US); Kimberly J Gietzen, San Jose, CA (US); Narinder K Chawla, Union City, CA (US); Joseph P Marquis, San Jose, CA (US); Shanya D Becha, Castro Valley, CA (US); Amy E Kable, Francisco, CA (US); Preeti G Lal, Santa Clara, CA (US); Thomas W Richardson, Redwood, CA (US); Soo Yeun Lee, Daly City, CA (US); Ernestine A Lee, Castro Valley, CA (US); Bao Tran, Santa Clara, CA (US)

Correspondence Address: INCYTE CORPORATION;EXPERIMENTAL STATION, ROUTE 141 & HENRY CLAY ROAD, BLDG. E336, WILMINGTON, DE, 19880, US

(21) Appl. No.: 10/491,468

(22) PCT Filed: Oct. 10, 2002
(86) PCT No.: PCT/US02/32852

§ 371 Date: Mar. 31, 2004

Related U.S. Application Data
(60) Provisional application No. 60/328,944, filed on Oct. 12, 2001.
(60) Provisional application No. 60/345,384, filed on Oct. 26, 2001.
(60) Provisional application No. 60/343,880, filed on Nov. 2, 2001.
(60) Provisional application No. 60/345,143, filed on Nov. 9, 2001.
(60) Provisional application No. 60/332,430, filed on Nov. 16, 2001.

(51) Int. Cl. C12P 21/06; C07H 21/04; C07K 14/47

(52) U.S. Cl. 435/69.1; 435/320.1; 435/325; 530/350; 536/23.5

(57) ABSTRACT

Various embodiments of the invention provide human molecules for disease detection and treatment (MDDT) and polynucleotides which identify and encode MDDT. Embodiments of the invention also provide expression vectors, host cells, antibodies, agonists, and antagonists. Other embodiments provide methods for diagnosing, treating, or preventing disorders associated with aberrant expression of MDDT.

### TECHNICAL FIELD

The invention relates to novel nucleic acids, molecules for disease detection and treatment encoded by these nucleic acids, and to the use of these nucleic acids and proteins in the diagnosis, treatment, and prevention of cell proliferative, autoimmune/inflammatory, developmental, and neurological disorders. The invention also relates to the assessment of the effects of exogenous compounds on the expression of nucleic acids and molecules for disease detection and treatment.

### BACKGROUND OF THE INVENTION

It is estimated that only 2% of mammalian DNA encodes proteins, and only a small fraction of the genes that encode proteins are actually expressed in a particular cell at any time. The various types of cells in a multicellular organism differ dramatically both in structure and function, and the identity of a particular cell is conferred by its unique pattern of gene expression. In addition, different cell types express overlapping but distinctive sets of genes throughout development. Cell growth and proliferation, cell differentiation, the immune response, apoptosis, and other processes that contribute to organismal development and survival are governed by regulation of gene expression. Appropriate gene regulation also ensures that cells function efficiently by expressing only those genes whose functions are required at a given time. Factors that influence gene expression include extracellular signals that mediate cell-cell communication and coordinate the activities of different cell types. Gene expression is regulated at the level of DNA and RNA transcription, and at the level of mRNA translation.

Aberrant expression or mutations in genes and their products may cause, or increase susceptibility to, a variety of human diseases such as cancer and other cell proliferative disorders. The identification of these genes and their products is the basis of an ever-expanding effort to find markers for early detection of diseases and targets for their prevention and treatment. For example, cancer represents a type of cell proliferative disorder that affects nearly every tissue in the body. The development of cancer, or oncogenesis, is often correlated with the conversion of a normal gene into a cancer-causing gene, or oncogene, through abnormal expression or mutation. Oncoproteins, the products of oncogenes, include a variety of molecules that influence cell proliferation, such as growth factors, growth factor receptors, intracellular signal transducers, nuclear transcription factors, and cell-cycle control proteins. In contrast, tumor-suppressor genes are involved in inhibiting cell proliferation. Mutations which reduce or abrogate the function of tumor-suppressor genes result in aberrant cell proliferation and cancer. Thus a wide variety of genes and their products have been found that are associated with cell proliferative disorders such as cancer, but many more may exist that are yet to be discovered.

DNA-based arrays can provide an efficient, high-throughput method to examine gene expression and genetic variability. For example, SNPs, or single nucleotide polymorphisms, are the most common type of human genetic variation. DNA-based arrays can dramatically accelerate the discovery of SNPs in hundreds and even thousands of genes. Likewise, such arrays can be used for SNP genotyping in which DNA samples from individuals or populations are assayed for the presence of selected SNPs. These approaches will ultimately lead to the systematic identification of all genetic variations in the human genome and the correlation of certain genetic variations with disease susceptibility, responsiveness to drug treatments, and other medically relevant information. (See, for example, Wang, D. G. et al. (1998) Science 280:1077-1082.)

DNA-based array technology is especially important for the rapid analysis of global gene expression patterns. For example, genetic predisposition, disease, or therapeutic treatment may directly or indirectly affect the expression of a large number of genes in a given tissue. In this case, it is useful to develop a profile, or transcript image, of all the genes that are expressed and the levels at which they are expressed in that particular tissue. A profile generated from an individual or population affected with a certain disease or undergoing a particular therapy may be compared with a profile generated from a control individual or population. Such analysis does not require knowledge of gene function, as the expression profiles can be subjected to mathematical analyses which simply treat each gene as a marker. Furthermore, gene expression profiles may help dissect biological pathways by identifying all the genes expressed, for example, at a certain developmental stage, in a particular tissue, or in response to disease or treatment. (See, for example, Lander, E. S. et al. (1996) Science 274:536-539.)

Certain genes are known to be associated with diseases because of their chromosomal location, such as the genes in the myotonic dystrophy (DM) regions of mouse and human. The mutation underlying DM has been localized to a gene encoding the DM-kinase protein, but another active gene, DMR-N9, is in close proximity to the DM-kinase gene (Jansen, G. et al. (1992) Nat. Genet. 1:261-266). DMR-N9 encodes a 650 amino acid protein that contains WD repeats, motifs found in cell signaling proteins. DMR-N9 is expressed in all neural tissues and in the testis, suggesting a role for DMR-N9 in the manifestation of mental and testicular symptoms in severe cases of DM (Jansen, G. et al. (1995) Hum. Mol. Genet. 4:843-852).

Other types of signaling proteins include the WW domain, which consists of 35-40 amino acids and is characterized by four well-conserved aromatic residues, two of which are tryptophan. The secondary structure of the WW domain consists of a slightly bent three-stranded antiparallel sheet. This domain has been reported in a wide variety of proteins, including human Pin 1 and Ras GAP-related protein. The presence of the WW domain in diverse proteins is involved in signaling, regulatory, and cytoskeletal functions. Defects in WW domains containing proteins are associated with human diseases such as Liddle Syndrome (Pirozzi, G. et al. (1997) J. Biol. Chem. 272:14611-14616). The tetratricopeptide repeat (TPR) is composed of a degenerate 34 amino acid sequence present in various proteins. Their array as multiple motifs enable formation of scaffolds that mediate protein-protein interactions and assembly of multiprotein complexes (Das, A. K. et al. (1998) EMBO J. 17:1192-1199). CheB methylesterase catalyzes hydrolysis of receptor glutamine or methylglutamate side-chains to glutamic acid, and belongs to a large family of response regulator proteins in which N-terminal regulatory domains control the activities of C-terminal effector domains.

Other genes are identified based upon their expression patterns or association with disease syndromes. For example, autoantibodies to subcellular organelles are found in patients with systemic rheumatic diseases. A recently identified protein, golgin-67, belongs to a family of Golgi autoantigens having alpha-helical coiled-coil domains (Eystathioy, T. et al. (2000) J. Autoimmun. 14:179-187). The Stac gene was identified as a brain specific, developmentally regulated gene. The Stac protein contains an SH3 domain, and is thought to be involved in neuron-specific signal transduction (Suzuki, H. et al. (1996) Biochem. Biophys. Res. Commun. 229:902-909).

Expression Profiling

Microarrays are analytical tools used in bioanalysis. A microarray has a plurality of molecules spatially distributed over, and stably associated with, the surface of a solid support. Microarrays of polypeptides, polynucleotides, and/or antibodies have been developed and find use in a variety of applications, such as gene sequencing, monitoring gene expression, gene mapping, bacterial identification, drug discovery, and combinatorial chemistry.

One area in particular in which microarrays find use is in gene expression analysis. Array technology can provide a simple way to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for identifying genes that are tissue specific, are affected by a substance being tested in a toxicology assay, are part of a signaling cascade, carry out housekeeping functions, or are specifically related to a particular genetic predisposition, condition, disease, or disorder.

There is a need in the art for new compositions, including nucleic acids and proteins, for the diagnosis, prevention, and treatment of cell proliferative, autoimmune/inflammatory, developmental, and neurological disorders.

### SUMMARY OF THE INVENTION

Various embodiments of the invention provide purified polypeptides, molecules for disease detection and treatment, referred to collectively as ‘MDDT’ and individually as ‘MDDT-1,’ ‘MDDT-2,’ ‘MDDT-3,’ ‘MDDT-4,’ ‘MDDT-5,’ ‘MDDT-6,’ ‘MDDT-7,’ ‘MDDT-8,’ ‘MDDT-9,’ ‘MDDT-10,’ ‘MDDT-11,’ ‘MDDT-12,’ ‘MDDT-13,’ ‘MDDT-14,’ ‘MDDT-15,’ ‘MDDT-16,’ ‘MDDT-17,’ ‘MDDT-18,’ ‘MDDT-19,’ ‘MDDT-20,’ ‘MDDT-21,’ ‘MDDT-22,’ ‘MDDT-23,’ ‘MDDT-24,’ ‘MDDT-25,’ ‘MDDT-26,’ ‘MDDT-27,’ ‘MDDT-28,’ ‘MDDT-29,’ ‘MDDT-30,’ ‘MDDT-31,’ ‘MDDT-32,’ ‘MDDT-33,’ ‘MDDT-34,’ ‘MDDT-35,’ ‘MDDT-36,’ ‘MDDT-37,’ ‘MDDT-38,’ ‘MDDT-39,’ ‘MDDT-40,’ ‘MDDT-41,’ ‘MDDT-42,’ ‘MDDT-43,’ ‘MDDT-44,’ ‘MDDT-45,’ ‘MDDT-46,’ ‘MDDT-47,’ and ‘MDDT-48’ and methods for using these proteins and their encoding polynucleotides for the detection, diagnosis, and treatment of diseases and medical conditions. Embodiments also provide methods for utilizing the purified molecules for disease detection and treatment and/or their encoding polynucleotides for facilitating the drug discovery process, including determination of efficacy, dosage, toxicity, and pharmacology. Related embodiments provide methods for utilizing the purified molecules for disease detection and treatment and/or their encoding polynucleotides for investigating the pathogenesis of diseases and medical conditions.

An embodiment provides an isolated polypeptide selected from the group consisting of a) a polypeptide comprising an amino acid sequence selected from the group consisting of SEQ ID NO:1-48, b) a polypeptide comprising a naturally occurring amino acid sequence at least 90% identical or at least about 90% identical to an amino acid sequence selected from the group consisting of SEQ ID NO:1-48, c) a biologically active fragment of a polypeptide having an amino acid sequence selected from the group consisting of SEQ ID NO:1-48, and d) an immunogenic fragment of a polypeptide having an amino acid sequence selected from the group consisting of SEQ ID NO:1-48. Another embodiment provides an isolated polypeptide comprising an amino acid sequence of SEQ ID NO:1-48.

Still another embodiment provides an isolated polynucleotide encoding a polypeptide selected from the group consisting of a) a polypeptide comprising an amino acid sequence selected from the group consisting of SEQ ID NO:1-48, b) a polypeptide comprising a naturally occurring amino acid sequence at least 90% identical or at least about 90% identical to an amino acid sequence selected from the group consisting of SEQ ID NO:1-48, c) a biologically active fragment of a polypeptide having an amino acid sequence selected from the group consisting of SEQ ID NO:1-48, and d) an immunogenic fragment of a polypeptide having an amino acid sequence selected from the group consisting of SEQ ID NO:1-48. In another embodiment, the polynucleotide encodes a polypeptide selected from the group consisting of SEQ ID NO:1-48. In an alternative embodiment, the polynucleotide is selected from the group consisting of SEQ ID NO:49-96.

Still another embodiment provides a recombinant polynucleotide comprising a promoter sequence operably linked to a polynucleotide encoding a polypeptide selected from the group consisting of a) a polypeptide comprising an amino acid sequence selected from the group consisting of SEQ ID NO:1-48, b) a polypeptide comprising a naturally occurring amino acid sequence at least 90% identical or at least about 90% identical to an amino acid sequence selected from the group consisting of SEQ ID NO:1-48, c) a biologically active fragment of a polypeptide having an amino acid sequence selected from the group consisting of SEQ ID NO:1-48, and d) an immunogenic fragment of a polypeptide having an amino acid sequence selected from the group consisting of SEQ ID NO:1-48. Another embodiment provides a cell transformed with the recombinant polynucleotide. Yet another embodiment provides a transgenic organism comprising the recombinant polynucleotide.

Another embodiment provides a method for producing a polypeptide selected from the group consisting of a) a polypeptide comprising an amino acid sequence selected from the group consisting of SEQ ID NO:1-48, b) a polypeptide comprising a naturally occurring amino acid sequence at least 90% identical or at least about 90% identical to an amino acid sequence selected from the group consisting of SEQ ID NO:1-48, c) a biologically active fragment of a polypeptide having an amino acid sequence selected from the group consisting of SEQ ID NO:1-48, and d) an immunogenic fragment of a polypeptide having an amino acid sequence selected from the group consisting of SEQ ID NO:1-48. The method comprises a) culturing a cell under conditions suitable for expression of the polypeptide, wherein said cell is transformed with a recombinant polynucleotide comprising a promoter sequence operably linked to a polynucleotide encoding the polypeptide, and b) recovering the polypeptide so expressed.

Yet another embodiment provides an isolated antibody which specifically binds to a polypeptide selected from the group consisting of a) a polypeptide comprising an amino acid sequence selected from the group consisting of SEQ ID NO:1-48, b) a polypeptide comprising a naturally occurring amino acid sequence at least 90% identical or at least about 90% identical to an amino acid sequence selected from the group consisting of SEQ ID NO:1-48, c) a biologically active fragment of a polypeptide having an amino acid sequence selected from the group consisting of SEQ ID NO:1-48, and d) an immunogenic fragment of a polypeptide having an amino acid sequence selected from the group consisting of SEQ ID NO:1-48.

Still yet another embodiment provides an isolated polynucleotide selected from the group consisting of a) a polynucleotide comprising a polynucleotide sequence selected from the group consisting of SEQ ID NO:49-96, b) a polynucleotide comprising a naturally occurring polynucleotide sequence at least 90% identical or at least about 90% identical to a polynucleotide sequence selected from the group consisting of SEQ ID NO:49-96, c) a polynucleotide complementary to the polynucleotide of a), d) a polynucleotide complementary to the polynucleotide of b), and e) an RNA equivalent of a)-d). In other embodiments, the polynucleotide can comprise at least about 20, 30, 40, 60, 80, or 100 contiguous nucleotides.

Yet another embodiment provides a method for detecting a target polynucleotide in a sample, said target polynucleotide being selected from the group consisting of a) a polynucleotide comprising a polynucleotide sequence selected from the group consisting of SEQ ID NO:49-96, b) a polynucleotide comprising a naturally occurring polynucleotide sequence at least 90% identical or at least about 90% identical to a polynucleotide sequence selected from the group consisting of SEQ ID NO:49-96, c) a polynucleotide complementary to the polynucleotide of a), d) a polynucleotide complementary to the polynucleotide of b), and e) an RNA equivalent of a)-d). The method comprises a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and b) detecting the presence or absence of said hybridization complex. In a related embodiment, the method can include detecting the amount of the hybridization complex. In still other embodiments, the probe can comprise at least about 20, 30, 40, 60, 80, or 100 contiguous nucleotides.

Still yet another embodiment provides a method for detecting a target polynucleotide in a sample, said target polynucleotide being selected from the group consisting of a) a polynucleotide comprising a polynucleotide sequence selected from the group consisting of SEQ ID NO:49-96, b) a polynucleotide comprising a naturally occurring polynucleotide sequence at least 90% identical or at least about 90% identical to a polynucleotide sequence selected from the group consisting of SEQ ID NO:49-96, c) a polynucleotide complementary to the polynucleotide of a), d) a polynucleotide complementary to the polynucleotide of b), and e) an RNA equivalent of a)-d). The method comprises a) amplifying said target polynucleotide or fragment thereof using polymerase chain reaction amplification, and b) detecting the presence or absence of said amplified target polynucleotide or fragment thereof. In a related embodiment, the method can include detecting the amount of the amplified target polynucleotide or fragment thereof.

Another embodiment provides a composition comprising an effective amount of a polypeptide selected from the group consisting of a) a polypeptide comprising an amino acid sequence selected from the group consisting of SEQ ID NO:1-48, b) a polypeptide comprising a naturally occurring amino acid sequence at least 90% identical or at least about 90% identical to an amino acid sequence selected from the group consisting of SEQ ID NO:1-48, c) a biologically active fragment of a polypeptide having an amino acid sequence selected from the group consisting of SEQ ID NO:1-48, and d) an immunogenic fragment of a polypeptide having an amino acid sequence selected from the group consisting of SEQ ID NO:1-48, and a pharmaceutically acceptable excipient. In one embodiment, the composition can comprise an amino acid sequence selected from the group consisting of SEQ ID NO:1-48. Other embodiments provide a method of treating a disease or condition associated with decreased or abnormal expression of functional MDDT, comprising administering to a patient in need of such treatment the composition.

Yet another embodiment provides a method for screening a compound for effectiveness as an agonist of a polypeptide selected from the group consisting of a) a polypeptide comprising an amino acid sequence selected from the group consisting of SEQ ID NO:1-48, b) a polypeptide comprising a naturally occurring amino acid sequence at least 90% identical or at least about 90% identical to an amino acid sequence selected from the group consisting of SEQ ID NO:1-48, c) a biologically active fragment of a polypeptide having an amino acid sequence selected from the group consisting of SEQ ID NO:1-48, and d) an immunogenic fragment of a polypeptide having an amino acid sequence selected from the group consisting of SEQ ID NO:1-48. The method comprises a) exposing a sample comprising the polypeptide to a compound, and b) detecting agonist activity in the sample. Another embodiment provides a composition comprising an agonist compound identified by the method and a pharmaceutically acceptable excipient. Yet another embodiment provides a method of treating a disease or condition associated with decreased expression of functional MDDT, comprising administering to a patient in need of such treatment the composition.

Still yet another embodiment provides a method for screening a compound for effectiveness as an antagonist of a polypeptide selected from the group consisting of a) a polypeptide comprising an amino acid sequence selected from the group consisting of SEQ ID NO:1-48, b) a polypeptide comprising a naturally occurring amino acid sequence at least 90% identical or at least about 90% identical to an amino acid sequence selected from the group consisting of SEQ ID NO:1-48, c) a biologically active fragment of a polypeptide having an amino acid sequence selected from the group consisting of SEQ ID NO:1-48, and d) an immunogenic fragment of a polypeptide having an amino acid sequence selected from the group consisting of SEQ ID NO:1-48. The method comprises a) exposing a sample comprising the polypeptide to a compound, and b) detecting antagonist activity in the sample. Another embodiment provides a composition comprising an antagonist compound identified by the method and a pharmaceutically acceptable excipient. Yet another embodiment provides a method of treating a disease or condition associated with overexpression of functional MDDT, comprising administering to a patient in need of such treatment the composition.

Another embodiment provides a method of screening for a compound that specifically binds to a polypeptide selected from the group consisting of a) a polypeptide comprising an amino acid sequence selected from the group consisting of SEQ ID NO:1-48, b) a polypeptide comprising a naturally occurring amino acid sequence at least 90% identical or at least about 90% identical to an amino acid sequence selected from the group consisting of SEQ ID NO:1-48, c) a biologically active fragment of a polypeptide having an amino acid sequence selected from the group consisting of SEQ ID NO:1-48, and d) an immunogenic fragment of a polypeptide having an amino acid sequence selected from the group consisting of SEQ ID NO:1-48. The method comprises a) combining the polypeptide with at least one test compound under suitable conditions, and b) detecting binding of the polypeptide to the test compound, thereby identifying a compound that specifically binds to the polypeptide.

Yet another embodiment provides a method of screening for a compound that modulates the activity of a polypeptide selected from the group consisting of a) a polypeptide comprising an amino acid sequence selected from the group consisting of SEQ ID NO:1-48, b) a polypeptide comprising a naturally occurring amino acid sequence at least 90% identical or at least about 90% identical to an amino acid sequence selected from the group consisting of SEQ ID NO:1-48, c) a biologically active fragment of a polypeptide having an amino acid sequence selected from the group consisting of SEQ ID NO:1-48, and d) an immunogenic fragment of a polypeptide having an amino acid sequence selected from the group consisting of SEQ ID NO:1-48. The method comprises a) combining the polypeptide with at least one test compound under conditions permissive for the activity of the polypeptide, b) assessing the activity of the polypeptide in the presence of the test compound, and c) comparing the activity of the polypeptide in the presence of the test compound with the activity of the polypeptide in the absence of the test compound, wherein a change in the activity of the polypeptide in the presence of the test compound is indicative of a compound that modulates the activity of the polypeptide.

Still yet another embodiment provides a method for screening a compound for effectiveness in altering expression of a target polynucleotide, wherein said target polynucleotide comprises a polynucleotide sequence selected from the group consisting of SEQ ID NO:49-96, the method comprising a) exposing a sample comprising the target polynucleotide to a compound, b) detecting altered expression of the target polynucleotide, and c) comparing the expression of the target polynucleotide in the presence of varying amounts of the compound and in the absence of the compound.

Another embodiment provides a method for assessing toxicity of a test compound, said method comprising a) treating a biological sample containing nucleic acids with the test compound; b) hybridizing the nucleic acids of the treated biological sample with a probe comprising at least 20 contiguous nucleotides of a polynucleotide selected from the group consisting of i) a polynucleotide comprising a polynucleotide sequence selected from the group consisting of SEQ ID NO:49-96, ii) a polynucleotide comprising a naturally occurring polynucleotide sequence at least 90% identical or at least about 90% identical to a polynucleotide sequence selected from the group consisting of SEQ ID NO:49-96, iii) a polynucleotide having a sequence complementary to i), iv) a polynucleotide complementary to the polynucleotide of ii), and v) an RNA equivalent of i)-iv). Hybridization occurs under conditions whereby a specific hybridization complex is formed between said probe and a target polynucleotide in the biological sample, said target polynucleotide selected from the group consisting of i) a polynucleotide comprising a polynucleotide sequence selected from the group consisting of SEQ ID NO:49-96, ii) a polynucleotide comprising a naturally occurring polynucleotide sequence at least 90% identical or at least about 90% identical to a polynucleotide sequence selected from the group consisting of SEQ ID NO:49-96, iii) a polynucleotide complementary to the polynucleotide of i), iv) a polynucleotide complementary to the polynucleotide of ii), and v) an RNA equivalent of i)-iv). Alternatively, the target polynucleotide can comprise a fragment of a polynucleotide selected from the group consisting of i)-v) above; c) quantifying the amount of hybridization complex; and d) comparing the amount of hybridization complex in the treated biological sample with the amount of hybridization complex in an untreated biological sample, wherein a difference in the amount of hybridization complex in the treated biological sample is indicative of toxicity of the test compound.

### BRIEF DESCRIPTION OF THE TABLES

Table 1 summarizes the nomenclature for full length polynucleotide and polypeptide embodiments of the invention.

Table 2 shows the GenBank identification number and annotation of the nearest GenBank homolog, and the PROTEOME database identification numbers and annotations of PROTEOME database homologs, for polypeptide embodiments of the invention. The probability scores for the matches between each polypeptide and its homolog(s) are also shown.

Table 3 shows structural features of polypeptide embodiments, including predicted motifs and domains, along with the methods, algorithms, and searchable databases used for analysis of the polypeptides.

Table 4 lists the cDNA and/or genomic DNA fragments which were used to assemble polynucleotide embodiments, along with selected fragments of the polynucleotides.

Table 5 shows representative cDNA libraries for polynucleotide embodiments.

Table 6 provides an appendix which describes the tissues and vectors used for construction of the cDNA libraries shown in Table 5.

Table 7 shows the tools, programs, and algorithms used to analyze polynucleotides and polypeptides, along with applicable descriptions, references, and threshold parameters.

### DESCRIPTION OF THE INVENTION

Before the present proteins, nucleic acids, and methods are described, it is understood that embodiments of the invention are not limited to the particular machines, instruments, materials, and methods described, as these may vary. It is also to be understood that the terminology used herein is for the purpose of describing particular embodiments only, and is not intended to limit the scope of the invention.

As used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with various embodiments of the invention. Nothing herein is to be construed as an admission that the invention is not entitled to antedate such disclosure by virtue of prior invention.

Definitions

“MDDT” refers to the amino acid sequences of substantially purified MDDT obtained from any species, particularly a mammalian species, including bovine, ovine, porcine, murine, equine, and human, and from any source, whether natural, synthetic, semi-synthetic, or recombinant.

The term “agonist” refers to a molecule which intensifies or mimics the biological activity of MDDT. Agonists may include proteins, nucleic acids, carbohydrates, small molecules, or any other compound or composition which modulates the activity of MDDT either by directly interacting with MDDT or by acting on components of the biological pathway in which MDDT participates.

An “allelic variant” is an alternative form of the gene encoding MDDT. Allelic variants may result from at least one mutation in the nucleic acid sequence and may result in altered mRNAs or in polypeptides whose structure or function may or may not be altered. A gene may have none, one, or many allelic variants of its naturally occurring form. Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding MDDT include those sequences with deletions, insertions, or substitutions of different nucleotides, resulting in a polypeptide the same as MDDT or a polypeptide with at least one functional characteristic of MDDT. Included within this definition are polymorphisms which may or may not be readily detectable using a particular oligonucleotide probe of the polynucleotide encoding MDDT, and improper or unexpected hybridization to allelic variants, with a locus other than the normal chromosomal locus for the polynucleotide encoding MDDT. The encoded protein may also be “altered,” and may contain deletions, insertions, or substitutions of amino acid residues which produce a silent change and result in a functionally equivalent MDDT. Deliberate amino acid substitutions may be made on the basis of one or more similarities in polarity, charge, solubility, hydrophobicity, hydrophilicity, and/or the amphipathic nature of the residues, as long as the biological or immunological activity of MDDT is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

The terms “amino acid” and “amino acid sequence” can refer to an oligopeptide, a peptide, a polypeptide, or a protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

“Amplification” relates to the production of additional copies of a nucleic acid. Amplification may be carried out using polymerase chain reaction (PCR) technologies or other nucleic acid amplification technologies well known in the art.

The term “antagonist” refers to a molecule which inhibits or attenuates the biological activity of MDDT. Antagonists may include proteins such as antibodies, anticalins, nucleic acids, carbohydrates, small molecules, or any other compound or composition which modulates the activity of MDDT either by directly interacting with MDDT or by acting on components of the biological pathway in which MDDT participates.

The term “antibody” refers to intact immunoglobulin molecules as well as to fragments thereof, such as Fab, F(ab′)₂, and Fv fragments, which are capable of binding an epitopic determinant. Antibodies that bind MDDT polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “antigenic determinant” refers to that region of a molecule (i.e., an epitope) that makes contact with a particular antibody. When a protein or a fragment of a protein is used to immunize a host animal, numerous regions of the protein may induce the production of antibodies which bind specifically to antigenic determinants (particular regions or three-dimensional structures on the protein). An antigenic determinant may compete with the intact antigen (i.e., the immunogen used to elicit the immune response) for binding to an antibody.

The term “aptamer” refers to a nucleic acid or oligonucleotide molecule that binds to a specific molecular target. Aptamers are derived from an in vitro evolutionary process (e.g., SELEX (Systematic Evolution of Ligands by EXponential Enrichment), described in U.S. Pat. No. 5,270,163), which selects for target-specific aptamer sequences from large combinatorial libraries. Aptamer compositions may be double-stranded or single-stranded, and may include deoxyribonucleotides, ribonucleotides, nucleotide derivatives, or other nucleotide-like molecules. The nucleotide components of an aptamer may have modified sugar groups (e.g., the 2′-OH group of a ribonucleotide may be replaced by 2′-F or 2′-NH₂), which may improve a desired property, e.g., resistance to nucleases or longer lifetime in blood. Aptamers may be conjugated to other molecules, e.g., a high molecular weight carrier to slow clearance of the aptamer from the circulatory system. Aptamers may be specifically cross-linked to their cognate ligands, e.g., by photo-activation of a cross-linker (Brody, E. N. and L. Gold (2000) J. Biotechnol. 74:5-13).

The term “intramer” refers to an aptamer which is expressed in vivo. For example, a vaccinia virus-based RNA expression system has been used to express specific RNA aptamers at high levels in the cytoplasm of leukocytes (Blind, M. et al. (1999) Proc. Natl. Acad. Sci. USA 96:3606-3610).

The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides.

The term “antisense” refers to any composition capable of base-pairing with the “sense” (coding) strand of a polynucleotide having a specific nucleic acid sequence. Antisense compositions may include DNA; RNA; peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules may be produced by any method including chemical synthesis or transcription. Once introduced into a cell, the complementary antisense molecule base-pairs with a naturally occurring nucleic acid sequence produced by the cell to form duplexes which block either transcription or translation. The designation “negative” or “minus” can refer to the antisense strand, and the designation “positive” or “plus” can refer to the sense strand of a reference DNA molecule.

The term “biologically active” refers to a protein having structural, regulatory, or biochemical functions of a naturally occurring molecule. Likewise, “immunologically active” or “immunogenic” refers to the capability of the natural, recombinant, or synthetic MDDT, or of any oligopeptide thereof, to induce a specific immune response in appropriate animals or cells and to bind with specific antibodies.

“Complementary” describes the relationship between two single-stranded nucleic acid sequences that anneal by base-pairing. For example, 5′-AGT-3′ pairs with its complement, 3′-TCA-5′.

A “composition comprising a given polynucleotide” and a “composition comprising a given polypeptide” can refer to any composition containing the given polynucleotide or polypeptide. The composition may comprise a dry formulation or an aqueous solution. Compositions comprising polynucleotides encoding MDDT or fragments of MDDT may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (e.g., sodium dodecyl sulfate; SDS), and other components (e.g., Denhardt's solution, dry milk, salmon sperm DNA, etc.).

“Consensus sequence” refers to a nucleic acid sequence which has been subjected to repeated DNA sequence analysis to resolve uncalled bases, extended using the XL-PCR kit (Applied Biosystems, Foster City Calif.) in the 5′ and/or the 3′ direction, and resequenced, or which has been assembled from one or more overlapping cDNA, EST, or genomic DNA fragments using a computer program for fragment assembly, such as the GELVIEW fragment assembly system (Accelrys, Burlington Mass.) or Phrap (University of Washington, Seattle Wash.). Some sequences have been both extended and assembled to produce the consensus sequence.

“Conservative amino acid substitutions” are those substitutions that are predicted to least interfere with the properties of the original protein, i.e., the structure and especially the function of the protein is conserved and not significantly changed by such substitutions. The table below shows amino acids which may be substituted for an original amino acid in a protein and which are regarded as conservative amino acid substitutions.

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid or nucleotide sequence that results in the absence of one or more amino acid residues or nucleotides.

The term “derivative” refers to a chemically modified polynucleotide or polypeptide. Chemical modifications of a polynucleotide can include, for example, replacement of hydrogen by an alkyl, acyl, hydroxyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A “detectable label” refers to a reporter molecule or enzyme that is capable of generating a measurable signal and is covalently or noncovalently joined to a polynucleotide or polypeptide.

“Differential expression” refers to increased or upregulated; or decreased, downregulated, or absent gene or protein expression, determined by comparing at least two different samples. Such comparisons may be carried out between, for example, a treated and an untreated sample, or a diseased and a normal sample.

“Exon shuffling” refers to the recombination of different coding regions (exons). Since an exon may represent a structural or functional domain of the encoded protein, new proteins may be assembled through the novel reassortment of stable substructures, thus allowing acceleration of the evolution of new protein functions.

A “fragment” is a unique portion of MDDT or a polynucleotide encoding MDDT which can be identical in sequence to, but shorter in length than, the parent sequence. A fragment may comprise up to the entire length of the defined sequence, minus one nucleotide/amino acid residue. For example, a fragment may comprise from about 5 to about 1000 contiguous nucleotides or amino acid residues. A fragment used as a probe, primer, antigen, therapeutic molecule, or for other purposes, may be at least 5, 10, 15, 16, 20, 25, 30, 40, 50, 60, 75, 100, 150, 250 or at least 500 contiguous nucleotides or amino acid residues in length. Fragments may be preferentially selected from certain regions of a molecule. For example, a polypeptide fragment may comprise a certain length of contiguous amino acids selected from the first 250 or 500 amino acids (or first 25% or 50%) of a polypeptide as shown in a certain defined sequence. Clearly these lengths are exemplary, and any length that is supported by the specification, including the Sequence Listing, tables, and figures, may be encompassed by the present embodiments.

A fragment of SEQ ID NO:49-96 can comprise a region of unique polynucleotide sequence that specifically identifies SEQ ID NO:49-96, for example, as distinct from any other sequence in the genome from which the fragment was obtained. A fragment of SEQ ID NO:49-96 can be employed in one or more embodiments of methods of the invention, for example, in hybridization and amplification technologies and in analogous methods that distinguish SEQ ID NO:49-96 from related polynucleotides. The precise length of a fragment of SEQ ID NO:49-96 and the region of SEQ ID NO:49-96 to which the fragment corresponds are routinely determinable by one of ordinary skill in the art based on the intended purpose for the fragment.

A fragment of SEQ ID NO:1-48 is encoded by a fragment of SEQ ID NO:49-96. A fragment of SEQ ID NO:1-48 can comprise a region of unique amino acid sequence that specifically identifies SEQ ID NO:1-48. For example, a fragment of SEQ ID NO:1-48 can be used as an immunogenic peptide for the development of antibodies that specifically recognize SEQ ID NO:1-48. The precise length of a fragment of SEQ ID NO:1-48 and the region of SEQ ID NO:1-48 to which the fragment corresponds can be determined based on the intended purpose for the fragment using one or more analytical methods described herein or otherwise known in the art.

A “full length” polynucleotide is one containing at least a translation initiation codon (e.g., methionine) followed by an open reading frame and a translation termination codon. A “full length” polynucleotide sequence encodes a “full length” polypeptide sequence.

“Homology” refers to sequence similarity or, alternatively, sequence identity, between two or more polynucleotide sequences or two or more polypeptide sequences.

The terms “percent identity” and “% identity,” as applied to polynucleotide sequences, refer to the percentage of identical residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using one or more computer algorithms or programs known in the art or described herein. For example, percent identity can be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and P. M. Sharp (1989; CABIOS 5:151-153) and in Higgins, D. G. et al. (1992; CABIOS 8:189-191). For pairwise alignments of polynucleotide sequences, the default parameters are set as follows: Ktuple=2, gap penalty=5, window=4, and “diagonals saved”=4. The “weighted” residue weight table is selected as the default.

Alternatively, a suite of commonly used and freely available sequence comparison algorithms which can be used is provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, Md., and on the Internet at http://www.ncbi.nlm.nih.gov/BLAST/. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at http://www.ncbi.nlm.nih.gov/gorf/bl2.html. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.12 (Apr.-21-2000) set at default parameters. Such default parameters may be, for example:
Matrix: BLOSUM62
Reward for match: 1
Penalty for mismatch: −2
Open Gap: 5 and Extension Gap: 2 penalties
Gap x drop-off: 50
Expect: 10
Word Size: 11
Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of identical residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. The phrases “percent similarity” and “% similarity,” as applied to polypeptide sequences, refer to the percentage of residue matches, including identical residue matches and conservative substitutions, between at least two polypeptide sequences aligned using a standardized algorithm. In contrast, conservative substitutions are not included in the calculation of percent identity between polypeptide sequences.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=1, gap penalty=3, window=5, and “diagonals saved”=5. The PAM250 matrix is selected as the default residue weight table.

Alternatively the NCBI BLAST software suite may be used. For example, for a pairwise comparison of two polypeptide sequences, one may use the “BLAST 2 Sequences” tool Version 2.0.12 (Apr.-21-2000) with blastp set at default parameters. Such default parameters may be, for example:
Matrix: BLOSUM62
Open Gap: 11 and Extension Gap: 1 penalties
Gap x drop-off: 50
Expect: 10
Word Size: 3
Filter: on

Percent identity may be measured over the length of an entire defined polypeptide sequence, for example, as defined by a particular SEQ D number, or may be measured over a shorter length, for example, over the length of a fragment taken from a larger, defined polypeptide sequence, for instance, a fragment of at least 15, at least 20, at least 30, at least 40, at least 50, at least 70 dr at least 150 contiguous residues. Such lengths are exemplary only, and it is understood that any fragment length supported by the sequences shown herein, in the tables, figures or Sequence Listing, may be used to describe a length over which percentage identity may be measured.

“Human artificial chromosomes” (HACs) are linear microchromosomes which may contain DNA sequences of about 6 kb to 10 Mb in size and which contain all of the elements required for chromosome replication, segregation and maintenance.

The term “humanized antibody” refers to an antibody molecule in which the amino acid sequence in the non-antigen binding regions has been altered so that the antibody more closely resembles a human antibody, and still retains its original binding ability.

“Hybridization” refers to the process by which a polynucleotide strand anneals with a complementary strand through base pairing under defined hybridization conditions. Specific hybridization is an indication that two nucleic acid sequences share a high degree of complementarity. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68° C. in the presence of about 6×SSC, about 1% (w/v) SDS, and about 100 μg/ml sheared, denatured salmon sperm DNA.

Generally, stringency of hybridization is expressed, in part, with reference to the temperature under which the wash step is carried out. Such wash temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook, J. and D. W. Russell (2001; Molecular Cloning: A Laboratory Manual, 3rd ed., vol. 1-3, Cold Spring Harbor Press, Cold Spring Harbor N.Y., ch. 9).

High stringency conditions for hybridization between polynucleotides of the present invention include wash conditions of 68° C. in the presence of about 0.2×SSC and about 0.1% SDS, for 1 hour. Alternatively, temperatures of about 65° C., 60° C., 55° C., or 42° C. may be used. SSC concentration may be varied from about 0.1 to 2×SSC, with SDS being present at about 0.1%. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art. Hybridization, particularly under high stringency conditions, may be suggestive of evolutionary similarity between the nucleotides. Such similarity is strongly indicative of a similar role for the nucleotides and their encoded polypeptides.

The term “hybridization complex” refers to a complex formed between two nucleic acids by virtue of the formation of hydrogen bonds between complementary bases. A hybridization complex may be formed in solution (e.g., C₀t or R₀t analysis) or formed between one nucleic acid present in solution and another nucleic acid immobilized on a solid support (e.g., paper, membranes, filters, chips, pins or glass slides, or any other appropriate substrate to which cells or their nucleic acids have been fixed).

The words “insertion” and “addition” refer to changes in an amino acid or polynucleotide sequence resulting in the addition of one or more amino acid residues or nucleotides, respectively.

“Immune response” can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

An “immunogenic fragment” is a polypeptide or oligopeptide fragment of MDDT which is capable of eliciting an immune response when introduced into a living organism, for example, a mammal. The term “immunogenic fragment” also includes any polypeptide or oligopeptide fragment of MDDT which is useful in any of the antibody production methods disclosed herein or known in the art.

The term “microarray” refers to an arrangement of a plurality of polynucleotides, polypeptides, antibodies, or other chemical compounds on a substrate.

The terms “element” and “array element” refer to a polynucleotide, polypeptide, antibody, or other chemical compound having a unique and defined position on a microarray.

The term “modulate” refers to a change in the activity of MDDT. For example, modulation may cause an increase or a decrease in protein activity, binding characteristics, or any other biological, functional, or immunological properties of MDDT.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

“Peptide nucleic acid” (PNA) refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

“Post-translational modification” of an MDDT may involve lipidation, glycosylation, phosphorylation, acetylation, racemization, proteolytic cleavage, and other modifications known in the art. These processes may occur synthetically or biochemically. Biochemical modifications will vary by cell type depending on the enzymatic milieu of MDDT.

“Probe” refers to nucleic acids encoding MDDT, their complements, or fragments thereof, which are used to detect identical, allelic or related nucleic acids. Probes are isolated oligonucleotides or polynucleotides attached to a detectable label or reporter molecule. Typical labels include radioactive isotopes, ligands, chemiluminescent agents, and enzymes. “Primers” are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 25, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the tables, figures, and Sequence Listing, may be used.

Methods for preparing and using probes and primers are described in, for example, Sambrook, J. and D. W. Russell (2001; Molecular Cloning: A Laboratory Manual, 3rd ed., vol. 1-3, Cold Spring Harbor Press, Cold Spring Harbor N.Y.), Ausubel, F. M. et al. (1999; Short Protocols in Molecular Biology, 4^{th} ed., John Wiley & Sons, New York N.Y.), and Innis, M. et al. (1990; PCR Protocols, A Guide to Methods and Applications, Academic Press, San Diego Calif.). PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer3 primer selection program (available to the public from the Whitehead Institute/MIT Center for Genome Research, Cambridge Mass.) allows the user to input a “mispriming library,” in which sequences to avoid as primer binding sites are user-specified. Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

A “recombinant nucleic acid” is a nucleic acid that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook and Russell (supra). The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that could be use to vaccinate a mammal wherein the recombinant nucleic acid is expressed, inducing a protective immunological response in the mammal.

A “regulatory element” refers to a nucleic acid sequence usually derived from untranslated regions of a gene and includes enhancers, promoters, introns, and 5′ and 3′ untranslated regions (UTRs). Regulatory elements interact with host or viral proteins which control transcription, translation, or RNA stability.

“Reporter molecules” are chemical or biochemical moieties used for labeling a nucleic acid, amino acid, or antibody. Reporter molecules include radionuclides; enzymes; fluorescent, chemiluminescent, or chromogenic agents; substrates; cofactors; inhibitors; magnetic particles; and other moieties known in the art.

An “RNA equivalent,” in reference to a DNA molecule, is composed of the same linear sequence of nucleotides as the reference DNA molecule with the exception that all occurrences of the nitrogenous base thymine are replaced with uracil, and the sugar backbone is composed of ribose instead of deoxyribose.

The term “sample” is used in its broadest sense. A sample suspected of containing MDDT, nucleic acids encoding MDDT, or fragments thereof may comprise a bodily fluid; an extract from a cell, chromosome, organelle, or membrane isolated from a cell; a cell; genomic DNA, RNA, or cDNA, in solution or bound to a substrate; a tissue; a tissue print; etc.

The terms “specific binding” and “specifically binding” refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope “A,” the presence of a polypeptide comprising the epitope A, or the presence of free unlabeled A, in a reaction containing free labeled A and the antibody will reduce the amount of labeled A that binds to the antibody.

The term “substantially purified” refers to nucleic acid or amino acid sequences that are removed from their natural environment and are isolated or separated, and are at least about 60% free, preferably at least about 75% free, and most preferably at least about 90% free from other components with which they are naturally associated.

A “substitution” refers to the replacement of one or more amino acid residues or nucleotides by different amino acid residues or nucleotides, respectively.

“Substrate” refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which polynucleotides or polypeptides are bound.

A “transcript image” or “expression profile” refers to the collective pattern of gene expression by a particular cell type or tissue under given conditions at a given time.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

A “transgenic organism,” as used herein, is any organism, including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. In another embodiment, the nucleic acid can be introduced by infection with a recombinant viral vector, such as a lentiviral vector (Lois, C. et al. (2002) Science 295:868-872). The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, plants and animals. The isolated DNA of the present invention can be introduced into the host by methods known in the art, for example infection, transfection, transformation or transconjugation. Techniques for transferring the DNA of the present invention into such organisms are widely known and provided in references such as Sambrook and Russell (supra).

A “variant” of a particular nucleic acid sequence is defined as a nucleic acid sequence having at least 40% sequence identity to the particular nucleic acid sequence over a certain length of one of the nucleic acid sequences using blastn with the “BLAST 2 Sequences” tool Version 2.0.9 (May-07-1999) set at default parameters. Such a pair of nucleic acids may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length. A variant may be described as, for example, an “allelic” (as defined above), “splice,” “species,” or “polymorphic” variant. A splice variant may have significant identity to a reference molecule, but will generally have a greater or lesser number of polynucleotides due to alternate splicing of exons during mRNA processing. The corresponding polypeptide may possess additional functional domains or lack domains that are present in the reference molecule. Species variants are polynucleotides that vary from one species to another. The resulting polypeptides will generally have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity or sequence similarity to the particular polypeptide sequence over a certain length of one of the polypeptide sequences using blastp with the “BLAST 2 Sequences” tool Version 2.0.9 (May β7-1999) set at default parameters. Such a pair of polypeptides may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity or sequence similarity over a certain defined length of one of the polypeptides.

### THE INVENTION

Various embodiments of the invention include new human molecules for disease detection and treatment (MDDT), the polynucleotides encoding MDDT, and the use of these compositions for the diagnosis, treatment, or prevention of cell proliferative, autoimmune/inflammatory, developmental, and neurological disorders.

Table 1 summarizes the nomenclature for the full length polynucleotide and polypeptide embodiments of the invention. Each polynucleotide and its corresponding polypeptide are correlated to a single Incyte project identification number (Incyte Project ID). Each polypeptide sequence is denoted by both a polypeptide sequence identification number (Polypeptide SEQ ID NO:) and an Incyte polypeptide sequence number (Incyte Polypeptide ID) as shown. Each polynucleotide sequence is denoted by both a polynucleotide sequence identification number (Polynucleotide SEQ ID NO:) and an Incyte polynucleotide consensus sequence number (Incyte Polynucleotide ID) as shown. Column 6 shows the Incyte ID numbers of physical, full length clones corresponding to the polypeptide and polynucleotide sequences of the invention. The full length clones encode polypeptides which have at least 95% sequence identity to the polypeptide sequences shown in column 3.

Table 2 shows sequences with homology to the polypeptides of the invention as identified by BLAST analysis against the GenBank protein (genpept) database and the PROTEOME database. Columns 1 and 2 show the polypeptide sequence identification number (Polypeptide SEQ ID NO:) and the corresponding Incyte polypeptide sequence number (Incyte Polypeptide ID) for polypeptides of the invention. Column 3 shows the GenBank identification number (GenBank ID NO:) of the nearest GenBank homolog and the PROTEOME database identification numbers (PROTEOME ID NO:) of the nearest PROTEOME database homologs. Column 4 shows the probability scores for the matches between each polypeptide and its homolog(s). Column 5 shows the annotation of the GenBank and PROTEOME database homolog(s) along with relevant citations where applicable, all of which are expressly incorporated by reference herein.

Table 3 shows various structural features of the polypeptides of the invention. Columns 1 and 2 show the polypeptide sequence identification number (SEQ ID NO:) and the corresponding Incyte polypeptide sequence number (Incyte Polypeptide ID) for each polypeptide of the invention. Column 3 shows the number of amino acid residues in each polypeptide. Column 4 shows potential phosphorylation sites, and column 5 shows potential glycosylation sites, as determined by the MOTIFS program of the GCG sequence analysis software package (Accelrys, Burlington Mass.). Column 6 shows amino acid residues comprising signature sequences, domains, and motifs. Column 7 shows analytical methods for protein structure/function analysis and in some cases, searchable databases to which the analytical methods were applied.

Together, Tables 2 and 3 summarize the properties of polypeptides of the invention, and these properties establish that the claimed polypeptides are molecules for disease detection and treatment.

For example, SEQ ID NO:26 is 74% identical, from residue M1 to residue P210, to human protein similar to WW domain binding protein 2 (GenBank ID g13938601) as determined by the Basic Local Alignment Search Tool (BLAST). (See Table 2.) The BLAST probability score is 2.0e-73, which indicates the probability of obtaining the observed polypeptide sequence alignment by chance. SEQ ID NO:26 is localized to the subcellular region, has binding protein function, and is a human WW domain binding protein as determined by BLAST analysis using the PROTEOME database. SEQ ID NO:26 also contains a GRAM domain as determined by searching for statistically significant matches in the hidden Markov model (HMM)-based PFAM database of conserved protein family domains. (See Table 3.) Data from BLAST-PRODOM analysis of the PRODOM database provides further corroborative evidence that SEQ ID NO:26 is a WW domain binding protein.

In another example, SEQ ID NO:40 is 100% identical, from residue M1 to residue R227 and from residue R228 to residue K342, to human NYD-SP6 (GenBank I) g13508446, residues M1-R227 and residues R267 to K381 respectively) as determined by the Basic Local Alignment Search Tool (BLAST). (See Table 2.) The BLAST probability score is 1.1e-195, which indicates the probability of obtaining the observed polypeptide sequence alignment by chance. Data from BLIMPS analysis provide further corroborative evidence that SEQ ID NO:40 is a PHD-finger-containing protein. SEQ ID NO:1-25, SEQ ID NO:27-39, and SEQ ID NO:41-48 were analyzed and annotated in a similar manner. The algorithms and parameters for the analysis of SEQ ID NO:1-48 are described in Table 7.

As shown in Table 4, the full length polynucleotide embodiments were assembled using cDNA sequences or coding (exon) sequences derived from genomic DNA, or any combination of these two types of sequences. Column 1 lists the polynucleotide sequence identification number (Polynucleotide SEQ ID NO:), the corresponding Incyte polynucleotide consensus sequence number (Incyte ID) for each polynucleotide of the invention, and the length of each polynucleotide sequence in basepairs. Column 2 shows the nucleotide start (5′) and stop (3′) positions of the cDNA and/or genomic sequences used to assemble the full length polynucleotide embodiments, and of fragments of the polynucleotides which are useful, for example, in hybridization or amplification technologies that identify SEQ ID NO:49-96 or that distinguish between SEQ ID NO:49-96 and related polynucleotides.

The polynucleotide fragments described in Column 2 of Table 4 may refer specifically, for example, to Incyte cDNAs derived from tissue-specific cDNA libraries or from pooled cDNA libraries. Alternatively, the polynucleotide fragments described in column 2 may refer to GenBank cDNAs or ESTs which contributed to the assembly of the full length polynucleotides. In addition, the polynucleotide fragments described in column 2 may identify sequences derived from the ENSEMBL (The Sanger Centre, Cambridge, UK) database (i.e., those sequences including the designation “ENST”). Alternatively, the polynucleotide fragments described in column 2 may be derived from the NCBI RefSeq Nucleotide Sequence Records Database (i.e., those sequences including the designation “NM” or “NT”) or the NCBI RefSeq Protein Sequence Records (i.e., those sequences including the designation “NP”). Alternatively, the polynucleotide fragments described in column 2 may refer to assemblages of both cDNA and Genscan-predicted exons brought together by an “exon stitching” algorithm. For example, a polynucleotide sequence identified as FL_XXXXXX_N_{1—}N_{2—}YYYYY_N_{3—}N₄ represents a “stitched” sequence in which XXXXXX is the identification number of the cluster of sequences to which the algorithm was applied, and YYYYY is the number of the prediction generated by the algorithm, and N_{1,2,3} . . . , if present, represent specific exons that may have been manually edited during analysis (See Example V). Alternatively, the polynucleotide fragments in column 2 may refer to assemblages of exons brought together by an “exon-stretching” algorithm. For example, a polynucleotide sequence identified as FLXXXXXX_gAAAAA_gBBBBB_{—}1_N is a “stretched” sequence, with XXXXXX being the Incyte project identification number, gAAAAA being the GenBank identification number of the human genomic sequence to which the “exon-stretching” algorithm was applied, gBBBBB being the GenBank identification number or NCBI RefSeq identification number of the nearest GenBank protein homolog, and N referring to specific exons (See Example V). In instances where a RefSeq sequence was used as a protein homolog for the “exon-stretching” algorithm, a RefSeq identifier (denoted by “NM,” “NP,” or “NT”) may be used in place of the GenBank identifier (i.e., gBBBBB).

Alternatively, a prefix identifies component sequences that were hand-edited, predicted from genomic DNA sequences, or derived from a combination of sequence analysis methods. The following Table lists examples of component sequence prefixes and corresponding sequence analysis methods associated with the prefixes (see Example IV and Example V).

| Prefix | Type of analysis and/or examples of programs |
|---|---|
| GNN, | Exon prediction from genomic sequences using, for example, |
| GFG, | GENSCAN (Stanford University, CA, USA) or FGENES |
| ENST | (Computer Genomics Group, The Sanger Centre, |
| | Cambridge, UK). |
| GBI | Hand-edited analysis of genomic sequences. |
| FL | Stitched or stretched genomic sequences (see Example V). |
| INCY | Full length transcript and exon prediction from mapping of |
| | EST sequences to the genome. Genomic location and |
| | EST composition data are combined to predict the exons |
| | and resulting transcript. |

In some cases, Incyte cDNA coverage redundant with the sequence coverage shown in Table 4 was obtained to confirm the final consensus polynucleotide sequence, but the relevant Incyte cDNA identification numbers are not shown.

Table 5 shows the representative cDNA libraries for those full length polynucleotides which were assembled using Incyte cDNA sequences. The representative cDNA library is the Incyte cDNA library which is most frequently represented by the Incyte cDNA sequences which were used to assemble and confirm the above polynucleotides. The tissues and vectors which were used to construct the cDNA libraries shown in Table 5 are described in Table 6.

The invention also encompasses MDDT variants. Various embodiments of MDDT variants can have at least about 80%, at least about 90%, or at least about 95% amino acid sequence identity to the MDDT amino acid sequence, and can contain at least one functional or structural characteristic of MDDT.

Various embodiments also encompass polynucleotides which encode MDDT. In a particular embodiment, the invention encompasses a polynucleotide sequence comprising a sequence selected from the group consisting of SEQ ID NO:49-96, which encodes MDDT. The polynucleotide sequences of SEQ ID NO:49-96, as presented in the Sequence Listing, embrace the equivalent RNA sequences, wherein occurrences of the nitrogenous base thymine are replaced with uracil, and the sugar backbone is composed of ribose instead of deoxyribose.

The invention also encompasses variants of a polynucleotide encoding MDDT. In particular, such a variant polynucleotide will have at least about 70%, or alternatively at least about 85%, or even at least about 95% polynucleotide sequence identity to a polynucleotide encoding MDDT. A particular aspect of the invention encompasses a variant of a polynucleotide comprising a sequence selected from the group consisting of SEQ ID NO:49-96 which has at least about 70%, or alternatively at least about 85%, or even at least about 95% polynucleotide sequence identity to a nucleic acid sequence selected from the group consisting of SEQ ID NO:49-96. Any one of the polynucleotide variants described above can encode a polypeptide which contains at least one functional or structural characteristic of MDDT.

In addition, or in the alternative, a polynucleotide variant of the invention is a splice variant of a polynucleotide encoding MDDT. A splice variant may have portions which have significant sequence identity to a polynucleotide encoding MDDT, but will generally have a greater or lesser number of polynucleotides due to additions or deletions of blocks of sequence arising from alternate splicing of exons during mRNA processing. A splice variant may have less than about 70%, or alternatively less than about 60%, or alternatively less than about 50% polynucleotide sequence identity to a polynucleotide encoding MDDT over its entire length; however, portions of the splice variant will have at least about 70%, or alternatively at least about 85%, or alternatively at least about 95%, or alternatively 100% polynucleotide sequence identity to portions of the polynucleotide encoding MDDT. For example, a polynucleotide comprising a sequence of SEQ ID NO:61 is a splice variant of a polynucleotide comprising a sequence of SEQ ID NO:56. Any one of the splice variants described above can encode a polypeptide which contains at least one functional or structural characteristic of MDDT.

It will be appreciated by those skilled in the art that as a result of the degeneracy of the genetic code, a multitude of polynucleotide sequences encoding MDDT, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring MDDT, and all such variations are to be considered as being specifically disclosed.

Although polynucleotides which encode MDDT and its variants are generally capable of hybridizing to polynucleotides encoding naturally occurring MDDT under appropriately selected conditions of stringency, it may be advantageous to produce polynucleotides encoding MDDT or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding MDDT and its derivatives without altering the encoded amino acid sequences include the production of RNA transcripts having more desirable properties, such as a greater half-life, than transcripts produced from the naturally occurring sequence.

The invention also encompasses production of polynucleotides which encode MDDT and MDDT derivatives, or fragments thereof, entirely by synthetic chemistry. After production, the synthetic polynucleotide may be inserted into any of the many available expression vectors and cell systems using reagents well known in the art. Moreover, synthetic chemistry may be used to introduce mutations into a polynucleotide encoding MDDT or any fragment thereof.

Embodiments of the invention can also include polynucleotides that are capable of hybridizing to the claimed polynucleotides, and, in particular, to those having the sequences shown in SEQ ID NO:49-96 and fragments thereof, under various conditions of stringency (Wahl, G. M. and S. L. Berger (1987) Methods Enzymol. 152:399-407; Kimmel, A. R. (1987) Methods Enzymol. 152:507-511). Hybridization conditions, including annealing and wash conditions, are described in “Definitions.”

Methods for DNA sequencing are well known in the art and may be used to practice any of the embodiments of the invention. The methods may employ such enzymes as the Klenow fragment of DNA polymerase I, SEQUENASE (US Biochemical, Cleveland Ohio), Taq polymerase (Applied Biosystems), thermostable T7 polymerase (Amersham Biosciences, Piscataway N.J.), or combinations of polymerases and proofreading exonucleases such as those found in the ELONGASE amplification system (Invitrogen, Carlsbad Calif.). Preferably, sequence preparation is automated with machines such as the MICROLAB 2200 liquid transfer system (Hamilton, Reno Nev.), PTC200 thermal cycler (MJ Research, Watertown Mass.) and ABI CATALYST 800 thermal cycler (Applied Biosystems). Sequencing is then carried out using either the ABI 373 or 377 DNA sequencing system (Applied Biosystems), the MEGABACE 1000 DNA sequencing system (Amersham Biosciences), or other systems known in the art. The resulting sequences are analyzed using a variety of algorithms which are well known in the art (Ausubel et al., supra, ch. 7; Meyers, R. A. (1995) Molecular Biology and Biotechnology, Wiley VCH, New York N.Y., pp. 856-853).

The nucleic acids encoding MDDT may be extended utilizing a partial nucleotide sequence and employing various PCR-based methods known in the art to detect upstream sequences, such as promoters and regulatory elements. For example, one method which may be employed, restriction-site PCR, uses universal and nested primers to amplify unknown sequence from genomic DNA within a cloning vector (Sarkar, G. (1993) PCR Methods Applic. 2:318-322). Another method, inverse PCR, uses primers that extend in divergent directions to amplify unknown sequence from a circularized template. The template is derived from restriction fragments comprising a known genomic locus and surrounding sequences (Triglia, T. et al. (1988) Nucleic Acids Res. 16:8186). A third method, capture PCR, involves PCR amplification of DNA fragments adjacent to known sequences in human and yeast artificial chromosome DNA (Lagerstrom, M. et al. (1991) PCR Methods Applic. 1:111-119). In this method, multiple restriction enzyme digestions and ligations may be used to insert an engineered double-stranded sequence into a region of unknown sequence before performing PCR. Other methods which may be used to retrieve unknown sequences are known in the art (Parker, J. D. et al. (1991) Nucleic Acids Res. 19:3055-3060). Additionally, one may use PCR, nested primers, and PROMOTERFINDER libraries (Clontech, Palo Alto Calif.) to walk genomic DNA. This procedure avoids the need to screen libraries and is useful in finding intron/exon junctions. For all PCR-based methods, primers may be designed using commercially available software, such as OLIGO 4.06 primer analysis software (National Biosciences, Plymouth Minn.) or another appropriate program, to be about 22 to 30 nucleotides in length, to have a GC content of about 50% or more, and to anneal to the template at temperatures of about 68° C. to 72° C.

When screening for full length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e.g., GENOTYPER and SEQUENCE NAVIGATOR, Applied Biosystems), and the entire process from loading of samples to computer analysis and electronic data display may be computer controlled. Capillary electrophoresis is especially preferable for sequencing small DNA fragments which may be present in limited amounts in a particular sample.

In another embodiment of the invention, polynucleotides or fragments thereof which encode MDDT may be cloned in recombinant DNA molecules that direct expression of MDDT, or fragments or functional equivalents thereof, in appropriate host cells. Due to the inherent degeneracy of the genetic code, other polynucleotides which encode substantially the same or a functionally equivalent polypeptides may be produced and used to express MDDT.

The polynucleotides of the invention can be engineered using methods generally known in the art in order to alter MDDT-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

The nucleotides of the present invention may be subjected to DNA shuffling techniques such as MOLECULARBREEDING (Maxygen Inc., Santa Clara Calif.; described in U.S. Pat. No. 5,837,458; Chang, C.-C. et al. (1999) Nat. Biotechnol. 17:793-797; Christians, F. C. et al. (1999) Nat. Biotechnol. 17:259-264; and Crameri, A. et al. (1996) Nat. Biotechnol. 14:315-319) to alter or improve the biological properties of MDDT, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

In another embodiment, polynucleotides encoding MDDT may be synthesized, in whole or in part, using one or more chemical methods well known in the art (Caruthers, M. H. et al. (1980) Nucleic Acids Symp. Ser. 7:215-223; Horn, T. et al. (1980) Nucleic Acids Symp. Ser. 7:225-232). Alternatively, MDDT itself or a fragment thereof may be synthesized using chemical methods known in the art. For example, peptide synthesis can be performed using various solution-phase or solid-phase techniques (Creighton, T. (1984) Proteins, Structures and Molecular Properties, WH Freeman, New York N.Y., pp. 55-60; Roberge, J. Y. et al. (1995) Science 269:202-204). Automated synthesis may be achieved using the ABI 431A peptide synthesizer (Applied Biosystems). Additionally, the amino acid sequence of MDDT, or any part thereof, may be altered during direct synthesis and/or combined with sequences from other proteins, or any part thereof, to produce a variant polypeptide or a polypeptide having a sequence of a naturally occurring polypeptide.

The peptide may be substantially purified by preparative high performance liquid chromatography (Chiez, R. M. and F. Z. Regnier (1990) Methods Enzymol. 182:392-421). The composition of the synthetic peptides may be confirmed by amino acid analysis or by sequencing (Creighton, supra, pp. 28-53).

In order to express a biologically active MDDT, the polynucleotides encoding MDDT or derivatives thereof may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′ untranslated regions in the vector and in polynucleotides encoding MDDT. Such elements may vary in their strength and specificity. Specific initiation signals may also be used to achieve more efficient translation of polynucleotides encoding MDDT. Such signals include the ATG initiation codon and adjacent sequences, e.g. the Kozak sequence. In cases where a polynucleotide sequence encoding MDDT and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used (Scharf, D. et al. (1994) Results Probl. Cell Differ. 20:125-162).

Methods which are well known to those skilled in the art may be used to construct expression vectors containing polynucleotides encoding MDDT and appropriate transcriptional and translational control elements. These methods include in vitro recombinant DNA techniques, synthetic techniques, and in vivo genetic recombination (Sambrook and Russell, supra, ch. 1-4, and 8; Ausubel et al., supra, ch. 1, 3, and 15).

A variety of expression vector/host systems may be utilized to contain and express polynucleotides encoding MDDT. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems (Sambrook and Russell, supra; Ausubel et al., supra; Van Heeke, G. and S. M. Schuster (1989) J. Biol. Chem. 264:5503-5509; Engelhard, E. K. et al. (1994) Proc. Natl. Acad. Sci. USA 91:3224-3227; Sandig, V. et al. (1996) Hum. Gene Ther. 7:1937-1945; Takamatsu, N. (1987) EMBO J. 6:307-311; The McGraw Hill Yearbook of Science and Technology (1992) McGraw Hill, New York N.Y., pp. 191-196; Logan, J. and T. Shenk (1984) Proc. Natl. Acad. Sci. USA 81:3655-3659; Harrington, J. J. et al. (1997) Nat. Genet. 15:345-355). Expression vectors derived from retroviruses, adenoviruses, or herpes or vaccinia viruses, or from various bacterial plasmids, may be used for delivery of polynucleotides to the targeted organ, tissue, or cell population (Di Nicola, M. et al. (1998) Cancer Gen. Ther. 5:350-356; Yu, M. et al. (1993) Proc. Natl. Acad. Sci. USA 90:6340-6344; Buller, R. M. et al. (1985) Nature 317:813-815; McGregor, D. P. et al. (1994) Mol. Immunol. 31:219-226; Verma, I. M. and N. Somia (1997) Nature 389:239-242). The invention is not limited by the host cell employed.

In bacterial systems, a number of cloning and expression vectors may be selected depending upon the use intended for polynucleotides encoding MDDT. For example, routine cloning, subcloning, and propagation of polynucleotides encoding MDDT can be achieved using a multifunctional E. coli vector such as PBLUESCRIPT (Stratagene, La Jolla Calif.) or PSPORT1 plasmid (Invitrogen). Ligation of polynucleotides encoding MDDT into the vector's multiple cloning site disrupts the lacZ gene, allowing a calorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence (Van Heeke, G. and S. M. Schuster (1989) J. Biol. Chem. 264:5503-5509). When large quantities of MDDT are needed, e.g. for the production of antibodies, vectors which direct high level expression of MDDT may be used. For example, vectors containing the strong, inducible SP6 or T7 bacteriophage promoter may be used.

Yeast expression systems may be used for production of MDDT. A number of vectors containing constitutive or inducible promoters, such as alpha factor, alcohol oxidase, and PGH promoters, may be used in the yeast Saccharomyces cerevisiae or Pichia pastoris. In addition, such vectors direct either the secretion or intracellular retention of expressed proteins and enable integration of foreign polynucleotide sequences into the host genome for stable propagation (Ausubel et al., supra; Bitter, G. A. et al. (1987) Methods Enzymol. 153:516-544; Scorer, C. A. et al. (1994) Bio/Technology 12:181-184).

Plant systems may also be used for expression of MDDT. Transcription of polynucleotides encoding MDDT may be driven by viral promoters, e.g., the ³⁵S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters may be used (Coruzzi, G. et al. (1984) EMBO J. 3:1671-1680; Broglie, R. et al. (1984) Science 224:838-843; Winter, J. et al. (1991) Results Probl. Cell Differ. 17:85-105). These constructs can be introduced into plant cells by direct DNA transformation or pathogen-mediated transfection (The McGraw Hill Yearbook of Science and Technology (1992) McGraw Hill, New York N.Y., pp. 191-196).

In mammalian cells, a number of viral-based expression systems may be utilized. In cases where an adenovirus is used as an expression vector, polynucleotides encoding MDDT may be ligated into an adenovirus transcription/translation complex consisting of the late promoter and tripartite leader sequence. Insertion in a non-essential E1 or E3 region of the viral genome may be used to obtain infective virus which expresses MDDT in host cells (Logan, J. and T. Shenk (1984) Proc. Natl. Acad. Sci. USA 81:3655-3659). In addition, transcription enhancers, such as the Rous sarcoma virus (RSV) enhancer, may be used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes (Harrington, J. J. et al. (1997) Nat. Genet. 15:345-355).

For long term production of recombinant proteins in mammalian systems, stable expression of MDDT in cell lines is preferred. For example, polynucleotides encoding MDDT can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk⁻ and apr⁻0 cells, respectively (Wigler, M. et al. (1977) Cell 11:223-232; Lowy, I. et al. (1980) Cell 22:817-823). Also, antimetabolite, antibiotic, or herbicide resistance can be used as the basis for selection. For example, dhfr confers resistance to methotrexate; neo confers resistance to the aminoglycosides neomycin and G-418; and als and pat confer resistance to chlorsulfuron and phosphinotricin acetyltransferase, respectively (Wigler, M. et al. (1980) Proc. Natl. Acad. Sci. USA 77:3567-3570; Colbere-Garapin, F. et al. (1981) J. Mol. Biol. 150:1-14). Additional selectable genes have been described, e.g., trpB and hisD, which alter cellular requirements for metabolites (Hartman, S. C. and R. C. Mulligan (1988) Proc. Natl. Acad. Sci. USA 85:8047-8051). Visible markers, e.g., anthocyanins, green fluorescent proteins (GFP; Clontech), β-glucuronidase and its substrate β-glucuronide, or luciferase and its substrate luciferin may be used. These markers can be used not only to identify transformants, but also to quantify the amount of transient or stable protein expression attributable to a specific vector system (Rhodes, C. A. (1995) Methods Mol. Biol. 55:121-131).

Although the presence/absence of marker gene expression suggests that the gene of interest is also present, the presence and expression of the gene may need to be confirmed. For example, if the sequence encoding MDDT is inserted within a marker gene sequence, transformed cells containing polynucleotides encoding MDDT can be identified by the absence of marker gene function. Alternatively, a marker gene can be placed in tandem with a sequence encoding MDDT under the control of a single promoter. Expression of the marker gene in response to induction or selection usually indicates expression of the tandem gene as well.

In general, host cells that contain the polynucleotide encoding MDDT and that express MDDT may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of MDDT using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on MDDT is preferred, but a competitive binding assay may be employed. These and other assays are well known in the art (Hampton, R. et al. (1990) Serological Methods, a Laboratory Manual, APS Press, St. Paul Minn., Sect. IV; Coligan, J. E. et al. (1997) Current Protocols in Immunology, Greene Pub. Associates and Wiley-Interscience, New York N.Y.; Pound, J. D. (1998) Immunochemical Protocols, Humana Press, Totowa N.J.).

A wide variety of labels and conjugation techniques are known by those skilled in the art and may be used in various nucleic acid and amino acid assays. Means for producing labeled hybridization or PCR probes for detecting sequences related to polynucleotides encoding MDDT include oligolabeling, nick translation, end-labeling, or PCR amplification using a labeled nucleotide. Alternatively, polynucleotides encoding MDDT, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Amersham Biosciences, Promega (Madison Wis.), and US Biochemical. Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with polynucleotides encoding MDDT may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode MDDT may be designed to contain signal sequences which direct secretion of MDDT through a prokaryotic or eukaryotic cell membrane.

In addition, a host cell strain may be chosen for its ability to modulate expression of the inserted polynucleotides or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” or “pro” form of the protein may also be used to specify protein targeting, folding, and/or activity. Different host cells which have specific cellular machinery and characteristic mechanisms for post-translational activities (e.g., CHO, HeLa, MDCK, HEK293, and WI38) are available from the American Type Culture Collection (ATCC, Manassas Va.) and may be chosen to ensure the correct modification and processing of the foreign protein.

In another embodiment of the invention, natural, modified, or recombinant polynucleotides encoding MDDT may be ligated to a heterologous sequence resulting in translation of a fusion protein in any of the aforementioned host systems. For example, a chimeric MDDT protein containing a heterologous moiety that can be recognized by a commercially available antibody may facilitate the screening of peptide libraries for inhibitors of MDDT activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the MDDT encoding sequence and the heterologous protein sequence, so that MDDT may be cleaved away from the heterologous moiety following purification. Methods for fusion protein expression and purification are discussed in Ausubel et al. (supra, ch. 10 and 16). A variety of commercially available kits may also be used to facilitate expression and purification of fusion proteins.

In another embodiment, synthesis of radiolabeled MDDT may be achieved in vitro using the TNT rabbit reticulocyte lysate or wheat germ extract system (Promega). These systems couple transcription and translation of protein-coding sequences operably associated with the T7, T3, or SP6 promoters. Translation takes place in the presence of a radiolabeled amino acid precursor, for example, ³⁵S-methionine.

MDDT, fragments of MDDT, or variants of MDDT may be used to screen for compounds that specifically bind to MDDT. One or more test compounds may be screened for specific binding to MDDT. In various embodiments, 1, 2, 3, 4, 5, 10, 20, 50, 100, or 200 test compounds can be screened for specific binding to MDDT. Examples of test compounds can include antibodies, anticalins, oligonucleotides, proteins (e.g., ligands or receptors), or small molecules.

In related embodiments, variants of MDDT can be used to screen for binding of test compounds, such as antibodies, to MDDT, a variant of MDDT, or a combination of MDDT and/or one or more variants MDDT. In an embodiment, a variant of MDDT can be used to screen for compounds that bind to a variant of MDDT, but not to MDDT having the exact sequence of a sequence of SEQ ID NO:1-48. MDDT variants used to perform such screening can have a range of about 50% to about 99% sequence identity to MDDT, with various embodiments having 60%, 70%, 75%, 80%, 85%, 90%, and 95% sequence identity.

In an embodiment, a compound identified in a screen for specific binding to MDDT can be closely related to the natural ligand of MDDT, e.g., a ligand or fragment thereof, a natural substrate, a structural or functional mimetic, or a natural binding partner (Coligan, J. E. et al. (1991) Current Protocols in Immunology 1(2):Chapter 5). In another embodiment, the compound thus identified can be a natural ligand of a receptor MDDT (Howard, A. D. et al. (2001) Trends Pharmacol. Sci.22: 132-140; Wise, A. et al. (2002) Drug Discovery Today 7:235-246).

In other embodiments, a compound identified in a screen for specific binding to MDDT can be closely related to the natural receptor to which MDDT binds, at least a fragment of the receptor, or a fragment of the receptor including all or a portion of the ligand binding site or binding pocket. For example, the compound may be a receptor for MDDT which is capable of propagating a signal, or a decoy receptor for MDDT which is not capable of propagating a signal (Ashkenazi, A. and V. M. Divit (1999) Curr. Opin. Cell Biol. 11:255-260; Mantovani, A. et al. (2001) Trends Immunol. 22:328-336). The compound can be rationally designed using known techniques. Examples of such techniques include those used to construct the compound etanercept (ENBREL; Amgen Inc., Thousand Oaks Calif.), which is efficacious for treating rheumatoid arthritis in humans. Etanercept is an engineered p75 tumor necrosis factor (TNF) receptor dimer linked to the Fc portion of human IgG₁ (Taylor, P. C. et al. (2001) Curr. Opin. Immunol. 13:611-616).

In one embodiment, two or more antibodies having similar or, alternatively, different specificities can be screened for specific binding to MDDT, fragments of MDDT, or variants of MDDT. The binding specificity of the antibodies thus screened can thereby be selected to identify particular fragments or variants of MDDT. In one embodiment, an antibody can be selected such that its binding specificity allows for preferential identification of specific fragments or variants of MDDT. In another embodiment, an antibody can be selected such that its binding specificity allows for preferential diagnosis of a specific disease or condition having increased, decreased, or otherwise abnormal production of MDDT.

In an embodiment, anticalins can be screened for specific binding to MDDT, fragments of MDDT, or variants of MDDT. Anticalins are ligand-binding proteins that have been constructed based on a lipocalin scaffold (Weiss, G. A. and H. B. Lowman (2000) Chem. Biol. 7:R177-R184; Skerra, A. (2001) J. Biotechnol. 74:257-275). The protein architecture of lipocalins can include a beta-barrel having eight antiparallel beta-strands, which supports four loops at its open end. These loops form the natural ligand-binding site of the lipocalins, a site which can be re-engineered in vitro by amino acid substitutions to impart novel binding specificities. The amino acid substitutions can be made using methods known in the art or described herein, and can include conservative substitutions (e.g., substitutions that do not alter binding specificity) or substitutions that modestly, moderately, or significantly alter binding specificity.

In one embodiment, screening for compounds which specifically bind to, stimulate, or inhibit MDDT involves producing appropriate cells which express MDDT, either as a secreted protein or on the cell membrane. Preferred cells can include cells from mammals, yeast, Drosophila, or E. coli. Cells expressing MDDT or cell membrane fractions which contain MDDT are then contacted with a test compound and binding, stimulation, or inhibition of activity of either MDDT or the compound is analyzed.

An assay may simply test binding of a test compound to the polypeptide, wherein binding is detected by a fluorophore, radioisotope, enzyme conjugate, or other detectable label. For example, the assay may comprise the steps of combining at least one test compound with MDDT, either in solution or affixed to a solid support, and detecting the binding of MDDT to the compound. Alternatively, the assay may detect or measure binding of a test compound in the presence of a labeled competitor. Additionally, the assay may be carried out using cell-free preparations, chemical libraries, or natural product mixtures, and the test compound(s) may be free in solution or affixed to a solid support.

An assay can be used to assess the ability of a compound to bind to its natural ligand and/or to inhibit the binding of its natural ligand to its natural receptors. Examples of such assays include radio-labeling assays such as those described in U.S. Pat. No. 5,914,236 and U.S. Pat. No. 6,372,724. In a related embodiment, one or more amino acid substitutions can be introduced into a polypeptide compound (such as a receptor) to improve or alter its ability to bind to its natural ligands (Matthews, D. J. and J. A. Wells. (1994) Chem. Biol. 1:25-30). In another related embodiment, one or more amino acid substitutions can be introduced into a polypeptide compound (such as a ligand) to improve or alter its ability to bind to its natural receptors (Cunningham, B. C. and J. A. Wells (1991) Proc. Natl. Acad. Sci. USA 88:3407-3411; Lowman, H. B. et al. (1991) J. Biol. Chem. 266:10982-10988).

MDDT, fragments of MDDT, or variants of MDDT may be used to screen for compounds that modulate the activity of MDDT. Such compounds may include agonists, antagonists, or partial or inverse agonists. In one embodiment, an assay is performed under conditions permissive for MDDT activity, wherein MDDT is combined with at least one test compound, and the activity of MDDT in the presence of a test compound is compared with the activity of MDDT in the absence of the test compound. A change in the activity of MDDT in the presence of the test compound is indicative of a compound that modulates the activity of MDDT. Alternatively, a test compound is combined with an in vitro or cell-free system comprising MDDT under conditions suitable for MDDT activity, and the assay is performed. In either of these assays, a test compound which modulates the activity of MDDT may do so indirectly and need not come in direct contact with the test compound. At least one and up to a plurality of test compounds may be screened.

In another embodiment, polynucleotides encoding MDDT or their mammalian homologs may be “knocked out” in an animal model system using homologous recombination in embryonic stem (ES) cells. Such techniques are well known in the art and are useful for the generation of animal models of human disease (see, e.g., U.S. Pat. No. 5,175,383 and U.S. Pat. No. 5,767,337). For example, mouse ES cells, such as the mouse 129/SvJ cell line, are derived from the early mouse embryo and grown in culture. The ES cells are transformed with a vector containing the gene of interest disrupted by a marker gene, e.g., the neomycin phosphotransferase gene (neo; Capecchi, M. R. (1989) Science 244:1288-1292). The vector integrates into the corresponding region of the host genome by homologous recombination. Alternatively, homologous recombination takes place using the Cre-loxP system to knockout a gene of interest in a tissue- or developmental stage-specific manner (Marth, J. D. (1996) Clin. Invest. 97:1999-2002; Wagner, K. U. et al. (1997) Nucleic Acids Res. 25:4323-4330). Transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Transgenic animals thus generated may be tested with potential therapeutic or toxic agents.

Polynucleotides encoding MDDT may also be manipulated in vitro in ES cells derived from human blastocysts. Human ES cells have the potential to differentiate into at least eight separate cell lineages including endoderm, mesoderm, and ectodermal cell types. These cell lineages differentiate into, for example, neural cells, hematopoietic lineages, and cardiomyocytes (Thomson, J. A. et al. (1998) Science 282:1145-1147).

Polynucleotides encoding MDDT can also be used to create “knockin” humanized animals (pigs) or transgenic animals (mice or rats) to model human disease. With knockin technology, a region of a polynucleotide encoding MDDT is injected into animal ES cells, and the injected sequence integrates into the animal cell genome. Transformed cells are injected into blastulae, and the blastulae are implanted as described above. Transgenic progeny or inbred lines are studied and treated with potential pharmaceutical agents to obtain information on treatment of a human disease. Alternatively, a mammal inbred to overexpress MDDT, e.g., by secreting MDDT in its milk, may also serve as a convenient source of that protein (Janne, J. et al. (1998) Biotechnol. Annu. Rev. 4:55-74).

Therapeutics

Chemical and structural similarity, e.g., in the context of sequences and motifs, exists between regions of MDDT and molecules for disease detection and treatment. In addition, examples of tissues expressing MDDT can be found in Table 6 and can also be found in Example XI. Therefore, MDDT appears to play a role in cell proliferative, autoimmune/inflammatory, developmental, and neurological disorders. In the treatment of disorders associated with increased MDDT expression or activity, it is desirable to decrease the expression or activity of MDDT. In the treatment of disorders associated with decreased MDDT expression or activity, it is desirable to increase the expression or activity of MDDT.

Therefore, in one embodiment, MDDT or a fragment or derivative thereof may be administered to a subject to treat or prevent a disorder associated with decreased expression or activity of MDDT. Examples of such disorders include, but are not limited to, a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss; and a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia.

In another embodiment, a vector capable of expressing MDDT or a fragment or derivative thereof may be administered to a subject to treat or prevent a disorder associated with decreased expression or activity of MDDT including, but not limited to, those described above.

In a further embodiment, a composition comprising a substantially purified MDDT in conjunction with a suitable pharmaceutical carrier may be administered to a subject to treat or prevent a disorder associated with decreased expression or activity of MDDT including, but not limited to, those provided above.

In still another embodiment, an agonist which modulates the activity of MDDT may be administered to a subject to treat or prevent a disorder associated with decreased expression or activity of MDDT including, but not limited to, those listed above.

In a further embodiment, an antagonist of MDDT may be administered to a subject to treat or prevent a disorder associated with increased expression or activity of MDDT. Examples of such disorders include, but are not limited to, those cell proliferative, autoimmune/inflammatory, developmental, and neurological disorders described above. In one aspect, an antibody which specifically binds MDDT may be used directly as an antagonist or indirectly as a targeting or delivery mechanism for bringing a pharmaceutical agent to cells or tissues which express MDDT.

In an additional embodiment, a vector expressing the complement of the polynucleotide encoding MDDT may be administered to a subject to treat or prevent a disorder associated with increased expression or activity of MDDT including, but not limited to, those described above.

In other embodiments, any protein, agonist, antagonist, antibody, complementary sequence, or vector embodiments may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of MDDT may be produced using methods which are generally known in the art. In particular, purified MDDT may be used to produce antibodies or to screen libraries of pharmaceutical agents to identify those which specifically bind MDDT. Antibodies to MDDT may also be generated using methods that are well known in the art. Such antibodies may include, but are not limited to, polyclonal, monoclonal, chimeric, and single chain antibodies, Fab fragments, and fragments produced by a Fab expression library. In an embodiment, neutralizing antibodies (i.e., those which inhibit dimer formation) can be used therapeutically. Single chain antibodies (e.g., from camels or llamas) may be potent enzyme inhibitors and may have application in the design of peptide mimetics, and in the development of immuno-adsorbents and biosensors (Muyldermans, S. (2001) J. Biotechnol. 74:277-302).

For the production of antibodies, various hosts including goats, rabbits, rats, mice, camels, dromedaries, llamas, humans, and others may be immunized by injection with MDDT or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol.

Among adjuvants used in humans, BCG (bacilli Calmette-Guerin) and Corynebacterium parvum are especially preferable.

It is preferred that the oligopeptides, peptides, or fragments used to induce antibodies to MDDT have an amino acid sequence consisting of at least about 5 amino acids, and generally will consist of at least about 10 amino acids. It is also preferable that these oligopeptides, peptides, or fragments are substantially identical to a portion of the amino acid sequence of the natural protein.

Short stretches of MDDT amino acids may be fused with those of another protein, such as KLH, and antibodies to the chimeric molecule may be produced. Monoclonal antibodies to MDDT may be prepared using any technique which provides for the production of antibody molecules by continuous cell lines in culture. These include, but are not limited to, the hybridoma technique, the human B-cell hybridoma technique, and the EBV-hybridoma technique (Kohler, G. et al. (1975) Nature 256:495-497; Kozbor, D. et al. (1985) J. Immunol. Methods 81:31-42; Cote, R. J. et al. (1983) Proc. Natl. Acad. Sci. USA 80:2026-2030; Cole, S. P. et al. (1984) Mol. Cell Biol. 62:109-120).

In addition, techniques developed for the production of “chimeric antibodies,” such as the splicing of mouse antibody genes to human antibody genes to obtain a molecule with appropriate antigen specificity and biological activity, can be used (Morrison, S. L. et al. (1984) Proc. Natl. Acad. Sci. USA 81:6851-6855; Neuberger, M. S. et al. (1984) Nature 312:604-608; Takeda, S. et al. (1985) Nature 314:452-454). Alternatively, techniques described for the production of single chain ntibodies may be adapted, using methods known in the art, to produce MDDT-specific single chain ntibodies. Antibodies with related specificity, but of distinct idiotypic composition, may be generated by chain shuffling from random combinatorial immunoglobulin libraries (Burton, D. R. (1991) Proc. Natl. Acad. Sci. USA 88:10134-10137).

Antibodies may also be produced by inducing in vivo production in the lymphocyte population or by screening immunoglobulin libraries or panels of highly specific binding reagents as disclosed in the literature (Orlandi, R. et al. (1989) Proc. Natl. Acad. Sci. USA 86:3833-3837; Winter, G. et al. (1991) Nature 349:293-299).

Antibody fragments which contain specific binding sites for MDDT may also be generated. For example, such fragments include, but are not limited to, F(ab′)₂ fragments produced by pepsin digestion of the antibody molecule and Fab fragments generated by reducing the disulfide bridges of the F(ab′)₂ fragments. Alternatively, Fab expression libraries may be constructed to allow rapid and easy identification of monoclonal Fab fragments with the desired specificity (Huse, W. D. et al. (1989) Science 246:1275-1281).

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between MDDT and its specific antibody. A two-site, monoclonal-based immunoassay utilizing monoclonal antibodies reactive to two non-interfering MDDT epitopes is generally used, but a competitive binding assay may also be employed (Pound, supra).

Various methods such as Scatchard analysis in conjunction with radioimmunoassay techniques may be used to assess the affinity of antibodies for MDDT. Affinity is expressed as an association constant, Kₐ, which is defined as the molar concentration of MDDT-antibody complex divided by the molar concentrations of free antigen and free antibody under equilibrium conditions. The Kₐ determined for a preparation of polyclonal antibodies, which are heterogeneous in their affinities for multiple MDDT epitopes, represents the average affinity, or avidity, of the antibodies for MDDT. The Kₐ determined for a preparation of monoclonal antibodies, which are monospecific for a particular MDDT epitope, represents a true measure of affinity. High-affinity antibody preparations with Kₐ ranging from about 10⁹ to 10¹² L/mole are preferred for use in immunoassays in which the MDDT-antibody complex must withstand rigorous manipulations. Low-affinity antibody preparations with Kₐ ranging from about 10⁶ to 10⁷ L/mole are preferred for use in immunopurification and similar procedures which ultimately require dissociation of MDDT, preferably in active form, from the antibody (Catty, D. (1988) Antibodies, Volume I: A Practical Approach, IRL Press, Washington D.C.; Liddell, J. E. and A. Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of MDDT-antibody complexes. Procedures for evaluating antibody specificity, titer, and avidity, and guidelines for antibody quality and usage in various applications, are generally available (Catty, supra; Coligan et al., supra).

In another embodiment of the invention, polynucleotides encoding MDDT, or any fragment or complement thereof, may be used for therapeutic purposes. In one aspect, modifications of gene expression can be achieved by designing complementary sequences or antisense molecules (DNA, RNA, PNA, or modified oligonucleotides) to the coding or regulatory regions of the gene encoding MDDT. Such technology is well known in the art, and antisense oligonucleotides or larger fragments can be designed from various locations along the coding or control regions of sequences encoding MDDT (Agrawal, S., ed. (1996) Antisense Therapeutics, Humana Press, Totawa N.J.).

In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the target protein (Slater, J. E. et al. (1998) J. Allergy Clin. Immunol. 102:469-475; Scanlon, K. J. et al. (1995) 9:1288-1296). Antisense sequences can also be introduced intracellularly through the use of viral vectors, such as retrovirus and adeno-associated virus vectors (Miller, A. D. (1990) Blood 76:271; Ausubel et al., supra; Uckert, W. and W. Walther (1994) Pharmacol. Ther. 63:323-347). Other gene delivery mechanisms include liposome-derived systems, artificial viral envelopes, and other systems known in the art (Rossi, J. J. (1995) Br. Med. Bull. 51:217-225; Boado, R. J. et al. (1998) J. Pharm. Sci. 87:1308-1315; Morris, M. C. et al. (1997) Nucleic Acids Res. 25:2730-2736).

In another embodiment of the invention, polynucleotides encoding MDDT may be used for somatic or germline gene therapy. Gene therapy may be performed to (i) correct a genetic deficiency (e.g., in the cases of severe combined immunodeficiency (SCID)-X1 disease characterized by X-linked inheritance (Cavazzana-Calvo, M. et al. (2000) Science 288:669-672), severe combined immunodeficiency syndrome associated with an inherited adenosine deaminase (ADA) deficiency (Blaese, R. M. et al. (1995) Science 270:475-480; Bordignon, C. et al. (1995) Science 270:470-475), cystic fibrosis (Zabner, J. et al. (1993) Cell 75:207-216; Crystal, R. G. et al. (1995) Hum. Gene Therapy 6:643-666; Crystal, R. G. et al. (1995) Hum. Gene Therapy 6:667-703), thalassamias, familial hypercholesterolemia, and hemophilia resulting from Factor VIII or Factor IX deficiencies (Crystal, R. G. (1995) Science 270:404-410; Verma, I. M. and N. Somia (1997) Nature 389:239-242)), (ii) express a conditionally lethal gene product (e.g., in the case of cancers which result from unregulated cell proliferation), or (iii) express a protein which affords protection against intracellular parasites (e.g., against human retroviruses, such as human immunodeficiency virus (HIV) (Baltimore, D. (1988) Nature 335:395-396; Poeschla, E. et al. (1996) Proc. Natl. Acad. Sci. USA 93:11395-11399), hepatitis B or C virus (HBV, HCV); fungal parasites, such as Candida albicans and Paracoccidioides brasiliensis; and protozoan parasites such as Plasmodium falciparum and Trypanosoma cruzi). In the case where a genetic deficiency in MDDT expression or regulation causes disease, the expression of MDDT from an appropriate population of transduced cells may alleviate the clinical manifestations caused by the genetic deficiency.

In a further embodiment of the invention, diseases or disorders caused by deficiencies in MDDT are treated by constructing mammalian expression vectors encoding MDDT and introducing these vectors by mechanical means into MDDT-deficient cells. Mechanical transfer technologies for use with cells in vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and W. F. Anderson (1993) Annu. Rev. Biochem. 62:191-217; Ivics, Z. (1997) Cell 91:501-510; Boulay, J.-L. and H. Récipon (1998) Curr. Opin. Biotechnol. 9:445-450).

Expression vectors that may be effective for the expression of MDDT include, but are not limited to, the PCDNA 3.1, EPITAG, PRCCMV2, PREP, PVAX, PCR2-TOPOTA vectors (Invitrogen, Carlsbad Calif.), PCMV-SCRIPT, PCMV-TAG, PEGSH/PERV (Stratagene, La Jolla Calif.), and PTET-OFF, PTET-ON, PTRE2, PTRE2-LUC, PTK-HYG (Clontech, Palo Alto Calif.). MDDT may be expressed using (i) a constitutively active promoter, (e.g., from cytomegalovirus (CMV), Rous sarcoma virus (RSV), SV40 virus, thymidine kinase (TK), or β-actin genes), (ii) an inducible promoter (e.g., the tetracycline-regulated promoter (Gossen, M. and H. Bujard (1992) Proc. Natl. Acad. Sci. USA 89:5547-5551; Gossen, M. et al. (1995) Science 268:1766-1769; Rossi, F. M. V. and H. M. Blau (1998) Curr. Opin. Biotechnol. 9:451-456), commercially available in the T-REX plasmid (Invitrogen)); the ecdysone-inducible promoter (available in the plasmids PVGRXR and PIND; Invitrogen); the FK506/rapamycin inducible promoter; or the RU486/mifepristone inducible promoter (Rossi, F. M. V. and H. M. Blau, supra)), or (iii) a tissue-specific promoter or the native promoter of the endogenous gene encoding MDDT from a normal individual.

Commercially available liposome transformation kits (e.g., the PERFECT LIPID TRANSFECTION KIT, available from Invitrogen) allow one with ordinary skill in the art to deliver polynucleotides to target cells in culture and require minimal effort to optimize experimental parameters. In the alternative, transformation is performed using the calcium phosphate method (Graham, F. L. and A. J. Eb (1973) Virology 52:456-467), or by electroporation (Neumann, E. et al. (1982) EMBO J. 1:841-845). The introduction of DNA to primary cells requires modification of these standardized mammalian transfection protocols.

In another embodiment of the invention, diseases or disorders caused by genetic defects with respect to MDDT expression are treated by constructing a retrovirus vector consisting of (i) the polynucleotide encoding MDDT under the control of an independent promoter or the retrovirus long terminal repeat (LTR) promoter, (ii) appropriate RNA packaging signals, and (iii) a Rev-responsive element (RRE) along with additional retrovirus cis-acting RNA sequences and coding sequences required for efficient vector propagation. Retrovirus vectors (e.g., PFB and PFBNEO) are commercially available (Stratagene) and are based on published data (Riviere, I. et al. (1995) Proc. Natl. Acad. Sci. USA 92:6733-6737), incorporated by reference herein. The vector is propagated in an appropriate vector producing cell line (VPCL) that expresses an envelope gene with a tropism for receptors on the target cells or a proniscuous envelope protein such as VSVg (Armentano, D. et al. (1987) J. Virol. 61:1647-1650; Bender, M. A. et al. (1987) J. Virol. 61:1639-1646; Adam, M. A. and A. D. Miller (1988) J. Virol. 62:3802-3806; Dull, T. et al. (1998) J. Virol. 72:8463-8471; Zufferey, R. et al. (1998) J. Virol. 72:9873-9880). U.S. Pat. No. 5,910,434 to Rigg (“Method for obtaining retrovirus packaging cell lines producing high transducing efficiency retroviral supernatant”) discloses a method for obtaining retrovirus packaging cell lines and is hereby incorporated by reference. Propagation of retrovirus vectors, transduction of a population of cells (e.g., CD4⁺ T-cells), and the return of transduced cells to a patient are procedures well known to persons skilled in the art of gene therapy and have been well documented (Ranga, U. et al. (1997) J. Virol. 71:7020-7029; Bauer, G. et al. (1997) Blood 89:2259-2267; Bonyhadi, M. L. (1997) J. Virol. 71:4707-4716; Ranga, U. et al. (1998) Proc. Natl. Acad. Sci. USA 95:1201-1206; Su, L. (1997) Blood 89:2283-2290).

In an embodiment, an adenovirus-based gene therapy delivery system is used to deliver polynucleotides encoding MDDT to cells which have one or more genetic abnormalities with respect to the expression of MDDT. The construction and packaging of adenovirus-based vectors are well known to those with ordinary skill in the art. Replication defective adenovirus vectors have proven to be versatile for importing genes encoding immunoregulatory proteins into intact islets in the pancreas (Csete, M. E. et al. (1995) Transplantation 27:263-268). Potentially useful adenoviral vectors are described in U.S. Pat. No. 5,707,618 to Armentano (“Adenovirus vectors for gene therapy”), hereby incorporated by reference. For adenoviral vectors, see also Antinozzi, P. A. et al. (1999; Annu. Rev. Nutr. 19:511-544) and Verma, I. M. and N. Somia (1997; Nature 18:389:239-242).

In another embodiment, a herpes-based, gene therapy delivery system is used to deliver polynucleotides encoding MDDT to target cells which have one or more genetic abnormalities with respect to the expression of MDDT. The use of herpes simplex virus (HSV)-based vectors may be especially valuable for introducing MDDT to cells of the central nervous system, for which HSV has a tropism. The construction and packaging of herpes-based vectors are well known to those with ordinary skill in the art. A replication-competent herpes simplex virus (HSV) type 1-based vector has been used to deliver a reporter gene to the eyes of primates (Liu, X. et al. (1999) Exp. Eye Res. 169:385-395). The construction of a HSV-1 virus vector has also been disclosed in detail in U.S. Pat. No. 5,804,413 to DeLuca (“Herpes simplex virus strains for gene transfer”), which is hereby incorporated by reference. U.S. Pat. No. 5,804,413 teaches the use of recombinant HSV d92 which consists of a genome containing at least one exogenous gene to be transferred to a cell under the control of the appropriate promoter for purposes including human gene therapy. Also taught by this patent are the construction and use of recombinant HSV strains deleted for ICP4, ICP27 and ICP22. For HSV vectors, see also Goins, W. F. et al. (1999; J. Virol. 73:519-532) and Xu, H. et al. (1994; Dev. Biol. 163:152-161). The manipulation of cloned herpesvirus sequences, the generation of recombinant virus following the transfection of multiple plasmids containing different segments of the large herpesvirus genomes, the growth and propagation of herpesvirus, and the infection of cells with herpesvirus are techniques well known to those of ordinary skill in the art.

In another embodiment, an alphavirus (positive, single-stranded RNA virus) vector is used to deliver polynucleotides encoding MDDT to target cells. The biology of the prototypic alphavirus, Semliki Forest Virus (SFV), has been studied extensively and gene transfer vectors have been based on the SFV genome (Garoff, H. and K.-J. Li (1998) Curr. Opin. Biotechnol. 9:464-469). During alphavirus RNA replication, a subgenomic RNA is generated that normally encodes the viral capsid proteins. This subgenomic RNA replicates to higher levels than the full length genomic RNA, resulting in the overproduction of capsid proteins relative to the viral proteins with enzymatic activity (e.g., protease and polymerase). Similarly, inserting the coding sequence for MDDT into the alphavirus genome in place of the capsid-coding region results in the production of a large number of MDDT-coding RNAs and the synthesis of high levels of MDDT in vector transduced cells. While alphavirus infection is typically associated with cell lysis within a few days, the ability to establish a persistent infection in hamster normal kidney cells (BHK-21) with a variant of Sindbis virus (SIN) indicates that the lytic replication of alphaviruses can be altered to suit the needs of the gene therapy application (Dryga, S. A. et al. (1997) Virology 228:74-83). The wide host range of alphaviruses will allow the introduction of MDDT into a variety of cell types. The specific transduction of a subset of cells in a population may require the sorting of cells prior to transduction. The methods of manipulating infectious cDNA clones of alphaviruses, performing alphavirus cDNA and RNA transfections, and performing alphavirus infections, are well known to those with ordinary skill in the art.

Oligonucleotides derived from the transcription initiation site, e.g., between about positions −10 and +10 from the start site, may also be employed to inhibit gene expression. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) in Huber, B. E. and B. I. Carr, Molecular and Immunologic Approaches, Futura Publishing, Mt. Kisco N.Y., pp. 163-177). A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of RNA molecules encoding MDDT.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA molecules encoding MDDT. Such DNA sequences may be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

In other embodiments of the invention, the expression of one or more selected polynucleotides of the present invention can be altered, inhibited, decreased, or silenced using RNA interference (RNAi) or post-transcriptional gene silencing (PTGS) methods known in the art. RNAi is a post-transcriptional mode of gene silencing in which double-stranded RNA (dsRNA) introduced into a targeted cell specifically suppresses the expression of the homologous gene (i.e., the gene bearing the sequence complementary to the dsRNA). This effectively knocks out or substantially reduces the expression of the targeted gene. PTGS can also be accomplished by use of DNA or DNA fragments as well. RNAi methods are described by Fire, A. et al. (1998; Nature 391:806-811) and Gura, T. (2000; Nature 404:804-808). PTGS can also be initiated by introduction of a complementary segment of DNA into the selected tissue using gene delivery and/or viral vector delivery methods described herein or known in the art.

RNAi can be induced in mammalian cells by the use of small interfering RNA also known as siRNA. SIRNA are shorter segments of dsRNA (typically about 21 to 23 nucleotides in length) that result in vivo from cleavage of introduced dsRNA by the action of an endogenous ribonuclease. SIRNA appear to be the mediators of the RNAi effect in mammals. The most effective siRNAs appear to be 21 nucleotide dsRNAs with 2 nucleotide 3′ overhangs. The use of siRNA for inducing RNAi in mammalian cells is described by Elbashir, S. M. et al. (2001; Nature 411:494-498).

SiRNA can either be generated indirectly by introduction of dsRNA into the targeted cell, or directly by mammalian transfection methods and agents described herein or known in the art (such as liposome-mediated transfection, viral vector methods, or other polynucleotide delivery/introductory methods). Suitable SiRNAs can be selected by examining a transcript of the target polynucleotide (e.g., mRNA) for nucleotide sequences downstream from the AUG start codon and recording the occurrence of each nucleotide and the 3′ adjacent 19 to 23 nucleotides as potential siRNA target sites, with sequences having a 21 nucleotide length being preferred. Regions to be avoided for target siRNA sites include the 5′ and 3′ untranslated regions (UTRs) and regions near the start codon (within 75 bases), as these may be richer in regulatory protein binding sites. UTR-binding proteins and/or translation initiation complexes may interfere with binding of the siRNP endonuclease complex. The selected target sites for siRNA can then be compared to the appropriate genome database (e.g., human, etc.) using BLAST or other sequence comparison algorithms known in the art. Target sequences with significant homology to other coding sequences can be eliminated from consideration. The selected SiRNAs can be produced by chemical synthesis methods known in the art or by in vitro transcription using commercially available methods and kits such as the SILENCER siRNA construction kit (Ambion, Austin Tex.).

In alternative embodiments, long-term gene silencing and/or RNAi effects can be induced in selected tissue using expression vectors that continuously express siRNA. This can be accomplished using expression vectors that are engineered to express hairpin RNAs (shRNAs) using methods known in the art (see, e.g., Brummelkamp, T. R. et al. (2002) Science 296:550-553; and Paddison, P. J. et al. (2002) Genes Dev. 16:948-958). In these and related embodiments, shRNAs can be delivered to target cells using expression vectors known in the art. An example of a suitable expression vector for delivery of siRNA is the PSILENCER1.0-U6 (circular) plasmid (Ambion). Once delivered to the target tissue, shRNAs are processed in vivo into siRNA-like molecules capable of carrying out gene-specific silencing.

In various embodiments, the expression levels of genes targeted by RNAi or PTGS methods can be determined by assays for mRNA and/or protein analysis. Expression levels of the mRNA of a targeted gene, can be determined by northern analysis methods using, for example, the NORTHERNMAX-GLY kit (Ambion); by microarray methods; by PCR methods; by real time PCR methods; and by other RNA/polynucleotide assays known in the art or described herein. Expression levels of the protein encoded by the targeted gene can be determined by Western analysis using standard techniques known in the art.

An additional embodiment of the invention encompasses a method for screening for a compound which is effective in altering expression of a polynucleotide encoding MDDT. Compounds which may be effective in altering expression of a specific polynucleotide may include, but are not limited to, oligonucleotides, antisense oligonucleotides, triple helix-forming oligonucleotides, transcription factors and other polypeptide transcriptional regulators, and non-macromolecular chemical entities which are capable of interacting with specific polynucleotide sequences. Effective compounds may alter polynucleotide expression by acting as either inhibitors or promoters of polynucleotide expression. Thus, in the treatment of disorders associated with increased MDDT expression or activity, a compound which specifically inhibits expression of the polynucleotide encoding MDDT may be therapeutically useful, and in the treatment of disorders associated with decreased MDDT expression or activity, a compound which specifically promotes expression of the polynucleotide encoding MDDT may be therapeutically useful.

In various embodiments, one or more test compounds may be screened for effectiveness in altering expression of a specific polynucleotide. A test compound may be obtained by any method commonly known in the art, including chemical modification of a compound known to be effective in altering polynucleotide expression; selection from an existing, commercially-available or proprietary library of naturally-occurring or non-natural chemical compounds; rational design of a compound based on chemical and/or structural properties of the target polynucleotide; and selection from a library of chemical compounds created combinatorially or randomly. A sample comprising a polynucleotide encoding MDDT is exposed to at least one test compound thus obtained. The sample may comprise, for example, an intact or permeabilized cell, or an in vitro cell-free or reconstituted biochemical system. Alterations in the expression of a polynucleotide encoding MDDT are assayed by any method commonly known in the art. Typically, the expression of a specific nucleotide is detected by hybridization with a probe having a nucleotide sequence complementary to the sequence of the polynucleotide encoding MDDT. The amount of hybridization may be quantified, thus forming the basis for a comparison of the expression of the polynucleotide both with and without exposure to one or more test compounds. Detection of a change in the expression of a polynucleotide exposed to a test compound indicates that the test compound is effective in altering the expression of the polynucleotide. A screen for a compound effective in altering expression of a specific polynucleotide can be carried out, for example, using a Schizosaccharomyces pombe gene expression system (Atkins, D. et al. (1999) U.S. Pat. No. 5,932,435; Arndt, G. M. et al. (2000) Nucleic Acids Res. 28:E15) or a human cell line such as HeLa cell (Clarke, M. L. et al. (2000) Biochem. Biophys. Res. Commun. 268:8-13). A particular embodiment of the present invention involves screening a combinatorial library of oligonucleotides (such as deoxyribonucleotides, ribonucleotides, peptide nucleic acids, and modified oligonucleotides) for antisense activity against a specific polynucleotide sequence (Bruice, T. W. et al. (1997) U.S. Pat. No. 5,686,242; Bruice, T. W. et al. (2000) U.S. Pat. No. 6,022,691).

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art (Goldman, C. K. et al. (1997) Nat. Biotechnol. 15:462-466).

Any of the therapeutic methods described above may be applied to any subject in need of such therapy, including, for example, mammals such as humans, dogs, cats, cows, horses, rabbits, and monkeys.

An additional embodiment of the invention relates to the administration of a composition which generally comprises an active ingredient formulated with a pharmaceutically acceptable excipient. Excipients may include, for example, sugars, starches, celluloses, gums, and proteins. Various formulations are commonly known and are thoroughly discussed in the latest edition of Remington's Pharmaceutical Sciences (Maack Publishing, Easton Pa.). Such compositions may consist of MDDT, antibodies to MDDT, and mimetics, agonists, antagonists, or inhibitors of MDDT.

In various embodiments, the compositions described herein, such as pharmaceutical compositions, may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means.

Compositions for pulmonary administration may be prepared in liquid or dry powder form. These compositions are generally aerosolized immediately prior to inhalation by the patient. In the case of small molecules (e.g. traditional low molecular weight organic drugs), aerosol delivery of fast-acting formulations is well-known in the art. In the case of macromolecules (e.g. larger peptides and proteins), recent developments in the field of pulmonary delivery via the alveolar region of the lung have enabled the practical delivery of drugs such as insulin to blood circulation (see, e.g., Patton, J. S. et al., U.S. Pat. No. 5,997,848). Pulmonary delivery allows administration without needle injection, and obviates the need for potentially toxic penetration enhancers.

Compositions suitable for use in the invention include compositions wherein the active ingredients are contained in an effective amount to achieve the intended purpose. The determination of an effective dose is well within the capability of those skilled in the art.

Specialized forms of compositions may be prepared for direct intracellular delivery of macromolecules comprising MDDT or fragments thereof. For example, liposome preparations containing a cell-impermeable macromolecule may promote cell fusion and intracellular delivery of the macromolecule. Alternatively, MDDT or a fragment thereof may be joined to a short cationic N-terminal portion from the HIV Tat-1 protein. Fusion proteins thus generated have been found to transduce into the cells of all tissues, including the brain, in a mouse model system (Schwarze, S. R. et al. (1999) Science 285:1569-1572).

For any compound, the therapeutically effective dose can be estimated initially either in cell culture assays, e.g., of neoplastic cells, or in animal models such as mice, rats, rabbits, dogs, monkeys, or pigs. An animal model may also be used to determine the appropriate concentration range and route of administration. Such information can then be used to determine useful doses and routes for administration in humans.

A therapeutically effective dose refers to that amount of active ingredient, for example MDDT or fragments thereof, antibodies of MDDT, and agonists, antagonists or inhibitors of MDDT, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of toxic to therapeutic effects is the therapeutic index, which can be expressed as the LD₅₀/ED₅₀ ratio. Compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting compositions may be administered every 3 to 4 days, every week, or biweekly depending on the half-life and clearance rate of the particular formulation.

Normal dosage amounts may vary from about 0.1 μg to 100,000 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Diagnostics

In another embodiment, antibodies which specifically bind MDDT may be used for the diagnosis of disorders characterized by expression of MDDT, or in assays to monitor patients being treated with MDDT or agonists, antagonists, or inhibitors of MDDT. Antibodies useful for diagnostic purposes may be prepared in the same manner as described above for therapeutics. Diagnostic assays for MDDT include methods which utilize the antibody and a label to detect MDDT in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring MDDT, including ELISAs, RIAs, and FACS, are known in the art and provide a basis for diagnosing altered or abnormal levels of MDDT expression. Normal or standard values for MDDT expression are established by combining body fluids or cell extracts taken from normal mammalian subjects, for example, human subjects, with antibodies to MDDT under conditions suitable for complex formation. The amount of standard complex formation may be quantitated by various methods, such as photometric means. Quantities of MDDT expressed in subject, control, and disease samples from biopsied tissues are compared with the standard values. Deviation between standard and subject values establishes the parameters for diagnosing disease.

In another embodiment of the invention, polynucleotides encoding MDDT may be used for diagnostic purposes. The polynucleotides which may be used include oligonucleotides, complementary RNA and DNA molecules, and PNAs. The polynucleotides may be used to detect and quantify gene expression in biopsied tissues in which expression of MDDT may be correlated with disease. The diagnostic assay may be used to determine absence, presence, and excess expression of MDDT, and to monitor regulation of MDDT levels during therapeutic intervention.

In one aspect, hybridization with PCR probes which are capable of detecting polynucleotides, including genomic sequences, encoding MDDT or closely related molecules may be used to identify nucleic acid sequences which encode MDDT. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding MDDT, allelic variants, or related sequences.

Probes may also be used for the detection of related sequences, and may have at least 50% sequence identity to any of the MDDT encoding sequences. The hybridization probes of the subject invention may be DNA or RNA and may be derived from the sequence of SEQ ID NO:49-96 or from genomic sequences including promoters, enhancers, and introns of the MDDT gene.

Means for producing specific hybridization probes for polynucleotides encoding MDDT include the cloning of polynucleotides encoding MDDT or MDDT derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³⁵S, or by enzymatic labels, such as alkaline phosphatase coupled to the probe via avidin/biotin coupling systems, and the like.

Polynucleotides encoding MDDT may be used for the diagnosis of disorders associated with expression of MDDT. Examples of such disorders include, but are not limited to, a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss; and a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia. Polynucleotides encoding MDDT may be used in Southern or northern analysis, dot blot, or other membrane-based technologies; in PCR technologies; in dipstick, pin, and multiformat ELISA-like assays; and in microarrays utilizing fluids or tissues from patients to detect altered MDDT expression. Such qualitative or quantitative methods are well known in the art.

In a particular embodiment, polynucleotides encoding MDDT may be used in assays that detect the presence of associated disorders, particularly those mentioned above. Polynucleotides complementary to sequences encoding MDDT may be labeled by standard methods and added to a fluid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of polynucleotides encoding MDDT in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of MDDT, a normal or standard profile for expression is established. This may be accomplished by combining body fluids or cell extracts taken from normal subjects, either animal or human, with a sequence, or a fragment thereof, encoding MDDT, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of an abnormal amount of transcript (either under- or overexpressed) in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier, thereby preventing the development or further progression of the cancer.

Additional diagnostic uses for oligonucleotides designed from the sequences encoding MDDT may involve the use of PCR. These oligomers may be chemically synthesized, generated enzymatically, or produced in vitro. Oligomers will preferably contain a fragment of a polynucleotide encoding MDDT, or a fragment of a polynucleotide complementary to the polynucleotide encoding MDDT, and will be employed under optimized conditions for identification of a specific gene or condition. Oligomers may also be employed under less stringent conditions for detection or quantification of closely related DNA or RNA sequences.

In a particular aspect, oligonucleotide primers derived from polynucleotides encoding MDDT may be used to detect single nucleotide polymorphisms (SNPs). SNPs are substitutions, insertions and deletions that are a frequent cause of inherited or acquired genetic disease in humans. Methods of SNP detection include, but are not limited to, single-stranded conformation polymorphism (SSCP) and fluorescent SSCP (fSSCP) methods. In SSCP, oligonucleotide primers derived from polynucleotides encoding MDDT are used to amplify DNA using the polymerase chain reaction (PCR). The DNA may be derived, for example, from diseased or normal tissue, biopsy samples, bodily fluids, and the like. SNPs in the DNA cause differences in the secondary and tertiary structures of PCR products in single-stranded form, and these differences are detectable using gel electrophoresis in non-denaturing gels. In fSCCP, the oligonucleotide primers are fluorescently labeled, which allows detection of the amplimers in high-throughput equipment such as DNA sequencing machines. Additionally, sequence database analysis methods, termed in silico SNP (is SNP), are capable of identifying polymorphisms by comparing the sequence of individual overlapping DNA fragments which assemble into a common consensus sequence. These computer-based methods filter out sequence variations due to laboratory preparation of DNA and sequencing errors using statistical models and automated analyses of DNA sequence chromatograms. In the alternative, SNPs may be detected and characterized by mass spectrometry using, for example, the high throughput MASSARRAY system (Sequenom, Inc., San Diego Calif.).

SNPs may be used to study the genetic basis of human disease. For example, at least 16 common SNPs have been associated with non-insulin-dependent diabetes mellitus. SNPs are also useful for examining differences in disease outcomes in monogenic disorders, such as cystic fibrosis, sickle cell anemia, or chronic granulomatous disease. For example, variants in the mannose-binding lectin, MBL2, have been shown to be correlated with deleterious pulmonary outcomes in cystic fibrosis. SNPs also have utility in pharmacogenomics, the identification of genetic variants that influence a patient's response to a drug, such as life-threatening toxicity. For example, a variation in N-acetyl transferase is associated with a high incidence of peripheral neuropathy in response to the anti-tuberculosis drug isoniazid, while a variation in the core promoter of the ALOX5 gene results in diminished clinical response to treatment with an anti-asthma drug that targets the 5-lipoxygenase pathway. Analysis of the distribution of SNPs in different populations is useful for investigating genetic drift, mutation, recombination, and selection, as well as for tracing the origins of populations and their migrations (Taylor, J. G. et al. (2001) Trends Mol. Med. 7:507-512; Kwok, P.-Y. and Z. Gu (1999) Mol. Med. Today 5:538-543; Nowotny, P. et al. (2001) Curr. Opin. Neurobiol. 11:637-641).

Methods which may also be used to quantify the expression of MDDT include radiolabeling or biotinylating nucleotides, coamplification of a control nucleic acid, and interpolating results from standard curves (Melby, P. C. et al. (1993) J. Immunol. Methods 159:235-244; Duplaa, C. et al. (1993) Anal. Biochem. 212:229-236). The speed of quantitation of multiple samples may be accelerated by running the assay in a high-throughput format where the oligomer or polynucleotide of interest is presented in various dilutions and a spectrophotometric or colorimetric response gives rapid quantitation.

In further embodiments, oligonucleotides or longer fragments derived from any of the polynucleotides described herein may be used as elements on a microarray. The microarray can be used in transcript imaging techniques which monitor the relative expression levels of large numbers of genes simultaneously as described below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient. For example, therapeutic agents which are highly effective and display the fewest side effects may be selected for a patient based on his/her pharmacogenomic profile.

In another embodiment, MDDT, fragments of MDDT, or antibodies specific for MDDT may be used as elements on a microarray. The microarray may be used to monitor or measure protein-protein interactions, drug-target interactions, and gene expression profiles, as described above.

A particular embodiment relates to the use of the polynucleotides of the present invention to generate a transcript image of a tissue or cell type. A transcript image represents the global pattern of gene expression by a particular tissue or cell type. Global gene expression patterns are analyzed by quantifying the number of expressed genes and their relative abundance under given conditions and at a given time (Seilhamer et al., “Comparative Gene Transcript Analysis,” U.S. Pat. No. 5,840,484; hereby expressly incorporated by reference herein). Thus a transcript image may be generated by hybridizing the polynucleotides of the present invention or their complements to the totality of transcripts or reverse transcripts of a particular tissue or cell type. In one embodiment, the hybridization takes place in high-throughput format, wherein the polynucleotides of the present invention or their complements comprise a subset of a plurality of elements on a microarray. The resultant transcript image would provide a profile of gene activity.

Transcript images may be generated using transcripts isolated from tissues, cell lines, biopsies, or other biological samples. The transcript image may thus reflect gene expression in vivo, as in the case of a tissue or biopsy sample, or in vitro, as in the case of a cell line.

Transcript images which profile the expression of the polynucleotides of the present invention may also be used in conjunction with in vitro model systems and preclinical evaluation of pharmaceuticals, as well as toxicological testing of industrial and naturally-occurring environmental compounds. All compounds induce characteristic gene expression patterns, frequently termed molecular fingerprints or toxicant signatures, which are indicative of mechanisms of action and toxicity (Nuwaysir, E. F. et al. (1999) Mol. Carcinog. 24:153-159; Steiner, S. and N. L. Anderson (2000) Toxicol. Lett. 112-113:467-471). If a test compound has a signature similar to that of a compound with known toxicity, it is likely to share those toxic properties. These fingerprints or signatures are most useful and refined when they contain expression information from a large number of genes and gene families. Ideally, a genome-wide measurement of expression provides the highest quality signature. Even genes whose expression is not altered by any tested compounds are important as well, as the levels of expression of these genes are used to normalize the rest of the expression data. The normalization procedure is useful for comparison of expression data after treatment with different compounds. While the assignment of gene function to elements of a toxicant signature aids in interpretation of toxicity mechanisms, knowledge of gene function is not necessary for the statistical matching of signatures which leads to prediction of toxicity (see, for example, Press Release 00-02 from the National Institute of Environmental Health Sciences, released Feb. 29, 2000, available at http://www.niehs.nih.gov/oc/news/toxchip.htm). Therefore, it is important and desirable in toxicological screening using toxicant signatures to include all expressed gene sequences.

In an embodiment, the toxicity of a test compound can be assessed by treating a biological sample containing nucleic acids with the test compound. Nucleic acids that are expressed in the treated biological sample are hybridized with one or more probes specific to the polynucleotides of the present invention, so that transcript levels corresponding to the polynucleotides of the present invention may be quantified. The transcript levels in the treated biological sample are compared with levels in an untreated biological sample. Differences in the transcript levels between the two samples are indicative of a toxic response caused by the test compound in the treated sample.

Another embodiment relates to the use of the polypeptides disclosed herein to analyze the proteome of a tissue or cell type. The term proteome refers to the global pattern of protein expression in a particular tissue or cell type. Each protein component of a proteome can be subjected individually to further analysis. Proteome expression patterns, or profiles, are analyzed by quantifying the number of expressed proteins and their relative abundance under given conditions and at a given time. A profile of a cell's proteome may thus be generated by separating and analyzing the polypeptides of a particular tissue or cell type. In one embodiment, the separation is achieved using two-dimensional gel electrophoresis, in which proteins from a sample are separated by isoelectric focusing in the first dimension, and then according to molecular weight by sodium dodecyl sulfate slab gel electrophoresis in the second dimension (Steiner and Anderson, supra). The proteins are visualized in the gel as discrete and uniquely positioned spots, typically by staining the gel with an agent such as Coomassie Blue or silver or fluorescent stains. The optical density of each protein spot is generally proportional to the level of the protein in the sample. The optical densities of equivalently positioned protein spots from different samples, for example, from biological samples either treated or untreated with a test compound or therapeutic agent, are compared to identify any changes in protein spot density related to the treatment. The proteins in the spots are partially sequenced using, for example, standard methods employing chemical or enzymatic cleavage followed by mass spectrometry. The identity of the protein in a spot may be determined by comparing its partial sequence, preferably of at least 5 contiguous amino acid residues, to the polypeptide sequences of interest. In some cases, further sequence data may be obtained for definitive protein identification.

A proteomic profile may also be generated using antibodies specific for MDDT to quantify the levels of MDDT expression. In one embodiment, the antibodies are used as elements on a microarray, and protein expression levels are quantified by exposing the microarray to the sample and detecting the levels of protein bound to each array element (Lueking, A. et al. (1999) Anal. Biochem. 270:103-111; Mendoze, L. G. et al. (1999) Biotechniques 27:778-788). Detection may be performed by a variety of methods known in the art, for example, by reacting the proteins in the sample with a thiol- or amino-reactive fluorescent compound and detecting the amount of fluorescence bound at each array element.

Toxicant signatures at the proteome level are also useful for toxicological screening, and should be analyzed in parallel with toxicant signatures at the transcript level. There is a poor correlation between transcript and protein abundances for some proteins in some tissues (Anderson, N. L. and J. Seilhamer (1997) Electrophoresis 18:533-537), so proteome toxicant signatures may be useful in the analysis of compounds which do not significantly affect the transcript image, but which alter the proteomic profile. In addition, the analysis of transcripts in body fluids is difficult, due to rapid degradation of mRNA, so proteomic profiling may be more reliable and informative in such cases.

In another embodiment, the toxicity of a test compound is assessed by treating a biological sample containing proteins with the test compound. Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the polypeptides of the present invention.

In another embodiment, the toxicity of a test compound is assessed by treating a biological sample containing proteins with the test compound. Proteins from the biological sample are incubated with antibodies specific to the polypeptides of the present invention. The amount of protein recognized by the antibodies is quantified. The amount of protein in the treated biological sample is compared with the amount in an untreated biological sample. A difference in the amount of protein between the two samples is indicative of a toxic response to the test compound in the treated sample.

Microarrays may be prepared, used, and analyzed using methods known in the art (Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. USA 94:2150-2155; Heller, M. J. et al. (1997) U.S. Pat. No. 5,605,662). Various types of microarrays are well known and thoroughly described in Schena, M., ed. (1999; DNA Microarrays: A Practical Approach, Oxford University Press, London).

In another embodiment of the invention, nucleic acid sequences encoding MDDT may be used to generate hybridization probes useful in mapping the naturally occurring genomic sequence. Either coding or noncoding sequences may be used, and in some instances, noncoding sequences may be preferable over coding sequences. For example, conservation of a coding sequence among members of a multi-gene family may potentially cause undesired cross hybridization during chromosomal mapping. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries (Harrington, J. J. et al. (1997) Nat. Genet. 15:345-355; Price, C. M. (1993) Blood Rev. 7:127-134; Trask, B. J. (1991) Trends Genet. 7:149-154). Once mapped, the nucleic acid sequences may be used to develop genetic linkage maps, for example, which correlate the inheritance of a disease state with the inheritance of a particular chromosome region or restriction fragment length polymorphism (RFLP) (Lander, E. S. and D. Botstein (1986) Proc. Natl. Acad. Sci. USA 83:7353-7357).

Fluorescent in situ hybridization (FISH) may be correlated with other physical and genetic map data (Heinz-Ulrich, et al. (1995) in Meyers, supra, pp. 965-968). Examples of genetic map data can be found in various scientific journals or at the Online Mendelian Inheritance in Man (OMIM) World Wide Web site. Correlation between the location of the gene encoding MDDT on a physical map and a specific disorder, or a predisposition to a specific disorder, may help define the region of DNA associated with that disorder and thus may further positional cloning efforts.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the exact chromosomal locus is not known. This information is valuable to investigators searching for disease genes using positional cloning or other gene discovery techniques. Once the gene or genes responsible for a disease or syndrome have been crudely localized by genetic linkage to a particular genomic region, e.g., ataxia-telangiectasia to 11q22-23, any sequences mapping to that area may represent associated or regulatory genes for further investigation (Gatti, R. A. et al. (1988) Nature 336:577-580). The nucleotide sequence of the instant invention may also be used to detect differences in the chromosomal location due to translocation, inversion, etc., among normal, carrier, or affected individuals.

In another embodiment of the invention, MDDT, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between MDDT and the agent being tested may be measured.

Another technique for drug screening provides for high throughput screening of compounds having suitable binding affinity to the protein of interest (Geysen, et al. (1984) PCT application WO84/03564). In this method, large numbers of different small test compounds are synthesized on a solid substrate. The test compounds are reacted with MDDT, or fragments thereof, and washed. Bound MDDT is then detected by methods well known in the art. Purified MDDT can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding MDDT specifically compete with a test compound for binding MDDT. In this manner, antibodies can be used to detect the presence of any peptide which shares one or more antigenic determinants with MDDT.

In additional embodiments, the nucleotide sequences which encode MDDT may be used in any molecular biology techniques that have yet to be developed, provided the new techniques rely on properties of nucleotide sequences that are currently known, including, but not limited to, such properties as the triplet genetic code and specific base pair interactions.

Without further elaboration, it is believed that one skilled in the art can, using the preceding description, utilize the present invention to its fullest extent. The following embodiments are, therefore, to be construed as merely illustrative, and not limitative of the remainder of the disclosure in any way whatsoever.

The disclosures of all patents, applications, and publications mentioned above and below, including U.S. Ser. No. 60/328,944, U.S. Ser. No. 60/332,430, U.S. Ser. No. 60/343,880, U.S. Ser. No. 60/345,143, and U.S. Ser. No. 60/345,384, are hereby expressly incorporated by reference.

### EXAMPLES

I. Construction of cDNA Libraries

Incyte cDNAs were derived from cDNA libraries described in the LIFESEQ GOLD database (Incyte Genomics, Palo Alto Calif.). Some tissues were homogenized and lysed in guanidinium isothiocyanate, while others were homogenized and lysed in phenol or in a suitable mixture of denaturants, such as TRIZOL (Invitrogen), a monophasic solution of phenol and guanidine isothiocyanate. The resulting lysates were centrifuged over CsCl cushions or extracted with chloroform. RNA was precipitated from the lysates with either isopropanol or sodium acetate and ethanol, or by other routine methods.

Phenol extraction and precipitation of RNA were repeated as necessary to increase RNA purity. In some cases, RNA was treated with DNase. For most libraries, poly(A)+ RNA was isolated using oligo d(T)-coupled paramagnetic particles (Promega), OLIGOTEX latex particles (QIAGEN, Chatsworth Calif.), or an OLIGOTEX mRNA purification kit (QIAGEN). Alternatively, RNA was isolated directly from tissue lysates using other RNA isolation kits, e.g., the POLY(A)PURE mRNA purification kit (Ambion, Austin Tex.).

In some cases, Stratagene was provided with RNA and constructed the corresponding cDNA libraries. Otherwise, cDNA was synthesized and cDNA libraries were constructed with the UNIZAP vector system (Stratagene) or SUPERSCRIPT plasmid system (Invitrogen), using the recommended procedures or similar methods known in the art (Ausubel et al., supra, ch. 5). Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S 1000, SEPHAROSE CL2B, or SEPHAROSE CL4B column chromatography (Amersham Biosciences) or preparative agarose gel electrophoresis. cDNAs were ligated into compatible restriction enzyme sites of the polylinker of a suitable plasmid, e.g., PBLUESCRIPT plasmid (Stratagene), PSPORT1 plasmid (Invitrogen, Carlsbad Calif.), PCDNA2.1 plasmid (Invitrogen), PBK-CMV plasmid (Stratagene), PCR2-TOPOTA plasmid (Invitrogen), PCMV-ICIS plasmid (Stratagene), pIGEN (Incyte Genomics, Palo Alto Calif.), pRARE (Incyte Genomics), or pINCY (Incyte Genomics), or derivatives thereof. Recombinant plasmids were transformed into competent E. coli cells including XL1-Blue, XL1-BlueMRF, or SOLR from Stratagene or DH5α, DH10B, or ElectroMAX DH10B from Invitrogen.

II. Isolation of cDNA Clones

Plasmids obtained as described in Example I were recovered from host cells by in vivo excision using the UNIZAP vector system (Stratagene) or by cell lysis. Plasmids were purified using at least one of the following: a Magic or WIZARD Minipreps DNA purification system (Promega); an AGTC Miniprep purification kit (Edge Biosystems, Gaithersburg Md.); and QIAWELL 8 Plasmid, QIAWELL 8 Plus Plasmid, QIAWELL 8 Ultra Plasmid purification systems or the R.E.A.L. PREP 96 plasmid purification kit from QIAGEN. Following precipitation, plasmids were resuspended in 0.1 ml of distilled water and stored, with or without lyophilization, at 4° C.

Alternatively, plasmid DNA was amplified from host cell lysates using direct link PCR in a high-throughput format (Rao, V. B. (1994) Anal. Biochem. 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes, Eugene Oreg.) and a FLUOROSKAN II fluorescence scanner (Labsystems Oy, Helsinki, Finland).

III. Sequencing and Analysis

Incyte cDNA recovered in plasmids as described in Example II were sequenced as follows. Sequencing reactions were processed using standard methods or high-throughput instrumentation such as the ABI CATALYST 800 (Applied Biosystems) thermal cycler or the PTC-200 thermal cycler (MJ Research) in conjunction with the HYDRA microdispenser (Robbins Scientific) or the MICROLAB 2200 (Hamilton) liquid transfer system. cDNA sequencing reactions were prepared using reagents provided by Amersham Biosciences or supplied in ABI sequencing kits such as the ABI PRISM BIGDYE Terminator cycle sequencing ready reaction kit (Applied Biosystems). Electrophoretic separation of cDNA sequencing reactions and detection of labeled polynucleotides were carried out using the MEGABACE 1000 DNA sequencing system (Amersham Biosciences); the ABI PRISM 373 or 377 sequencing system (Applied Biosystems) in conjunction with standard ABI protocols and base calling software; or other sequence analysis systems known in the art. Reading frames within the cDNA sequences were identified using standard methods (Ausubel et al., supra, ch. 7). Some of the cDNA sequences were selected for extension using the techniques disclosed in Example VIII.

The polynucleotide sequences derived from Incyte cDNAs were validated by removing vector, linker, and poly(A) sequences and by masking ambiguous bases, using algorithms and programs based on BLAST, dynamic programming, and dinucleotide nearest neighbor analysis. The Incyte cDNA sequences or translations thereof were then queried against a selection of public databases such as the GenBank primate, rodent, mammalian, vertebrate, and eukaryote databases, and BLOCKS, PRINTS, DOMO, PRODOM; PROTEOME databases with sequences from Homo sapiens, Rattus norvegicus, Mus musculus, Caenorhabditis elegans, Saccharomyces cerevisiae, Schizosaccharomyces pombe, and Candida albicans (Incyte Genomics, Palo Alto Calif.); hidden Markov model (HMM)-based protein family databases such as PFAM, INCY, and TIGRFAM (Haft, D. H. et al. (2001) Nucleic Acids Res. 29:41-43); and HMM-based protein domain databases such as SMART (Schultz, J. et al. (1998) Proc. Natl. Acad. Sci. USA 95:5857-5864; Letunic, I. et al. (2002) Nucleic Acids Res. 30:242-244). (HMM is a probabilistic approach which analyzes consensus primary structures of gene families; see, for example, Eddy, S. R. (1996) Curr. Opin. Struct. Biol. 6:361-365.) The queries were performed using programs based on BLAST, FASTA, BLIMPS, and HMMER. The Incyte cDNA sequences were assembled to produce full length polynucleotide sequences. Alternatively, GenBank cDNAs, GenBank ESTs, stitched sequences, stretched sequences, or Genscan-predicted coding sequences (see Examples IV and V) were used to extend Incyte cDNA assemblages to full length. Assembly was performed using programs based on Phred, Phrap, and Consed, and cDNA assemblages were screened for open reading frames using programs based on GeneMark, BLAST, and FASTA. The full length polynucleotide sequences were translated to derive the corresponding full length polypeptide sequences. Alternatively, a polypeptide may begin at any of the methionine residues of the full length translated polypeptide. Full length polypeptide sequences were subsequently analyzed by querying against databases such as the GenBank protein databases (genpept), SwissProt, the PROTEOME databases, BLOCKS, PRINTS, DOMO, PRODOM, Prosite, hidden Markov model (HMM)-based protein family databases such as PFAM, INCY, and TIGRFAM; and HMM-based protein domain databases such as SMART. Full length polynucleotide sequences are also analyzed using MACDNASIS PRO software (MiraiBio, Alameda Calif.) and LASERGENE software (DNASTAR). Polynucleotide and polypeptide sequence alignments are generated using default parameters specified by the CLUSTAL algorithm as incorporated into the MEGALIGN multisequence alignment program (DNASTAR), which also calculates the percent identity between aligned sequences.

Table 7 summarizes the tools, programs, and algorithms used for the analysis and assembly of Incyte cDNA and full length sequences and provides applicable descriptions, references, and threshold parameters. The first column of Table 7 shows the tools, programs, and algorithms used, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score or the lower the probability value, the greater the identity between two sequences).

The programs described above for the assembly and analysis of full length polynucleotide and polypeptide sequences were also used to identify polynucleotide sequence fragments from SEQ ID NO:49-96. Fragments from about 20 to about 4000 nucleotides which are useful in hybridization and amplification technologies are described in Table 4, column 2.

IV. Identification and Editing of Coding Sequences from Genomic DNA

Putative molecules for disease detection and treatment were initially identified by running the Genscan gene identification program against public genomic sequence databases (e.g., gbpri and gbhtg). Genscan is a general-purpose gene identification program which analyzes genomic DNA sequences from a variety of organisms (Burge, C. and S. Karlin (1997) J. Mol. Biol. 268:78-94; Burge, C. and S. Karlin (1998) Curr. Opin. Struct. Biol. 8:346-354). The program concatenates predicted exons to form an assembled cDNA sequence extending from a methionine to a stop codon. The output of Genscan is a FASTA database of polynucleotide and polypeptide sequences. The maximum range of sequence for Genscan to analyze at once was set to 30 kb. To determine which of these Genscan predicted cDNA sequences encode molecules for disease detection and treatment, the encoded polypeptides were analyzed by querying against PFAM models for molecules for disease detection and treatment. Potential molecules for disease detection and treatment were also identified by homology to Incyte cDNA sequences that had been annotated as molecules for disease detection and treatment. These selected Genscan-predicted sequences were then compared by BLAST analysis to the genpept and gbpri public databases. Where necessary, the Genscan-predicted sequences were then edited by comparison to the top BLAST hit from genpept to correct errors in the sequence predicted by Genscan, such as extra or omitted exons. BLAST analysis was also used to find any Incyte cDNA or public cDNA coverage of the Genscan-predicted sequences, thus providing evidence for transcription. When Incyte cDNA coverage was available, this information was used to correct or confirm the Genscan predicted sequence. Full length polynucleotide sequences were obtained by assembling Genscan-predicted coding sequences with Incyte cDNA sequences and/or public cDNA sequences using the assembly process described in Example III. Alternatively, full length polynucleotide sequences were derived entirely from edited or unedited Genscan-predicted coding sequences.

V. Assembly of Genomic Sequence Data with cDNA Sequence Data

“Stitched” Sequences

Partial cDNA sequences were extended with exons predicted by the Genscan gene identification program described in Example IV. Partial cDNAs assembled as described in Example m were mapped to genomic DNA and parsed into clusters containing related cDNAs and Genscan exon predictions from one or more genomic sequences. Each cluster was analyzed using an algorithm based on graph theory and dynamic programming to integrate cDNA and genomic information, generating possible splice variants that were subsequently confirmed, edited, or extended to create a full length sequence. Sequence intervals in which the entire length of the interval was present on more than one sequence in the cluster were identified, and intervals thus identified were considered to be equivalent by transitivity. For example, if an interval was present on a cDNA and two genomic sequences, then all three intervals were considered to be equivalent. This process allows unrelated but consecutive genomic sequences to be brought together, bridged by cDNA sequence. Intervals thus identified were then “stitched” together by the stitching algorithm in the order that they appear along their parent sequences to generate the longest possible sequence, as well as sequence variants. Linkages between intervals which proceed along one type of parent sequence (cDNA to cDNA or genomic sequence to genomic sequence) were given preference over linkages which change parent type (cDNA to genomic sequence). The resultant stitched sequences were translated and compared by BLAST analysis to the genpept and gbpri public databases. Incorrect exons predicted by Genscan were corrected by comparison to the top BLAST hit from genpept. Sequences were further extended with additional cDNA sequences, or by inspection of genomic DNA, when necessary.

“Stretched” Sequences

Partial DNA sequences were extended to full length with an algorithm based on BLAST analysis. First, partial cDNAs assembled as described in Example III were queried against public databases such as the GenBank primate, rodent, mammalian, vertebrate, and eukaryote databases using the BLAST program. The nearest GenBank protein homolog was then compared by BLAST analysis to either Incyte cDNA sequences or GenScan exon predicted sequences described in Example IV. A chimeric protein was generated by using the resultant high-scoring segment pairs (HSPs) to map the translated sequences onto the GenBank protein homolog. Insertions or deletions may occur in the chimeric protein with respect to the original GenBank protein homolog. The GenBank protein homolog, the chimeric protein, or both were used as probes to search for homologous genomic sequences from the public human genome databases. Partial DNA sequences were therefore “stretched” or extended by the addition of homologous genomic sequences. The resultant stretched sequences were examined to determine whether it contained a complete gene.

VI. Chromosomal Mapping of MDDT Encoding Polynucleotides

The sequences which were used to assemble SEQ ID NO:49-96 were compared with sequences from the Incyte LIFESEQ database and public domain databases using BLAST and other implementations of the Smith-Waterman algorithm. Sequences from these databases that matched SEQ ID NO:49-96 were assembled into clusters of contiguous and overlapping sequences using assembly algorithms such as Phrap (Table 7). Radiation hybrid and genetic mapping data available from public resources such as the Stanford Human Genome Center (SHGC), Whitehead Institute for Genome Research (WIGR), and Généthon were used to determine if any of the clustered sequences had been previously mapped. Inclusion of a mapped sequence in a cluster resulted in the assignment of all sequences of that cluster, including its particular SEQ ID NO:, to that map location.

Map locations are represented by ranges, or intervals, of human chromosomes. The map position of an interval, in centiMorgans, is measured relative to the terminus of the chromosome's p-arm. (The centiMorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Généthon which provide boundaries for radiation hybrid markers whose sequences were included in each of the clusters. Human genome maps and other resources available to the public, such as the NCBI “GeneMap'99” World Wide Web site (http://www.ncbi.nln.nih.gov/genemap/), can be employed to determine if previously identified disease genes map within or in proximity to the intervals indicated above.

VII. Analysis of Polynucleotide Expression

Northern analysis is a laboratory technique used to detect the presence of a transcript of a gene and involves the hybridization of a labeled nucleotide sequence to a membrane on which RNAs from a particular cell type or tissue have been bound (Sambrook and Russell, supra, ch. 7; Ausubel et al., supra, ch. 4).

Analogous computer techniques applying BLAST were used to search for identical or related molecules in databases such as GenBank or LIFESEQ (Incyte Genomics). This analysis is much faster than multiple membrane-based hybridizations. In addition, the sensitivity of the computer search can be modified to determine whether any particular match is categorized as exact or similar. The basis of the search is the product score, which is defined as:

$$\frac{BLAST Score \times Percent Identity}{5 \times minimum \{ length ( Seq .1 ) , length ( Seq .2 ) \}}$$

The product score takes into account both the degree of similarity between two sequences and the length of the sequence match. The product score is a normalized value between 0 and 100, and is calculated as follows: the BLAST score is multiplied by the percent nucleotide identity and the product is divided by (5 times the length of the shorter of the two sequences). The BLAST score is calculated by assigning a score of +5 for every base that matches in a high-scoring segment pair (HSP), and −4 for every mismatch. Two sequences may share more than one HSP (separated by gaps). If there is more than one HSP, then the pair with the highest BLAST score is used to calculate the product score. The product score represents a balance between fractional overlap and quality in a BLAST alignment. For example, a product score of 100 is produced only for 100% identity over the entire length of the shorter of the two sequences being compared. A product score of 70 is produced either by 100% identity and 70% overlap at one end, or by 88% identity and 100% overlap at the other. A product score of 50 is produced either by 100% identity and 50% overlap at one end, or 79% identity and 100% overlap.

Alternatively, polynucleotides encoding MDDT are analyzed with respect to the tissue sources from which they were derived. For example, some full length sequences are assembled, at least in part, with overlapping Incyte cDNA sequences (see Example III). Each cDNA sequence is derived from a cDNA library constructed from a human tissue. Each human tissue is classified into one of the following organ/tissue categories: cardiovascular system; connective tissue; digestive system; embryonic structures; endocrine system; exocrine glands; genitalia, female; genitalia, male; germ cells; hemic and immune system; liver; musculoskeletal system; nervous system; pancreas; respiratory system; sense organs; skin; stomatognathic system; unclassified/mixed; or urinary tract. The number of libraries in each category is counted and divided by the total number of libraries cross all categories. Similarly, each human tissue is classified into one of the following disease/condition categories: cancer, cell line, developmental, inflammation, neurological, trauma, cardiovascular, pooled, and other, and the number of libraries in each category is counted and divided by the total number of libraries across all categories. The resulting percentages reflect the tissue- and disease-specific expression of cDNA encoding MDDT. cDNA sequences and cDNA library/tissue information are found in the LIFESEQ GOLD database (Incyte Genomics, Palo Alto Calif.).

VIII. Extension of MDDT Encoding Polynucleotides

Full length polynucleotides are produced by extension of an appropriate fragment of the full length molecule using oligonucleotide primers designed from this fragment. One primer was synthesized to initiate 5′ extension of the known fragment, and the other primer was synthesized to initiate 3′ extension of the known fragment. The initial primers were designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed.

High fidelity amplification was obtained by PCR using methods well known in the art. PCR was performed in 96-well plates using the PTC-200 thermal cycler (MJ Research, Inc.). The reaction mix contained DNA template, 200 mmol of each primer, reaction buffer containing Mg²⁺, (NH₄)₂SO₄, and 2-mercaptoethanol, Taq DNA polymerase (Amersham Biosciences), ELONGASE enzyme (Invitrogen), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair 17 and SK+ were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well was determined by dispensing 100 μl PICOGREEN quantitation reagent (0.25% (v/v) PICOGREEN; Molecular Probes, Eugene Oreg.) dissolved in 1×TE and 0.5 μl of undiluted PCR product into each well of an opaque fluorimeter plate (Corning Costar, Acton Mass.), allowing the DNA to bind to the reagent. The plate was scanned in a Fluoroskan II (Labsystems Oy, Helsinki, Finland) to measure the fluorescence of the sample and to quantify the concentration of DNA. A 5 μl to 10 μl aliquot of the reaction mixture was analyzed by electrophoresis on a 1% agarose gel to determine which reactions were successful in extending the sequence.

The extended nucleotides were desalted and concentrated, transferred to 384-well plates, digested with CviJI cholera virus endonuclease (Molecular Biology Research, Madison Wis.), and sonicated or sheared prior to religation into pUC 18 vector (Amersham Biosciences). For shotgun sequencing, the digested nucleotides were separated on low concentration (0.6 to 0.8%) agarose gels, fragments were excised, and agar digested with Agar ACE (Promega). Extended clones were religated using T4 ligase (New England Biolabs, Beverly Mass.) into pUC 18 vector (Amersham Biosciences), treated with Pfu DNA polymerase (Stratagene) to fill-in restriction site overhangs, and transfected into competent E. coli cells. Transformed cells were selected on antibiotic-containing media, and individual colonies were picked and cultured overnight at 37° C. in 384-well plates in LB/2× carb liquid media.

The cells were lysed, and DNA was amplified by PCR using Taq DNA polymerase (Amersham Biosciences) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified by PICOGREEN reagent (Molecular Probes) as described above. Samples with low DNA recoveries were reamplified using the same conditions as described above. Samples were diluted with 20% dimethysulfoxide (1:2, v/v), and sequenced using DYENAMIC energy transfer sequencing primers and the DYENAMIC DIRECT kit (Amersham Biosciences) or the ABI PRISM BIGDYE Terminator cycle sequencing ready reaction kit (Applied Biosystems).

In like manner, full length polynucleotides are verified using the above procedure or are used to obtain 5′ regulatory sequences using the above procedure along with oligonucleotides designed for such extension, and an appropriate genomic library.

IX. Identification of Single Nucleotide Polymorphisms in MDDT Encoding Polynucleotides

Common DNA sequence variants known as single nucleotide polymorphisms (SNPs) were identified in SEQ ID NO:49-96 using the LIFESEQ database (Incyte Genomics). Sequences from the same gene were clustered together and assembled as described in Example III, allowing the identification of all sequence variants in the gene. An algorithm consisting of a series of filters was used to distinguish SNPs from other sequence variants. Preliminary filters removed the majority of basecall errors by requiring a minimum Phred quality score of 15, and removed sequence alignment errors and errors resulting from improper trimming of vector sequences, chimeras, and splice variants. An automated procedure of advanced chromosome analysis analysed the original chromatogram files in the vicinity of the putative SNP. Clone error filters used statistically generated algorithms to identify errors introduced during laboratory processing, such as those caused by reverse transcriptase, polymerase, or somatic mutation. Clustering error filters used statistically generated algorithms to identify errors resulting from clustering of close homologs or pseudogenes, or due to contamination by non-human sequences. A final set of filters removed duplicates and SNPs found in immunoglobulins or T-cell receptors.

Certain SNPs were selected for further characterization by mass spectrometry using the high throughput MASSARRAY system (Sequenom, Inc.) to analyze allele frequencies at the SNP sites in four different human populations. The Caucasian population comprised 92 individuals (46 male, 46 female), including 83 from Utah, four French, three Venezualan, and two Amish individuals. The African population comprised 194 individuals (97 male, 97 female), all African Americans. The Hispanic population comprised 324 individuals (162 male, 162 female), all Mexican Hispanic. The Asian population comprised 126 individuals (64 male, 62 female) with a reported parental breakdown of 43% Chinese, 31% Japanese, 13% Korean, 5% Vietnamese, and 8% other Asian. Allele frequencies were first analyzed in the Caucasian population; in some cases those SNPs which showed no allelic variance in this population were not further tested in the other three populations.

X. Labeling and Use of Individual Hybridization Probes

Hybridization probes derived from SEQ ID NO:49-96 are employed to screen cDNAs, genomic DNAs, or mRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 software (National Biosciences) and labeled by combining 50 pmol of each oligomer, 250 μCi of [γ-³²P] adenosine triphosphate (Amersham Biosciences), and T4 polynucleotide kinase (DuPont NEN, Boston Mass.). The labeled oligonucleotides are substantially purified using a SEPHADEX G-25 superfine size exclusion dextran bead column (Amersham Biosciences). An aliquot containing 10⁷ counts per minute of the labeled probe is used in a typical membrane-based hybridization analysis of human genomic DNA digested with one of the following endonucleases: Ase I, Bgl II, Eco RI, Pst I, Xba I, or Pvu II (DuPont NEN).

The DNA from each digest is fractionated on a 0.7% agarose gel and transferred to nylon membranes (Nytran Plus, Schleicher & Schuell, Durham N.H.). Hybridization is carried out for 16 hours at 40° C. To remove nonspecific signals, blots are sequentially washed at room temperature under conditions of up to, for example, 0.1× saline sodium citrate and 0.5% sodium dodecyl sulfate. Hybridization patterns are visualized using autoradiography or an alternative imaging means and compared.

XI. Microarrays

The linkage or synthesis of array elements upon a microarray can be achieved utilizing photolithography, piezoelectric printing (inkjet printing; see, e.g., Baldeschweiler et al., supra), mechanical microspotting technologies, and derivatives thereof. The substrate in each of the aforementioned technologies should be uniform and solid with a non-porous surface (Schena, M., ed. (1999) DNA Microarrays: A Practical Approach, Oxford University Press, London). Suggested substrates include silicon, silica, glass slides, glass chips, and silicon wafers. Alternatively, a procedure analogous to a dot or slot blot may also be used to arrange and link elements to the surface of a substrate using thermal, UV, chemical, or mechanical bonding procedures. A typical array may be produced using available methods and machines well known to those of ordinary skill in the art and may contain any appropriate number of elements (Schena, M. et al. (1995) Science 270:467-470; Shalon, D. et al. (1996) Genome Res. 6:639-645; Marshall, A. and J. Hodgson (1998) Nat. Biotechnol. 16:27-31).

Full length cDNAs, Expressed Sequence Tags (ESTs), or fragments or oligomers thereof may comprise the elements of the microarray. Fragments or oligomers suitable for hybridization can be selected using software well known in the art such as LASERGENE software (DNASTAR). The array elements are hybridized with polynucleotides in a biological sample. The polynucleotides in the biological sample are conjugated to a fluorescent label or other molecular tag for ease of detection. After hybridization, nonhybridized nucleotides from the biological sample are removed, and a fluorescence scanner is used to detect hybridization at each array element. Alternatively, laser desorbtion and mass spectrometry may be used for detection of hybridization. The degree of complementarity and the relative abundance of each polynucleotide which hybridizes to an element on the microarray may be assessed. In one embodiment, microarray preparation and usage is described in detail below.

Tissue or Cell Sample Preparation

Total RNA is isolated from tissue samples using the guanidinium thiocyanate method and poly(A)⁺ RNA is purified using the oligo-(dT) cellulose method. Each poly(A)⁺ RNA sample is reverse transcribed using MMLV reverse-transcriptase, 0.05 pg/μl oligo-(dT) primer (21mer), 1× first strand buffer, 0.03 units/μl RNase inhibitor, 500 μM dATP, 500 μM dGTP, 500 μM dTTP, 40 μM dCTP, 40 μM dCTP-Cy3 (BDS) or dCTP-Cy5 (Amersham Biosciences). The reverse transcription reaction is performed in a 25 ml volume containing 200 ng poly(A)⁺ RNA with GEMBRIGHT kits (Incyte Genomics). Specific control poly(A)⁺ RNAs are synthesized by in vitro transcription from non-coding yeast genomic DNA. After incubation at 37° C. for 2 hr, each reaction sample (one with Cy3 and another with Cy5 labeling) is treated with 2.5 ml of 0.5M sodium hydroxide and incubated for 20 minutes at 85° C. to the stop the reaction and degrade the RNA. Samples are purified using two successive CHROMA SPIN 30 gel filtration spin columns (Clontech, Palo Alto Calif.) and after combining, both reaction samples are ethanol precipitated using 1 ml of glycogen (1 mg/ml), 60 ml sodium acetate, and 300 ml of 100% ethanol. The sample is then dried to completion using a SpeedVAC (Savant Instruments Inc., Holbrook N.Y.) and resuspended in 14 μl 5×SSC/0.2% SDS.

Microarray Preparation

Sequences of the present invention are used to generate array elements. Each array element is amplified from bacterial cells containing vectors with cloned cDNA inserts. PCR amplification uses primers complementary to the vector sequences flanking the cDNA insert. Array elements are amplified in thirty cycles of PCR from an initial quantity of 1-2 ng to a final quantity greater than 5 μg. Amplified array elements are then purified using SEPHACRYL-400 (Amersham Biosciences).

Purified array elements are immobilized on polymer-coated glass slides. Glass microscope slides (Corning) are cleaned by ultrasound in 0.1% SDS and acetone, with extensive distilled water washes between and after treatments. Glass slides are etched in 4% hydrofluoric acid (VWR Scientific Products Corporation (VWR), West Chester Pa.), washed extensively in distilled water, and coated with 0.05% aminopropyl silane (Sigma) in 95% ethanol. Coated slides are cured in a 110° C. oven.

Array elements are applied to the coated glass substrate using a procedure described in U.S. Pat. No. 5,807,522, incorporated herein by reference. 1 μl of the array element DNA, at an average concentration of 100 ng/μl, is loaded into the open capillary printing element by a high-speed robotic apparatus. The apparatus then deposits about 5 nl of array element sample per slide.

Microarrays are UV-crosslinked using a STRATALINKER UV-crosslinker (Stratagene). Microarrays are washed at room temperature once in 0.2% SDS and three times in distilled water. Non-specific binding sites are blocked by incubation of microarrays in 0.2% casein in phosphate buffered saline (PBS) (Tropix, Inc., Bedford Mass.) for 30 minutes at 60° C. followed by washes in 0.2% SDS and distilled water as before.

Hybridization

Hybridization reactions contain 9 μl of sample mixture consisting of 0.2 μg each of Cy3 and Cy5 labeled cDNA synthesis products in 5×SSC, 0.2% SDS hybridization buffer. The sample mixture is heated to 65° C. for 5 minutes and is aliquoted onto the microarray surface and covered with an 1.8 cm² coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hours at 60° C. The arrays are washed for 10 min at 45° C. in a first wash buffer (1×SSC, 0.1% SDS), three times for 10 minutes each at 45° C. in a second wash buffer (0.1×SSC), and dried.

Detection

Reporter-labeled hybridization complexes are detected with a microscope equipped with an Innova 70 mixed gas 10 W laser (Coherent, Inc., Santa Clara Calif.) capable of generating spectral lines at 488 nm for excitation of Cy3 and at 632 nm for excitation of Cy5. The excitation laser light is focused on the array using a 20× microscope objective (Nikon, Inc., Melville N.Y.). The slide containing the array is placed on a computer-controlled X-Y stage on the microscope and raster-scanned past the objective. The 1.8 cm×1.8 cm array used in the present example is scanned with a resolution of 20 micrometers.

In two separate scans, a mixed gas multiline laser excites the two fluorophores sequentially. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. Each array is typically scanned twice, one scan per fluorophore using the appropriate filters at the laser source, although the apparatus is capable of recording the spectra from both fluorophores simultaneously.

The sensitivity of the scans is typically calibrated using the signal intensity generated by a cDNA control species added to the sample mixture at a known concentration. A specific location on the array contains a complementary DNA sequence, allowing the intensity of the signal at that location to be correlated with a weight ratio of hybridizing species of 1:100,000. When two samples from different sources (e.g., representing test and control cells), each labeled with a different fluorophore, are hybridized to a single array for the purpose of identifying genes that are differentially expressed, the calibration is done by labeling samples of the calibrating cDNA with the two fluorophores and adding identical amounts of each to the hybridization mixture.

The output of the photomultiplier tube is digitized using a 12-bit RTI-835H analog-to-digital (A/D) conversion board (Analog Devices, Inc., Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using each fluorophore's emission spectrum.

A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS gene expression analysis program (Incyte Genomics). Array elements that exhibit at least about a two-fold change in expression, a signal-to-background ratio of at least about 2.5, and an element spot size of at least about 40%, are considered to be differentially expressed.

Expression

SEQ ID NO:58 showed differential expression in mild Alzheimer's Disease as determined by microarray analysis. Alzheimer's Disease (AD) is a progressive dementia characterized neuropathologically by the presence of amyloid beta-peptide-containing plaques and neurofibrillary tangles in specific brain regions. In addition, neurons and synapses are lost and inflammatory responses are activated in microglia and astrocytes. A cross comparison of normal posterior cingulate brain tissue to posterior cingulate brain tissue showing mild AD was carried out. SEQ ID NO:58 showed at least two-fold increased expression in the tissue of a 68-year-old female donor with mild AD, as compared to the tissue from a 61-year-old female donor with no AD. This experiment indicates that SEQ ID NO:58 is useful in diagnostic assays and disease staging for AD and as a potential biological marker and therapeutic agent in the treatment of AD.

For example, SEQ ID NO:82 showed differential expression in breast cancer tissue, as determined by microarray analysis. Histological and molecular evaluation of breast tumors has revealed that the development of breast cancer evolves through a multi-step process whereby pre-malignant mammary epithelial cells undergo a relatively defined sequence of events leading to tumor formation. Early in tumor development ductal hyperplasia is observed. Cells undergoing rapid neoplastic growth gradually progress to invasive carcinoma and become metastatic to the lung, bone and potentially other organs. Several factors, ranging from, but not limited to, environmental to genetic, influence tumor progression and malignant transformation. In order to better determine the molecular and phenotypic characteristics associated with different stages of breast cancer, breast carcinoma cell lines at various stages of tumor progression were compared to primary human breast epithelial cells. MDA-mb-231, a breast tumor cell line isolated from the pleural effusion of a 51-year-old female, which forms poorly differentiated adenocarcinoma in nude mice and expresses the Wnt3 oncogene, EGF and TGF-α, was compared to two non-cancerous cell lines, HMEC and MCF-10A. The primary mammary epithelial cell line HMEC was derived from normal human mammary tissue (Clonetics, San Diego, Calif.). MCF-10A is a breast mammary gland cell line isolated from a 36-year-old female with fibrocystic disease. The expression of SEQ ID NO:82 was increased by at least two-fold in MDA-mb-231 cells relative to HMEC and MCF-10A cells. Therefore, SEQ ID NO:82 can be useful in diagnostic and staging assays for breast cancer and as a potential biological marker and therapeutic agent in the treatment of breast cancer.

In another example, SEQ ID NO: 86 showed differential expression, as determined by microarray analysis, in inflammatory responses. Human peripheral blood mononuclear cells (PBMCs) (52% lymphocytes, 20% NK cells, 25% monocytes, and 3% various cells that include dendritic and progenitor cells) were treated with the pro-inflammatory cytokines interleukin-1β, interleukin-2, interleukin-6, interleukin-8, interleukin-12, interleukin-18, tumor necrosis factor-α and interferon-γ, for 2 and 4 hours. The expression of SEQ ID NO:86 was increased by at least two-fold at both time points, as compared to untreated PBMCs. Therefore, SEQ ID NO:86 is useful in diagnostic assays for inflammatory responses and as a potential biological marker and therapeutic agent in the treatment of inflammatory responses.

SEQ ID NO:86 also showed differential expression in Alzheimer's Disease (AD), as determined by microarray analysis. AD is a progressive neurodegenerative disorder that is characterized by the formation of senile plaques and neurofibrillary tangles containing amyloid beta peptide. These plaques are found in limbic and association cortices of the brain. The hippocampus is part of the limbic system and plays an important role in learning and memory. In subjects with AD, accumulating plaques damage the neuronal architecture in limbic areas and eventually cripple the memory process. In a comparison of cingulate posterior brain tissue from a 68-year-old female with mild AD to anterior hippocampal tissue from a normal 61-year-old female, the expression of SEQ ID NO:86 was increased at least four-fold. Therefore, SEQ ID NO:86 is useful in diagnostic assays for AD and as a potential biological marker and therapeutic agent in the treatment of AD.

For example. SEQ ID NO:96 showed differential expression in certain prostate carcinoma cell lines versus normal prostate epithelial cells as determined by microarray analysis. The prostate carcinoma cell lines include DU 145, LNCaP, and PC-3. DU 145 was isolated from a metastatic site in the brain of a 69 year old male with widespread metastatic prostate carcinoma. DU 145 has no detectable sensitivity to hormones; forms colonies in semi-solid medium; is only weakly positive for acid phosphatase; and cells are negative for prostate, specific antigen (PSA). LNCaP is a prostate carcinoma cell line isolated from a lymph node biopsy of a 50 year old male with metastatic prostate carcinoma. LNCaP expresses PSA, produces prostate acid phosphatase, and expresses androgen receptors. PC-3, a prostate adenocarcinoma cell line, was isolated from a metastatic site in the bone of a 62 year old male with grade IV prostate adenocarcinoma. The normal epithelial cell line, PrEC, is a primary prostate epithelial cell line isolated from a normal donor. This experiment showed that the expression of SEQ ID NO:96 was decreased by at least two fold in both DU 145 and LNCaP cells compared to PrEC cells. Therefore, SEQ ID NO:96 is useful as a diagnostic marker or as a potential therapeutic target for certain prostate cancers.

XII. Complementary Polynucleotides

Sequences complementary to the MDDT-encoding sequences, or any parts thereof, are used to detect, decrease, or inhibit expression of naturally occurring MDDT. Although use of oligonucleotides comprising from about 15 to 30 base pairs is described, essentially the same procedure is used with smaller or with larger sequence fragments. Appropriate oligonucleotides are designed using OLIGO 4.06 software (National Biosciences) and the coding sequence of MDDT. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the MDDT-encoding transcript.

XIII. Expression of MDDT

Expression and purification of MDDT is achieved using bacterial or virus-based expression systems. For expression of MDDT in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into suitable bacterial hosts, e.g., BL21(DE3). Antibiotic resistant bacteria express MDDT upon induction with isopropyl beta-D-thiogalactopyranoside (IPTG). Expression of MDDT in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding MDDT by either homologous recombination or bacterial-mediated transposition involving transfer plasmid intermediates. Viral infectivity is maintained and the strong polyhedrin promoter drives high levels of cDNA transcription. Recombinant baculovirus is used to infect Spodoptera frugiperda (Sf9) insect cells in most cases, or human hepatocytes, in some cases. Infection of the latter requires additional genetic modifications to baculovirus (Engelhard, E. K. et al. (1994) Proc. Natl. Acad. Sci. USA 91:3224-3227; Sandig, V. et al. (1996) Hum. Gene Ther. 7:1937-1945).

In most expression systems, MDDT is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutathione under conditions that maintain protein activity and antigenicity (Amersham Biosciences). Following purification, the GST moiety can be proteolytically cleaved from MDDT at specifically engineered sites. FLAG, an 8-amino acid peptide, enables immunoaffinity purification using commercially available monoclonal and polyclonal anti-FLAG antibodies (Eastman Kodak). 6-His, a stretch of six consecutive histidine residues, enables purification on metal-chelate resins (QIAGEN). Methods for protein expression and purification are discussed in Ausubel et al. (supra, ch. 10 and 16). Purified MDDT obtained by these methods can be used directly in the assays shown in Examples XVII and XVM, where applicable.

XIV. Functional Assays

MDDT function is assessed by expressing the sequences encoding MDDT at physiologically elevated levels in mammalian cell culture systems. cDNA is subcloned into a mammalian expression vector containing a strong promoter that drives high levels of cDNA expression. Vectors of choice include PCMV SPORT plasmid (Invitrogen, Carlsbad Calif.) and PCR3.1 plasmid (Invitrogen), both of which contain the cytomegalovirus promoter. 5-10 μg of recombinant vector are transiently transfected into a human cell line, for example, an endothelial or hematopoietic cell line, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP; Clontech), CD64, or a CD64-GFP fusion protein. Flow cytometry (FCM), an automated, laser optics-based technique, is used to identify transfected cells expressing GFP or CD64-GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994; Flow Cytometry, Oxford, N.Y. N.Y.).

The influence of MDDT on gene expression can be assessed using highly purified populations of cells transfected with sequences encoding MDDT and either CD64 or CD64-GFP. CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Lake Success N.Y.). mRNA can be purified from the cells using methods well known by those of skill in the art. Expression of mRNA encoding MDDT and other genes of interest can be analyzed by northern analysis or microarray techniques.

XV. Production of MDDT Specific Antibodies

MDDT substantially purified using polyacrylamide gel electrophoresis (PAGE; see, e.g., Harrington, M. G. (1990) Methods Enzymol. 182:488-495), or other purification techniques, is used to immunize animals (e.g., rabbits, mice, etc.) and to produce antibodies using standard protocols.

Alternatively, the MDDT amino acid sequence is analyzed using LASERGENE software (DNASTAR) to determine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art (Ausubel et al., supra, ch. 11).

Typically, oligopeptides of about 15 residues in length are synthesized using an ABI 431A peptide synthesizer (Applied Biosystems) using FMOC chemistry and coupled to KLH (Sigma-Aldrich, St. Louis Mo.) by reaction with N-maleimidobenzoyl-N-hydroxysuccinimide ester (MBS) to increase immunogenicity (Ausubel et al., supra). Rabbits are immunized with the oligopeptide-KLH complex in complete Freund's adjuvant. Resulting antisera are tested for antipeptide and anti-MDDT activity by, for example, binding the peptide or MDDT to a substrate, blocking with 1% BSA, reacting with rabbit antisera, washing, and reacting with radio-iodinated goat anti-rabbit IgG.

XVI. Purification of Naturally Occurring MDDT Using Specific Antibodies

Naturally occurring or recombinant MDDT is substantially purified by immunoaffinity chromatography using antibodies specific for MDDT. An immunoaffinity column is constructed by covalently coupling anti-MDDT antibody to an activated chromatographic resin, such as CNBr-activated SEPHAROSE (Amersham Biosciences). After the coupling, the resin is blocked and washed according to the manufacturer's instructions.

Media containing MDDT are passed over the immunoaffinity column, and the column is washed under conditions that allow the preferential absorbance of MDDT (e.g., high ionic strength buffers in the presence of detergent). The column is eluted under conditions that disrupt antibody/MDDT binding (e.g., a buffer of pH 2 to pH 3, or a high concentration of a chaotrope, such as urea or thiocyanate ion), and MDDT is collected.

XVII. Identification of Molecules Which Interact with MDDT

MDDT, or biologically active fragments thereof, are labeled with ¹²⁵I Bolton-Hunter reagent (Bolton, A. E. and W. M. Hunter (1973) Biochem. J. 133:529-539). Candidate molecules previously arrayed in the wells of a multi-well plate are incubated with the labeled MDDT, washed, and any wells with labeled MDDT complex are assayed. Data obtained using different concentrations of MDDT are used to calculate values for the number, affinity, and association of MDDT with the candidate molecules.

Alternatively, molecules interacting with MDDT are analyzed using the yeast two-hybrid system as described in Fields, S. and O. Song (1989; Nature 340:245-246), or using commercially available kits based on the two-hybrid system, such as the MATCHMAKER system (Clontech).

MDDT may also be used in the PATHCALLING process (CuraGen Corp., New Haven Conn.) which employs the yeast two-hybrid system in a high-throughput manner to determine all interactions between the proteins encoded by two large libraries of genes (Nandabalan, K. et al. (2000) U.S. Pat. No. 6,057,101).

XVIII. Demonstration of MDDT Activity

Phorbol ester binding activity of MDDT is measured using an assay based on the fluorescent phorbol ester sapinotoxin-D (SAPD). Binding of SAPD to MDDT is quantified by measuring the resonance energy transfer from MDDT tryptophans to the 2-(N-methylamino)benzoyl fluorophore of the phorbol ester, as described by Slater et al. ((1996) J. Biol. Chem. 271:4627-4631).

Various modifications and variations of the described compositions, methods, and systems of the invention will be apparent to those skilled in the art without departing from the scope and spirit of the invention. It will be appreciated that the invention provides novel and useful proteins, and their encoding polynucleotides, which can be used in the drug discovery process, as well as methods for using these compositions for the detection, diagnosis, and treatment of diseases and conditions. Although the invention has been described in connection with certain embodiments, it should be understood that the invention as claimed should not be unduly limited to such specific embodiments. Nor should the description of such embodiments be considered exhaustive or limit the invention to the precise forms disclosed. Furthermore, elements from one embodiment can be readily recombined with elements from one or more other embodiments. Such combinations can form a number of embodiments within the scope of the invention. It is intended that the scope of the invention be defined by the following claims and their equivalents.

TABLE 1

| | | | | Incyte | |
|---|---|---|---|---|---|
| | Polypeptide | Incyte | Polynucleotide | Polynucleotide | |
| Incyte Project ID | SEQ ID NO: | Polypeptide ID | SEQ ID NO: | ID | Incyte Full Length Clones |
| 1629602 | 1 | 1629602CD1 | 49 | 1629602CB1 | 3272877CA2, 3428715CA2, 90132952CA2, 90132960CA2, |
| | | | | | 90132968CA2, 90132976CA2, 90132984CA2, 90132992CA2, |
| | | | | | 90133060CA2, 90133076CA2, 90133084CA2, 90133092CA2, |
| | | | | | 90133415CA2 |
| 2100360 | 2 | 2100360CD1 | 50 | 2100360CB1 | 90150905CA2, 90150929CA2, 90151005CA2, 90151029CA2, |
| | | | | | 90151037CA2 |
| 5166833 | 3 | 5166833CD1 | 51 | 5166833CB1 | 90189805CA2 |
| 7494963 | 4 | 7494963CD1 | 52 | 7494963CB1 | |
| 7644881 | 5 | 7644881CD1 | 53 | 7644881CB1 | 2280823CA2, 3529912CA2 |
| 3790383 | 6 | 3790383CD1 | 54 | 3790383CB1 | |
| 3846110 | 7 | 3846110CD1 | 55 | 3846110CB1 | |
| 1878279 | 8 | 1878279CD1 | 56 | 1878279CB1 | |
| 1848891 | 9 | 1848891CD1 | 57 | 1848891CB1 | |
| 2500251 | 10 | 2500251CD1 | 58 | 2500251CB1 | |
| 55026561 | 11 | 55026561CD1 | 59 | 55026561CB1 | |
| 7502593 | 12 | 7502593CD1 | 60 | 7502593CB1 | 90173235CA2, 90173236CA2, 90173237CA2, 90173335CA2, |
| | | | | | 90173343CA2, 90173403CA2, 90173411CA2 |
| 7503957 | 13 | 7503957CD1 | 61 | 7503957CB1 | 90185944CA2 |
| 7504415 | 14 | 7504415CD1 | 62 | 7504415CB1 | |
| 7504074 | 15 | 7504074CD1 | 63 | 7504074CB1 | 90147069CA2 |
| 7502257 | 16 | 7502257CD1 | 64 | 7502257CB1 | |
| 1315136 | 17 | 1315136CD1 | 65 | 1315136CB1 | 6246033CA2 |
| 1379785 | 18 | 1379785CD1 | 66 | 1379785CB1 | |
| 2011166 | 19 | 2011166CD1 | 67 | 2011166CB1 | 90177514CA2, 90177777CA2 |
| 3434684 | 20 | 3434684CD1 | 68 | 3434684CB1 | |
| 5134056 | 21 | 5134056CD1 | 69 | 5134056CB1 | |
| 5281724 | 22 | 5281724CD1 | 70 | 5281724CB1 | 5281724CA2 |
| 7502391 | 23 | 7502391CD1 | 71 | 7502391CB1 | 90174050CA2 |
| 7502544 | 24 | 7502544CD1 | 72 | 7502544CB1 | 3597185CA2 |
| 2858465 | 25 | 2858465CD1 | 73 | 2858465CB1 | |
| 7503455 | 26 | 7503455CD1 | 74 | 7503455CB1 | |
| 7503479 | 27 | 7503479CD1 | 75 | 7503479CB1 | 6054744CA2 |
| 7218127 | 28 | 7218127CD1 | 76 | 7218127CB1 | |
| 1688943 | 29 | 1688943CD1 | 77 | 1688943CB1 | 90186339CA2 |
| 2369350 | 30 | 2369350CD1 | 78 | 2369350CB1 | 90177158CA2, 90177266CA2, 90177290CA2 |
| 2722979 | 31 | 2722979CD1 | 79 | 2722979CB1 | 90186531CA2 |
| 60140470 | 32 | 60140470CD1 | 80 | 60140470CB1 | 90173175CA2, 90173191CA2, 90173275CA2 |
| 70623603 | 33 | 70623603CD1 | 81 | 70623603CB1 | 6975830CA2, 90173141CA2, 90173225CA2, 90173241CA2, |
| | | | | | 90173249CA2, 90173281CA2 |
| 7161479 | 34 | 7161479CD1 | 82 | 7161479CB1 | |
| 7502313 | 35 | 7502313CD1 | 83 | 7502313CB1 | 6538221CA2, 90166509CA2, 90166517CA2, 90166601CA2, |
| | | | | | 90166617CA2 |
| 7502390 | 36 | 7502390CD1 | 84 | 7502390CB1 | |
| 7502872 | 37 | 7502872CD1 | 85 | 7502872CB1 | |
| 7505443 | 38 | 7505443CD1 | 86 | 7505443CB1 | 5675081CA2 |
| 8032443 | 39 | 8032443CD1 | 87 | 8032443CB1 | |
| 7704916 | 40 | 7704916CD1 | 88 | 7704916CB1 | 90110946CA2, 90111022CA2, 90111038CA2, 90111046CA2 |
| 2013440 | 41 | 2013440CD1 | 89 | 2013440CB1 | 2013440CA2, 90177303CA2, 90177311CA2, 90177335CA2, |
| | | | | | 90177419CA2, 90177443CA2 |
| 2503512 | 42 | 2503512CD1 | 90 | 2503512CB1 | 2483074CA2, 6534995CA2 |
| 277396 | 43 | 277396CD1 | 91 | 277396CB1 | 90186329CA2 |
| 3044046 | 44 | 3044046CD1 | 92 | 3044046CB1 | 3044046CA2 |
| 3808420 | 45 | 3808420CD1 | 93 | 3808420CB1 | 90173389CA2, 90173457CA2, 90173465CA2, 90173473CA2 |
| 7504028 | 46 | 7504028CD1 | 94 | 7504028CB1 | |
| 7766880 | 47 | 7766880CD1 | 95 | 7766880CB1 | |
| 90089609 | 48 | 90089609CD1 | 96 | 90089609CB1 | 2950810CA2, 90089525CA2, 90089609CA2 |

TABLE 2

| Polypeptide | Incyte | GenBank ID NO: | | |
|---|---|---|---|---|
| SEQ | Polypeptide | or PROTEOME | Probability | |
| ID NO: | ID | ID NO: | Score | Annotation |
| 6 | 3790383CD1 | g1039447 | 9.2E−17 | [Saccharomyces cerevisiae] Lpb1p |
| | | | | Yang, E. and Friedberg, E. C. (1984) Molecular cloning and nucleotide sequence |
| | | | | analysis of the Saccharomyces cerevisiae RAD1 gene. Mol. Cell. Biol. 4: 2161-2169 |
| | | | | Stepien, P. P. et al. (1992) The yeast nuclear gene suv3 affecting mitochondrial |
| | | | | post-transcriptional processes encodes a putative ATP-dependent RNA helicase. |
| | | | | Proc. Natl. Acad. Sci. U.S.A. 89: 6813-6817 |
| | | | | Hiser, L. et al. (1994) ERG10 from Saccharomyces cerevisiae encodes acetoacetyl |
| | | | | CoA thiolase. J. Biol. Chem. 269: 31383-31389 |
| | | | | Bussey, H. et al. (1997) The nucleotide sequence of Saccharomyces cerevisiae |
| | | | | chromosome XVI. Nature 387: 103-105 |
| 7 | 3846110CD1 | g13603885 | 0.0 | [Homo sapiens] testis protein TEX14 |
| | | | | Wang, P. J. et al. (2001) An abundance of X-linked genes expressed in |
| | | | | spermatogonia. Nat. Genet. 27: 422-426 |
| | | 709947\|Tex14 | 2.2E−287 | [Mus musculus] Protein whose corresponding gene isexpressed only in |
| | | | | spermatagonia |
| | | | | Wang, P. J. et al. (2001) (supra) |
| 14 | 7504415CD1 | 244986\|F41E6.3 | 6.2E−141 | [Caenorhabditis elegans] Protein with weak similarity to S. cerevisiae Ynl201p, a |
| | | | | protein involved in regulation of carbon metabolism |
| 15 | 7504074CD1 | g6434857 | 1.7E−10 | [Homo sapiens] pallid |
| | | | | Huang, L., et al. (1999) The pallid gene encodes a novel, syntaxin 13-interacting |
| | | | | protein involved in platelet storage pool deficiency. Nat. Genet. 23: 329-332 |
| 16 | 7502257CD1 | g5917666 | 9.5E−27 | [Zea mays] extensin-like protein. |
| | | | | Stratford, S. et al. A leucine-rich repeat region is conserved in pollen extensin- |
| | | | | like(Pex) proteins in monocots and dicots. Plant Mol. Biol. 46 (1), 43-56 (2001) |
| | | 605696\| | 1.8E−230 | [Homo sapiens] Protein of unknown function, has a region of weak similarity to a |
| | | LOC56905 | | region of murine Mtap 6, which is a neuronal protein that stabilizes microtubules |
| | | | | and is regulated by calmodulin. |
| | | 746755\|Acz | 1.4E−16 | [Mus musculus][Cytoplasmic; Cytoskeletal] Aczonin, a neuronal zinc finger |
| | | | | protein related to bassoon (Bsn) that interacts with profilins (Pfn1 and Pfn2), may |
| | | | | be involved in presynaptic calcium sensing or in the organization of the synaptic |
| | | | | active zone. |
| | | | | Wang, X. et al. (1999) Aczonin, a 550-kD putative scaffolding protein of |
| | | | | presynaptic active zones, shares homology regions with Rim and Bassoon and |
| | | | | binds profilin. J. Cell. Biol. 147: 151-62 |
| | | 339212\| | 4.7E−16 | [Homo sapiens][Regulatory subunit; Inhibitor or repressor] Inhibitor of G1- |
| | | CDKN1C | | specific CDK-cyclin complexes that may be involved in exit from the cell cycle |
| | | | | and terminal differentiation; mutations in the corresponding gene are associated |
| | | | | with Beckwith-Wiedemannsyndrome (BWS). |
| | | | | Lee, M. H. et al. (1995) Cloning of p57KIP2, a cyclin-dependent kinase inhibitor |
| | | | | with unique domain structure and tissue distribution. Genes And Development |
| | | | | 9: 639-49 |
| | | | | Hatada, I. et al. (1996) An imprinted gene p57KIP2 is mutated in Beckwith- |
| | | | | Wiedemann syndrome. Nat. Genet. 14: 171-3 |
| | | | | Matsuoka, S. et al. (1995) p57KIP2, a structurally distinct member of the |
| | | | | p21CIP1 Cdk inhibitor family, is a candidate tumor suppressor gene. Genes And |
| | | | | Development 9: 650-62 |
| 25 | 2858465CD1 | g6175185 | 9.1E−14 | [Arabidopsis thaliana] ankyrin-like protein |
| | | 717407\|1elw_A | 8.2E−10 | [Protein Data Bank] Tpr1-Domain Of Hop |
| 26 | 7503455CD1 | g13938601 | 2.0E−73 | [Homo sapiens] Similar to WW domain binding protein 2 |
| | | 428111\| | 1.7E−74 | [Homo sapiens] [Small molecule-binding protein] Protein containing a PY motif |
| | | WBP2 | | that binds with high affinity to the WW domain contained in YAP. |
| | | | | Chen, H. I. and Sudol, M. (1995) The WW domain of Yes-associated protein |
| | | | | binds a proline-rich ligand that differs from the consensus established for Src |
| | | | | homology 3-binding modules. Proc. Natl. Acad. Sci. U.S.A. 92: 7819-23 |
| | | 587853\|Wbp2 | 1.6E−73 | [Mus musculus][Ligand] Proline-rich protein that acts as a ligand of the WW |
| | | | | domain of the Yes proto-oncoprotein |
| 28 | 7218127CD1 | 423815\|KIAA0819 | 3.0E−22 | [Homo sapiens] has weak similarity to a region of rat Nefh (heavy subunit of |
| | | | | neurofilament), which contains 52 repeats of a Lys-Ser-Pro motif that is a kinase |
| | | | | recognition site |
| | 7218127CD1 | 338388\|TAF2C1 | 5.7E−15 | [Homo sapiens][Activator; DNA-binding protein; Transcription factor; Small |
| | | | | molecule-binding protein][Nuclear] TATA box binding protein (TBP) associated |
| | | | | factor RNA polymerase II C1 130 kD, component of TFIID complex, |
| | | | | transcriptional coactivator for retinoic acid, thyroid hormone, and vitamin D3 |
| | | | | receptors and Sp1, mediates interaction of CREB1 and TFIID complex |
| 28 | 7218127CD1 | 342642\|PRG4 | 1.1E−14 | [Homo sapiens][Structural protein][Extracellular (excluding cell wall)] |
| | | | | Megakaryocyte stimulating factor (superficial zone protein), a secreted |
| | | | | proteoglycan; mutations in the gene cause camptodactyly-arthropathy-coxa vara- |
| | | | | pericarditis syndrome, a joint disorder |
| | | | | Marcelino J, et al. (1999) CACP, encoding a secreted proteoglycan, is mutated in |
| | | | | camptodactyly-arthropathy-coxa vara-pericarditis syndrome. Nat. Genet. 23: 319-322 |
| 34 | 7161479CD1 | 424208\|KIAA0136 | 9.5E−13 | [Homo sapiens] Protein with low similarity to microrchidias, which are male germ- |
| | | | | line specific proteins that act during gametogenesis and which contain two |
| | | | | predicted coiled-coil structures |
| 40 | 7704916CD1 | g13508446 | 1.1E−195 | [Homo sapiens] NYD-SP6 |
| | | | | Xiao, J. et al. (2002) NYD-SP6, a novel gene potentially involved in regulating |
| | | | | testicular development/spermatogenesis. Biochem. Biophys. Res. Commun. |
| | | | | 291: 101-110 |

TABLE 3

| SEQ | Incyte | Amino | Potential | Potential | | |
|---|---|---|---|---|---|---|
| ID | Polypeptide | Acid | Phosphorylation | Glycosylation | | Analytical Methods |
| NO: | ID | Residues | Sites | Sites | Signature Sequences, Domains and Motifs | and Databases |
| 1 | 1629602CD1 | 93 | S38 S48 | | | |
| 2 | 2100360CD1 | 281 | S24 S179 S218 | N177 | Glycosyl hydrolases family 5 signature: V84-D93 | MOTIFS |
| 3 | 5166833CD1 | 292 | S39 S123 S215 | | Cytosolic domain: Q290-Y292 | TMHMMER |
| | | | S219 T129 T237 | | Transmembrane domain: T267-L289 | |
| | | | | | Non-cytosolic domain: M1-I266 | |
| 4 | 7494963CD1 | 270 | S45 S46 S55 S63 | N34 N208 | Leucine zipper pattern: L130-L151 | MOTIFS |
| | | | S77 S87 S94 S123 | | | |
| | | | S256 T102 T154 | | | |
| | | | | | Cell attachment sequence: R251-D253 | MOTIFS |
| 5 | 7644881CD1 | 447 | S219 S264 S282 | N159 N371 | | |
| | | | S315 T23 T161 | | | |
| | | | T223 T358 T444 | | | |
| 6 | 3790383CD1 | 757 | S89 S144 S171 | N210 N422 N633 | PROTEIN LPB1P CODED FOR BY | BLAST_PRODOM |
| | | | S241 S358 S459 | | C ELEGANS CDNA YK121E3.3 | |
| | | | S463 S495 S510 | | PD043484: S295-P407, F413-K504, I9-R37 | |
| | | | S515 S526 S542 | | | |
| | | | S630 S635 S646 | | | |
| | | | S757 T198 T324 | | | |
| | | | T354 T398 T406 | | | |
| | | | T496 T658 T751 | | | |
| | | | Y264 | | | |
| | | | | | CODED FOR BY C. ELEGANS CDNA | BLAST_PRODOM |
| | | | | | YK121E3.3 PD145099: F589-H740 | |
| 7 | 3846110CD1 | 1014 | S85 S128 S207 | N260 N281 N419 | | |
| | | | S222 S239 S244 | | | |
| | | | S245 S252 S267 | | | |
| | | | S271 S283 S447 | | | |
| | | | S462 S522 S587 | | | |
| | | | S592 S623 S642 | | | |
| | | | S670 S682 S692 | | | |
| | | | S709 S714 S777 | | | |
| | | | S806 S896 S921 | | | |
| | | | S947 S976 S983 | | | |
| | | | S1001 T284 T337 | | | |
| | | | T389 T414 T671 | | | |
| | | | T678 T717 T770 | | | |
| | | | T790 T828 T875 | | | |
| | | | T882 T916 T922 | | | |
| | | | T982 | | | |
| 8 | 1878279CD1 | 342 | S28 S37 S42 S72 | N52 N99 N189 | | |
| | | | S73 S137 S167 | N203 | | |
| | | | S176 S187 S224 | | | |
| | | | S253 S259 S262 | | | |
| | | | S275 S285 S316 | | | |
| | | | T54 T94 T154 | | | |
| | | | T171 T195 T222 | | | |
| | | | Y69 | | | |
| 9 | 1848891CD1 | 415 | S110 S162 S181 | | Leucine zipper pattern: L72-L93 | MOTIFS |
| | | | S218 S253 S318 | | | |
| | | | S333 S361 T327 | | | |
| | | | T403 | | | |
| 10 | 2500251CD1 | 665 | S41 S125 S134 | N152 N495 N596 | COSMID F54C1 PD140119: R217-R448 | BLAST_PRODOM |
| | | | S148 S200 S512 | | | |
| | | | S532 S592 T273 | | | |
| | | | T350 T370 T394 | | | |
| | | | T645 Y27 Y533 | | | |
| | | | Y584 | | | |
| | | | | | W03G9.7 PROTEIN PD147457: M470-E636 | BLAST_PRODOM |
| 11 | 55026561CD1 | 622 | S93 S94 S108 S143 | N11 N142 N508 | | |
| | | | S171 S177 S189 | N586 | | |
| | | | S234 S263 S298 | | | |
| | | | S333 S339 S352 | | | |
| | | | S354 S359 S364 | | | |
| | | | S367 S395 S458 | | | |
| | | | S476 S559 S594 | | | |
| | | | T37 T126 T318 | | | |
| | | | T373 T388 T465 | | | |
| | | | T523 T620 Y313 | | | |
| 12 | 7502593CD1 | 242 | S22 S42 S115 S125 | N62 | | |
| | | | S139 T37 T101 | | | |
| | | | T188 | | | |
| 13 | 7503957CD1 | 408 | S28 S37 S42 S72 | N52 N99 N189 | | |
| | | | S73 S137 S167 | N203 | | |
| | | | S176 S187 S224 | | | |
| | | | S253 S259 S262 | | | |
| | | | S275 S285 S316 | | | |
| | | | T54 T94 T154 | | | |
| | | | T171 T195 T222 | | | |
| | | | T369 Y69 Y407 | | | |
| 14 | 7504415CD1 | 820 | S73 S83 S117 S138 | N203 N453 N479 | PROTEIN F41E6.3 SPX19GCR2 INTERGENIC | BLAST_PRODOM |
| | | | S253 S279 S486 | N577 N722 N742 | REGION | |
| | | | S645 S685 S687 | N774 | PD044038: D79-D408, A497-R526 | |
| | | | S724 S730 S813 T4 | | PD044039: D444-E598 | |
| | | | T12 T243 T264 | | | |
| | | | T323 T425 T441 | | | |
| | | | T455 T480 T623 | | | |
| | | | T661 T672 T690 | | | |
| | | | T782 Y620 | | | |
| | | | | | F41E6.3 PROTEIN | BLAST_PRODOM |
| | | | | | PD147990: D599-S813 | |
| 15 | 7504074CD1 | 34 | S7 | | N-6 Adenine-specific DNA methylases | PROFILESCAN |
| | | | | | signature: M1-L32 | |
| 16 | 7502257CD1 | 938 | S25 S96 S108 S199 | | NUCLEAR LOCALIZATION SIGNAL | BLAST_DOMO |
| | | | S444 S567 S577 | | DOMAIN DM07752\|P49918\|173-315: | |
| | | | S737 S773 S785 | | A713-Q765 | |
| | | | T42 T55 T364 | | | |
| | | | T525 T582 T828 | | | |
| | | | T877 T893 | | | |
| 17 | 1315136CD1 | 253 | S47 S122 S147 | | | |
| | | | S157 T247 | | | |
| 18 | 1379785CD1 | 723 | S45 S68 S83 S87 | N59 N118 | Adenylate kinase signature: L455-E519 | PROFILESCAN |
| | | | S101 S141 S183 | | | |
| | | | T11 T17 T50 T174 | | | |
| | | | T284 T614 Y310 | | | |
| | | | Y611 | | | |
| | | | | | ATP/GTP-binding site motif A (P-loop): | |
| | | | | | G374-S381 | MOTIFS |
| 19 | 2011166CD1 | 253 | S11 S102 S166 | | | |
| | | | T178 T188 | | | |
| 20 | 3434684CD1 | 154 | S41 S144 T148 | | | |
| 21 | 5134056CD1 | 566 | S76 S374 S413 | N488 N504 N514 | | |
| | | | T268 T369 T387 | | | |
| 22 | 5281724CD1 | 234 | S30 S160 S167 | | | |
| | | | S184 S218 S228 | | | |
| | | | T41 T46 T141 | | | |
| | | | Y156 | | | |
| 23 | 7502391CD1 | 268 | S89 S233 T11 | N261 | | |
| 24 | 7502544CD1 | 694 | S110 S342 S393 | N274 N312 N336 | Vitamin K-dependent carboxylation | MOTIFS |
| | | | S428 S440 S447 | N575 | domain: W634-W671 | |
| | | | S460 S600 T58 | | | |
| | | | T216 T301 T451 | | | |
| | | | T517 | | | |
| 25 | 2858465CD1 | 519 | S16 S69 S94 S135 | N344 | TPR Domain: S305-D338, | HMMER_PFAM |
| | | | S206 S251 S288 | | P373-P406, H339-W372 (P = 7.7e−10) | |
| | | | S404 S453 S490 | | | |
| | | | T152 T204 T367 | | | |
| | | | T399 | | | |
| | | | | | TPR REPEAT DM00408\|P31948\|1-147: | BLAST_DOMO |
| | | | | | L302-R401 | |
| 26 | 7503455CD1 | 216 | S109 S164 T31 T45 | N6 N16 | GRAM domain: M1-Q84 | HMMER_PFAM |
| | | | T49 T95 | | | |
| | | | | | PROTEIN D2013.6 C29B12.11C | BLAST_PRODOM |
| | | | | | CHROMOSOME I WW DOMAIN | |
| | | | | | BINDING SIMILAR C ELEGANS PD016518: | |
| | | | | | M1-Q132 | |
| 27 | 7503479CD1 | 110 | S76 S94 T20 T22 | | | |
| | | | T34 | | | |
| 28 | 7218127CD1 | 642 | S23 S105 S171 | | NEUROFILAMENT; TRIPLET; | BLAST_DOMO |
| | | | S185 S199 S242 | | DM04498\|P12036\|434-1019: S10-P448 | |
| | | | S250 S313 S336 | | | |
| | | | S363 S431 S474 | | | |
| | | | S568 S595 S624 | | | |
| | | | S633 T68 T341 | | | |
| | | | T382 T585 Y538 | | | |
| | | | | | Leucine zipper pattern: L515-L536 | MOTIFS |
| | | | | | ATP/GTP-binding site motif A (P-loop): | MOTIFS |
| | | | | | A296-S303 | |
| 29 | 1688943CD1 | 489 | S57 S77 S121 S133 | N116 N131 N339 | | MOTIFS |
| | | | S187 S192 S208 | N360 N384 | | |
| | | | S342 S344 T45 T74 | | | |
| | | | T152 T226 T287 | | | |
| | | | T473 | | | |
| 30 | 2369350CD1 | 184 | S38 S80 T33 T39 | | | |
| | | | T97 | | | |
| 31 | 2722979CD1 | 520 | S50 S168 S204 | N482 | Poly(ADP-ribose) polymerase zinc finger domain | BLIMPS_BLOCKS |
| | | | S399 S405 S410 | | proteins BL00347: G245-A295 | |
| | | | T24 T193 | | | |
| 32 | 60140470CD1 | 255 | S4 S25 S69 S76 | N67 | | |
| | | | S108 S133 S242 | | | |
| | | | T34 T141 T221 | | | |
| | | | Y62 | | | |
| 33 | 70623603CD1 | 231 | S27 S84 S91 S130 | N125 | Leucine zipper pattern: L96-L117, L156-L177 | MOTIFS |
| | | | S154 S162 S169 | | | |
| | | | S184 S200 S209 | | | |
| | | | T142 T188 T198 | | | |
| | | | Y227 | | | |
| 34 | 7161479CD1 | 492 | S20 S93 S116 S170 | N77 | | |
| | | | S172 S229 S373 | | | |
| | | | S383 S431 T28 T43 | | | |
| | | | T68 T315 T331 | | | |
| | | | T404 T420 T483 | | | |
| | | | Y372 | | | |
| 35 | 7502313CD1 | 85 | S66 | N9 | | |
| 36 | 7502390CD1 | 178 | S86 S104 S115 T4 | | signal_cleavage: M1-G51 | SPSCAN |
| | | | T11 T56 T76 T145 | | | |
| | | | T164 | | | |
| 37 | 7502872CD1 | 665 | S49 S85 S162 S205 | | Leucine zipper pattern: L394-L415, L401-L422 | MOTIFS |
| | | | S257 S329 S382 | | | |
| | | | S400 S443 S534 | | | |
| | | | S582 S645 T27 | | | |
| | | | T130 T192 T491 | | | |
| | | | T631 T634 | | | |
| 38 | 7505443CD1 | 551 | S90 S152 S163 | | Leucine Rich Repeat: G55-T76, A77-H98 | HMMER_PFAM |
| | | | S216 S250 S272 | | | |
| | | | S286 S291 S359 | | | |
| | | | S360 S427 S440 | | | |
| | | | S464 S501 S527 | | | |
| | | | T38 T76 T159 | | | |
| | | | T248 T329 | | | |
| | | | | | Leucine zipper pattern: L454-L475 | MOTIFS |
| 39 | 8032443CD1 | 148 | T10 Y116 | | signal_cleavage: M1-C43 | SPSCAN |
| | | | | | IQ calmodulin-binding motif: R12-L32, | HMMER_PFAM |
| | | | | | E68-C88, A35-K55, L91-F111 | |
| 40 | 7704916CD1 | 342 | S16 S28 S65 S74 | N54 | PHD-finger. PF00628: K112-E126 | BLIMPS_PFAM |
| | | | S94 S139 S197 | | | |
| | | | S248 S253 S303 | | | |
| | | | S311 S318 S326 | | | |
| | | | S337 T3 T19 T23 | | | |
| | | | T242 T295 | | | |
| 41 | 2013440CD1 | 194 | S23 S96 S175 T39 | | PROTEIN ZINC-FINGER META | BLIMPS_PRODOM |
| | | | T42 T97 T152 Y79 | | PD00066: H81-Y93 | |
| 42 | 2503512CD1 | 126 | S71 S80 S84 S91 | | | |
| | | | S101 S113 T9 T12 | | | |
| | | | T21 T75 | | | |
| 43 | 277396CD1 | 474 | S52 S72 S130 S164 | N169 N270 | | |
| | | | S187 S243 S254 | | | |
| | | | S321 S349 S388 | | | |
| | | | S423 S458 T28 T71 | | | |
| | | | T104 T138 T172 | | | |
| | | | T239 T339 T340 | | | |
| | | | T373 T413 T431 | | | |
| | | | Y127 | | | |
| 44 | 3044046CD1 | 341 | S2 S36 S103 S117 | N68 N78 N102 | PROTEIN COS41.4 R01H10.6 | BLAST_PRODOM |
| | | | S208 S246 T80 | N169 N194 N198 | PD152654: N85-D287, I76-M340 | |
| | | | T104 T133 Y96 | | PD024709: G27-R82 | |
| 45 | 3808420CD1 | 287 | S5 S114 S155 S251 | | | |
| | | | S272 T40 T105 | | | |
| | | | T206 | | | |
| 46 | 7504028CD1 | 644 | S7 S355 S418 S434 | N91 | C01B10.8 PROTEIN PD142070: P5-L429 | BLAST_PRODOM |
| | | | S463 S473 S485 | | | |
| | | | S487 T34 T93 | | | |
| | | | T106 T129 T291 | | | |
| | | | T620 T640 Y632 | | | |
| | | | | | Leucine zipper pattern: L136-L157 | MOTIFS |
| 47 | 7766880CD1 | 914 | S9 S28 S160 S166 | N54 N96 N353 | | |
| | | | S189 S200 S235 | N455 N487 | | |
| | | | S299 S331 S390 | | | |
| | | | S392 S409 S410 | | | |
| | | | S436 S489 S493 | | | |
| | | | S527 S622 S643 | | | |
| | | | S661 S690 S694 | | | |
| | | | S701 S702 S703 | | | |
| | | | S730 S751 S783 | | | |
| | | | S790 S834 T44 T52 | | | |
| | | | T162 T355 T430 | | | |
| | | | T442 T520 T548 | | | |
| | | | T555 T632 T656 | | | |
| | | | T727 T824 T827 | | | |
| | | | T831 | | | |
| 48 | 90089609CD1 | 148 | S7 S128 | | Signal cleavage: M14-V69 | SPSCAN |
| | | | | | Ribosomal protein S3 proteins | BLIMPS_BLOCKS |
| | | | | | BL00548: G22-H51 | |

TABLE 4

| Polynucleotide | |
|---|---|
| SEQ | |
| ID NO:/Incyte ID/ | |
| Sequence Length | Sequence Fragments |
| 49/1629602CB1/ | 1-184, 1-223, 1-605, 1-876, 2-195, 2-689, 4-247, 4-288, 5-301, 6-644, 10-301, 27-415, 41-415, 74-685, 114-367, 119-691, |
| 882 | 174-415, 174-678, 174-724, 179-633, 238-685, 241-414, 256-414, 275-839, 314-882, 337-414, 429-685, 429-839, |
| | 429-842, 449-839, 450-839, 453-685, 453-839, 453-848, 523-841, 740-838 |
| 50/2100360CB1/ | 1-525, 30-651, 31-570, 31-805, 33-556, 35-294, 64-310, 76-370, 102-903, 103-775, 112-767, 121-782, 125-381, 125-676, |
| 2489 | 214-350, 223-948, 223-2473, 350-603, 352-671, 495-898, 614-1276, 722-1343, 756-1416, 892-1479, 894-1180, |
| | 1114-1336, 1123-1366, 1136-1644, 1136-1657, 1136-1721, 1249-1592, 1262-1858, 1265-1716, 1315-1537, 1315-1897, |
| | 1336-1686, 1343-1860, 1355-1502, 1366-1527, 1381-2027, 1470-2136, 1506-1776, 1533-1855, 1592-2063, |
| | 1615-2303, 1622-2070, 1622-2127, 1622-2223, 1627-2105, 1694-2255, 1725-1997, 1725-2202, 1735-2372, 1745-1893, |
| | 1747-2219, 1800-2441, 1806-2450, 1807-2071, 1829-2434, 1851-2074, 1895-2475, 1944-2242, 1961-2221, |
| | 1984-2455, 2004-2452, 2007-2442, 2007-2465, 2012-2285, 2026-2480, 2033-2455, 2039-2463, 2046-2463, 2054-2447, |
| | 2117-2464, 2135-2462, 2156-2471, 2168-2428, 2168-2469, 2240-2489, 2277-2449, 2277-2464 |
| 51/5166833CB1/ | 1-137, 1-159, 1-391, 1-544, 1-555, 10-609, 20-361, 26-134, 26-506, 26-523, 86-206, 86-286, 86-297, 86-717, 86-781, |
| 1115 | 111-1115, 117-471, 118-741, 144-737, 225-819, 255-376, 257-361, 278-888, 285-891, 314-715, 331-914, 341-914, |
| | 350-875, 462-692, 462-837, 748-939, 754-939 |
| 52/7494963CB1/ | 1-553, 1-2257, 6-679, 520-647, 520-1112, 611-1138, 682-959, 682-1064, 682-1069, 682-1103, 682-1166, 682-1172, |
| 2434 | 682-1205, 682-1211, 682-1338, 682-1341, 682-1387, 682-1404, 682-1432, 682-1456, 682-1458, 746-1405, 752-1412, |
| | 909-1630, 914-1788, 926-1471, 939-1622, 977-1183, 979-1467, 1006-1071, 1012-1222, 1024-1632, 1071-1772, |
| | 1091-1609, 1105-1700, 1108-1753, 1111-1873, 1134-1679, 1144-1759, 1147-1858, 1151-1766, 1154-1408, |
| | 1190-1756, 1191-1772, 1203-1485, 1214-1972, 1225-1621, 1252-1912, 1257-1506, 1268-2036, 1271-1782, 1319-1957, |
| | 1320-1945, 1324-1485, 1324-1710, 1324-1767, 1324-1798, 1324-1907, 1331-1867, 1332-1959, 1333-1579, |
| | 1338-2050, 1356-1564, 1358-2156, 1360-2150, 1362-1951, 1366-1650, 1371-1979, 1380-2041, 1392-2116, 1405-2017, |
| | 1411-1885, |
| | 1445-2039, 1454-2052, 1455-2017, 1462-2052, 1472-1837, 1472-1852, 1475-1780, 1478-2094, 1484-2089, 1485-2102, |
| | 1486-1758, 1490-2205, 1493-1973, 1496-2124, 1500-2011, 1514-2048, 1514-2105, 1544-2153, 1578-2188, |
| | 1588-2148, 1588-2222, 1605-1782, 1612-1945, 1616-2281, 1632-2121, 1633-2208, 1640-2218, 1642-1886, 1650-2205, |
| | 1681-2292, 1690-2307, 1704-2325, 1709-2315, 1726-2219, 1729-2345, 1732-2207, 1737-2342, 1785-2289, |
| | 1787-2323, 1793-2356, 1799-2124, 1802-1927, 1813-2192, 1823-2139, 1824-2197, 1825-2309, 1867-2145, 1867-2147, |
| | 1867-2355, 1874-2182, 1874-2194, 1878-2311, 1922-2051, 1978-2233, 1979-2291, 1986-2199, 1991-2284, |
| | 1994-2281, 2010-2220, 2031-2292, 2034-2220, 2036-2300, 2036-2302, 2036-2326, 2055-2277, 2069-2286, 2178-2434 |
| 53/7644881CB1/ | 1-769, 480-1096, 480-1309, 525-1020, 717-1029, 745-984, 745-1304, 775-1456, 840-1427, 851-1361, 868-1611, |
| 3492 | 876-1421, 884-1392, 896-1357, 898-1665, 913-1637, 922-1450, 927-1511, 968-1330, 976-1614, 979-1838, 994-1852, |
| | 1000-1489, 1005-1787, 1005-3492, 1015-1603, 1021-1598, 1027-1783, 1049-1794, 1052-1841, 1061-1628, |
| | 1068-1634, 1082-1700, 1113-1734, 1113-1965, 1116-1794, 1117-1799, 1122-1654, 1127-1700, 1132-1748, 1143-1838, |
| | 1144-1833, 1162-1470, 1189-1662, 1196-1795, 1199-1872, 1201-1970, 1203-1896, 1205-1972, 1210-1847, |
| | 1222-1833, 1234-1796, 1271-1912, 1273-1871, 1278-1868, 1278-1870, 1285-1989, 1286-1925, 1287-1957, 1295-2008, |
| | 1321-2024, 1323-1987, |
| | 1324-1904, 1328-1736, 1332-1959, 1336-2087, 1343-1746, 1370-2014, 1372-2157, 1374-2024, 1378-1668, 1391-1987, |
| | 1392-1942, 1394-1987, 1395-1829, 1397-1927, 1399-2008, 1406-1989, 1411-2024, 1415-1971, 1417-1767, |
| | 1422-1944, 1431-2021, 1444-2215, 1448-1980, 1483-2019, 1484-2071, 1538-1928, 1558-2252, 1578-2241, 1600-2152, |
| | 1601-2077, 1614-2270, 1651-1899, 1661-2272, 1698-2272, 1723-2152, 1727-2080, 1736-2128, 1766-2261, |
| | 1778-2152, 1783-2152, 1784-2045, 1784-2217, 1784-2331, 1815-2191, 1968-2272, 2246-2546, 2251-2540, 2288-2700, |
| | 2360-2700, 2369-2709, 2381-2623, 2406-2911, 2406-2964, 2446-2668, 2450-2721, 2525-2866, 2526-2879, |
| | 2544-3164, 2590-3083, 2756-3324, 2758-3492, 2793-3492, 2811-3411, 2906-3128, 2906-3471, 2917-3177, 2917-3418, |
| | 2956-3477 |
| 54/3790383CB1/ | 1-888, 243-538, 243-888, 281-1041, 281-3097, 604-1281, 708-1405, 1032-1297, 1124-1410, 1124-1745, 1224-1388, |
| 3141 | 1472-1646, 1488-2135, 1510-1794, 1510-2114, 1510-2132, 1570-2256, 1618-2151, 1699-2323, 1714-2109, 1739-2313, |
| | 1802-2096, 1814-2115, 1818-2107, 1855-2245, 1889-2471, 1925-2504, 1959-2223, 1967-2580, 2059-2694, |
| | 2085-2331, 2106-2769, 2165-2760, 2185-2425, 2195-2770, 2200-2451, 2207-2815, 2213-2886, 2225-2786, 2232-2786, |
| | 2236-2720, 2276-2656, 2276-2814, 2312-3073, 2344-2641, 2355-2936, 2362-2918, 2372-2949, 2376-2686, |
| | 2382-2658, 2404-3074, 2410-2641, 2472-3130, 2491-2765, 2569-3013, 2573-3062, 2603-3014, 2656-3058, 2722-3099, |
| | 2935-3141 |
| 55/3846110CB1/ | 1-387, 247-3214, 304-1846, 450-664, 1663-1873, 1663-2155, 2112-2483, 2528-2784, 2528-3205, 2777-3354, 2796-3491, |
| 3491 | 2797-3235, 2797-3309, 2797-3310, 2797-3329, 2797-3398, 2798-3115, 2872-3155, 2891-3447, 2938-3247, |
| | 2964-3180, 2984-3237, 2991-3464, 3005-3456, 3018-3237, 3026-3470, 3028-3462, 3038-3463, 3041-3456, 3045-3469, |
| | 3051-3469, 3062-3315, 3114-3458, 3166-3456, 3276-3469 |
| 56/1878279CB1/ | 1-939, 71-683, 71-761, 190-872, 346-937, 369-657, 369-897, 369-909, 369-942, 369-952, 369-961, 446-1006, 481-984, |
| 4312 | 500-1050, 508-978, 527-1071, 562-863, 564-990, 594-4284, 614-1192, 628-1216, 629-1094, 722-1216, 794-1396, |
| | 841-1455, 848-1486, 874-1459, 874-1528, 924-1393, 934-1842, 940-1593, 1004-1485, 1016-1569, 1054-1597, |
| | 1088-1216, 1102-1369, 1116-1589, 1136-1214, 1160-1214, 1243-1474, 1243-1540, 1243-1589, 1243-1592, |
| | 1243-1594, 1243-1598, 1243-1609, 1243-1698, 1243-1735, 1245-1309, 1245-1312, 1247-1591, 1250-1589, 1254-1484, |
| | 1262-2015, 1347-2006, 1352-1975, 1452-2163, 1777-2163, 1787-2433, 1852-2310, 1852-2399, 1854-2527, |
| | 1982-2647, 1992-2586, 2198-2806, 2206-2788, 2272-2847, 2323-2567, 2323-2871, 2379-2570, 2455-2741, 2455-3012, |
| | 2480-2731, 2552-2702, 2612-2716, 2612-2717, 2660-2717, 2684-3266, 2841-3093, 2947-3017, 2988-3249, |
| | 2988-3487, 2988-3638, 3001-3596, 3012-3277, 3018-3476, 3027-3275, 3027-3540, 3034-3222, 3034-3301, 3034-3465, |
| | 3056-3649, 3061-3343, 3066-3283, 3066-3679, 3097-3261, 3122-3404, 3138-3966, 3202-3453, |
| | 3221-3814, 3231-3672, 3247-3518, 3261-3571, 3292-3547, 3318-3794, 3335-3633, 3339-3610, 3339-3964, 3347-3651, |
| | 3369-3665, 3370-3470, 3379-3929, 3397-3652, 3397-3665, 3399-4251, 3400-3638, 3424-3821, 3435-3715, |
| | 3439-3832, 3449-3735, 3460-3643, 3479-3855, 3489-3963, 3517-4219, 3527-3805, 3530-3981, 3544-3812, 3554-3690, |
| | 3556-4218, 3590-3875, 3598-4213, 3598-4287, 3604-4234, 3609-3839, 3611-3858, 3619-4266, 3628-3897, |
| | 3633-3832, 3635-3897, 3635-4076, 3643-4269, 3646-3912, 3660-4299, 3661-3850, 3661-3887, 3661-3936, 3682-3941, |
| | 3684-4124, 3695-4037, 3716-4281, 3781-4281, 3800-4282, 3802-4030, 3809-3957, 3812-4282, 3813-4282, |
| | 3823-4074, 3823-4224, 3829-4281, 3829-4286, 3835-4282, 3836-4253, 3837-4275, 3838-4295, 3842-4282, 3845-4046, |
| | 3849-4281, 3850-4282, 3852-4312, 3861-4081, 3865-4206, 3866-4282, 3869-4271, 3870-4154, 3870-4287, |
| | 3879-4284, 3892-4282, 3898-4152, 3901-4288, 3905-4164, 3937-4281, 3946-4282, 3955-4295, 3979-4244, 4014-4282, |
| | 4060-4305, 4070-4269, 4093-4291, 4125-4279, 4153-4282 |
| 57/1848891CB1/ | 1-726, 348-598, 435-1095, 452-546, 452-624, 463-721, 463-772, 463-781, 463-793, 463-837, 463-908, 463-930, 465-1034, |
| 3860 | 474-709, 514-1037, 514-1084, 514-1125, 514-1173, 514-1176, 514-1188, 514-1200, 514-1201, 514-1216, 514-1223, |
| | 514-1226, 514-1249, 514-1266, 514-1287, 514-1289, 514-1349, 517-1345, 518-1215, 521-1277, 521-1409, |
| | 527-1327, 548-1376, 552-1239, 552-1263, 556-1303, 558-1279, 564-1219, 575-1333, 575-1358, 601-1304, 604-1338, |
| | 636-1333, 719-1114, 729-1249, 729-1289, 729-1448, 782-1679, 790-1330, 844-1432, 850-1372, 872-1337, |
| | 885-1714, 897-1366, 904-1170, 908-1367, 913-1143, 913-1332, 921-1333, 937-1209, 940-1367, 940-1479, 949-1764, |
| | 956-1351, 961-1469, 975-1100, 988-1366, 992-1459, 995-1324, 999-1104, 999-1567, 1034-1767, 1064-1759, |
| | 1127-1806, 1189-1949, 1204-1806, 1215-1730, 1244-1804, 1297-1575, 1299-1803, 1314-1949, 1325-1832, 1338-1923, |
| | 1341-1949, 1349-1835, 1355-1927, 1365-1891, 1372-1947, 1373-2076, 1374-1949, 1378-1879, 1379-1766, 1379-1933, |
| | 1387-1822, 1387-1949, 1402-1651, 1402-1998, 1402-2079, 1404-1664, 1439-1947, 1442-1969, 1449-2103, |
| | 1456-1759, 1476-1945, 1485-1945, 1487-1973, 1503-1886, 1508-1901, 1522-2256, 1523-2259, 1535-1781, 1543-2257, |
| | 1550-1796, 1593-2260, 1604-2231, 1663-2263, 1717-2263, 1743-2157, 1788-2219, 1789-2258, 1804-2252, |
| | 1805-2272, 1818-2268, 1833-2255, 1834-2270, 1850-2255, 1858-2261, 1861-2255, 1863-2254, 1877-2268, 1883-2252, |
| | 1883-2255, 1905-2181, 1907-2267, 1953-2255, 1963-2256, 1970-2255, 1974-2255, 2059-2237, 2076-2255, |
| | 2094-2305, 2106-2204, 2108-2269, 2126-2397, 2222-2511, 2246-2494, 2246-2755, 2311-2485, 2351-2637, 2353-2635, |
| | 2372-2621, 2372-2919, 2373-2615, 2378-2422, 2391-3017, 2396-2772, 2397-2679, 2397-2974, 2399-2659, |
| | 2399-2794, 2404-2674, 2406-2602, 2415-2666, 2420-2750, 2420-2954, 2421-2589, 2447-2937, 2539-2791, |
| | 2561-3047, 2605-2865, 2605-3174, 2614-2818, 2614-2862, 2614-2869, 2617-2753, 2625-2893, 2625-2927, 2627-2880, |
| | 2628-3214, 2646-2939, 2647-2845, 2677-2959, 2679-2827, 2680-2804, 2692-3076, 2707-2982, 2740-2943, |
| | 2746-3360, 2756-3012, 2774-3036, 2775-3403, 2839-3099, 2839-3403, 2840-2965, 2840-3069, 2840-3087, 2840-3255, |
| | 2840-3290, 2840-3295, 2840-3336, 2840-3340, 2840-3344, 2840-3351, 2840-3353, 2840-3417, 2840-3419, |
| | 2840-3440, 2840-3479, 2848-3337, 2862-3437, 2883-3141, 2889-3291, 2889-3425, 2902-3187, 2902-3208, 2904-3129, |
| | 2910-3343, 2917-3444, 2952-3396, 2952-3456, 2954-3454, 2968-3787, 2970-3233, 3026-3265, 3026-3282, |
| | 3027-3307, 3061-3358, 3061-3585, 3063-3769, 3074-3203, 3077-3658, 3110-3754, 3122-3742, 3133-3664, 3139-3671, |
| | 3140-3609, 3145-3460, 3155-3423, 3166-3370, 3175-3555, 3198-3744, 3199-3837, 3222-3469, 3222-3651, |
| | 3226-3806, 3227-3483, 3246-3507, 3246-3510, 3247-3441, 3248-3483, 3255-3860, 3258-3785, 3262-3860, |
| | 3267-3517, 3267-3538, 3268-3828, 3274-3551, 3276-3627, 3276-3832, 3278-3755, 3278-3860, 3286-3556, 3291-3509, |
| | 3307-3849, 3308-3860, 3323-3587, 3330-3843, 3337-3860, 3358-3625, 3360-3585, 3360-3840, 3367-3860, |
| | 3370-3772, 3370-3860, 3377-3837, 3378-3629, 3379-3843, 3381-3838, 3384-3792, 3384-3846, 3387-3843, 3391-3847, |
| | 3391-3851, 3392-3613, 3393-3847, 3393-3850, 3394-3613, 3397-3844, 3400-3526, 3408-3704, 3409-3847, |
| | 3411-3858, 3411-3860, 3414-3695, 3418-3846, 3422-3860, 3429-3849, 3432-3860, 3433-3844, 3436-3842, 3437-3700, |
| | 3438-3846, 3446-3839, 3454-3837, 3456-3843, 3456-3860, 3463-3680, 3464-3734, 3489-3757, 3489-3812, |
| | 3489-3843, 3502-3843, 3520-3860, 3522-3845, 3531-3843, 3556-3738, 3558-3844, 3582-3838, 3585-3846, 3592-3846, |
| | 3594-3846, 3609-3818, 3644-3846, 3698-3860, 3758-3843, 3761-3835, 3767-3832, 3769-3847, 3791-3832 |
| 58/2500251CB1/ | 1-271, 1-492, 1-542, 1-805, 11-639, 16-564, 20-611, 256-634, 293-936, 334-788, 342-1017, 359-716, 385-1081, 419-899, |
| 3742 | 453-1060, 474-1183, 481-788, 486-794, 543-1230, 549-920, 552-1148, 553-1021, 601-1265, 604-1200, 615-1029, |
| | 691-1238, 730-1300, 769-1295, 809-1402, 826-1400, 834-1138, 916-1506, 930-1588, 938-1504, 1000-1634, |
| | 1086-1628, 1278-1551, 1297-1630, 1358-1601, 1358-1936, 1358-1996, 1361-1621, 1361-1912, 1392-1822, 1404-2053, |
| | 1542-2252, 1615-1878, 1615-2219, 1730-1930, 1768-2308, 1797-2436, 1851-2515, 1864-2458, 1925-2474, |
| | 1965-2493, 2062-2462, 2122-2456, 2128-2465, 2215-2823, 2245-2772, 2247-2702, 2325-2725, 2349-2690, 2351-2598, |
| | 2351-2895, 2351-2903, 2451-2725, 2637-3213, 2639-3199, 2748-3391, 2778-3214, 2884-3742, 2926-3174, |
| | 2926-3317 |
| 59/55026561CB1/ | 1-491, 1-2160, 1366-1943, 1462-2115, 1722-2160, 1854-2044, 1854-2138 |
| 2160 | |
| 60/7502593CB1/ | 1-485, 1-555, 1-594, 1-684, 1-1840, 17-301, 405-681, 1097-1529, 1554-1671 |
| 1840 | |
| 61/7503957CB1/ | 1-848, 71-683, 71-761, 190-770, 346-937, 369-657, 369-755, 369-897, 369-942, 369-952, 369-961, 500-1050, 508-978, |
| 1808 | 527-1071, 540-984, 540-1006, 562-863, 564-990, 614-1192, 628-1221, 629-1094, 722-1284, 794-1368, 841-1427, |
| | 848-1458, 874-1431, 874-1500, 924-1365, 934-1808, 940-1555, 1004-1457, 1054-1569, 1084-1541, 1088-1290, |
| | 1116-1561, 1134-1570, 1160-1566, 1196-1570, 1201-1581, 1202-1659, 1205-1446, 1205-1659, 1214-1564, |
| | 1215-1561, 1215-1563, 1219-1563, 1222-1561, 1224-1519 |
| 62/7504415CB1/ | 1-835, 5-765, 89-779, 286-883, 431-1121, 497-1192, 527-733, 545-996, 576-1382, 600-850, 600-1054, 610-1118, |
| 3941 | 670-1095, 708-1445, 744-1071, 817-1591, 914-1510, 928-1175, 1010-1464, 1016-1448, 1335-2187, 1336-1968, |
| | 1402-2108, 1527-2112, 1529-2267, 1550-2173, 1550-2229, 1588-2202, 1604-2111, 1606-2143, 1627-2272, 1631-1888, |
| | 1643-1937, 1706-1974, 1738-2428, 1761-2020, 1863-1995, 1863-1998, 1863-2021, 1863-2321, 1863-2332, |
| | 1894-1994, 1897-2364, 1917-2185, 1923-2537, 1936-2275, 1957-2186, 1969-2437, 2008-2233, 2009-2256, 2048-2659, |
| | 2093-2366, 2159-2407, 2167-2762, 2253-2946, 2261-2920, 2261-2971, 2279-2850, 2298-2512, 2300-2563, |
| | 2308-2919, 2329-2854, 2364-3026, 2365-2924, 2368-2927, 2386-2979, 2391-2761, 2395-3032, 2404-2748, 2406-2792, |
| | 2414-2683, 2414-2978, 2416-3069, 2426-2692, 2433-2732, 2433-2751, 2434-3135, 2438-2725, 2465-3143, |
| | 2466-3065, |
| | 2480-2671, 2480-3138, 2528-2810, 2536-2809, 2536-3387, 2551-2955, 2556-2754, 2556-2784, 2556-2786, 2556-2788, |
| | 2568-2760, 2573-2692, 2578-3096, 2580-3144, 2594-2876, 2600-3221, 2631-2884, 2634-2908, 2638-2885, |
| | 2666-3123, 2667-2942, 2667-2969, 2673-3290, 2685-3276, 2715-3289, 2740-3281, 2761-3255, 2787-3401, 2998-3282, |
| | 3120-3713, 3269-3750, 3271-3938, 3273-3642, 3295-3486, 3295-3510, 3304-3675, 3360-3941, 3385-3885, |
| | 3392-3621, 3400-3713, 3413-3718, 3443-3717, 3445-3717, 3450-3718, 3469-3717, 3481-3718, 3507-3717, 3579-3713 |
| 63/7504074CB1/ | 1-780, 85-920, 93-324, 226-787, 251-627, 251-664, 251-747, 251-763, 251-767, 256-767, 274-897, 297-869, 302-924, |
| 1933 | 328-747, 354-767, 431-971, 434-893, 434-935, 442-893, 447-724, 464-743, 464-767, 476-951, 477-1138, 493-803, |
| | 564-1015, 577-1026, 592-1115, 613-1177, 613-1411, 622-1072, 641-1085, 641-1164, 668-1083, 672-1298, 696-980, |
| | 702-966, 702-1303, 726-1297, 737-1353, 746-1353, 787-1425, 814-1314, 847-1413, 852-1258, 880-1398, 889-1560, |
| | 894-1314, 896-1703, 901-1427, 903-1170, 907-1336, 917-1360, 939-1247, 950-1424, 967-1419, 967-1427, |
| | 972-1425, 992-1516, 994-1201, 994-1239, 1019-1250, 1101-1411, 1103-1393, 1109-1392, 1122-1425, 1135-1400, |
| | 1137-1714, 1147-1933, 1149-1425, 1163-1560, 1208-1425, 1219-1472, 1228-1394, 1228-1724, 1231-1592, 1240-1425, |
| | 1248-1682, 1254-1425, 1284-1425, 1291-1425 |
| 64/7502257CB1/ | 1-584, 1-716, 1-4460, 24-754, 29-777, 29-798, 29-816, 29-827, 31-787, 49-615, 346-1033, 353-1022, 496-1087, 545-1033, |
| 4460 | 572-994, 598-1087, 767-1014, 803-1232, 805-1232, 979-1624, 1081-1669, 1159-1792, 1228-1627, 1264-1692, |
| | 1303-1692, 1366-1921, 1392-1862, 1481-2198, 1524-1812, 1541-2201, 1631-2197, 1654-1909, 1688-1966, |
| | 1688-2250, 1688-2286, 1688-2376, 1692-2361, 1699-1967, 1717-2005, 1726-2481, 1740-1974, 1742-2322, 1767-2563, |
| | 1779-2563, 1788-2315, 1801-2073, 1805-2563, 1819-2565, 1820-2061, 1833-2149, 1835-2092, 1839-2407, |
| | 1845-2465, 1850-2563, 1859-2$$63, 1860-2479, 1860-2563, 1862-2563, 1866-2395, 1866-2581, 1877-2563, 1881-2532, |
| | 1915-2197, 1921-2563, |
| | 1932-2701, 1933-2563, 2002-2300, 2007-2408, 2042-2744, 2079-2777, 2128-2760, 2147-2656, 2172-2752, 2235-2753, |
| | 2307-2862, 2315-2940, 2328-2813, 2328-3000, 2329-2854, 2335-2823, 2335-2991, 2391-3130, 2400-3007, |
| | 2415-2947, 2429-2914, 2442-2869, 2468-2769, 2474-3238, 2478-3138, 2493-3134, 2510-3169, 2511-3264, 2512-3251, |
| | 2513-2805, 2529-3134, 2542-2779, 2543-2984, 2547-3291, 2567-3236, 2600-3254, 2602-3236, 2606-2914, |
| | 2619-3145, 2629-2866, 2659-3038, 2665-2998, 2666-3043, 2672-3026, 2680-3290, 2697-3570, 2704-3318, 2728-2988, |
| | 2736-3289, 2765-3015, 2773-3457, 2783-3238, 2783-3574, 2797-3454, 2816-3245, 2816-3318, 2816-3366, |
| | 2832-3303, 2832-3383, 2832-3576, 2850-3352, 2850-3431, 2850-3439, 2856-3137, 2856-3265, 2857-3399, 2859-3772, |
| | 2867-3419, |
| | 2867-3721, 2888-3600, 2888-3632, 2889-3570, 2898-3369, 2901-3386, 2910-3223, 2910-3283, 2911-3525, 2914-3221, |
| | 2918-3760, 2936-3357, 2944-3474, 2987-3574, 3039-3482, 3039-3768, 3049-3544, 3051-3589, 3052-3589, |
| | 3054-3358, 3054-3494, 3058-3342, 3060-3435, 3065-3317, 3065-3655, 3067-3366, 3081-3620, 3126-3573, 3162-3501, |
| | 3166-3448, 3179-3414, 3206-3862, 3258-3497, 3258-3778, 3315-3814, 3400-4048, 3443-4033, 3468-3814, |
| | 3480-4130, 3551-4023, 3580-4406, 3655-4388, 3664-4406, 3701-4460, 3714-4183, 3736-4305, 3736-4339, 3774-4245, |
| | 3775-4313, 3775-4446, 3777-4310, 3780-4260, 3780-4326, 3784-4314, 3789-4153, 3795-4282, 3800-4261, |
| | 3804-4311, 3846-4411, 3904-4314, 3904-4337, 3934-4456, 3946-4338, 3988-4460, 3994-4460, 4004-4460, 4009-4456, |
| | 4026-4460, 4065-4455, 4088-4460, 4090-4460, 4124-4460, 4130-4460, 4141-4460 |
| 65/1315136CB1/ | 1-430, 57-95, 100-138, 273-820, 275-469, 305-453, 581-814, 582-845, 590-801, 594-858, 594-860, 595-846, 600-849, |
| 1214 | 605-1098, 632-903, 643-846, 645-909, 647-1064, 663-811, 706-928, 707-1127, 707-1214, 712-1213, 718-948, |
| | 718-967, 718-1214, 736-1191, 759-1196, 767-1214, 773-1196, 815-1055, 815-1202, 822-1199, 826-1059, 836-1087, |
| | 836-1190, 836-1212, 843-1199, 920-1162, 933-1169, 939-1163, 939-1213, 940-1209, 993-1214, 1053-1199 |
| 66/1379785CB1/ | 1-642, 197-285, 207-953, 254-445, 254-448, 254-453, 254-456, 313-1110, 735-1271, 735-1307, 735-1309, 735-1401, |
| 2843 | 735-1539, 735-1544, 735-1592, 735-1600, 735-1603, 735-1611, 736-1613, 744-1613, 753-1613, 809-2833, |
| | 1338-1725, 2015-2259, 2015-2508, 2093-2334, 2221-2828, 2756-2843, 2757-2843 |
| 67/2011166CB1/ | 1-770, 84-1021, 188-946, 566-1021, 570-1021, 576-1021, 608-1021, 609-1021, 615-1021, 620-1021, 633-891, 633-1005, |
| 1021 | 633-1017, 633-1021, 634-1015, 638-1021, 641-1017, 641-1020, 641-1021, 648-976, 680-1019, 719-1020, 777-956, |
| | 778-956 |
| 68/3434684CB1/ | 1-704, 483-1085, 1006-1606, 1006-1666, 1194-1630, 1311-1931, 1311-2038, 1486-2257, 1524-1974, 1524-2219, |
| 4074 | 1555-2222, 1569-2332, 1573-2331, 1581-1827, 1581-2331, 1616-2195, 1620-2329, 1636-2216, 1654-2286, 1685-2393, |
| | 1695-2331, 1739-2011, 1739-2394, 1743-2330, 1751-2045, 1754-2331, 1754-2332, 1759-2530, 1766-2469, |
| | 1795-2400, 1813-2330, 1818-1962, 1858-2287, 1880-2479, 1950-2314, 2072-2346, 2072-2461, 2077-2664, 2099-2376, |
| | 2105-2543, 2105-2810, 2112-2616, 2144-2803, 2166-2728, 2172-2758, 2198-2457, 2199-2445, 2248-2911, |
| | 2307-3077, 2361-2764, 2482-2776, 2497-2717, 2504-2697, 2532-3207, 2546-3184, 2547-2911, 2559-3136, 2572-2813, |
| | 2591-2635, 2626-3109, |
| | 2653-3124, 2653-3128, 2658-2951, 2661-3469, 2677-3265, 2682-2944, 2688-3265, 2689-2936, 2689-3222, 2698-3378, |
| | 2741-2988, 2741-2994, 2753-3288, 2774-3343, 2815-3471, 2823-3414, 2831-3282, 2840-3093, 2878-3054, |
| | 2896-3051, 2896-3086, 2921-3415, 2940-3182, 3022-3570, 3039-3549, 3046-3477, 3050-3666, 3081-3364, 3129-3667, |
| | 3136-3391, 3137-3469, 3138-3469, 3146-3399, 3154-3826, 3171-3770, 3175-3511, 3178-3452, 3244-3505, |
| | 3271-3755, 3271-3836, 3288-3579, 3347-3589, 3364-3632, 3364-3643, 3393-3673, 3418-3678, 3436-3874, 3459-4074 |
| 69/5134056CB1/ | 1-27, 1-261, 17-238, 20-607, 26-594, 88-201, 100-575, 154-733, 391-937, 431-975, 431-1010, 526-1080, 658-1255, |
| 2551 | 832-1445, 957-1501, 1014-1118, 1118-1391, 1174-1776, 1179-1427, 1254-1652, 1259-1703, 1333-1581, 1386-1667, |
| | 1386-1880, 1410-2036, 1515-2102, 1575-1776, 1575-1993, 1586-2091, 1611-1882, 1647-1956, 1662-2156, |
| | 1675-1977, 1681-2231, 1708-2084, 1715-2011, 1718-2009, 1724-2259, 1736-2420, 1745-2332, 1747-2399, 1768-2131, |
| | 1772-2460, 1783-2450, 1787-2235, 1795-2435, 1799-2434, 1804-2281, 1806-2108, 1807-2381, 1814-2365, |
| | 1822-2073, 1822-2109, 1845-2132, 1845-2143, 1854-2183, 1885-2463, 1886-2158, 1893-2495, 1905-2178, 1915-2473, |
| | 1922-2490, 1927-2490, 1947-2435, 1949-2430, 1983-2199, 1983-2386, 1983-2490, 2017-2434, 2021-2243, |
| | 2021-2507, 2021-2508, 2035-2302, 2059-2279, 2062-2238, 2065-2319, 2074-2331, 2081-2365, 2094-2357, 2145-2415, |
| | 2145-2425, 2145-2430, 2271-2551 |
| 70/5281724CB1/ | 1-472, 20-261, 25-323, 47-472, 184-403, 184-659, 188-284, 188-472, 224-400, 345-950, 473-562, 473-659, 540-1008, |
| 1115 | 547-647, 553-1008, 590-1011, 641-1008, 661-1009, 692-1011, 705-1011, 731-1008, 736-1018, 822-1115 |
| 71/7502391CB1/ | 1-591, 1-643, 1-677, 1-720, 1-790, 1-793, 1-840, 1-842, 1-845, 1-903, 1-937, 1-945, 1-953, 1-970, 3-508, 3-545, 6-503, |
| 1334 | 8-670, 9-626, 14-623, 16-454, 16-643, 18-618, 24-655, 25-733, 29-783, 51-423, 94-853, 134-689, 152-577, 158-617, |
| | 171-771, 185-680, 189-749, 203-494, 220-765, 227-633, 227-657, 229-951, 232-1008, 257-1040, 299-1025, |
| | 319-575, 369-915, 377-702, 385-963, 431-664, 436-1163, 439-698, 439-700, 447-924, 449-738, 466-1323, 471-756, |
| | 480-1179, 507-1324, 686-1334, 753-1317, 1035-1334 |
| 72/7502544CB1/ | 1-301, 1-309, 1-457, 1-469, 1-474, 1-493, 1-530, 1-532, 1-558, 1-559, 1-696, 1-699, 1-753, 1-800, 2-474, 4-453, 4-739, |
| 2387 | 11-558, 12-687, 22-907, 23-559, 23-900, 24-308, 24-555, 25-895, 32-513, 34-661, 35-825, 36-857, 48-483, 51-1017, |
| | 56-340, 56-345, 56-428, 72-340, 100-365, 100-637, 100-687, 100-714, 100-813, 101-407, 101-775, 101-778, |
| | 104-353, 104-648, 108-558, 110-677, 113-815, 122-771, 124-756, 155-842, 278-529, 320-558, 341-620, 361-683, |
| | 368-687, 379-684, 407-558, 703-1387, 777-1418, 1351-2027, 1361-1972, 1368-2204, 1378-2144, 1381-2126, 1386-2087, |
| | 1388-2126, 1394-1999, 1396-2065, 1397-2209, 1404-2036, 1457-2191, 1464-2387, 1465-2205, 1465-2385, |
| | 1469-2137, 1474-2186, 1488-2271, 1490-2337, 1521-2132, 1523-2185, 1593-2203, 1600-2306, 1602-2325, 1607-2384, |
| | 1624-1856, 1632-2265 |
| 73/2858465CB1/ | 1-284, 1-301, 1-749, 1-771, 1-775, 1-787, 12-664, 13-811, 15-268, 15-344, 15-772, 23-645, 27-327, 36-736, 44-638, |
| 2919 | 44-645, 109-139, 137-241, 137-530, 195-509, 202-420, 214-1024, 216-1087, 241-472, 271-795, 293-706, 310-594, |
| | 363-610, 435-694, 435-1245, 468-1261, 494-920, 507-1260, 518-1196, 521-1232, 563-1153, 685-1245, 725-1199, |
| | 725-1384, 759-864, 777-1362, 792-1402, 832-1134, 879-1484, 943-1556, 954-1452, 954-1889, 961-1579, 968-1452, |
| | 1056-1823, 1057-1865, 1061-1646, 1062-1579, 1062-1585, 1078-1816, 1121-1736, 1125-1751, 1168-1951, 1192-2035, |
| | 1218-1809, 1261-1728, 1276-1939, 1283-1897, 1314-2071, 1325-1420, 1327-1871, 1362-2016, 1384-2139, |
| | 1388-1819, 1388-1879, |
| | 1443-1942, 1469-2034, 1489-2042, 1493-1907, 1507-2099, 1514-1939, 1529-2075, 1536-1978, 1576-1842, 1584-1869, |
| | 1586-2407, 1611-1787, 1619-1889, 1621-1905, 1635-1921, 1637-1739, 1663-1924, 1669-2256, 1673-2270, |
| | 1690-1977, 1699-2303, 1711-2351, 1725-1984, 1730-2013, 1766-2035, 1772-2223, 1784-2088, 1794-2107, 1794-2214, |
| | 1822-2162, 1828-2366, 1834-2127, 1836-2282, 1840-2346, 1842-2530, 1845-2112, 1847-2210, 1847-2303, |
| | 1853-2438, 1865-2131, 1913-2832, 1918-2074, 1922-2459, 1923-2387, 1938-2648, 1943-2184, 1947-2199, 1947-2608, |
| | 1964-2361, 1968-2133, 1973-2470, 2013-2535, 2026-2836, 2058-2529, 2063-2331, 2070-2316, 2071-2529, |
| | 2071-2832, 2092-2752, 2098-2472, 2104-2360, 2106-2918, 2112-2896, 2114-2888, 2124-2657, 2131-2785, 2136-2424, |
| | 2137-2412, 2150-2834, 2150-2860, 2157-2586, 2172-2908, 2174-2398, 2177-2526, 2177-2699, 2214-2919, |
| | 2221-2478, |
| | 2234-2710, 2234-2886, 2240-2713, 2244-2908, 2259-2835, 2280-2485, 2294-2753, 2302-2908, 2306-2919, 2322-2590, |
| | 2322-2727, 2326-2867, 2327-2900, 2348-2629, 2355-2901, 2371-2828, 2376-2658, 2378-2919, 2381-2602, |
| | 2385-2869, 2393-2909, 2396-2895, 2424-2901, 2430-2710, 2434-2615, 2434-2690, 2434-2860, 2436-2840, 2438-2919, |
| | 2440-2910, 2441-2694, 2442-2908, 2443-2906, 2444-2727, 2448-2877, 2448-2891, 2452-2910, 2454-2907, |
| | 2456-2907, 2459-2908, 2462-2908, 2463-2909, 2463-2911, 2467-2919, 2472-2913, 2473-2913, 2475-2919, 2477-2908, |
| | 2479-2904, 2479-2908, 2482-2910, 2485-2919, 2491-2919, 2493-2919, 2499-2728, 2499-2906, 2499-2907, |
| | 2503-2912, 2504-2908, 2506-2908, 2509-2744, 2509-2908, 2525-2596, 2525-2764, 2525-2912, 2525-2914, 2526-2897, |
| | 2527-2908, 2567-2912, 2572-2909, 2576-2919, 2578-2911, 2582-2905, 2612-2906, 2620-2910, 2627-2908, |
| | 2632-2841, 2640-2914, 2651-2912, 2661-2897 |
| 74/7503455CB1/ | 1-253, 1-263, 1-272, 1-273, 1-290, 1-294, 1-295, 2-196, 2-1414, 9-289, 11-248, 15-273, 15-300, 18-227, 18-456, 24-287, |
| 1414 | 28-296, 31-271, 31-306, 32-287, 34-172, 34-259, 34-274, 34-276, 34-283, 34-286, 34-290, 34-300, 34-312, 34-314, |
| | 34-322, 34-327, 34-405, 34-444, 35-217, 35-282, 35-307, 36-258, 36-269, 36-276, 36-279, 36-282, 36-293, 36-297, |
| | 36-325, 37-279, 37-283, 37-288, 37-304, 37-370, 37-469, 38-209, 38-215, 38-245, 38-247, 38-251, 38-258, 38-259, |
| | 38-265, 38-267, 38-269, 38-276, 38-284, 38-286, 38-287, 38-288, 38-292, 38-296, 38-298, 38-302, 38-306, 38-307, |
| | 38-310, 38-311, 38-313, 38-317, 38-319, 38-325, 38-328, 38-332, 38-356, 38-456, 39-265, 39-276, 39-336, 39-419, |
| | 40-182, 41-285, 41-291, |
| | 41-294, 41-314, 44-292, 46-299, 46-324, 48-337, 48-365, 53-292, 53-300, 53-311, 53-313, 53-341, 53-348, 53-351, |
| | 54-206, 55-309, 56-324, 59-348, 60-282, 60-311, 60-319, 60-343, 60-355, 60-442, 61-277, 61-300, 61-308, 61-311, |
| | 61-313, 61-314, 61-356, 61-391, 62-228, 62-235, 62-265, 62-276, 62-292, 62-310, 62-313, 62-325, 62-336, 62-339, |
| | 62-361, 62-372, 62-385, 63-297, 63-306, 63-319, 63-341, 63-345, 63-349, 63-351, 63-376, 64-297, 64-321, 64-336, |
| | 64-347, 64-351, 64-373, 64-421, 65-307, 65-317, 65-340, 69-607, 69-611, 71-298, 71-306, 73-611, 75-343, 89-391, |
| | 92-348, 93-355, 96-305, 96-314, 98-320, 104-370, 111-264, 123-426, 130-401, 151-386, 151-426, 166-299, 168-387, |
| | 171-393, 176-441, 176-451, |
| | 189-405, 189-440, 193-420, 205-363, 280-306, 357-972, 382-938, 456-986, 456-1086, 457-1086, 458-921, 475-609, |
| | 475-734, 480-701, 493-1082, 497-1017, 516-1063, 536-1201, 539-1083, 541-1091, 546-1079, 550-1204, 559-1188, |
| | 563-1241, 571-1018, 571-1110, 571-1185, 571-1205, 584-740, 587-973, 587-1270, 591-1231, 595-1159, 607-930, |
| | 609-1175, 621-838, 627-908, 634-1298, 638-890, 641-898, 643-848, 643-888, 648-935, 653-900, 656-917, 662-906, |
| | 663-951, 665-896, 673-879, 674-958, 677-817, 677-933, 680-901, 685-1029, 687-934, 687-953, 687-1272, 692-956, |
| | 696-941, 697-941, 697-993, 698-1136, 701-850, 701-1015, 702-975, 703-1013, 705-979, 707-947, 707-951, 709-949, |
| | 710-936, 712-942, 718-1016, 727-986, 732-986, 733-1016, 741-1061, 742-998, 744-896, 747-832, 750-1042, |
| | 757-980, 757-983, 757-1002, 757-1026, 761-1005, 762-1031, 763-998, 763-1017, 765-870, 766-1042, 773-1007, |
| | 775-1024, 775-1034, 778-999, 778-1005, 778-1028, 788-1031, 788-1053, 791-1091, 794-874, 795-1052, 796-1093, |
| | 797-1072, 798-1126, 801-990, 805-1021, 806-1062, 807-1103, 809-969, 811-1258, 820-1134, 820-1310, 822-1078, |
| | 822-1107, 823-1065, 827-1026, 827-1055, 827-1071, 827-1081, 827-1121, 827-1159, 830-1045, 830-1097, 830-1173, |
| | 830-1191, 831-1061, 839-1096, 843-1123, 843-1132, 844-1100, 845-1101, 845-1126, 846-1028, 846-1127, |
| | 846-1247, 847-1106, 848-1110, 850-1170, 851-1058, 851-1085, 851-1146, 855-1062, 857-1014, 857-1080, 857-1121, |
| | 859-1129, 862-1023, 863-1101, 868-1171, 876-1157, 877-1149, 877-1178, 878-1298, 887-1173, 893-1128, |
| | 897-1350, 897-1414, 907-1109, 908-1140, 910-1190, 911-1046, 921-1296, 922-1214, 929-1195, 938-1249, 940-1189, |
| | 946-1304, 950-1220, 950-1414, 956-1200, 958-1203, 959-1191, 961-1270, 963-1208, 965-1260, 973-1265, |
| | 974-1229, 977-1171, 977-1191, 981-1232, 986-1256, 987-1268, 994-1248, 995-1247, 1004-1139, 1008-1196, 1008-1210, |
| | 1013-1259, 1013-1263, 1016-1284, 1021-1249, 1021-1257, 1022-1242, 1030-1150, 1030-1293, 1046-1266 |
| 75/7503479CB1/ | 1-219, 1-246, 1-530, 11-246, 25-246, 27-246, 30-246, 31-246, 33-246, 33-672, 35-111, 38-244, 38-536, 43-162, 44-246, |
| 672 | 45-246, 46-246, 48-246, 49-191, 50-154, 51-183, 53-244, 54-203, 67-243, 67-246, 73-245, 107-191, 245-527, |
| | 249-524, 251-528, 268-467, 282-527, 293-527, 366-527, 431-527 |
| 76/7218127CB1/ | 1-591, 71-537, 77-591, 82-536, 90-591, 265-591, 533-1254, 533-1274, 552-1244, 703-1209, 708-1368, 716-926, 729-1299, |
| 3056 | 767-1340, 790-1128, 833-1152, 861-1270, 874-1494, 916-2972, 931-1299, 999-1674, 1069-1357, 1088-1347, |
| | 1228-1418, 1228-1717, 1245-1887, 1296-1620, 1299-1532, 1311-1371, 1335-1838, 1353-1686, 1409-1652, 1531-2114, |
| | 1566-2066, 1597-2149, 1597-2155, 1669-1911, 1675-2258, 1677-2268, 1686-1937, 1708-2278, 1751-2408, |
| | 1840-2114, 1840-2130, 1869-2388, 1897-2152, 2002-2360, 2002-2364, 2377-2880, 2405-3056 |
| 77/1688943CB1/ | 1-606, 1-681, 5-553, 7-121, 273-845, 304-845, 417-720, 469-788, 589-792, 636-1737, 713-845, 845-1035, 845-1127, |
| 1891 | 845-1370, 885-1524, 917-1442, 990-1182, 1225-1778, 1323-1868, 1444-1729, 1454-1589, 1485-1729, 1536-1888, |
| | 1554-1886, 1572-1888, 1589-1803, 1589-1819, 1589-1888, 1638-1729, 1729-1891, 1744-1887 |
| 78/2369350CB1/ | 1-423, 1-460, 186-442, 186-657, 202-624, 286-884, 359-613, 373-498, 630-1358, 761-1361, 1089-1742, 1104-1659, |
| 2221 | 1182-1431, 1195-1836, 1201-1665, 1217-1524, 1292-1547, 1310-1526, 1310-1808, 1314-1506, 1324-1516, 1327-1554, |
| | 1327-1846, 1343-1592, 1346-1501, 1395-1900, 1494-1749, 1520-1799, 1585-1851, 1593-1902, 1611-1902, |
| | 1672-1902, 1673-1902, 1678-1854, 1678-1870, 1681-1902, 1686-1902, 1690-1902, 1694-2221, 1698-1903, 1721-1902, |
| | 1728-1854, 1728-1902, 1764-1814, 1898-1941, 1903-1941 |
| 79/2722979CB1/ | 1-522, 27-603, 27-614, 30-353, 53-489, 53-508, 59-473, 66-167, 66-439, 66-495, 66-496, 66-497, 66-505, 66-510, |
| 2039 | 66-519, 95-292, 324-720, 349-777, 360-540, 361-610, 361-805, 425-837, 519-769, 649-859, 762-1295, 762-2034, |
| | 931-1189, 931-1423, 936-1177, 936-1602, 1057-1347, 1128-1349, 1147-1661, 1157-1257, 1210-1408, 1395-2021, |
| | 1436-1742, 1436-1992, 1436-2000, 1469-2018, 1471-1769, 1521-1761, 1534-2030, 1550-1978, 1558-1812, 1560-1980, |
| | 1560-2006, 1560-2031, 1567-2028, 1574-2034, 1593-2028, 1616-2033, 1620-2034, 1627-2036, 1632-2039, |
| | 1640-2020, 1650-1850, 1650-1983, 1650-2018, 1663-2029, 1699-1871, 1741-2034, 1749-1961, 1775-2002, 1775-2028, |
| | 1812-2039, 1820-1993, 1856-2039 |
| 80/60140470CB1/ | 1-796, 21-595, 34-613, 34-683, 40-741, 143-390, 379-786, 379-820, 404-1066, 539-1037, 556-824, 732-1205, 963-1254 |
| 1254 | |
| 81/70623603CB1/ | 1-274, 32-248, 52-303, 52-623, 57-738, 59-324, 60-298, 60-659, 61-328, 61-360, 64-611, 65-333, 65-348, 66-361, |
| 1879 | 66-594, 68-404, 69-362, 74-382, 74-622, 74-753, 75-385, 75-846, 76-312, 77-369, 78-354, 81-348, 81-350, 81-613, |
| | 81-679, 82-331, 82-369, 82-372, 82-375, 82-857, 83-380, 87-854, 91-537, 91-747, 101-886, 108-376, 108-378, 146-349, |
| | 186-669, 224-475, 302-430, 493-781, 567-1268, 586-1084, 586-1170, 586-1195, 586-1269, 608-862, 626-893, |
| | 640-894, 640-1183, 720-1013, 749-980, 759-1385, 771-1414, 909-1117, 909-1118, 909-1402, 929-1378, 962-1698, |
| | 1006-1611, 1060-1750, 1063-1654, 1080-1293, 1089-1324, 1089-1574, 1092-1724, 1125-1720, 1129-1723, 1130-1750, |
| | 1138-1790, 1142-1691, 1201-1808, 1203-1465, 1203-1668, 1215-1481, 1219-1747, 1219-1765, 1222-1658, |
| | 1232-1449, 1232-1792, 1263-1705, 1263-1797, 1276-1490, 1278-1655, 1333-1765, 1391-1600, 1410-1698, 1410-1797, |
| | 1410-1809, 1523-1790, 1567-1741, 1597-1879 |
| 82/7161479CB1/ | 1-769, 132-707, 400-860, 408-843, 460-589, 462-1163, 507-1160, 522-1019, 525-755, 547-952, 567-816, 567-821, |
| 2767 | 760-1019, 813-1379, 840-1450, 888-1176, 911-1221, 911-1407, 1004-1221, 1035-1276, 1094-1373, 1100-1315, |
| | 1123-1379, 1179-1763, 1270-1494, 1339-1383, 1444-1695, 1497-1783, 1497-1945, 1497-2008, 1497-2055, 1497-2152, |
| | 1499-1799, 1499-2034, 1499-2286, 1505-1775, 1512-1660, 1524-1693, 1547-1940, 1567-1989, 1568-2104, |
| | 1607-1791, 1632-2189, 1637-1769, 1637-2221, 1637-2234, 1639-2072, 1639-2134, 1674-2191, 1682-2340, 1697-1941, |
| | 1759-2310, 1763-1985, 1763-2366, 1787-1856, 1878-2030, 1899-2048, 1942-2100, 1957-2097, 1959-2115, |
| | 1959-2116, 1959-2201, 1959-2227, 1959-2231, 1959-2234, 1960-2233, 1961-2211, 1961-2222, 2128-2767, 2234-2719 |
| 83/7502313CB1/ | 1-647, 27-680, 211-787, 318-595, 339-1049, 441-972, 441-1013, 447-1939, 449-769, 449-829, 449-925, 449-927, |
| 2364 | 466-1087, 916-1226, 1019-1715, 1035-1488, 1045-1243, 1066-1624, 1067-1354, 1155-1890, 1173-1288, 1173-1314, |
| | 1298-1901, 1338-1621, 1340-1633, 1340-1641, 1488-1942, 1499-1931, 1501-1942, 1512-1939, 1518-1935, |
| | 1520-1936, 1561-1823, 1562-1767, 1563-1938, 1583-1941, 1584-1941, 1588-2159, 1703-1934, 1726-1929, 1794-2364 |
| 84/7502390CB1/ | 1-372, 208-2282, 304-868, 372-776, 372-803, 372-816, 372-896, 373-625, 373-662, 373-701, 373-799, 373-919, 373-1134, |
| 2597 | 376-865, 380-811, 536-1236, 576-1077, 598-1263, 634-1198, 658-1224, 668-1226, 704-1148, 704-1213, 709-1255, |
| | 730-1007, 766-1235, 774-1425, 775-1259, 782-1057, 782-1137, 782-1162, 782-1187, 782-1189, 782-1232, |
| | 782-1249, 782-1269, 782-1295, 782-1303, 782-1320, 782-1333, 782-1361, 782-1395, 782-1398, 782-1444, 785-1487, |
| | 794-1368, 796-1335, 812-1489, 829-1458, 830-1488, 831-1334, 843-1169, 870-1123, 870-1336, 870-1539, |
| | 870-1543, 870-1640, 870-1672, 870-1709, 881-1333, 884-1244, 884-1252, 885-1317, 889-1665, 893-1170, 904-1454, |
| | 914-1444, 915-1436, 918-1302, 929-1556, 933-1617, 944-1640, 950-1438, 950-1443, 955-1297, 967-1472, |
| | 969-1495, 969-1606, 974-1368, 984-1523, 993-1514, 1010-1552, 1031-1470, 1033-1298, 1070-1656, 1089-1508, |
| | 1100-1409, |
| | 1111-1747, 1118-1391, 1120-1400, 1120-1675, 1129-1662, 1129-1684, 1133-1429, 1142-2041, 1150-1821, 1164-1861, |
| | 1166-1638, 1166-1707, 1197-1879, 1200-1899, 1209-1707, 1209-1712, 1234-1662, 1234-1701, 1247-1721, |
| | 1249-1770, 1256-1522, 1262-1906, 1269-1555, 1286-1850, 1287-1567, 1292-1903, 1299-1742, 1304-1821, 1317-2244, |
| | 1323-1888, 1324-1983, 1333-1871, 1334-1874, 1354-1825, 1356-1947, 1356-1993, 1357-1957, 1363-2070, |
| | 1375-1895, 1377-1857, 1385-1666, 1385-1878, 1385-1972, 1386-1941, 1392-1955, 1399-1928, 1400-1880, 1400-1899, |
| | 1402-1899, 1404-1878, 1404-1903, 1412-2002, 1414-1963, 1417-1874, 1421-1998, 1427-1940, 1431-2073, |
| | 1435-1617, 1454-1878, 1457-2000, 1463-2002, 1463-2059, 1464-2279, 1473-2045, 1475-2085, 1477-1947, 1478-2044, |
| | 1482-2100, 1484-1908, 1484-1909, 1485-2086, 1501-2008, 1512-1778, 1512-1824, 1514-1888, 1522-2016, |
| | 1524-2036, |
| | 1531-2182, 1534-2244, 1536-1976, 1536-2185, 1536-2204, 1541-1888, 1549-2251, 1554-2263, 1556-2181, 1557-2095, |
| | 1558-2251, 1560-1957, 1563-2262, 1563-2277, 1577-2058, 1584-2149, 1596-2257, 1601-1868, 1601-2276, |
| | 1606-2266, 1606-2276, 1608-2060, 1619-2263, 1619-2276, 1626-2279, 1635-2242, 1636-2211, 1642-2126, 1648-2226, |
| | 1655-1935, 1662-2279, 1663-2238, 1667-2240, 1669-2238, 1686-2269, 1699-2136, 1703-2088, 1709-2279, |
| | 1710-1974, 1711-2276, 1711-2279, 1715-2276, 1716-2231, 1726-2279, 1735-2279, 1748-2279, 1758-2276, 1773-2276, |
| | 1787-2279, 1794-2267, 1800-2320, 1803-2077, 1804-2076, 1804-2276, 1810-2279, 1812-2276, 1828-2279, |
| | 1830-2122, 1830-2278, 1832-2280, 1835-2279, 1839-2280, 1845-2597, 1846-2280, 1848-2279, 1857-2283, 1861-2256, |
| | 1866-2280, 1869-2137, 1878-2137, 1882-2282, 1886-2280, 1905-2282, 1915-2276, 1916-2254, 1941-2280, |
| | 1948-2280, 1987-2490, 2003-2282, 2007-2280, 2013-2282, 2496-2542, 2551-2597 |
| 85/7502872CB1/ | 1-547, 4-871, 17-608, 17-665, 37-719, 156-919, 156-935, 156-944, 156-950, 156-952, 273-1050, 290-688, 485-1185, |
| 2229 | 580-817, 585-829, 593-840, 593-856, 593-1143, 647-921, 715-1011, 715-1218, 718-876, 934-1657, 934-1665, |
| | 934-1689, 934-1696, 934-1713, 934-1735, 934-1737, 934-1839, 934-1855, 944-1200, 983-1560, 991-1530, 991-1575, |
| | 1000-1563, 1014-1332, 1035-1634, 1050-1337, 1057-1630, 1071-1349, 1074-1825, 1078-1598, 1084-1608, |
| | 1101-1634, 1101-1697, 1106-1533, 1109-1898, 1120-1761, 1124-1638, 1152-1762, 1168-1422, 1180-1782, 1199-1377, |
| | 1206-1535, 1209-1346, 1221-1796, 1222-1831, 1245-1685, 1322-1934, 1322-2014, 1327-1995, 1341-1880, |
| | 1348-1880, 1355-1554, 1435-1893, 1436-1950, 1452-2185, 1470-1994, 1473-1731, 1473-1979, 1478-1612, 1480-2139, |
| | 1490-1773, 1496-2030, 1500-1981, 1502-2149, 1503-2049, 1508-2003, 1517-2229 |
| 86/7505443CB1/ | 1-296, 1-437, 1-489, 1-493, 1-594, 1-645, 2-304, 13-270, 171-396, 182-794, 254-561, 278-833, 278-956, 281-965, |
| 2504 | 303-948, 369-622, 380-761, 515-1110, 534-1227, 539-1168, 570-1167, 581-1162, 592-1168, 599-1080, 609-1163, |
| | 641-1265, 651-1227, 652-1322, 669-1334, 682-1228, 727-1282, 766-1287, 772-1306, 881-1649, 963-1423, 1082-1299, |
| | 1268-1798, 1289-1831, 1389-2045, 1390-1961, 1424-2044, 1441-2063, 1447-2030, 1452-2067, 1459-1731, |
| | 1471-2067, 1603-1837, 1628-2001, 1669-2060, 1672-2067, 1842-2504 |
| 87/8032443CB1/ | 1-701, 2-478, 24-482, 30-488, 38-482, 59-482, 135-487, 155-389, 193-383, 284-487, 285-484, 311-482 |
| 701 | |
| 88/7704916CB1/ | 1-255, 1-1566, 177-977, 180-873, 180-941, 180-973, 180-1065, 184-873, 211-399, 551-1105, 577-879, 594-856, 669-1558, |
| 1569 | 677-1559, 680-1559, 739-1558, 883-1119, 892-1385, 915-1130, 979-1443, 1056-1325, 1111-1296, 1348-1569, |
| | 1354-1569, 1378-1569, 1410-1569 |
| 89/2013440CB1/ | 1-590, 11-542, 25-240, 25-411, 25-495, 30-486, 36-629, 44-488, 93-589, 134-293, 187-502, 273-811, 322-925, 349-926, |
| 1052 | 377-592, 377-925, 380-1013, 388-920, 404-906, 418-897, 424-901, 429-1037, 439-1015, 522-1052, 546-1048, |
| | 559-1047, 575-1026, 581-1049, 590-1031, 609-1023, 610-1026, 626-1023, 639-858, 653-1049, 677-1023, 698-1026, |
| | 700-1045 |
| 90/2503512CB1/ | 1-283, 111-349, 111-653, 260-528, 260-533, 460-751, 460-987, 467-711, 687-931, 687-982, 694-1304, 709-1304, |
| 1325 | 715-1325, 938-1213, 956-1224, 977-1325, 978-1311, 1057-1311, 1059-1228, 1084-1325 |
| 91/277396CB1/ | 1-494, 18-2110, 375-732, 375-836, 375-950, 472-1027, 478-924, 555-1187, 556-1112, 573-1068, 633-1229, 727-1332, |
| 2110 | 735-1324, 735-1330, 796-1436, 822-1416, 846-1436, 860-1484, 891-1543, 894-1482, 916-1570, 947-1461, |
| | 1084-1448, 1095-1724, 1105-1489, 1106-1620, 1168-1823, 1191-1799, 1290-1395, 1339-1816, 1349-1813, 1350-1813, |
| | 1390-1875, 1420-1828, 1425-1875, 1468-1875, 1476-1875, 1518-1875, 1565-1874, 1591-2007, 1666-2038, |
| | 1670-1878, 1670-2106, 1678-1875, 1699-1875, 1834-2110 |
| 92/3044046CB1/ | 1-277, 19-256, 31-317, 32-556, 45-195, 54-290, 54-583, 54-617, 90-609, 164-804, 164-837, 414-1085, 491-749, 493-685, |
| 1927 | 520-1085, 531-1254, 643-1149, 645-1072, 647-991, 681-1146, 686-1149, 704-1145, 725-1158, 728-1145, 745-1141, |
| | 824-983, 854-1342, 854-1446, 865-1378, 885-1406, 922-1123, 949-1564, 954-1482, 962-1643, 1002-1375, |
| | 1082-1570, 1088-1568, 1182-1679, 1275-1538, 1292-1927 |
| 93/3808420CB1/ | 1-240, 22-291, 24-520, 45-296, 172-466, 172-474, 172-476, 181-455, 273-601, 273-684, 274-575, 340-617, 362-606, |
| 1051 | 362-952, 374-1005, 428-883, 439-630, 455-1018, 497-1024, 509-1006, 526-987, 532-1045, 550-1051, 558-1019, |
| | 620-1022, 622-1029, 657-1019, 663-1024, 670-954, 670-965, 672-1022, 676-1018, 686-1019, 711-944, 711-976, |
| | 740-1015, 740-1019, 740-1021, 741-1024, 762-1020, 762-1021, 763-1042, 813-1022, 819-1019, 957-1040 |
| 94/7504028CB1/ | 1-642, 5-273, 7-500, 9-687, 9-705, 9-774, 9-795, 9-816, 9-825, 10-752, 12-515, 15-629, 21-651, 23-339, 28-267, 28-326, |
| 2328 | 28-515, 28-592, 28-678, 28-825, 34-616, 41-517, 52-277, 52-666, 53-443, 61-690, 69-561, 73-550, 166-473, |
| | 169-411, 169-446, 256-343, 256-550, 256-717, 298-876, 367-506, 403-616, 413-1030, 427-992, 441-941, 454-954, |
| | 468-993, 480-1256, 502-1047, 502-1053, 569-812, 583-1188, 593-1222, 608-908, 611-1260, 618-905, 674-993, 683-957, |
| | 689-970, 717-1395, 762-1327, 765-1337, 778-1553, 789-1083, 839-1107, 868-1090, 888-1178, 908-1596, 909-1523, |
| | 911-1523, 925-1523, 947-1340, 970-1293, 1083-1226, 1083-1480, 1105-1328, 1105-1360, 1111-1393, 1159-1752, |
| | 1181-1553, 1208-1391, 1227-1629, 1268-1842, 1282-1513, 1301-1840, 1370-1509, 1497-1763, 1599-1818, |
| | 1616-2175, 1667-2202, 1794-2126, 1895-2304, 1922-2128, 1973-2181, 2055-2328, 2056-2325 |
| 95/7766880CB1/ | 1-503, 1-4782, 42-224, 143-581, 143-583, 146-689, 156-607, 346-1087, 463-647, 559-1293, 756-1039, 756-1313, |
| 4782 | 924-1502, 944-1428, 952-1291, 963-1660, 1036-1441, 1229-1539, 1284-1575, 1372-1667, 1487-1771, 1532-1684, |
| | 1579-1996, 1644-1996, 1650-2069, 1713-2009, 2157-2349, 2165-2739, 2225-2725, 2275-2528, 2276-2728, 2276-2844, |
| | 2276-2846, 2312-2553, 2312-2569, 2312-2725, 2459-2735, 2535-3099, 2555-2723, 2564-3153, 2597-3132, |
| | 2607-2857, 2701-2979, 2701-3243, 2701-3275, 2701-3308, 2701-3349, 2703-3216, 2724-3029, 2741-3222, 2742-3306, |
| | 2742-3333, 2744-3360, 2772-3279, 2869-3406, 2944-3223, 2978-3548, 2981-3445, 3008-3600, 3039-3666, |
| | 3073-3252, 3073-3551, 3087-3579, 3129-3560, 3132-3490, 3149-3705, 3167-3670, 3168-3741, 3173-3787, 3181-3748, |
| | 3191-3743, |
| | 3205-3778, 3213-3527, 3259-3749, 3266-3537, 3291-3883, 3293-3944, 3305-3914, 3318-3839, 3329-3933, 3351-3937, |
| | 3351-3939, 3358-3891, 3372-3599, 3376-3835, 3392-3917, 3399-3751, 3432-3926, 3434-3959, 3439-4010, |
| | 3476-3959, 3482-3955, 3592-3959, 3616-3953, 3639-3899, 3705-3958, 3779-4076, 3907-4106, 3907-4572, 3922-4164, |
| | 3950-4616, 3982-4253, 4008-4599, 4015-4627, 4370-4635, 4498-4782 |
| 96/90089609CB1/ | 1-754, 64-482, 77-507, 164-391, 177-725, 178-630, 178-671, 178-716, 178-816, 178-820, 178-908, 178-914, 181-414, |
| 1410 | 186-732, 192-934, 248-962, 280-570, 425-636, 453-1077, 569-1404, 595-1056, 680-1126, 689-1404, 690-1404, |
| | 695-1406, 699-1404, 716-945, 716-1269, 739-1404, 741-1385, 746-1404, 746-1406, 747-1406, 763-1406, 769-1036, |
| | 780-1404, 806-1385, 819-1080, 845-1254, 865-1389, 932-1410, 948-1086, 959-1410, 972-1403, 990-1404, 1007-1404, |
| | 1010-1410, 1023-1406, 1043-1290, 1046-1410, 1064-1410, 1066-1332, 1079-1249, 1082-1409, 1198-1410 |

TABLE 5

| Polynucleotide | | |
|---|---|---|
| SEQ ID NO: | Incyte Project ID: | Representative Library |
| 49 | 1629602CB1 | COLNPOT01 |
| 50 | 2100360CB1 | BRAHNON05 |
| 51 | 5166833CB1 | LUNGNOT31 |
| 52 | 7494963CB1 | NOSETUE01 |
| 53 | 7644881CB1 | NERDTDN03 |
| 54 | 3790383CB1 | BRSTNOT28 |
| 55 | 3846110CB1 | DENDNOT01 |
| 56 | 1878279CB1 | ENDMUNE01 |
| 57 | 1848891CB1 | OVARNOT03 |
| 58 | 2500251CB1 | BRAVUNT02 |
| 59 | 55026561CB1 | LUNGDIS03 |
| 60 | 7502593CB1 | BRANDIN01 |
| 61 | 7503957CB1 | OVARTUT04 |
| 62 | 7504415CB1 | THP1NOB01 |
| 63 | 7504074CB1 | NEUTFMT01 |
| 64 | 7502257CB1 | MCLRNOC01 |
| 65 | 1315136CB1 | LUNGNOT09 |
| 66 | 1379785CB1 | LUNGNOT10 |
| 67 | 2011166CB1 | TESTNOT03 |
| 68 | 3434684CB1 | OVARDIR01 |
| 69 | 5134056CB1 | PROSNOT14 |
| 70 | 5281724CB1 | ADRETUR01 |
| 71 | 7502391CB1 | LUNGNOT38 |
| 72 | 7502544CB1 | KIDNTUE01 |
| 73 | 2858465CB1 | DRGCNOT01 |
| 74 | 7503455CB1 | BRAENOT04 |
| 75 | 7503479CB1 | 293TF2T01 |
| 76 | 7218127CB1 | SINTNOR01 |
| 77 | 1688943CB1 | THP1NOT03 |
| 78 | 2369350CB1 | BRAITUT02 |
| 79 | 2722979CB1 | HNT2AZS07 |
| 80 | 60140470CB1 | MIXDUNB01 |
| 81 | 70623603CB1 | BRSTUNF01 |
| 82 | 7161479CB1 | LIVRNON08 |
| 83 | 7502313CB1 | BONRFET01 |
| 84 | 7502390CB1 | THYRNOT03 |
| 85 | 7502872CB1 | LUNGTUT08 |
| 86 | 7505443CB1 | 293TF2T01 |
| 87 | 8032443CB1 | TESTNOF01 |
| 88 | 7704916CB1 | TESTNOT03 |
| 89 | 2013440CB1 | LUNGAST01 |
| 90 | 2503512CB1 | CONUTUT01 |
| 91 | 277396CB1 | TESTNOT03 |
| 92 | 3044046CB1 | TONSDIE01 |
| 93 | 3808420CB1 | LUNGNOT04 |
| 94 | 7504028CB1 | BRSTTUT08 |
| 95 | 7766880CB1 | BRAINON01 |
| 96 | 90089609CB1 | LUNGTUT08 |

TABLE 6

| Library | Vector | Library Description |
|---|---|---|
| 293TF2T01 | pINCY | Library was constructed using RNA isolated from a treated, |
| | | transformed embryonal cell line (293-EBNA) derived from |
| | | kidney epithelial tissue. The cells were treated with 5-aza-2′-deoxycytidine |
| | | and transformed with adenovirus 5 DNA. |
| ADRETUR01 | PCDNA2.1 | This random primed library was constructed using RNA isolated |
| | | from left upper pole, adrenal gland tumor tissue removed |
| | | from a 52-year-old Caucasian male during nephroureterectomy |
| | | and local destruction of renal lesion. Pathology indicated |
| | | grade 3 adrenal cortical carcinoma forming a mass that infiltrated |
| | | almost the whole adrenal parenchyma and extended to |
| | | adjacent adipose tissue. A metastatic tumor nodule was identified |
| | | in the hilar region. The renal vein was infiltrated by |
| | | tumor and the neoplastic process was present at the resection |
| | | margin of the renal vein. Fragments of adrenal cortical |
| | | carcinoma and thrombus were found in the inferior vena cava. |
| | | Patient history included abnormal weight loss. Family |
| | | history included skin cancer, type I diabetes, and neurotic depression. |
| BONRFET01 | pINCY | Library was constructed using RNA isolated from rib bone |
| | | tissue removed from a Caucasian male fetus, who died from |
| | | Patau's syndrome (trisomy 13) at 20-weeks' gestation. |
| BRAENOT04 | pINCY | Library was constructed using RNA isolated from inferior |
| | | parietal cortex tissue removed from the brain of a 35-year-old |
| | | Caucasian male who died from cardiac failure. Pathology indicated |
| | | moderate leptomeningeal fibrosis and multiple |
| | | microinfarctions of the cerebral neocortex. Patient history |
| | | included dilated cardiomyopathy, congestive heart failure, |
| | | cardiomegaly and an enlarged spleen and liver. |
| BRAHNON05 | pINCY | This normalized hippocampus tissue library was |
| | | constructed from 1.6 million independent clones from a hippocampus |
| | | tissue library. Starting RNA was made from posterior hippocampus |
| | | removed from a 35-year-old Caucasian male who died |
| | | from cardiac failure. Pathology indicated moderate leptomeningeal |
| | | fibrosis and multiple microinfarctions of the cerebral |
| | | neocortex. The cerebral hemisphere revealed moderate fibrosis |
| | | of the leptomeninges with focal calcifications. There was |
| | | evidence of shrunken and slightly eosinophilic pyramidal |
| | | neurons throughout the cerebral hemispheres. There were small |
| | | microscopic areas of cavitation with gliosis, scattered through |
| | | the cerebral cortex. Patient history included cardiomyopathy, |
| | | CHF, cardiomegaly, an enlarged spleen and liver. Patient |
| | | medications included simethicone, Lasix, Digoxin, Colace, |
| | | Zantac, captopril, and Vasotec. The library was normalized |
| | | in two rounds using conditions adapted from Soares et al., |
| | | PNAS (1994) 91: 9228 and Bonaldo et al., Genome Research |
| | | 6 (1996): 791, except that a significantly longer (48 |
| | | hours/round) reannealing hybridization was used. |
| BRAINON01 | PSPORT1 | Library was constructed and normalized from 4.88 |
| | | million independent clones from a brain tissue library. RNA was made |
| | | from brain tissue removed from a 26-year-old Caucasian |
| | | male during cranioplasty and excision of a cerebral meningeal |
| | | lesion. Pathology for the associated tumor tissue indicated a |
| | | grade 4 oligoastrocytoma in the right fronto-parietal part of the |
| | | brain. The normalization and hybridization conditions were |
| | | adapted from Soares et al., PNAS (1994) 91: 9228, except that |
| | | a significantly longer (48-hour) reannealing hybridization was used. |
| BRAITUT02 | PSPORT1 | Library was constructed using RNA isolated from brain |
| | | tumor tissue removed from the frontal lobe of a 58-year-old |
| | | Caucasian male during excision of a cerebral meningeal |
| | | lesion. Pathology indicated a grade 2 metastatic hypernephroma. |
| | | Patient history included a grade 2 renal cell carcinoma, insomnia, |
| | | and chronic airway obstruction. Family history included a |
| | | malignant neoplasm of the kidney. |
| BRANDIN01 | pINCY | This normalized pineal gland tissue library was constructed |
| | | from .4 million independent clones from a pineal gland tissue |
| | | library from two different donors. Starting RNA was made |
| | | from pooled pineal gland tissue removed from two Caucasian |
| | | females: a 68-year-old (donor A) who died from congestive |
| | | heart failure and a 79-year-old (donor B) who died from |
| | | pneumonia. Neuropathology for donor A indicated mild to |
| | | moderate Alzheimer disease, atherosclerosis, and multiple |
| | | infarctions. Neuropathology for donor B indicated severe |
| | | Alzheimer disease, arteriolosclerosis, cerebral amyloid |
| | | angiopathy and multiple infarctions. There were diffuse and |
| | | neuritic amyloid plaques and neurofibrillary tangles throughout |
| | | the brain sections examined in both donors. Patient history |
| | | included diabetes mellitus, rheumatoid arthritis, |
| | | hyperthyroidism, amyloid heart disease, and dementia |
| | | in donor A; and pseudophakia, gastritis with bleeding, glaucoma, |
| | | peripheral vascular disease, COPD, delayed onset tonic/clonic |
| | | seizures, and transient ischemic attack in donor B. |
| | | The library was normalized in one round using conditions |
| | | adapted from Soares et al., PNAS (1994) 91: 9228-9232 and |
| | | Bonaldo et al., Genome Research 6 (1996): 791, except that |
| | | a significantly longer (48 hours/round) reannealing |
| | | hybridization was used. |
| BRAVUNT02 | PSPORT1 | Library was constructed using pooled RNA isolated |
| | | from separate populations of unstimulated astrocytes. |
| BRSTNOT28 | pINCY | Library was constructed using RNA isolated from |
| | | diseased right breast tissue removed from a 40-year-old Caucasian |
| | | female during a bilateral reduction mammoplasty. Pathology |
| | | indicated bilateral mild fibrocystic and proliferative changes. |
| | | Patient history included pure hypercholesterolemia. Family |
| | | history included acute myocardial infarction, atherosclerotic |
| | | coronary artery disease, type II diabetes, and prostate cancer. |
| BRSTTUT08 | pINCY | Library was constructed using RNA isolated from breast |
| | | tumor tissue removed from a 45-year-old Caucasian female during |
| | | unilateral extended simple mastectomy. Pathology indicated |
| | | invasive nuclear grade 2-3 adenocarcinoma, ductal type, with |
| | | 3 of 23 lymph nodes positive for metastatic disease. Greater |
| | | than 50% of the tumor volume was in situ, both comedo and |
| | | non-comedo types. Immunostains were positive for estrogen/ |
| | | progesterone receptors, and uninvolved tissue showed |
| | | proliferative changes. The patient concurrently underwent a |
| | | total abdominal hysterectomy. Patient history included |
| | | valvuloplasty of mitral valve without replacement, rheumatic |
| | | mitral insufficiency, and rheumatic heart disease. Family |
| | | history included acute myocardial infarction, atherosclerotic |
| | | coronary artery disease, and type II diabetes. |
| BRSTUNF01 | pRARE | This 5′ cap isolated full-length library was constructed |
| | | using RNA isolated from an untreated T47D cell line derived from |
| | | breast tumor tissue removed from a 54-year-old female. |
| COLNPOT01 | pINCY | Library was constructed using RNA isolated from |
| | | colon polyp tissue removed from a 40-year-old Caucasian female during |
| | | a total colectomy. Pathology indicated an inflammatory pseudopolyp; |
| | | this tissue was associated with a focally invasive |
| | | grade 2 adenocarcinoma and multiple tubuvillous adenomas. |
| | | Patient history included a benign neoplasm of the bowel. |
| CONUTUT01 | pINCY | Library was constructed using RNA isolated from sigmoid |
| | | mesentery tumor tissue obtained from a 61-year-old female |
| | | during a total abdominal hysterectomy and bilateral |
| | | salpingo-oophorectomy with regional lymph node excision. Pathology |
| | | indicated a metastatic grade 4 malignant mixed mullerian |
| | | tumor present in the sigmoid mesentery at two sites. |
| DENDNOT01 | pINCY | Library was constructed using RNA isolated from |
| | | untreated dendritic cells from peripheral blood. |
| DRGCNOT01 | pINCY | Library was constructed using RNA isolated from |
| | | dorsal root ganglion tissue removed from the cervical spine of a 32-year- |
| | | old Caucasian male who died from acute pulmonary |
| | | edema and bronchopneumonia, bilateral pleural and pericardial |
| | | effusions, and malignant lymphoma (natural killer cell type). |
| | | Patient history included probable cytomegalovirus infection, |
| | | hepatic congestion and steatosis, splenomegaly, hemorrhagic |
| | | cystitis, thyroid hemorrhage, and Bell's palsy. Surgeries |
| | | included colonoscopy, large intestine biopsy, adenotonsillectomy, |
| | | and nasopharyngeal endoscopy and biopsy; treatment |
| | | included radiation therapy. |
| ENDMUNE01 | pINCY | This 5′ biased random primed library was constructed |
| | | using RNA isolated from untreated umbilical artery endothelial cell |
| | | tissue removed from a Caucasian male (Clonetics) newborn. |
| HNT2AZS07 | PSPORT1 | This subtracted library was constructed from RNA |
| | | isolated from an hNT2 cell line (derived from a human teratocarcinoma |
| | | that exhibited properties characteristic of a committed |
| | | neuronal precursor) treated for three days with 0.35 micromolar AZ. |
| | | The hybridization probe for subtraction was derived from a |
| | | similarly constructed library from untreated hNT2 cells. 3.08M |
| | | clones from the AZ-treated library were subjected to three |
| | | rounds of subtractive hybridization with 3.04M clones from the |
| | | untreated library. Subtractive hybridization conditions were |
| | | based on the methodologies of Swaroop et al. (NAR (1991) |
| | | 19: 1954) and Bonaldo et al. (Genome Research (1996) 6: 791). |
| KIDNTUE01 | PCDNA2.1 | This 5′ biased random primed library was |
| | | constructed using RNA isolated from kidney tumor tissue removed from a 46- |
| | | year-old Caucasian male during nephroureterectomy. |
| | | Pathology indicated grade 2 renal cell carcinoma, clear-cell type, |
| | | forming a mass in the upper pole. The patient presented |
| | | with kidney cancer, backache, headache, malignant hypertension, |
| | | nausea, and vomiting. Previous surgeries included repair |
| | | of indirect inguinal hernia. Patient medications included Lasix, |
| | | Inderal, and Procardia. Family history included cerebrovascular |
| | | accident in the mother; acute myocardial infarction and |
| | | atherosclerotic coronary artery disease in the father; |
| | | and type II diabetes in the sibling(s). |
| LIVRNON08 | pINCY | This normalized library was constructed from 5.7 million |
| | | independent clones from a pooled liver tissue library. Starting |
| | | RNA was made from pooled liver tissue removed from |
| | | a 4-year-old Hispanic male who died from anoxia and a 16 week |
| | | female fetus who died after 16-weeks gestation from |
| | | anencephaly. Serologies were positive for cytolomegalovirus in the 4- |
| | | year-old. Patient history included asthma in the 4-year-old. |
| | | Family history included taking daily prenatal vitamins and |
| | | mitral valve prolapse in the mother of the fetus. The |
| | | library was normalized in 2 rounds using conditions adapted from |
| | | Soares et al., PNAS (1994) 91: 9228 and Bonaldo et al., |
| | | Genome Research 6 (1996): 791, except that a significantly longer |
| | | (48 hours/round) reannealing hybridization was used. |
| LUNGAST01 | PSPORT1 | Library was constructed using RNA isolated |
| | | from the lung tissue of a 17-year-old Caucasian male, who died from head |
| | | trauma. Patient history included asthma. |
| LUNGDIS03 | pINCY | Library was constructed using diseased |
| | | lung tissue. 0.76 million clones from a diseased lung tissue library were subjected |
| | | to two rounds of subtraction hybridization with 5.1 million |
| | | clones from a normal lung tissue library. The starting library for |
| | | subtraction was constructed using polyA RNA isolated |
| | | from diseased lung tissue. Patient history included idiopathic |
| | | pulmonary disease. Subtractive hybridization conditions |
| | | were based on the methodologies of Swaroop et al. (1991) Nucleic |
| | | Acids Res. 19: 1954; and Bonaldo et al. Genome Res. (1996) 6: 791. |
| LUNGNOT04 | PSPORT1 | Library was constructed using RNA isolated |
| | | from the lung tissue of a 2-year-old Hispanic male, who died from cerebral |
| | | anoxia. |
| LUNGNOT09 | pINCY | Library was constructed using RNA isolated from the lung |
| | | tissue of a 23-week-old Caucasian male fetus. The pregnancy |
| | | was terminated following a diagnosis by ultrasound of infantile |
| | | polycystic kidney disease. |
| LUNGNOT10 | pINCY | Library was constructed using RNA isolated from the |
| | | lung tissue of a Caucasian male fetus, who died at 23 weeks' |
| | | gestation. |
| LUNGNOT31 | pINCY | Library was constructed using RNA isolated from right |
| | | middle lobe lung tissue removed from a 63-year-old Caucasian |
| | | male. Pathology for the associated tumor indicated grade |
| | | 3 adenocarcinoma. Patient history included an abdominal aortic |
| | | aneurysm, cardiac dysrhythmia, atherosclerotic coronary artery |
| | | disease, hiatal hernia, chronic sinusitis, and lupus. Family |
| | | history included acute myocardial infarction and atherosclerotic |
| | | coronary artery disease. |
| LUNGNOT38 | pINCY | Library was constructed using RNA isolated from |
| | | diseased lung tissue removed from a 15-year-old Caucasian male who |
| | | died from a gunshot wound to the head. Serology was |
| | | positive for cytomegalovirus. Patient history included asthma. |
| LUNGTUT08 | pINCY | Library was constructed using RNA isolated from |
| | | lung tumor tissue removed from a 63-year-old Caucasian male during a |
| | | right upper lobectomy with fiberoptic bronchoscopy. |
| | | Pathology indicated a grade 3 adenocarcinoma. Patient history |
| | | included atherosclerotic coronary artery disease, an |
| | | acute myocardial infarction, rectal cancer, an asymtomatic abdominal |
| | | aortic aneurysm, tobacco abuse, and cardiac dysrhythmia. |
| | | Family history included congestive heart failure, stomach cancer, |
| | | and lung cancer, type II diabetes, atherosclerotic coronary |
| | | artery disease, and an acute myocardial infarction. |
| MCLRNOC01 | pINCY | This large size-fractionated library was constructed |
| | | using RNA isolated from mononuclear cells obtained from the |
| | | umbilical cord blood of multiple individuals of mixed age |
| | | and sex. The cells were treated with G-CSF. |
| MIXDUNB01 | pINCY | Library was constructed using RNA isolated |
| | | from myometrium removed from a 41-year-old Caucasian female (A) during |
| | | vaginal hysterectomy with a dilatation and curettage and |
| | | untreated smooth muscle cells removed from the renal vein of a 57 |
| | | year-old Caucasian male. Pathology for donor A |
| | | indicated the myometrium and cervix were unremarkable. The |
| | | endometrium was secretory and contained fragments |
| | | of endometrial polyps. Benign endo- and ectocervical mucosa were |
| | | identified in the endocervix. Pathology for the associated |
| | | tumor tissue indicated uterine leiomyoma. Medical history |
| | | included an unspecified menstrual disorder, ventral |
| | | hernia, normal delivery, a benign ovarian neoplasm, and tobacco abuse |
| | | in donor A. Previous surgeries included a bilateral |
| | | destruction of fallopian tubes, removal of a solitary ovary, and an |
| | | exploratory laparotomy in donor A. Medications |
| | | included ferrous sulfate in donor A. |
| NERDTDN03 | pINCY | This normalized dorsal root ganglion tissue library |
| | | was constructed from 1.05 million independent clones from a dorsal |
| | | root ganglion tissue library. Starting RNA was made |
| | | from dorsal root ganglion tissue removed from the cervical spine of a |
| | | 32-year-old Caucasian male who died from acute |
| | | pulmonary edema, acute bronchopneumonia, bilateral pleural effusions, |
| | | pericardial effusion, and malignant lymphoma (natural |
| | | killer cell type). The patient presented with pyrexia of unknown |
| | | origin, malaise, fatigue, and gastrointestinal bleeding. |
| | | Patient history included probable cytomegalovirus infection, liver |
| | | congestion, and steatosis, splenomegaly, hemorrhagic |
| | | cystitis, thyroid hemorrhage, respiratory failure, pneumonia of the |
| | | left lung, natural killer cell lymphoma of the pharynx, |
| | | Bell's palsy, and tobacco and alcohol abuse. Previous surgeries |
| | | included colonoscopy, closed colon biopsy, adenotonsillectomy, |
| | | and nasopharyngeal endoscopy and biopsy. Patient |
| | | medications included Diflucan (fluconazole), Deltasone |
| | | (prednisone), hydrocodone, |
| | | Lortab, Alprazolam, Reazodone, ProMace-Cytabom, |
| | | Etoposide, Cisplatin, Cytarabine, and dexamethasone. The patient |
| | | received radiation therapy and multiple blood transfusions. |
| | | The library was normalized in 2 rounds using conditions |
| | | adapted from Soares et al., PNAS (1994) 91: 9228-9232 |
| | | and Bonaldo et al., Genome Research 6 (1996): 791, except that a |
| | | significantly longer (48 hours/round) reannealing hybridization was used. |
| NEUTFMT01 | PBLUESCRIPT | Library was constructed using total RNA isolated |
| | | from peripheral blood granulocytes collected by density gradient |
| | | centrifugation through Ficoll-Hypaque. The cells were |
| | | isolated from buffy coat units obtained from unrelated male and |
| | | female donors. Cells were cultured in 10 nM fMLP for 30 |
| | | minutes, lysed in GuSCN, and spun through CsCl to obtain RNA |
| | | for library construction. Because this library was made from |
| | | total RNA, it has an unusually high proportion of unique |
| | | singleton sequences, which may not all come from |
| | | polyA RNA species. |
| NOSETUE01 | PCDNA2.1 | This 5′ biased random primed library was constructed |
| | | using RNA isolated from nasal and cribriform tumor tissue removed |
| | | from a 45-year-old Caucasian male during total face ostectomy |
| | | with reconstruction, rhinotomy and craniotomy. Pathology |
| | | indicated olfactory neuroblastoma in the nasal cavity and |
| | | cribriform region. The patient presented with cancer of the head, |
| | | face and neck, and epistaxis. Patient history included extrinsic |
| | | asthma, cancer of the head, face and neck, and epistaxis. |
| | | Previous surgeries included total face ostectomy with |
| | | reconstruction. Patient medications included Biaxin, Atessalon, and |
| | | Valium. The patient received radiation treatments. Family |
| | | history included chronic sinusitis in the mother and type II |
| | | diabetes in the father. |
| OVARDIR01 | PCDNA2.1 | This random primed library was constructed using |
| | | RNA isolated from right ovary tissue removed from a 45-year-old |
| | | Caucasian female during total abdominal hysterectomy, bilateral |
| | | salpingo-oophorectomy, vaginal suspension and fixation, |
| | | and incidental appendectomy. Pathology indicated stromal |
| | | hyperthecosis of the right and left ovaries. Pathology for the |
| | | matched tumor tissue indicated a dermoid cyst (benign cystic |
| | | teratoma) in the left ovary. Multiple (3) intramural |
| | | leiomyomata were identified. The cervix showed squamous |
| | | metaplasia. Patient history included metrorrhagia, female stress |
| | | incontinence, alopecia, depressive disorder, pneumonia, |
| | | normal delivery, and deficiency anemia. Family history included |
| | | benign hypertension, atherosclerotic coronary artery disease, |
| | | hyperlipidemia, and primary tuberculous complex. |
| OVARNOT03 | PSPORT1 | Library was constructed using RNA isolated from |
| | | ovarian tissue removed from a 43-year-old Caucasian female during |
| | | removal of the fallopian tubes and ovaries. Pathology for |
| | | the associated tumor tissue indicated grade 2 mucinous |
| | | cystadenocarcinoma. Patient history included mitral valve |
| | | disorder, pneumonia, and viral hepatitis. Family history included |
| | | atherosclerotic coronary artery disease, pancreatic cancer, |
| | | stress reaction, cerebrovascular disease, breast cancer, and |
| | | uterine cancer. |
| OVARTUT04 | pINCY | Library was constructed using RNA isolated from ovarian |
| | | tumor tissue removed from a 53-year-old Caucasian female |
| | | during a total abdominal hysterectomy, removal of the fallopian |
| | | tubes and ovaries, regional lymph node excision, |
| | | peritoneal tissue destruction, and incidental appendectomy. |
| | | Pathology indicated grade 1 transitional cell carcinoma of the |
| | | right ovary. The left ovary had a hemorrhagic corpus luteum. |
| | | The uterus had multiple leiomyomas (1 submucosal, 11 |
| | | intramural), and the endometrium was inactive. The cul-de-sac |
| | | contained abundant histiocytes and rare clusters of |
| | | mesothelial cells. Patient history included breast fibrosclerosis |
| | | and chronic stomach ulcer. Family history included acute |
| | | stomach ulcer with perforation, breast cancer, bladder cancer, |
| | | rectal/anal cancer, benign hypertension, coronary |
| | | angioplasty, and hyperlipidemia. |
| PROSNOT14 | pINCY | Library was constructed using RNA isolated from diseased |
| | | prostate tissue removed from a 60-year-old Caucasian male |
| | | during radical prostatectomy and regional lymph node excision. |
| | | Pathology indicated adenofibromatous hyperplasia. |
| | | Pathology for the associated tumor tissue indicated an |
| | | adenocarcinoma (Gleason grade 3 + 4). The patient presented with |
| | | elevated prostate specific antigen (PSA). Patient history included |
| | | a kidney cyst and hematuria. Family history included |
| | | benign hypertension, cerebrovascular disease, and |
| | | arteriosclerotic coronary artery disease. |
| SINTNOR01 | PCDNA2.1 | This random primed library was constructed using RNA |
| | | isolated from small intestine tissue removed from a 31-year-old |
| | | Caucasian female during Roux-en-Y gastric bypass. |
| | | Patient history included clinical obesity. |
| TESTNOF01 | PSPORT1 | This 5′ cap isolated full-length library was |
| | | constructed using RNA isolated from testis tissue removed from a 26-year-old |
| | | Caucasian male who died from head trauma due to a motor |
| | | vehicle accident. Serologies were negative. Patient history |
| | | included a hernia at birth, tobacco use (1½ ppd), marijuana |
| | | use, and daily alcohol use (beer and hard liquor). |
| TESTNOT03 | PBLUESCRIPT | Library was constructed using RNA isolated |
| | | from testicular tissue removed from a 37-year-old Caucasian male, who died |
| | | from liver disease. Patient history included cirrhosis, |
| | | jaundice, and liver failure. |
| THP1NOB01 | PBLUESCRIPT | Library was constructed using RNA isolated from |
| | | cultured, unstimulated THP-1 cells. THP-1 is a human promonocyte line |
| | | derived from the peripheral blood of a 1-year-old Caucasian |
| | | male with acute monocytic leukemia (ref: Int. J. Cancer (1980) |
| | | 26: 171). |
| THP1NOT03 | pINCY | Library was constructed using RNA isolated from untreated |
| | | THP-1 cells. THP-1 is a human promonocyte line derived from |
| | | the peripheral blood of a 1-year-old Caucasian male with |
| | | acute monocytic leukemia (ref: Int. J. Cancer (1980) 26: 171). |
| THYRNOT03 | pINCY | Library was constructed using RNA isolated from thyroid |
| | | tissue removed from the left thyroid of a 28-year-old Caucasian |
| | | female during a complete thyroidectomy. Pathology indicated |
| | | a small nodule of adenomatous hyperplasia present in the left |
| | | thyroid. Pathology for the associated tumor tissue indicated |
| | | dominant follicular adenoma, forming a well-encapsulated |
| | | mass in the left thyroid. |
| TONSDIE01 | PCDNA2.1 | This 5′ biased random primed library was constructed |
| | | using RNA isolated from diseased left tonsil tissue removed from a 6 |
| | | year-old Caucasian male during adenotonsillectomy. Pathology |
| | | indicated reactive lymphoid hyperplasia, bilaterally. The |
| | | patient presented with sleep apnea. Patient history included |
| | | a bacterial infection. Previous surgeries included myringotomy |
| | | with tube insertion. The patient was not taking any medications. |
| | | Family history included benign hypertension, myocardial |
| | | infarction, and atherosclerotic coronary artery disease in the grandparent(s). |

TABLE 7

| Program | Description | Reference | Parameter Threshold |
|---|---|---|---|
| ABI FACTURA | A program that removes vector sequences and masks | Applied Biosystems, Foster City, CA. | |
| | ambiguous bases in nucleic acid sequences. | | |
| ABI/PARACEL | A Fast Data Finder useful in comparing and | Applied Biosystems, Foster City, CA; | Mismatch <50% |
| FDF | annotating amino acid or nucleic acid sequences. | Paracel Inc., Pasadena, CA. | |
| ABI | A program that assembles nucleic acid sequences. | Applied Biosystems, Foster City, CA. | |
| AutoAssembler | | | |
| BLAST | A Basic Local Alignment Search Tool useful in | Altschul, S. F. et al. (1990) J. Mol. Biol. | ESTs: Probability |
| | sequence similarity search for amino acid and nucleic | 215: 403-410; Altschul, S. F. et al. (1997) | value = 1.0E−8 |
| | acid sequences. BLAST includes five functions: | Nucleic Acids Res. 25: 3389-3402. | or less; Full Length |
| | blastp, blastn, blastx, tblastn, and tblastx. | | sequences: |
| | | | Probability |
| | | | value = 1.0E−10 |
| | | | or less |
| FASTA | A Pearson and Lipman algorithm that searches for | Pearson, W. R. and D. J. Lipman (1988) Proc. | ESTs: fasta E |
| | similarity between a query sequence and a group of | Natl. Acad Sci. USA 85: 2444-2448; Pearson, W. R. | value = 1.06E−6; |
| | sequences of the same type. FASTA comprises as | (1990) Methods Enzymol. 183: 63-98; | Assembled ESTs: |
| | least five functions: fasta, tfasta, fastx, tfastx, and | and Smith, T. F. and M. S. Waterman (1981) | fasta Identity = |
| | ssearch. | Adv. Appl. Math. 2: 482-489. | 95% or greater and |
| | | | Match length = |
| | | | 200 bases or |
| | | | greater; fastx |
| | | | E value = 1.0E−8 |
| | | | or less; Full Length |
| | | | sequences: fastx |
| | | | score = 100 or |
| | | | greater |
| BLIMPS | A BLocks IMProved Searcher that matches a | Henikoff, S. and J. G. Henikoff (1991) | Probability value = |
| | sequence against those in BLOCKS, PRINTS, | Nucleic Acids Res. 19: 6565-6572; Henikoff, J. G. | 1.0E−3 or less |
| | DOMO, PRODOM, and PFAM databases to search | and S. Henikoff (1996) Methods | |
| | for gene families, sequence homology, and structural | Enzymol. 266: 88-105; and Attwood, T. K. et | |
| | fingerprint regions. | al. (1997) J. Chem. Inf. Comput. Sci. 37: 417-424. | |
| HMMER | An algorithm for searching a query sequence against | Krogh, A. et al. (1994) J. Mol. Biol. | PFAM, INCY, |
| | hidden Markov model (HMM)-based databases of | 235: 1501-1531; Sonnhammer, E. L. L. et al. | SMART or |
| | protein family consensus sequences, such as PFAM, | (1988) Nucleic Acids Res. 26: 320-322; | TIGRFAM hits: |
| | INCY, SMART and TIGRFAM. | Durbin, R. et al. (1998) Our World View, in | Probability value = |
| | | a Nutshell, Cambridge Univ. Press, pp. 1-350. | 1.0E−3 or less; |
| | | | Signal peptide |
| | | | hits: Score = |
| | | | 0 or greater |
| ProfileScan | An algorithm that searches for structural and | Gribskov, M. et al. (1988) CABIOS 4: 61-66; | Normalized quality |
| | sequence motifs in protein sequences that match | Gribskov, M. et al. (1989) Methods | score ≧ GCG |
| | sequence patterns defined in Prosite. | Enzymol. 183: 146-159; Bairoch, A. et al. | specified “HIGH” |
| | | (1997) Nucleic Acids Res. 25: 217-221. | value for that |
| | | | particular Prosite |
| | | | motif. Generally, |
| | | | score = 1.4-2.1. |
| Phred | A base-calling algorithm that examines automated | Ewing, B. et al. (1998) Genome Res. 8: 175-185; | |
| | sequencer traces with high sensitivity and probability. | Ewing, B. and P. Green (1998) Genome | |
| | | Res. 8: 186-194. | |
| Phrap | A Phils Revised Assembly Program including | Smith, T. F. and M. S. Waterman (1981) Adv. | Score = 120 or |
| | SWAT and CrossMatch, programs based on efficient | Appl. Math. 2: 482-489; Smith, T. F. and | greater; Match |
| | implementation of the Smith-Waterman algorithm, | M. S. Waterman (1981) J. Mol. Biol. 147: 195-197; | length = 56 or |
| | useful in searching sequence homology and | and Green, P., University of | greater |
| | assembling DNA sequences. | Washington, Seattle, WA. | |
| Consed | A graphical tool for viewing and editing Phrap | Gordon, D. et al. (1998) Genome Res. 8: 195-202. | |
| | assemblies. | | |
| SPScan | A weight matrix analysis program that scans protein | Nielson, H. et al. (1997) Protein Engineering | Score = 3.5 or |
| | sequences for the presence of secretory signal | 10: 1-6; Claverie, J. M. and S. Audic (1997) | greater |
| | peptides. | CABIOS 12: 431-439. | |
| TMAP | A program that uses weight matrices to delineate | Persson, B. and P. Argos (1994) J. Mol. Biol. | |
| | transmembrane segments on protein sequences and | 237: 182-192; Persson, B. and P. Argos | |
| | determine orientation. | (1996) Protein Sci. 5: 363-371. | |
| TMHMMER | A program that uses a hidden Markov model (HMM) | Sonnhammer, E. L. et al. (1998) Proc. Sixth | |
| | to delineate transmembrane segments on protein | Intl. Conf. On Intelligent Systems for Mol. | |
| | sequences and determine orientation. | Biol., Glasgow et al., eds., The Am. Assoc. | |
| | | for Artificial Intelligence (AAAI) Press, | |
| | | Menlo Park, CA, and MIT Press, Cambridge, | |
| | | MA, pp. 175-182. | |
| Motifs | A program that searches amino acid sequences for | Bairoch, A. et al. (1997) Nucleic Acids Res. | |
| | patterns that matched those defined in Prosite. | 25: 217-221; Wisconsin Package Program | |
| | | Manual, version 9, page M51-59, Genetics | |
| | | Computer Group, Madison, WI. | |

1. An isolated polypeptide selected from the group consisting of:
a) a polypeptide comprising an amino acid sequence selected from the group consisting of SEQ ID NO:1-48,
b) a polypeptide comprising a naturally occurring amino acid sequence at least 90% identical to an amino acid sequence selected from the group consisting of SEQ ID NO:2, SEQ ID NO:4-6, SEQ ID NO:8-9, SEQ ID NO:11, SEQ ID NO:13-22, SEQ ID NO:24-27, SEQ ID NO:29-33, SEQ ID NO:35-36, SEQ ID NO:39, SEQ ID NO:41-43, and SEQ ID NO:46-48,
c) a polypeptide comprising a naturally occurring amino acid sequence at least 95% identical to an amino acid sequence selected from the group consisting of SEQ ID NO:1 and SEQ ID NO:7,
d) a polypeptide comprising a naturally occurring amino acid sequence at least 99% identical to an amino acid sequence selected from the group consisting of SEQ ID NO:3 and SEQ ID NO:23,
e) a polypeptide comprising a naturally occurring amino acid sequence at least 91% identical to an amino acid sequence selected from the group consisting of SEQ ID NO:10 and SEQ ID NO:34,
f) a polypeptide comprising a naturally occurring amino acid sequence at least 98% identical to the amino acid sequence of SEQ ID NO:12,
g) a polypeptide comprising a naturally occurring amino acid sequence at least 93% identical to the amino acid sequence of SEQ ID NO:37,
h) a polypeptide comprising a naturally occurring amino acid sequence at least 97% identical to an amino acid sequence selected from the group consisting of SEQ ID NO:44,
i) a polypeptide consisting essentially of a naturally occurring amino acid sequence at least 90% identical to an amino acid sequence selected from the group consisting of SEQ ID NO:38 and SEQ ID NO:40,
j) a biologically active fragment of a polypeptide having an amino acid sequence selected from the group consisting of SEQ ID NO:1-48, and
k) an immunogenic fragment of a polypeptide having an amino acid sequence selected from the group consisting of SEQ ID NO:1-48.

2. An isolated polypeptide of claim 1 comprising an amino acid sequence selected from the group consisting of SEQ ID NO:1-48.

3. An isolated polynucleotide encoding a polypeptide of claim 1.

4. An isolated polynucleotide encoding a polypeptide of claim 2.

5. An isolated polynucleotide of claim 4 comprising a polynucleotide sequence selected from the group consisting of SEQ ID NO:49-96.

6. A recombinant polynucleotide comprising a promoter sequence operably linked to a polynucleotide of claim 3.

7. A cell transformed with a recombinant polynucleotide of claim 6.

8. (canceled)

9. A method of producing a polypeptide of claim 1, the method comprising:
a) culturing a cell under conditions suitable for expression of the polypeptide, wherein said cell is transformed with a recombinant polynucleotide, and said recombinant polynucleotide comprises a promoter sequence operably linked to a polynucleotide encoding the polypeptide of claim 1, and
b) recovering the polypeptide so expressed.

10. A method of claim 9, wherein the polypeptide comprises an amino acid sequence selected from the group consisting of SEQ ID NO:1-48.

11. An isolated antibody which specifically binds to a polypeptide of claim 1.

12. An isolated polynucleotide selected from the group consisting of:
a) a polynucleotide comprising a polynucleotide sequence selected from the group consisting of SEQ ID NO:49-96,
b) a polynucleotide comprising a naturally occurring polynucleotide sequence at least 90% identical to a polynucleotide sequence selected from the group consisting of SEQ ID NO:49-50, SEQ ID NO:52-54, SEQ ID NO:56-70, SEQ ID NO:73-88, SEQ ID NO:90-92, and SEQ ID NO:94-96,
c) a polynucleotide comprising a naturally occurring polynucleotide sequence at least 91% identical to the polynucleotide sequence of SEQ ID NO:51,
d) a polynucleotide comprising a naturally occurring polynucleotide sequence at least 94% identical to the polynucleotide sequence of SEQ ID NO:55,
e) a polynucleotide comprising a naturally occurring polynucleotide sequence at least 95% identical to the polynucleotide sequence of SEQ ID NO:71,
f) a polynucleotide comprising a naturally occurring polynucleotide sequence at least 96% identical to the polynucleotide sequence of SEQ ID NO:72,
g) a polynucleotide comprising a naturally occurring polynucleotide sequence at least 97% identical to the polynucleotide sequence of SEQ ID NO: 89,
h) a polynucleotide comprising a naturally occurring polynucleotide sequence at least 99% identical to the polynucleotide sequence of SEQ ID NO:93,
i) a polynucleotide complementary to a polynucleotide of a),
j) a polynucleotide complementary to a polynucleotide of b),
k) a polynucleotide complementary to a polynucleotide of c),
l) a polynucleotide complementary to a polynucleotide of d),
m) a polynucleotide complementary to a polynucleotide of e),
n) a polynucleotide complementary to a polynucleotide of f),
o) a polynucleotide complementary to a polynucleotide of g),
p) a polynucleotide complementary to a polynucleotide of h), and
q) an RNA equivalent of a)-p).

13. (canceled)

14. A method of detecting a target polynucleotide in a sample, said target polynucleotide having a sequence of a polynucleotide of claim 12, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

15. (canceled)

16. A method of detecting a target polynucleotide in a sample, said target polynucleotide having a sequence of a polynucleotide of claim 12, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

17. A composition comprising a polypeptide of claim 1 and a pharmaceutically acceptable excipient.

18. A composition of claim 17, wherein the polypeptide comprises an amino acid sequence selected from the group consisting of SEQ ID NO:1-48.

19. (canceled)

20. A method of screening a compound for effectiveness as an agonist of a polypeptide of claim 1, the method comprising:
a) exposing a sample comprising a polypeptide of claim 1 to a compound, and
b) detecting agonist activity in the sample.

21-22. (canceled)

23. A method of screening a compound for effectiveness as an antagonist of a polypeptide of claim 1, the method comprising:
a) exposing a sample comprising a polypeptide of claim 1 to a compound, and
b) detecting antagonist activity in the sample.

24-25. (canceled)

26. A method of screening for a compound that specifically binds to the polypeptide of claim 1, the method comprising:
a) combining the polypeptide of claim 1 with at least one test compound under suitable conditions, and
b) detecting binding of the polypeptide of claim 1 to the test compound, thereby identifying a compound that specifically binds to the polypeptide of claim 1.

27. (canceled)

28. A method of screening a compound for effectiveness in altering expression of a target polynucleotide, wherein said target polynucleotide comprises a sequence of claim 5, the method comprising:
a) exposing a sample comprising the target polynucleotide to a compound, under conditions suitable for the expression of the target polynucleotide,
b) detecting altered expression of the target polynucleotide, and
c) comparing the expression of the target polynucleotide in the presence of varying amounts of the compound and in the absence of the compound.

29. A method of assessing toxicity of a test compound, the method comprising:
a) treating a biological sample containing nucleic acids with the test compound,
b) hybridizing the nucleic acids of the treated biological sample with a probe comprising at least 20 contiguous nucleotides of a polynucleotide of claim 12 under conditions whereby a specific hybridization complex is formed between said probe and a target polynucleotide in the biological sample, said target polynucleotide comprising a polynucleotide sequence of a polynucleotide of claim 12 or fragment thereof,
c) quantifying the amount of hybridization complex, and
d) comparing the amount of hybridization complex in the treated biological sample with the amount of hybridization complex in an untreated biological sample, wherein a difference in the amount of hybridization complex in the treated biological sample is indicative of toxicity of the test compound.

30-151. (canceled)
